(19) United States
(12) Patent Application Publication
(10) Pub. No.: US 2019/0198780 A1
(43) Pub. Date: Jun. 27, 2019

(54) ORGANIC COMPOUND AND COMPOSITION AND ORGANIC OPTOELECTRONIC DEVICE AND DISPLAY DEVICE

(71) Applicant: SAMSUNG SDI CO., LTD., Yongin-si (KR)

(72) Inventors: Byungku KIM, Suwon-si (KR); Giwook KANG, Suwon-si (KR); Sunha PARK, Suwon-si (KR); Yongtak YANG, Suwon-si (KR); Hanill LEE, Suwon-si (KR); Kipo JANG, Suwon-si (KR); Sung-Hyun JUNG, JR., Suwon-si (KR); Ho Kuk JUNG, Suwon-si (KR)

(21) Appl. No.: 16/232,304

(22) Filed: Dec. 26, 2018

(30) Foreign Application Priority Data
Dec. 27, 2017 (KR) 10-2017-0181464

(51) Int. Cl. H01L 51/00; C07D 405/14; C07D 209/86; C07D 409/14; C07D 487/04

(52) U.S. Cl. CPC H01L 51/0073; C07D 405/14; C07D 209/86; H01L 51/0067; H01L 51/5016; H01L 51/0074; H01L 51/0072; C07D 487/04; C07D 409/14

(57) ABSTRACT

Disclosed are an organic compound represented by Chemical Formula 1, a composition including the same, an organic optoelectronic device, and a display device. In Chemical Formula 1, X¹ to X³, Y¹ to Y³, and R¹ to R¹⁸ are the same as described in the detailed description.

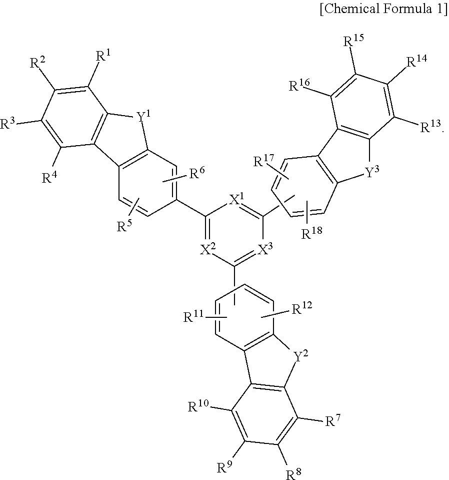

### CROSS-REFERENCE TO RELATED APPLICATION

Korean Patent Application No. 10-2017-0181464, filed on Dec. 27, 2017, in the Korean Intellectual Property Office, and entitled: “Organic Compound and Composition and Organic Optoelectronic Device and Display Device,” is incorporated by reference herein in its entirety.

### BACKGROUND

### 1. Field

An organic compound, a composition, an organic optoelectronic device, and a display device are disclosed.

### 2. Description of the Related Art

An organic optoelectronic device is a device that converts electrical energy into photoenergy, and vice versa.

An organic optoelectronic device may be classified as follows in accordance with its driving principles. One is a photoelectric device where excitons are generated by photoenergy, separated into electrons and holes, and are transferred to different electrodes to generate electrical energy, and the other is a light emitting device where a voltage or a current is supplied to an electrode to generate photoenergy from electrical energy.

Examples of the organic optoelectronic device may be an organic photoelectric device, an organic light emitting diode, an organic solar cell, and an organic photo conductor drum.

Of these, an organic light emitting diode (OLED) has recently drawn attention due to an increase in demand for flat panel displays. The organic light emitting diode converts electrical energy into light by applying current to an organic light emitting material.

### SUMMARY

Embodiments are directed to an organic compound represented by Chemical Formula 1:

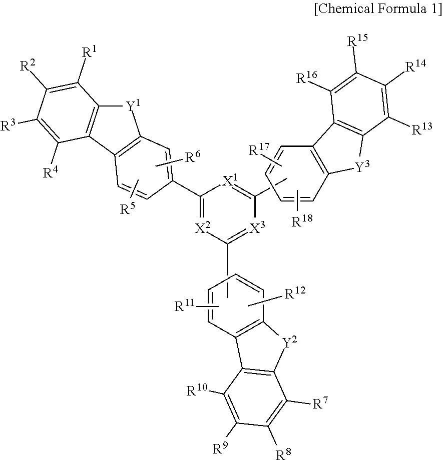

In Chemical Formula 1,

X¹ to X³ may independently N or CR^{a},

at least two of X¹ to X³ may be N,

Y¹ to Y³ may independently be O or S,

R¹ to R¹⁸ and R^{a} may independently be hydrogen, deuterium, a substituted or unsubstituted C1 to C20 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof, and

R¹ to R¹⁸ may independently be present or adjacent groups of R¹ to R¹⁸ are linked with each other to form a ring.

Embodiments are also directed to a composition that includes the organic compound as a first organic compound, and includes a second organic compound including a carbazole moiety represented by Chemical Formula 7.

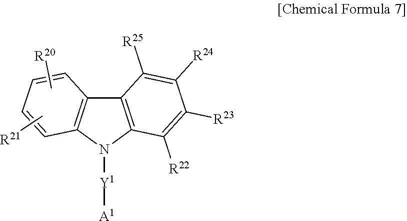

In Chemical Formula 7,

Y¹ may be a single bond, a substituted or unsubstituted C6 to C30 arylene group, or a divalent substituted or unsubstituted C2 to C30 heterocyclic group,

A¹ may be a substituted or unsubstituted C6 to C30 aryl group, or a substituted or unsubstituted C2 to C30 heterocyclic group,

R²⁰ to R²⁵ may independently be hydrogen, deuterium, a substituted or unsubstituted C1 to C20 alkyl group, a substituted or unsubstituted C6 to C30 aryl group, or a substituted or unsubstituted C2 to C30 heterocyclic group, and

R²² to R²⁵ may independently be present or adjacent groups of R²² to R²⁵ may be linked with each other to form a ring.

Embodiments are also directed to an organic optoelectronic device that includes an anode and a cathode facing each other, and an organic layer between the anode and the cathode, wherein the organic layer includes the organic compound or the composition.

Embodiments are also directed to a display device that includes the organic optoelectronic device.

### BRIEF DESCRIPTION OF THE DRAWINGS

Features will become apparent to those of skill in the art by describing in detail example embodiments with reference to the attached drawings in which:

FIGS. 1 and 2 illustrate cross-sectional views showing organic light emitting diodes according to embodiments.

### DETAILED DESCRIPTION

Example embodiments will now be described more fully hereinafter with reference to the accompanying drawings; however, they may be embodied in different forms and should not be construed as limited to the embodiments set forth herein. Rather, these embodiments are provided so that this disclosure will be thorough and complete, and will fully convey example implementations to those skilled in the art. In the drawing figures, the dimensions of layers and regions may be exaggerated for clarity of illustration. Like reference numerals refer to like elements throughout.

As used herein, when a definition is not otherwise provided, the “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heterocyclic group, a C1 to C20 alkoxy group, a C1 to C10 trifluoroalkyl group, a cyano group, or a combination thereof.

In one example of the present disclosure, the “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, or a C2 to C30 heterocyclic group. In addition, in specific examples of the present disclosure, the “substituted” refers to replacement of at least one hydrogen of a substituent or a compound by deuterium, a C1 to C20 alkyl group, a C6 to C30 aryl group, or a C2 to C30 heterocyclic group. In addition, in specific examples of the present disclosure, the “substituted” refers to replacement of at least one hydrogen of a substituent or a compound by deuterium, a C1 to C5 alkyl group, a C6 to C18 aryl group, a pyridinyl group, a quinolinyl group, an isoquinolinyl group, a dibenzofuranyl group, a dibenzothiophenyl group, or a carbazolyl group. In addition, in specific examples of the present disclosure, the “substituted” refers to replacement of at least one hydrogen of a substituent or a compound by deuterium, a C1 to C5 alkyl group, a C6 to C18 aryl group, a dibenzofuranyl group, or a dibenzothiophenyl group. In addition, in specific examples of the present disclosure, the “substituted” refers to replacement of at least one hydrogen of a substituent or a compound by deuterium, a methyl group, an ethyl group, a propyl group, a butyl group, a phenyl group, a biphenyl group, a terphenyl group, a naphthyl group, a triphenyl group, a dibenzofuranyl group, or a dibenzothiophenyl group.

As used herein, when a definition is not otherwise provided, “hetero” refers to one including one to three heteroatoms selected from N, O, S, P, and Si, and remaining carbons in one functional group.

As used herein, the “aryl group” refers to a group including at least one hydrocarbon aromatic moiety, and all the elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quarterphenyl group, and the like, and two or more hydrocarbon aromatic moieties are fused directly or indirectly to provide a non-aromatic fused ring, for example a fluorenyl group.

The aryl group may include a monocyclic, polycyclic or fused ring polycyclic (i.e., rings sharing adjacent pairs of carbon atoms) functional group.

As used herein, the “heterocyclic group” is a generic concept including a heteroaryl group, and may include at least one hetero atom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, the “heteroaryl group” may refer to an aryl group including at least one hetero atom selected from N, O, S, P. and Si. Two or more heteroaryl groups are linked by a sigma bond directly, or when the C2 to C60 heteroaryl group includes two or more rings, the two or more rings may be fused. When the heteroaryl group is a fused ring, each ring may include 1 to 3 hetero atoms.

Specific examples of the heterocyclic group may be a pyridinyl group, a pyrimidinyl group, a pyrazinyl group, a pyridazinyl group, a triazinyl group, a quinolinyl group, an isoquinolinyl group, and the like.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but are not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted dibenzofuranyl group, or a substituted or unsubstituted dibenzothiophenyl group, or a combination thereof, but are not limited thereto.

In the present specification, hole characteristics refer to an ability to donate an electron to form a hole when an electric field is applied, and that a hole formed in the anode may be easily injected into a light emitting layer, and a hole formed in a light emitting layer may be easily transported into an anode and transported in the light emitting layer due to conductive characteristics according to a highest occupied molecular orbital (HOMO) level.

In addition, electron characteristics refer to an ability to accept an electron when an electric field is applied, and that an electron formed in a cathode may be easily injected into a light emitting layer, and an electron formed in a light emitting layer may be easily transported into a cathode and transported in the light emitting layer due to conductive characteristics according to a lowest unoccupied molecular orbital (LUMO) level.

Hereinafter, an organic compound according to an embodiment is described.

An organic compound according to an embodiment is represented by Chemical Formula 1.

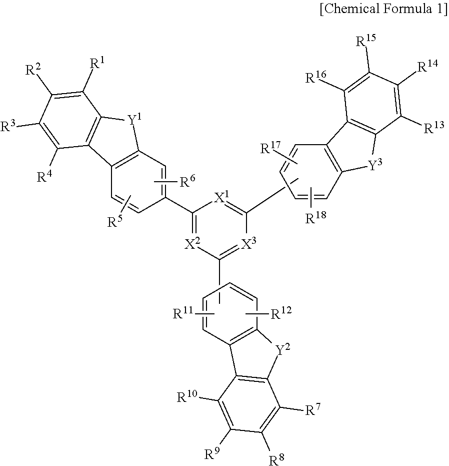

In Chemical Formula 1,

X¹ to X³ may independently be N or CR^{a},

at least two of X¹ to X³ may be N,

Y¹ to Y³ may independently be O or S,

R¹ to R¹⁸ and R^{a} may independently be hydrogen, deuterium, a substituted or unsubstituted C1 to C20 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof, and

R¹ to R¹⁸ may independently be present, or adjacent groups of R¹ to R¹⁸ may be linked with each other to form a ring.

The organic compound represented by Chemical Formula 1 has a pyrimidine or triazine ring and thus may show high electron transport characteristics. In addition, the organic compound represented by Chemical Formula 1 has a structure of being directly substituted with three dibenzofuranyl groups and/or dibenzothiophenyl groups in the pyrimidine or triazine ring, and thus may show much higher electron transport characteristics. Accordingly, when the organic compound is applied to a device, the device may have a low driving voltage and high efficiency. Herein, as shown in Chemical Formula 1, a dibenzofuranyl group or a dibenzothiophenyl group may be combined at the position No. 3 thereof with the pyrimidine or triazine ring, and accordingly, the organic compound may have fast electron mobility. Accordingly, the organic compound may contribute to significant improvement of a life-span as well as lowering a driving voltage.

In addition, the organic compound represented by Chemical Formula 1 may have a relatively high glass transition temperature. Accordingly, the organic compound represented by Chemical Formula 1 may resist degradation during a manufacturing process for a device and/or driving of the device, and thus may provide enhanced thermal stability and improved life-span for the device. For example, the organic compound may have a glass transition temperature of about 50° C. to about 300° C.

In example embodiments, X¹ to X³ may each be N.

In example embodiments, two of X¹ to X³ may be N and one may be CH.

In example embodiments, Y¹ to Y³ may each be O.

In example embodiments, Y¹ to Y³ may each be S.

In example embodiments, two of Y¹ to Y³ may be S and one of Y¹ to Y³ may be 0.

In example embodiments, two of Y¹ to Y³ may be 0 and one of Y¹ to Y³ may be S.

In example embodiments, R¹ to R¹⁸ and R^{a} may independently be hydrogen, deuterium, a substituted or unsubstituted C1 to C20 alkyl group, a substituted or unsubstituted C6 to C30 aryl group, or a combination thereof. For example R¹ to R¹⁸ and R^{a} may independently be hydrogen or a substituted or unsubstituted C6 to C30 aryl group. For example R¹ to R¹⁸ and R^{a} may each be hydrogen.

The organic compound may be for example represented by Chemical Formula 2 or 3.

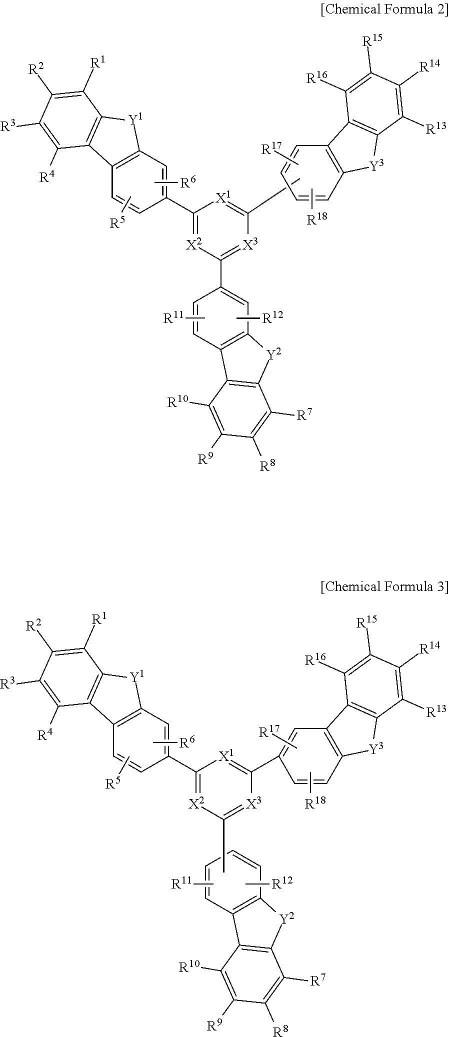

In Chemical Formula 2 or 3, X¹ to X³, Y¹ to Y³ and R¹ to R¹⁸ are the same as described above.

The organic compound may be for example represented by one of Chemical Formulae 4 to 6.

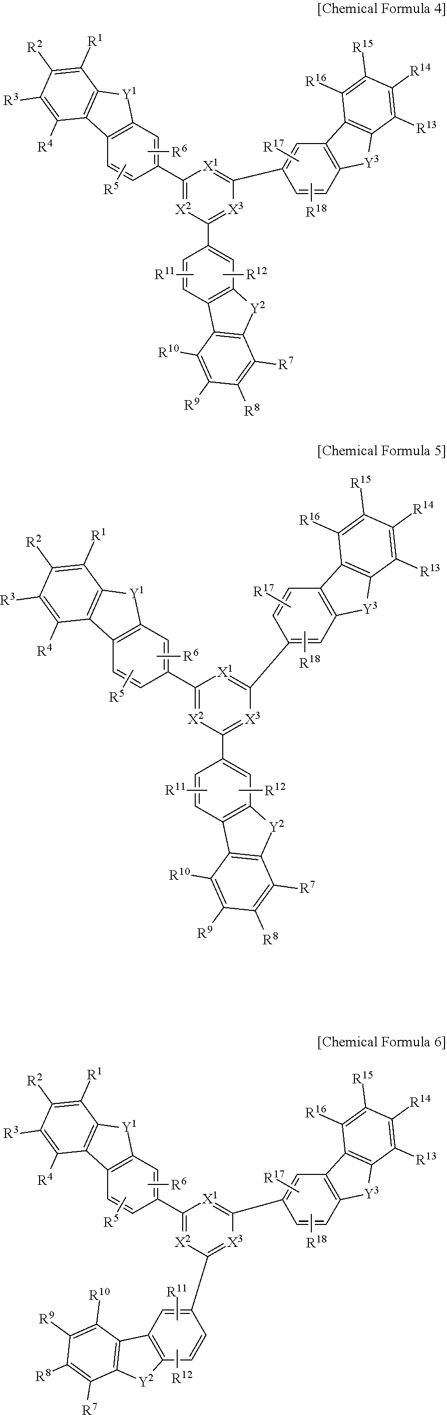

In Chemical Formulae 4 to 6, X¹ to X³, Y¹ to Y³ and R¹ to R¹⁸ are the same as described above.

The organic compound may be for example selected from compounds listed in Group 1.

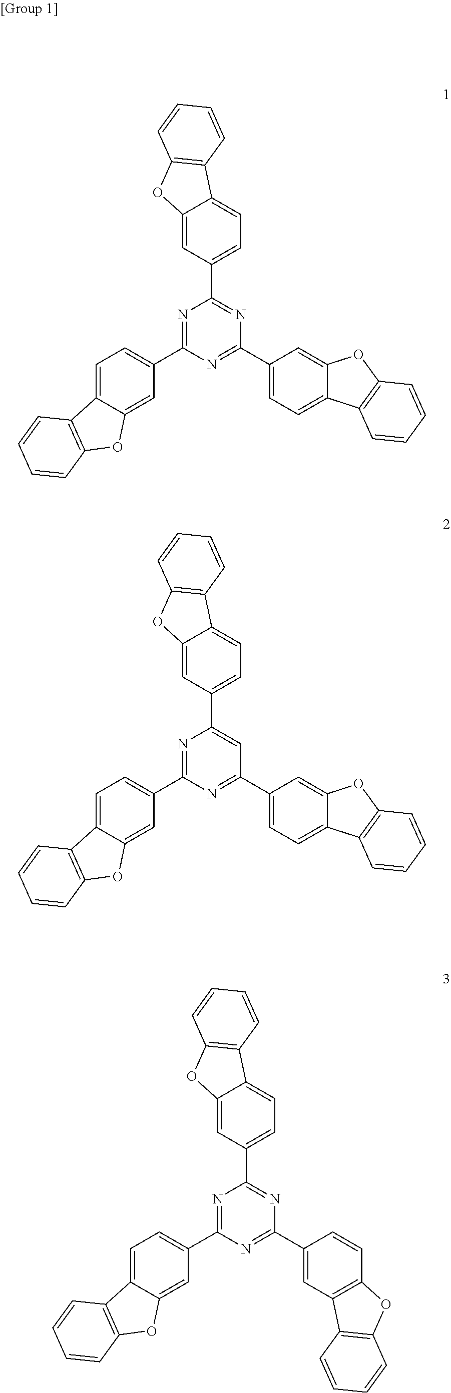 [SMILES: c1ccc2c(c1)oc1cc(-c3nc(-c4ccc5c(c4)oc4ccccc45)nc(-c4ccc5c(c4)oc4ccccc45)n3)ccc12 ; c1ccc2c(c1)oc1cc(-c3cc(-c4ccc5c(c4)oc4ccccc45)nc(-c4ccc5c(c4)oc4ccccc45)n3)ccc12 ; c1ccc2c(c1)oc1cc(-c3nc(-c4ccc5c(c4)oc4ccccc45)nc(-c4ccc5oc6ccccc6c5c4)n3)ccc12]

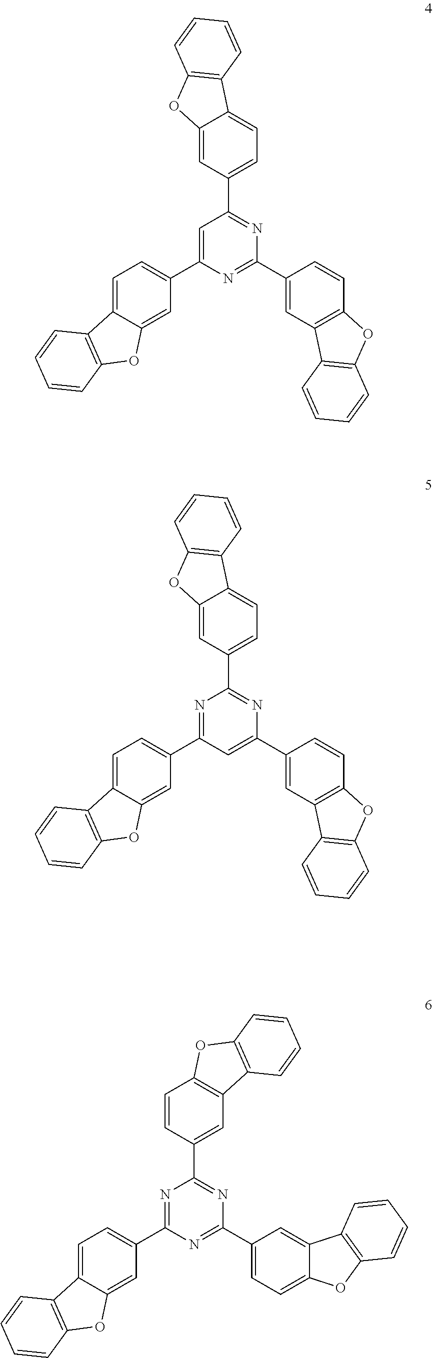 [SMILES: c1ccc2c(c1)oc1cc(-c3cc(-c4ccc5c(c4)oc4ccccc45)nc(-c4ccc5oc6ccccc6c5c4)n3)ccc12 ; c1ccc2c(c1)oc1cc(-c3cc(-c4ccc5oc6ccccc6c5c4)nc(-c4ccc5c(c4)oc4ccccc45)n3)ccc12 ; c1ccc2c(c1)oc1cc(-c3nc(-c4ccc5oc6ccccc6c5c4)nc(-c4ccc5oc6ccccc6c5c4)n3)ccc12]

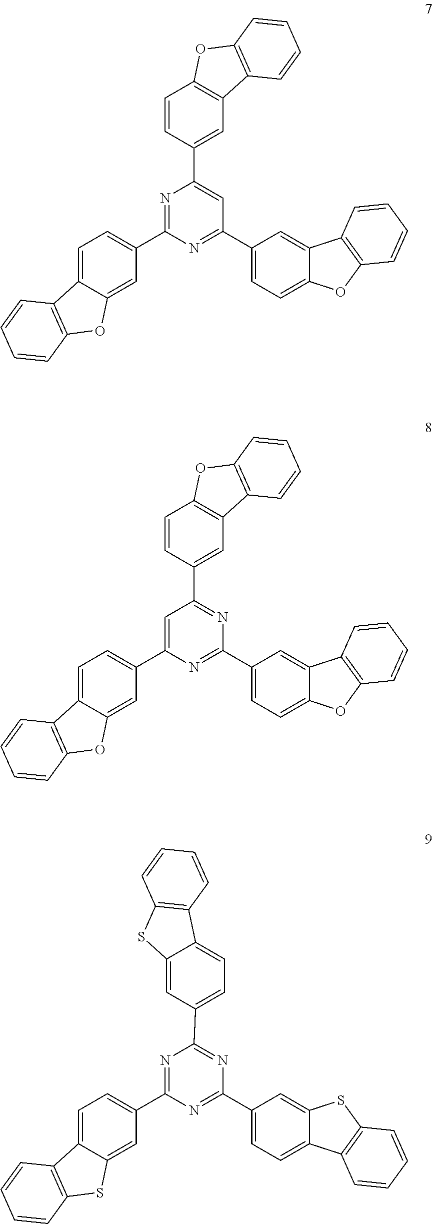 [SMILES: c1ccc2c(c1)oc1cc(-c3nc(-c4ccc5oc6ccccc6c5c4)cc(-c4ccc5oc6ccccc6c5c4)n3)ccc12 ; c1ccc2c(c1)oc1cc(-c3cc(-c4ccc5oc6ccccc6c5c4)nc(-c4ccc5oc6ccccc6c5c4)n3)ccc12 ; c1ccc2c(c1)sc1cc(-c3nc(-c4ccc5c(c4)sc4ccccc45)nc(-c4ccc5c(c4)sc4ccccc45)n3)ccc12]

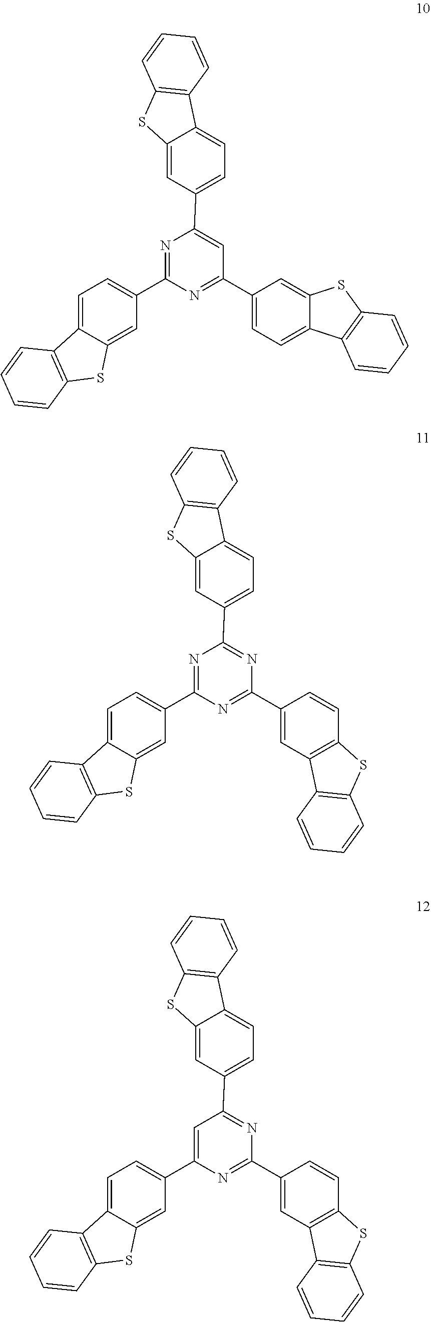 [SMILES: c1ccc2c(c1)sc1cc(-c3cc(-c4ccc5c(c4)sc4ccccc45)nc(-c4ccc5c(c4)sc4ccccc45)n3)ccc12 ; c1ccc2c(c1)sc1cc(-c3nc(-c4ccc5c(c4)sc4ccccc45)nc(-c4ccc5sc6ccccc6c5c4)n3)ccc12 ; c1ccc2c(c1)sc1cc(-c3cc(-c4ccc5c(c4)sc4ccccc45)nc(-c4ccc5sc6ccccc6c5c4)n3)ccc12]

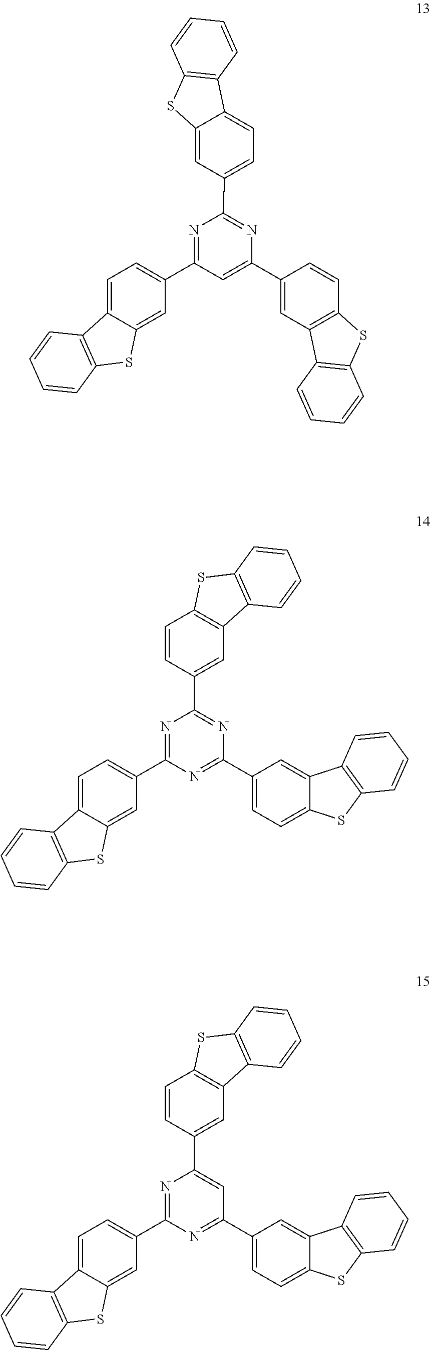 [SMILES: c1ccc2c(c1)sc1cc(-c3cc(-c4ccc5sc6ccccc6c5c4)nc(-c4ccc5c(c4)sc4ccccc45)n3)ccc12 ; c1ccc2c(c1)sc1cc(-c3nc(-c4ccc5sc6ccccc6c5c4)nc(-c4ccc5sc6ccccc6c5c4)n3)ccc12 ; c1ccc2c(c1)sc1cc(-c3nc(-c4ccc5sc6ccccc6c5c4)cc(-c4ccc5sc6ccccc6c5c4)n3)ccc12]

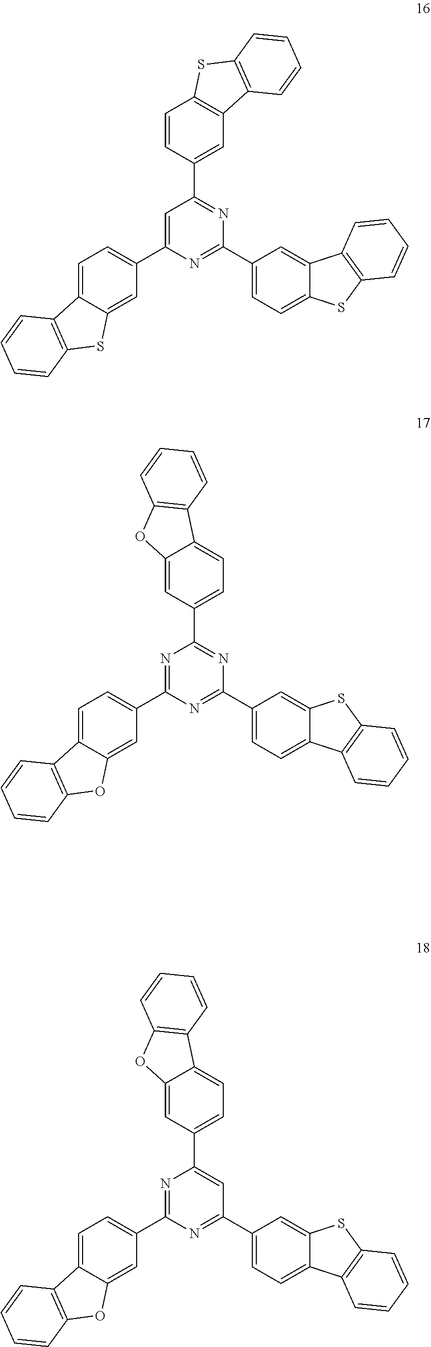 [SMILES: c1ccc2c(c1)sc1cc(-c3cc(-c4ccc5sc6ccccc6c5c4)nc(-c4ccc5sc6ccccc6c5c4)n3)ccc12 ; c1ccc2c(c1)oc1cc(-c3nc(-c4ccc5c(c4)oc4ccccc45)nc(-c4ccc5c(c4)sc4ccccc45)n3)ccc12 ; c1ccc2c(c1)oc1cc(-c3cc(-c4ccc5c(c4)sc4ccccc45)nc(-c4ccc5c(c4)oc4ccccc45)n3)ccc12]

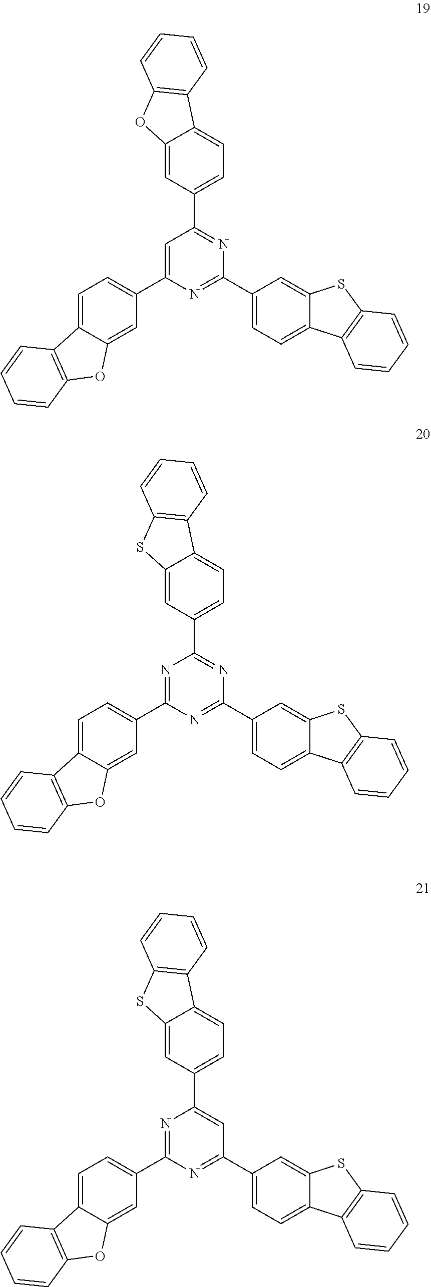 [SMILES: c1ccc2c(c1)oc1cc(-c3cc(-c4ccc5c(c4)oc4ccccc45)nc(-c4ccc5c(c4)sc4ccccc45)n3)ccc12 ; c1ccc2c(c1)oc1cc(-c3nc(-c4ccc5c(c4)sc4ccccc45)nc(-c4ccc5c(c4)sc4ccccc45)n3)ccc12 ; c1ccc2c(c1)oc1cc(-c3nc(-c4ccc5c(c4)sc4ccccc45)cc(-c4ccc5c(c4)sc4ccccc45)n3)ccc12]

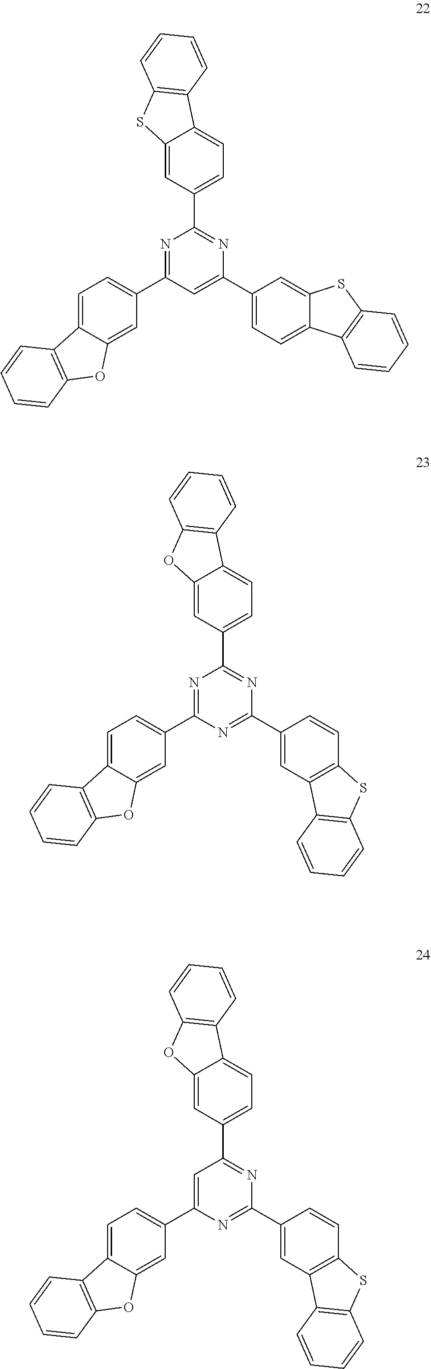 [SMILES: c1ccc2c(c1)oc1cc(-c3cc(-c4ccc5c(c4)sc4ccccc45)nc(-c4ccc5c(c4)sc4ccccc45)n3)ccc12 ; c1ccc2c(c1)oc1cc(-c3nc(-c4ccc5c(c4)oc4ccccc45)nc(-c4ccc5sc6ccccc6c5c4)n3)ccc12 ; c1ccc2c(c1)oc1cc(-c3cc(-c4ccc5c(c4)oc4ccccc45)nc(-c4ccc5sc6ccccc6c5c4)n3)ccc12]

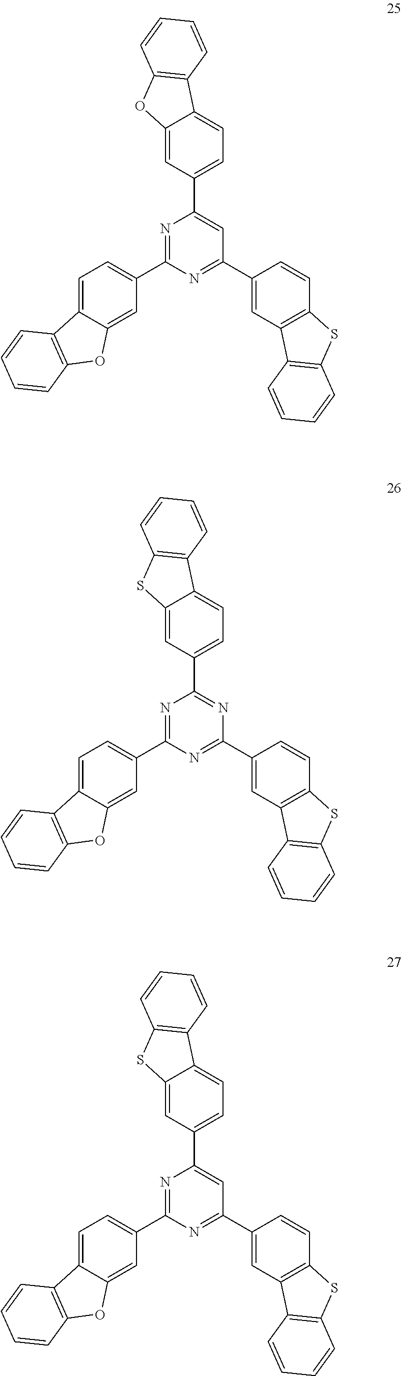 [SMILES: c1ccc2c(c1)oc1cc(-c3cc(-c4ccc5sc6ccccc6c5c4)nc(-c4ccc5c(c4)oc4ccccc45)n3)ccc12 ; c1ccc2c(c1)oc1cc(-c3nc(-c4ccc5c(c4)sc4ccccc45)nc(-c4ccc5sc6ccccc6c5c4)n3)ccc12 ; c1ccc2c(c1)oc1cc(-c3nc(-c4ccc5c(c4)sc4ccccc45)cc(-c4ccc5sc6ccccc6c5c4)n3)ccc12]

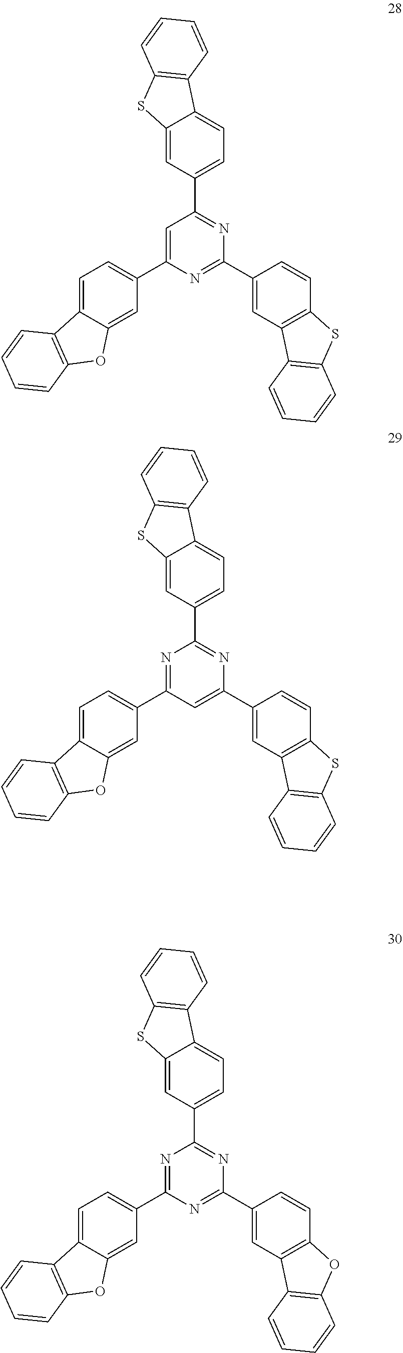 [SMILES: c1ccc2c(c1)oc1cc(-c3cc(-c4ccc5c(c4)sc4ccccc45)nc(-c4ccc5sc6ccccc6c5c4)n3)ccc12 ; c1ccc2c(c1)oc1cc(-c3cc(-c4ccc5sc6ccccc6c5c4)nc(-c4ccc5c(c4)sc4ccccc45)n3)ccc12 ; c1ccc2c(c1)oc1cc(-c3nc(-c4ccc5c(c4)sc4ccccc45)nc(-c4ccc5oc6ccccc6c5c4)n3)ccc12]

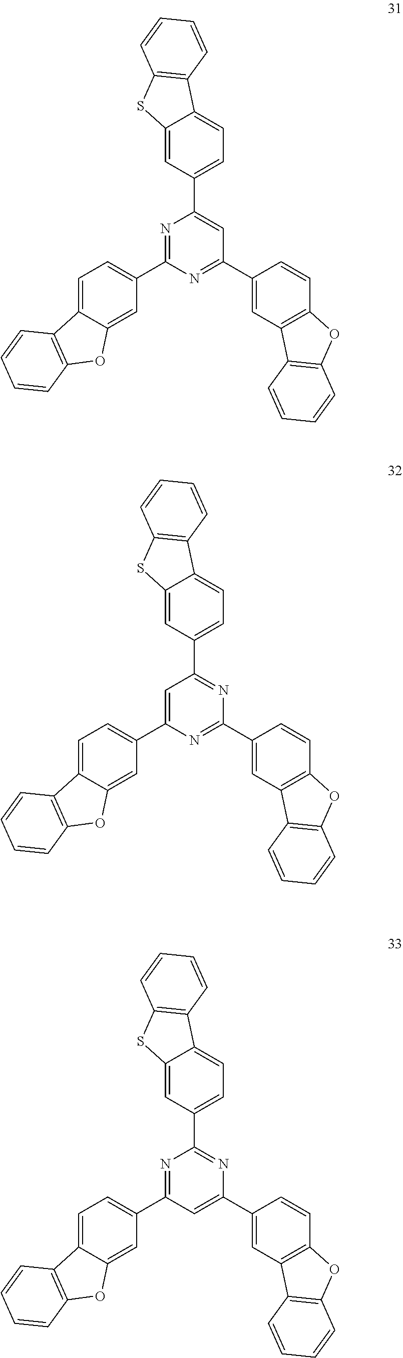 [SMILES: c1ccc2c(c1)oc1cc(-c3nc(-c4ccc5c(c4)sc4ccccc45)cc(-c4ccc5oc6ccccc6c5c4)n3)ccc12 ; c1ccc2c(c1)oc1cc(-c3cc(-c4ccc5c(c4)sc4ccccc45)nc(-c4ccc5oc6ccccc6c5c4)n3)ccc12 ; c1ccc2c(c1)oc1cc(-c3cc(-c4ccc5oc6ccccc6c5c4)nc(-c4ccc5c(c4)sc4ccccc45)n3)ccc12]

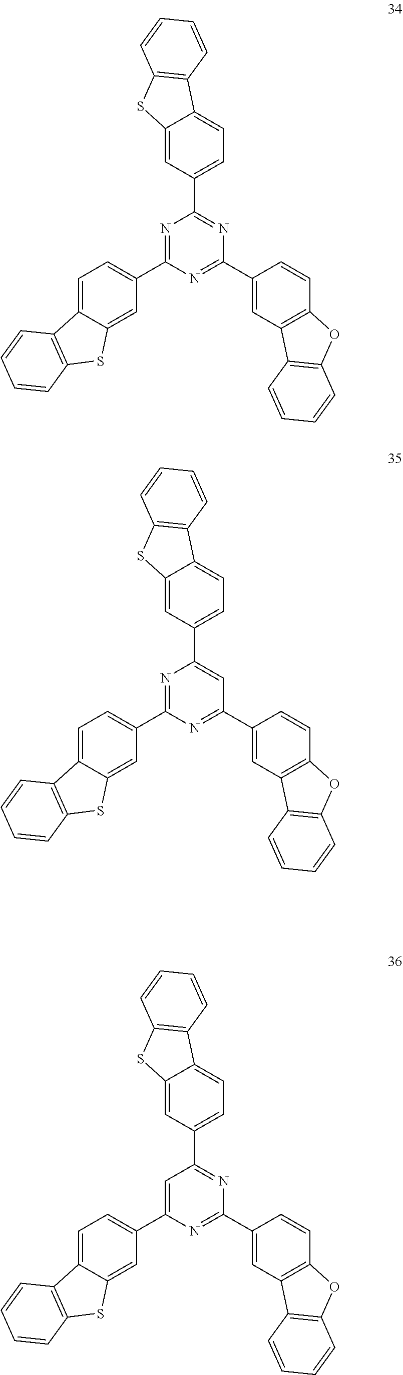 [SMILES: c1ccc2c(c1)oc1ccc(-c3nc(-c4ccc5c(c4)sc4ccccc45)nc(-c4ccc5c(c4)sc4ccccc45)n3)cc12 ; c1ccc2c(c1)oc1ccc(-c3cc(-c4ccc5c(c4)sc4ccccc45)nc(-c4ccc5c(c4)sc4ccccc45)n3)cc12 ; c1ccc2c(c1)oc1ccc(-c3nc(-c4ccc5c(c4)sc4ccccc45)cc(-c4ccc5c(c4)sc4ccccc45)n3)cc12]

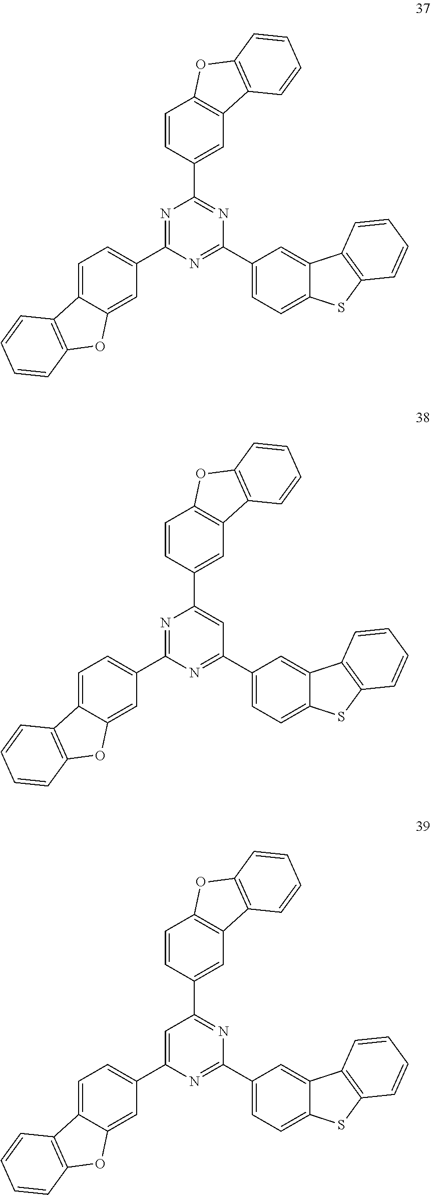 [SMILES: c1ccc2c(c1)oc1cc(-c3nc(-c4ccc5oc6ccccc6c5c4)nc(-c4ccc5sc6ccccc6c5c4)n3)ccc12 ; c1ccc2c(c1)oc1cc(-c3nc(-c4ccc5oc6ccccc6c5c4)cc(-c4ccc5sc6ccccc6c5c4)n3)ccc12 ; c1ccc2c(c1)oc1cc(-c3cc(-c4ccc5oc6ccccc6c5c4)nc(-c4ccc5sc6ccccc6c5c4)n3)ccc12]

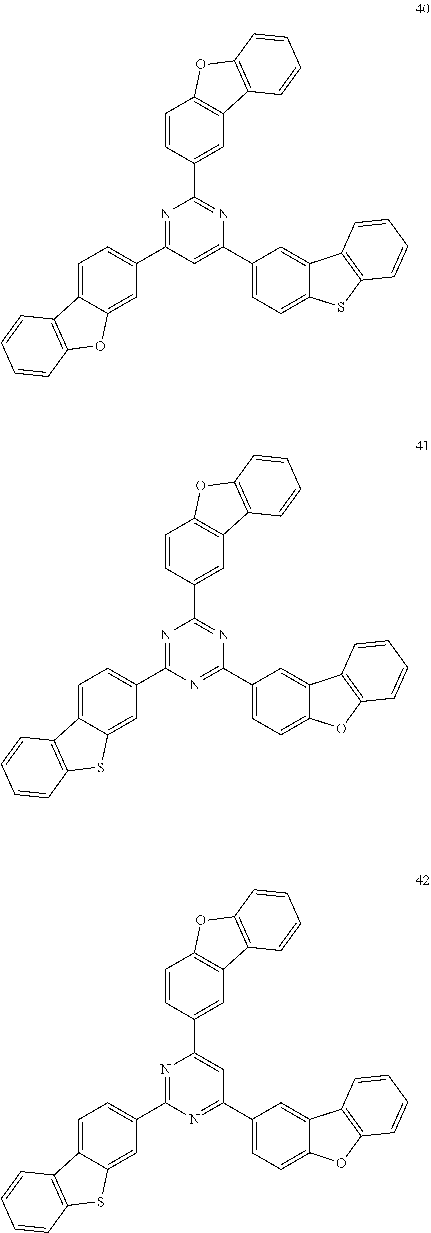 [SMILES: c1ccc2c(c1)oc1cc(-c3cc(-c4ccc5sc6ccccc6c5c4)nc(-c4ccc5oc6ccccc6c5c4)n3)ccc12 ; c1ccc2c(c1)oc1ccc(-c3nc(-c4ccc5c(c4)sc4ccccc45)nc(-c4ccc5oc6ccccc6c5c4)n3)cc12 ; c1ccc2c(c1)oc1ccc(-c3cc(-c4ccc5oc6ccccc6c5c4)nc(-c4ccc5c(c4)sc4ccccc45)n3)cc12]

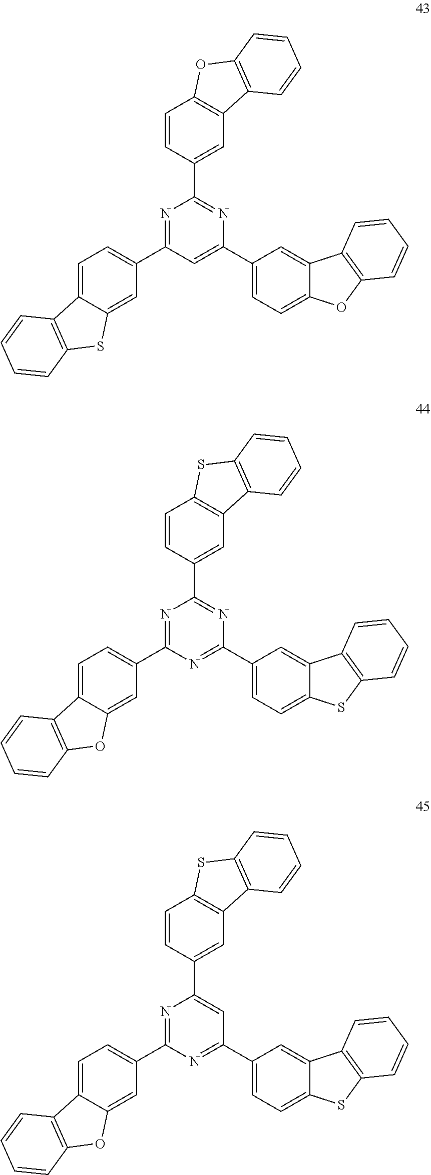 [SMILES: c1ccc2c(c1)oc1ccc(-c3cc(-c4ccc5c(c4)sc4ccccc45)nc(-c4ccc5oc6ccccc6c5c4)n3)cc12 ; c1ccc2c(c1)oc1cc(-c3nc(-c4ccc5sc6ccccc6c5c4)nc(-c4ccc5sc6ccccc6c5c4)n3)ccc12 ; c1ccc2c(c1)oc1cc(-c3nc(-c4ccc5sc6ccccc6c5c4)cc(-c4ccc5sc6ccccc6c5c4)n3)ccc12]

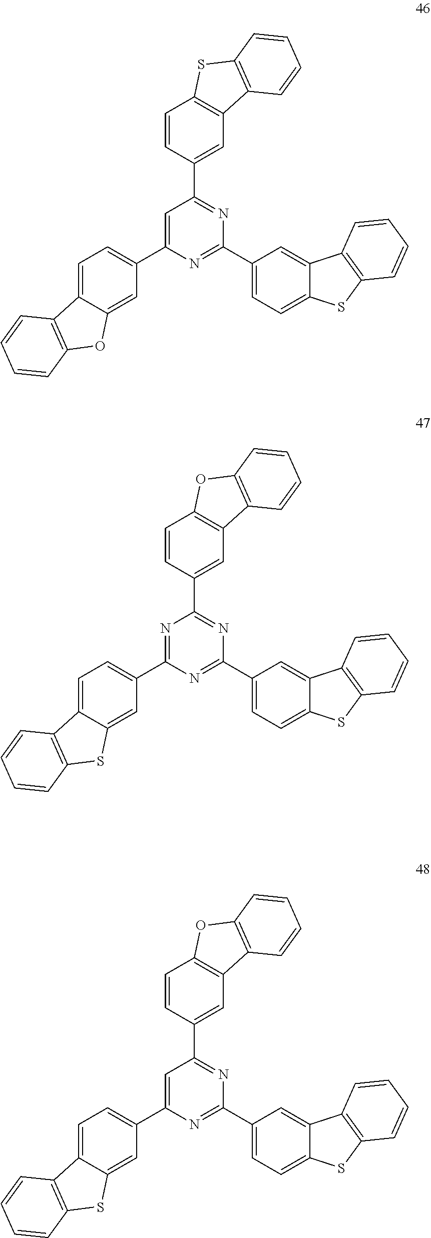 [SMILES: c1ccc2c(c1)oc1cc(-c3cc(-c4ccc5sc6ccccc6c5c4)nc(-c4ccc5sc6ccccc6c5c4)n3)ccc12 ; c1ccc2c(c1)oc1ccc(-c3nc(-c4ccc5c(c4)sc4ccccc45)nc(-c4ccc5sc6ccccc6c5c4)n3)cc12 ; c1ccc2c(c1)oc1ccc(-c3cc(-c4ccc5c(c4)sc4ccccc45)nc(-c4ccc5sc6ccccc6c5c4)n3)cc12]

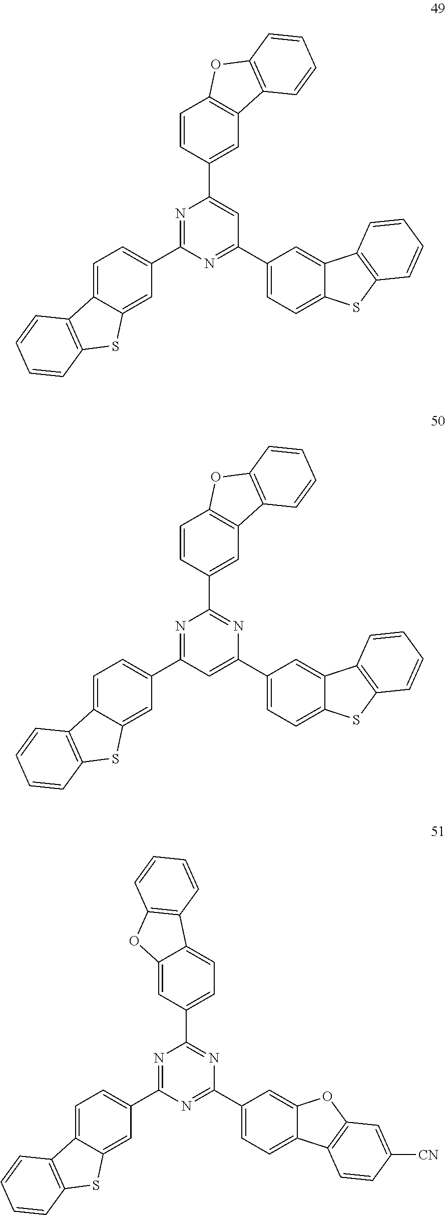 [SMILES: c1ccc2c(c1)oc1ccc(-c3cc(-c4ccc5sc6ccccc6c5c4)nc(-c4ccc5c(c4)sc4ccccc45)n3)cc12 ; c1ccc2c(c1)oc1ccc(-c3nc(-c4ccc5c(c4)sc4ccccc45)cc(-c4ccc5sc6ccccc6c5c4)n3)cc12 ; N#Cc1ccc2c(c1)oc1cc(-c3nc(-c4ccc5c(c4)oc4ccccc45)nc(-c4ccc5c(c4)sc4ccccc45)n3)ccc12]

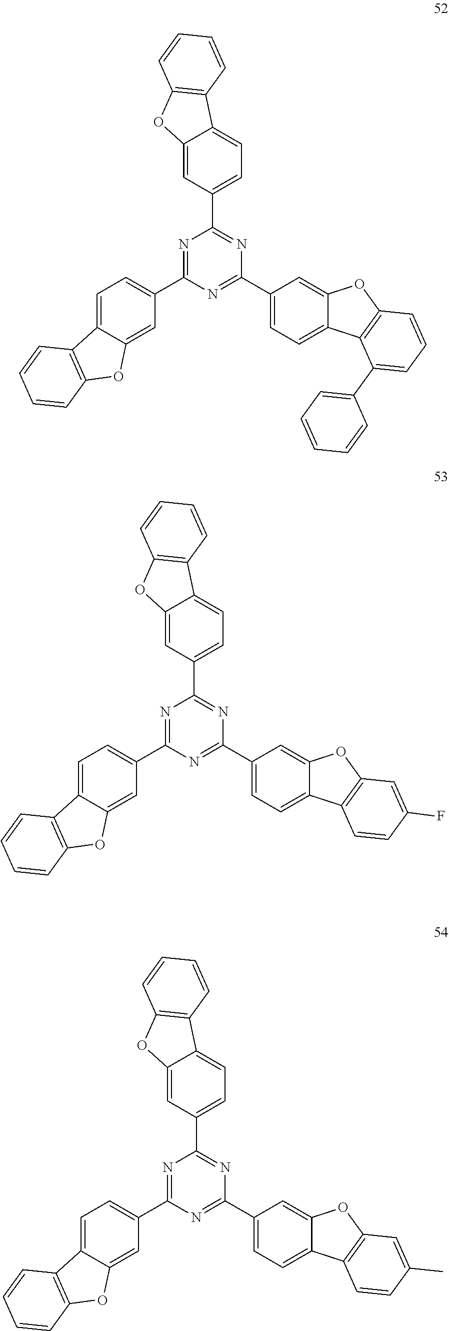 [SMILES: c1ccc(-c2cccc3oc4cc(-c5nc(-c6ccc7c(c6)oc6ccccc67)nc(-c6ccc7c(c6)oc6ccccc67)n5)ccc4c23)cc1 ; Fc1ccc2c(c1)oc1cc(-c3nc(-c4ccc5c(c4)oc4ccccc45)nc(-c4ccc5c(c4)oc4ccccc45)n3)ccc12 ; Cc1ccc2c(c1)oc1cc(-c3nc(-c4ccc5c(c4)oc4ccccc45)nc(-c4ccc5c(c4)oc4ccccc45)n3)ccc12]

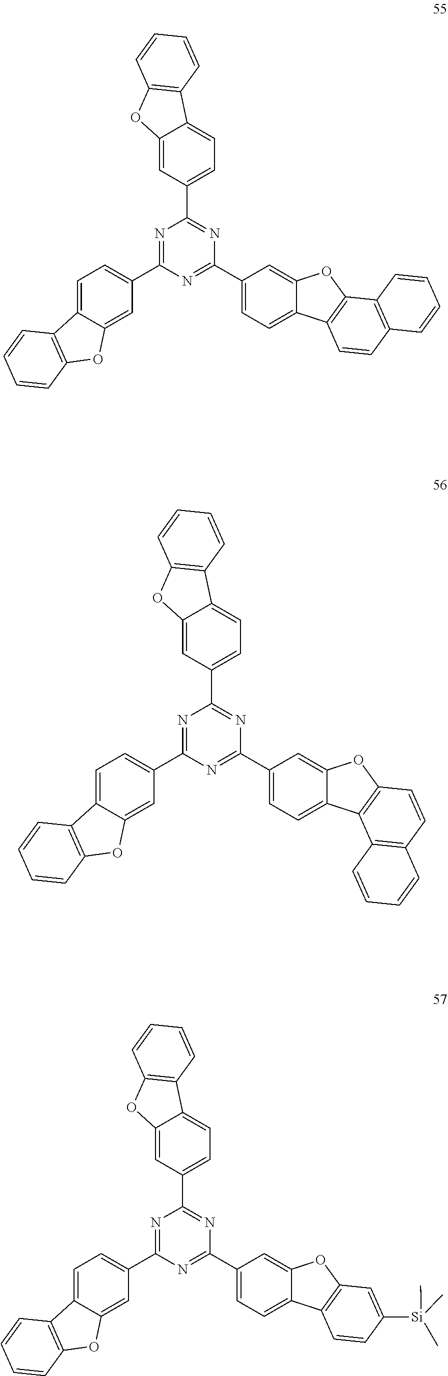 [SMILES: c1ccc2c(c1)ccc1c3ccc(-c4nc(-c5ccc6c(c5)oc5ccccc56)nc(-c5ccc6c(c5)oc5ccccc56)n4)cc3oc21 ; c1ccc2c(c1)ccc1oc3cc(-c4nc(-c5ccc6c(c5)oc5ccccc56)nc(-c5ccc6c(c5)oc5ccccc56)n4)ccc3c12 ; C[Si](C)(C)c1ccc2c(c1)oc1cc(-c3nc(-c4ccc5c(c4)oc4ccccc45)nc(-c4ccc5c(c4)oc4ccccc45)n3)ccc12]

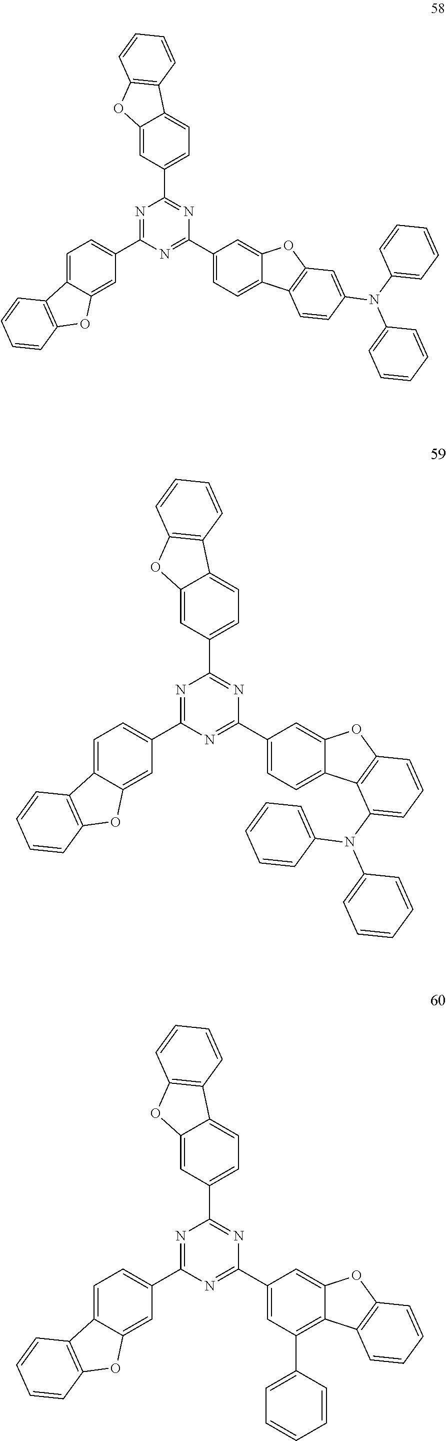 [SMILES: c1ccc(N(c2ccccc2)c2ccc3c(c2)oc2cc(-c4nc(-c5ccc6c(c5)oc5ccccc56)nc(-c5ccc6c(c5)oc5ccccc56)n4)ccc23)cc1 ; c1ccc(N(c2ccccc2)c2cccc3oc4cc(-c5nc(-c6ccc7c(c6)oc6ccccc67)nc(-c6ccc7c(c6)oc6ccccc67)n5)ccc4c23)cc1 ; c1ccc(-c2cc(-c3nc(-c4ccc5c(c4)oc4ccccc45)nc(-c4ccc5c(c4)oc4ccccc45)n3)cc3oc4ccccc4c23)cc1]

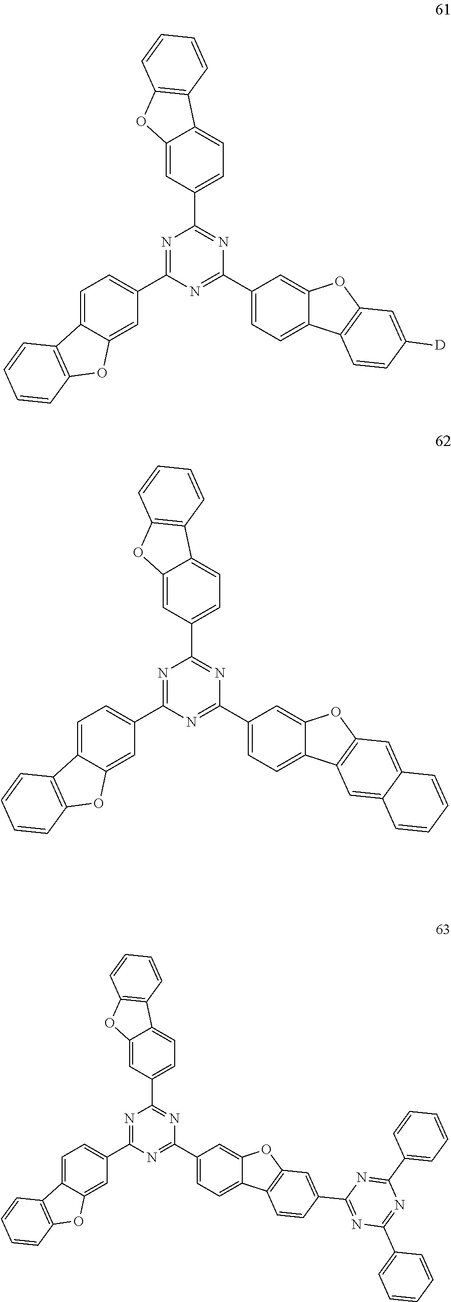 [SMILES: [2H]c1ccc2c(c1)oc1cc(-c3nc(-c4ccc5c(c4)oc4ccccc45)nc(-c4ccc5c(c4)oc4ccccc45)n3)ccc12 ; c1ccc2cc3c(cc2c1)oc1cc(-c2nc(-c4ccc5c(c4)oc4ccccc45)nc(-c4ccc5c(c4)oc4ccccc45)n2)ccc13 ; c1ccc(-c2nc(-c3ccccc3)nc(-c3ccc4c(c3)oc3cc(-c5nc(-c6ccc7c(c6)oc6ccccc67)nc(-c6ccc7c(c6)oc6ccccc67)n5)ccc34)n2)cc1]

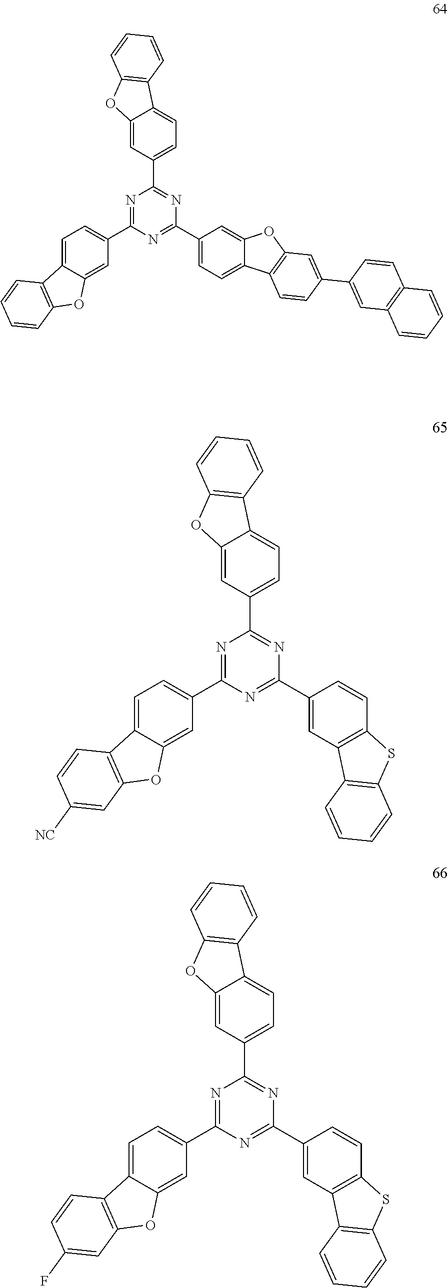 [SMILES: c1ccc2cc(-c3ccc4c(c3)oc3cc(-c5nc(-c6ccc7c(c6)oc6ccccc67)nc(-c6ccc7c(c6)oc6ccccc67)n5)ccc34)ccc2c1 ; N#Cc1ccc2c(c1)oc1cc(-c3nc(-c4ccc5c(c4)oc4ccccc45)nc(-c4ccc5sc6ccccc6c5c4)n3)ccc12 ; Fc1ccc2c(c1)oc1cc(-c3nc(-c4ccc5c(c4)oc4ccccc45)nc(-c4ccc5sc6ccccc6c5c4)n3)ccc12]

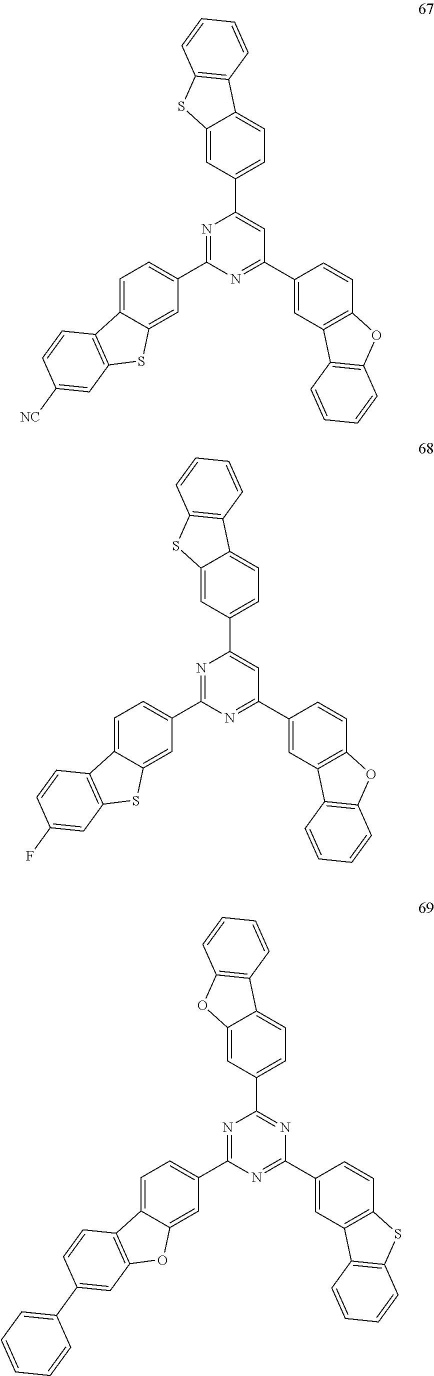 [SMILES: N#Cc1ccc2c(c1)sc1cc(-c3nc(-c4ccc5c(c4)sc4ccccc45)cc(-c4ccc5oc6ccccc6c5c4)n3)ccc12 ; Fc1ccc2c(c1)sc1cc(-c3nc(-c4ccc5c(c4)sc4ccccc45)cc(-c4ccc5oc6ccccc6c5c4)n3)ccc12 ; c1ccc(-c2ccc3c(c2)oc2cc(-c4nc(-c5ccc6c(c5)oc5ccccc56)nc(-c5ccc6sc7ccccc7c6c5)n4)ccc23)cc1]

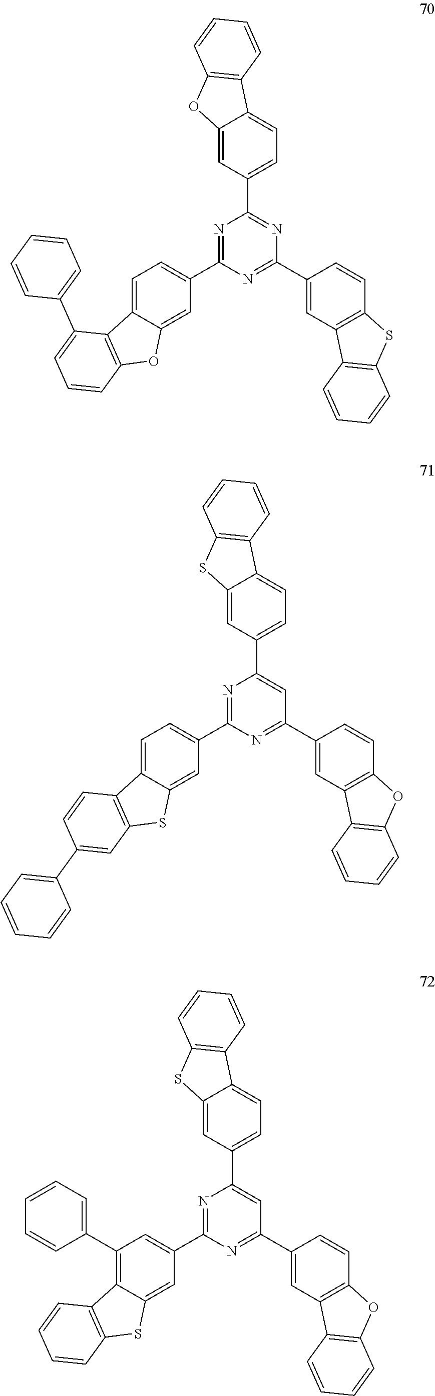 [SMILES: c1ccc(-c2cccc3oc4cc(-c5nc(-c6ccc7c(c6)oc6ccccc67)nc(-c6ccc7sc8ccccc8c7c6)n5)ccc4c23)cc1 ; c1ccc(-c2ccc3c(c2)sc2cc(-c4nc(-c5ccc6c(c5)sc5ccccc56)cc(-c5ccc6oc7ccccc7c6c5)n4)ccc23)cc1 ; c1ccc(-c2cc(-c3nc(-c4ccc5c(c4)sc4ccccc45)cc(-c4ccc5oc6ccccc6c5c4)n3)cc3sc4ccccc4c23)cc1]

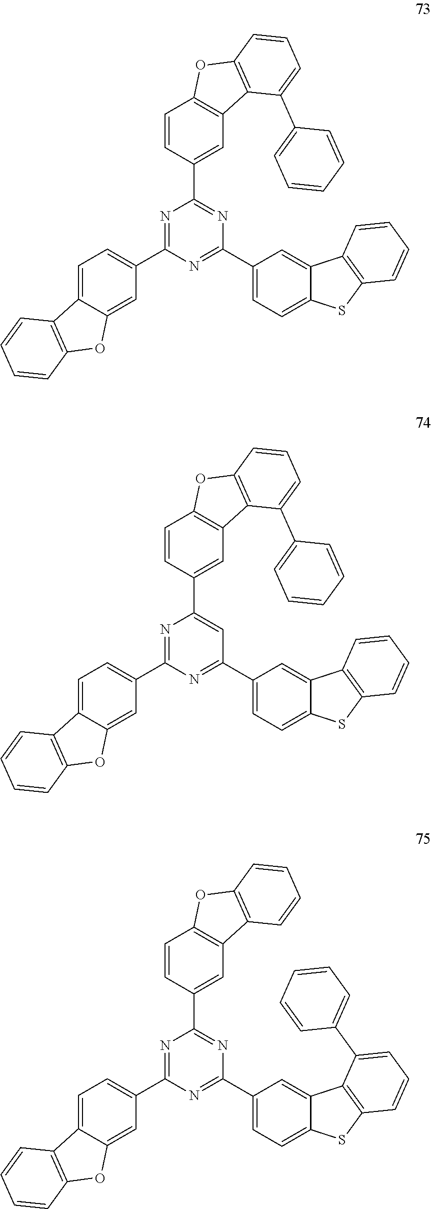 [SMILES: c1ccc(-c2cccc3oc4ccc(-c5nc(-c6ccc7c(c6)oc6ccccc67)nc(-c6ccc7sc8ccccc8c7c6)n5)cc4c23)cc1 ; c1ccc(-c2cccc3oc4ccc(-c5cc(-c6ccc7sc8ccccc8c7c6)nc(-c6ccc7c(c6)oc6ccccc67)n5)cc4c23)cc1 ; c1ccc(-c2cccc3sc4ccc(-c5nc(-c6ccc7c(c6)oc6ccccc67)nc(-c6ccc7oc8ccccc8c7c6)n5)cc4c23)cc1]

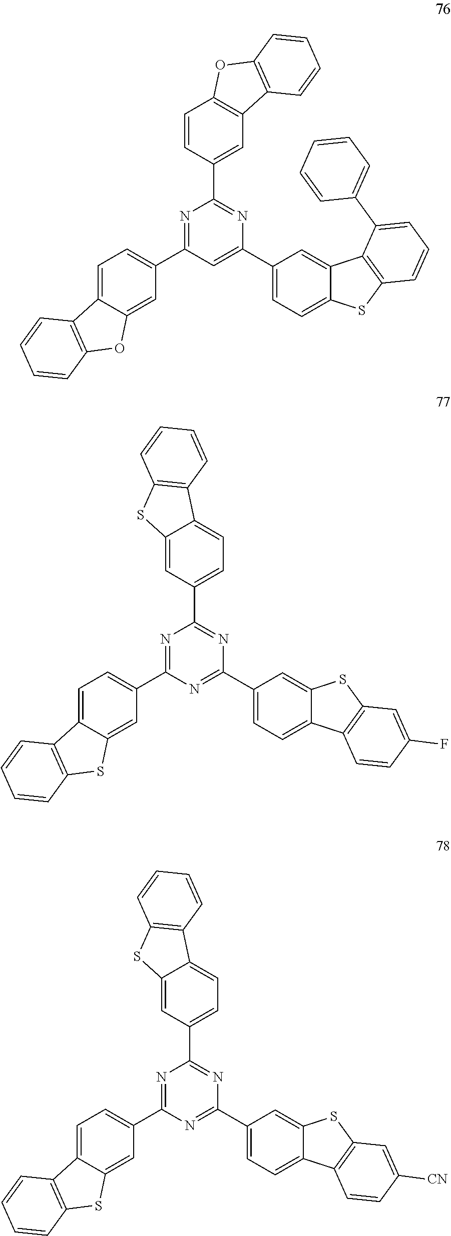 [SMILES: c1ccc(-c2cccc3sc4ccc(-c5cc(-c6ccc7c(c6)oc6ccccc67)nc(-c6ccc7oc8ccccc8c7c6)n5)cc4c23)cc1 ; Fc1ccc2c(c1)sc1cc(-c3nc(-c4ccc5c(c4)sc4ccccc45)nc(-c4ccc5c(c4)sc4ccccc45)n3)ccc12 ; N#Cc1ccc2c(c1)sc1cc(-c3nc(-c4ccc5c(c4)sc4ccccc45)nc(-c4ccc5c(c4)sc4ccccc45)n3)ccc12]

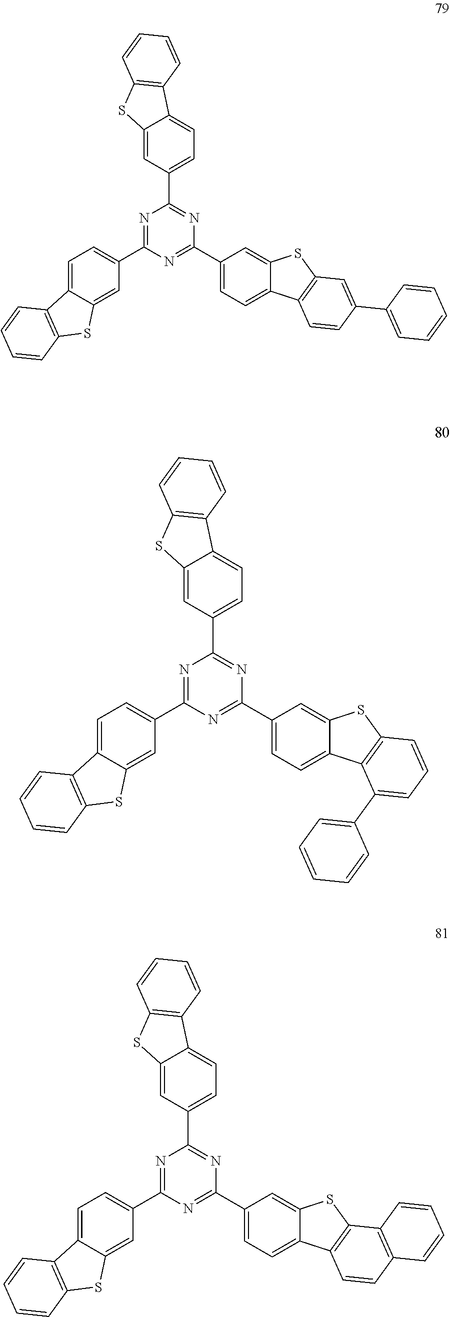 [SMILES: c1ccc(-c2ccc3c(c2)sc2cc(-c4nc(-c5ccc6c(c5)sc5ccccc56)nc(-c5ccc6c(c5)sc5ccccc56)n4)ccc23)cc1 ; c1ccc(-c2cccc3sc4cc(-c5nc(-c6ccc7c(c6)sc6ccccc67)nc(-c6ccc7c(c6)sc6ccccc67)n5)ccc4c23)cc1 ; c1ccc2c(c1)ccc1c3ccc(-c4nc(-c5ccc6c(c5)sc5ccccc56)nc(-c5ccc6c(c5)sc5ccccc56)n4)cc3sc21]

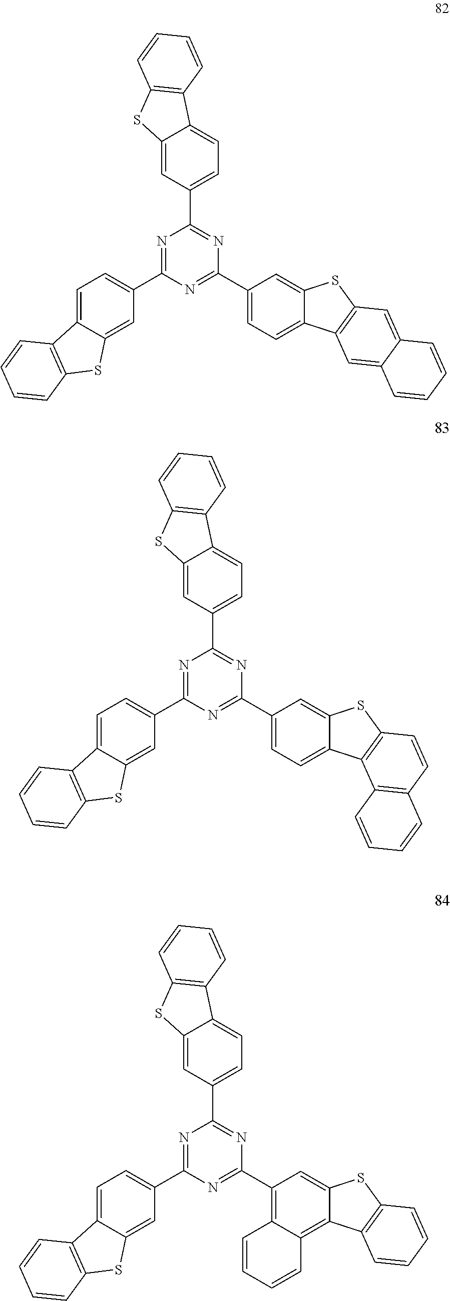 [SMILES: c1ccc2cc3c(cc2c1)sc1cc(-c2nc(-c4ccc5c(c4)sc4ccccc45)nc(-c4ccc5c(c4)sc4ccccc45)n2)ccc13 ; c1ccc2c(c1)ccc1sc3cc(-c4nc(-c5ccc6c(c5)sc5ccccc56)nc(-c5ccc6c(c5)sc5ccccc56)n4)ccc3c12 ; c1ccc2c(c1)sc1cc(-c3nc(-c4ccc5c(c4)sc4ccccc45)nc(-c4cc5sc6ccccc6c5c5ccccc45)n3)ccc12]

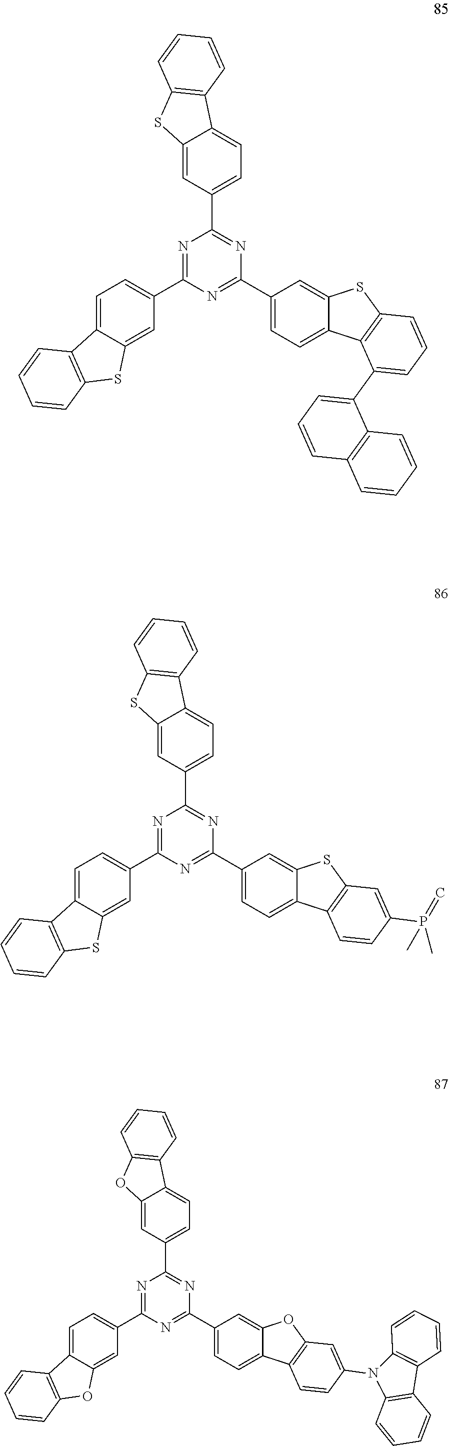 [SMILES: c1ccc2c(-c3cccc4sc5cc(-c6nc(-c7ccc8c(c7)sc7ccccc78)nc(-c7ccc8c(c7)sc7ccccc78)n6)ccc5c34)cccc2c1 ; C=P(C)(C)c1ccc2c(c1)sc1cc(-c3nc(-c4ccc5c(c4)sc4ccccc45)nc(-c4ccc5c(c4)sc4ccccc45)n3)ccc12 ; c1ccc2c(c1)oc1cc(-c3nc(-c4ccc5c(c4)oc4ccccc45)nc(-c4ccc5c(c4)oc4cc(-n6c7ccccc7c7ccccc76)ccc45)n3)ccc12]

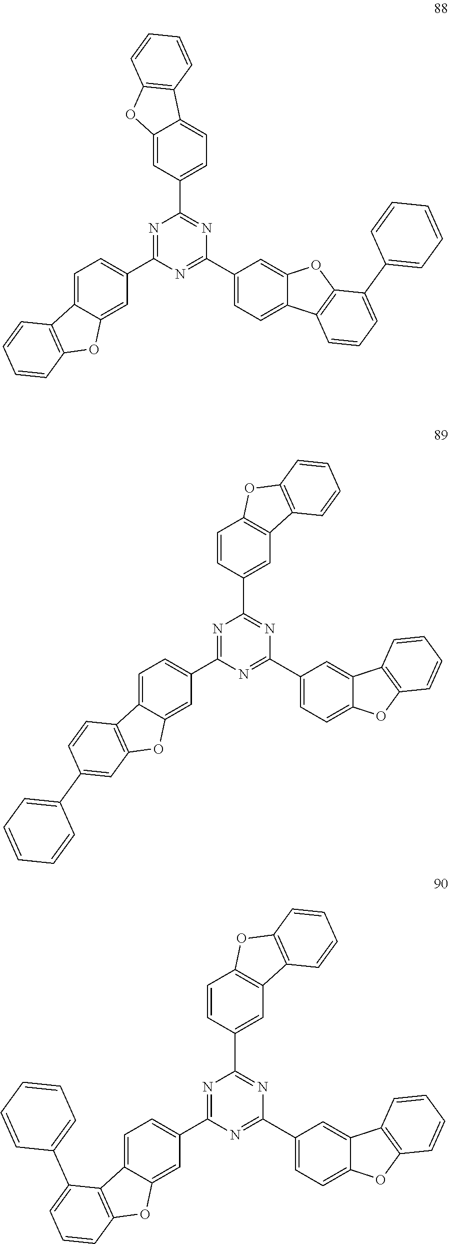 [SMILES: c1ccc(-c2cccc3c2oc2cc(-c4nc(-c5ccc6c(c5)oc5ccccc56)nc(-c5ccc6c(c5)oc5ccccc56)n4)ccc23)cc1 ; c1ccc(-c2ccc3c(c2)oc2cc(-c4nc(-c5ccc6oc7ccccc7c6c5)nc(-c5ccc6oc7ccccc7c6c5)n4)ccc23)cc1 ; c1ccc(-c2cccc3oc4cc(-c5nc(-c6ccc7oc8ccccc8c7c6)nc(-c6ccc7oc8ccccc8c7c6)n5)ccc4c23)cc1]

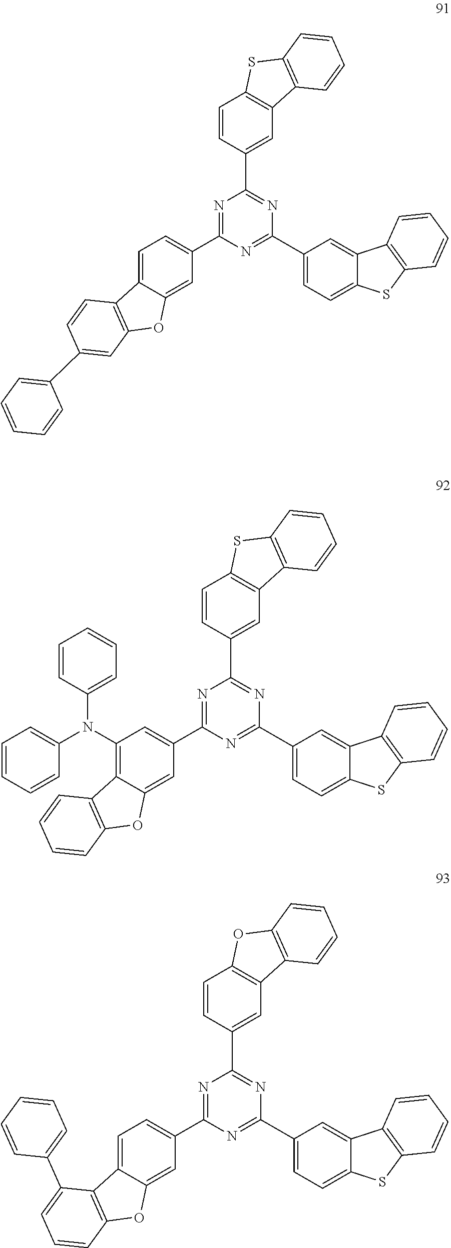 [SMILES: c1ccc(-c2ccc3c(c2)oc2cc(-c4nc(-c5ccc6sc7ccccc7c6c5)nc(-c5ccc6sc7ccccc7c6c5)n4)ccc23)cc1 ; c1ccc(N(c2ccccc2)c2cc(-c3nc(-c4ccc5sc6ccccc6c5c4)nc(-c4ccc5sc6ccccc6c5c4)n3)cc3oc4ccccc4c23)cc1 ; c1ccc(-c2cccc3oc4cc(-c5nc(-c6ccc7oc8ccccc8c7c6)nc(-c6ccc7sc8ccccc8c7c6)n5)ccc4c23)cc1]

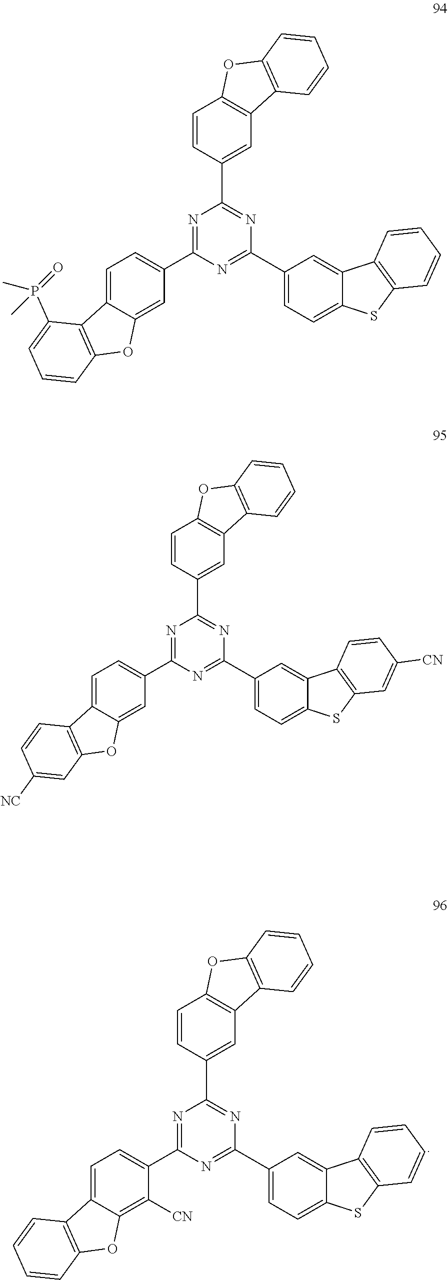 [SMILES: CP(C)(=O)c1cccc2oc3cc(-c4nc(-c5ccc6oc7ccccc7c6c5)nc(-c5ccc6sc7ccccc7c6c5)n4)ccc3c12 ; N#Cc1ccc2c(c1)oc1cc(-c3nc(-c4ccc5oc6ccccc6c5c4)nc(-c4ccc5sc6cc(C#N)ccc6c5c4)n3)ccc12 ; N#Cc1c(-c2nc(-c3ccc4oc5ccccc5c4c3)nc(-c3ccc4sc5ccccc5c4c3)n2)ccc2c1oc1ccccc12]

The organic compound may be applied in an organic optoelectronic device alone or with other compounds. When the organic compound is applied with other compounds, it may be applied in a form of a composition.

Hereinafter, a composition according to an embodiment is described.

A composition according to an embodiment may include the organic compound (hereinafter, referred to as “a first organic compound”) and an organic compound having hole characteristics (hereinafter, referred to as “a second organic compound”).

The second organic compound may include for example a carbazole moiety, for example a substituted or unsubstituted carbazole compound, a substituted or unsubstituted biscarbazole compound, or a substituted or unsubstituted indolocarbazole compound.

For example, the second organic compound may be or may include a carbazole moiety represented by Chemical Formula 7.

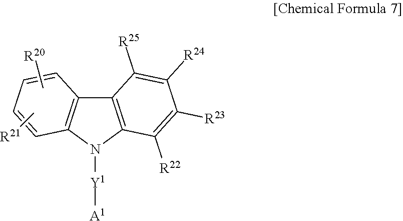

In Chemical Formula 7,

Y¹ may be a single bond, a substituted or unsubstituted C6 to C30 arylene group, or a divalent substituted or unsubstituted C2 to C30 heterocyclic group,

A¹ may be a substituted or unsubstituted C6 to C30 aryl group, or a substituted or unsubstituted C2 to C30 heterocyclic group,

R²⁰ to R²⁵ may independently be hydrogen, deuterium, a substituted or unsubstituted C1 to C20 alkyl group, a substituted or unsubstituted C6 to C30 aryl group, or a substituted or unsubstituted C2 to C30 heterocyclic group, and

R²² to R²⁵ may independently be present or adjacent groups of R²² to R²⁵ may be linked with each other to form a ring.

For example, in Chemical Formula 7, “substituted” refers to replacement of at least one hydrogen by deuterium, a C1 to C10 alkyl group, a C6 to C12 aryl group or a C2 to C10 heterocyclic group, for example refers to replacement of at least one hydrogen by deuterium, a phenyl group, an ortho-biphenyl group, a meta-biphenyl group, a para-biphenyl group, a terphenyl group, a naphthyl group, a dibenzofuranyl group, or a dibenzothiophenyl group.

For example, the second organic compound may be a compound represented by Chemical Formula 7A.

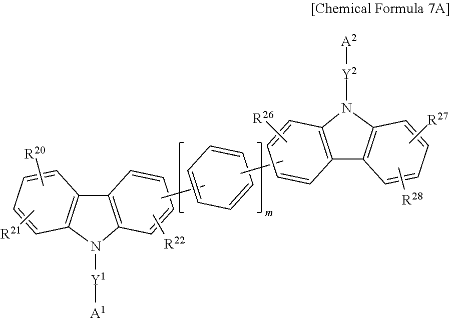

In Chemical Formula 7A,

Y¹ and Y² may independently be a single bond, a substituted or unsubstituted C6 to C30 arylene group, divalent substituted or unsubstituted C2 to C30 heterocyclic group, or a combination thereof,

A¹ and A² may independently be a substituted or unsubstituted C6 to C30 aryl group, a substituted or unsubstituted C2 to C30 heterocyclic group, or a combination thereof,

R²⁰ to R²² and R²⁶ to R²⁸ may independently be hydrogen, deuterium, a substituted or unsubstituted C1 to C20 alkyl group, a substituted or unsubstituted C6 to C30 aryl group, a substituted or unsubstituted C2 to C30 heterocyclic group, or a combination thereof, and

m may be an integer of 0 to 2.

For example, Y¹ and Y² of Chemical Formula 7A may independently be a single bond, a substituted or unsubstituted phenylene group or a substituted or unsubstituted biphenylene group, for example a single bond, a meta-phenylene group, a para-phenylene group, a meta-biphenylene group, or a para-biphenylene group.

For example, A¹ and A² of Chemical Formula 7A may independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, or a substituted or unsubstituted triphenylene group, a substituted or unsubstituted pyridinyl group, a substituted or unsubstituted dibenzothiophenyl group, a substituted or unsubstituted dibenzofuranyl group, a substituted or unsubstituted carbazolyl group, a substituted or unsubstituted fluorenyl group, or a combination thereof. For example, A¹ and A² of Chemical Formula 7A may independently be a substituted or unsubstituted phenyl group, a substituted or unsubstituted biphenyl group, a substituted or unsubstituted dibenzothiophenyl group, a substituted or unsubstituted dibenzofuranyl group, or a substituted or unsubstituted carbazolyl group.

For example, R²⁰ to R²² and R²⁶ to R²⁸ of Chemical Formula 7A may be hydrogen, a substituted or unsubstituted C6 to C30 aryl group, or a substituted or unsubstituted C2 to C30 heterocyclic group, or may be, for example, all hydrogen.

For example, m of Chemical Formula 7A may be 0 or 1, or for example m may be 0.

For example, in Chemical Formula 7A, a binding position of two carbazole groups may be a 2,3-bond, 3,3-bond, or 2,2-bond, for example a 3,3-bond.

For example, the compound represented by Chemical Formula 7A may be represented by Chemical Formula 7A-1.

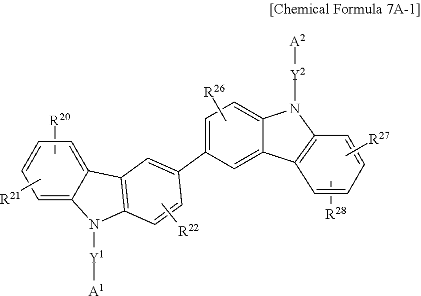

In Chemical Formula 7A-1, Y¹, Y², A¹, A², R²⁰ to R²² and R²⁶ to R²⁸ are the same as described above.

For example, the compound represented by Chemical Formula 7A may be a compound including a combination of one of carbazole cores listed in Group 2 and substituents (*—Y′-A′ and *—Y²-A²) listed in Group 3.

[Group 2]

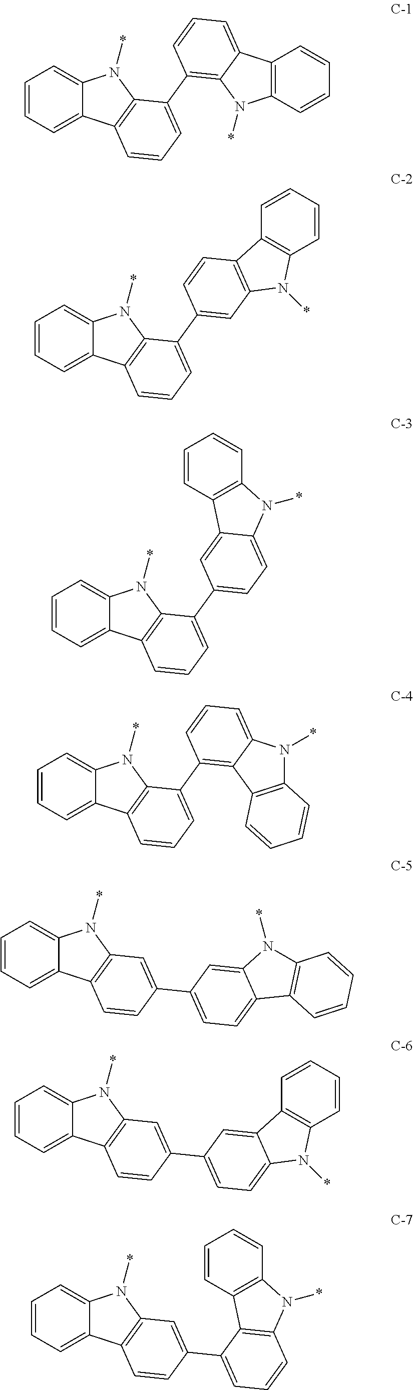

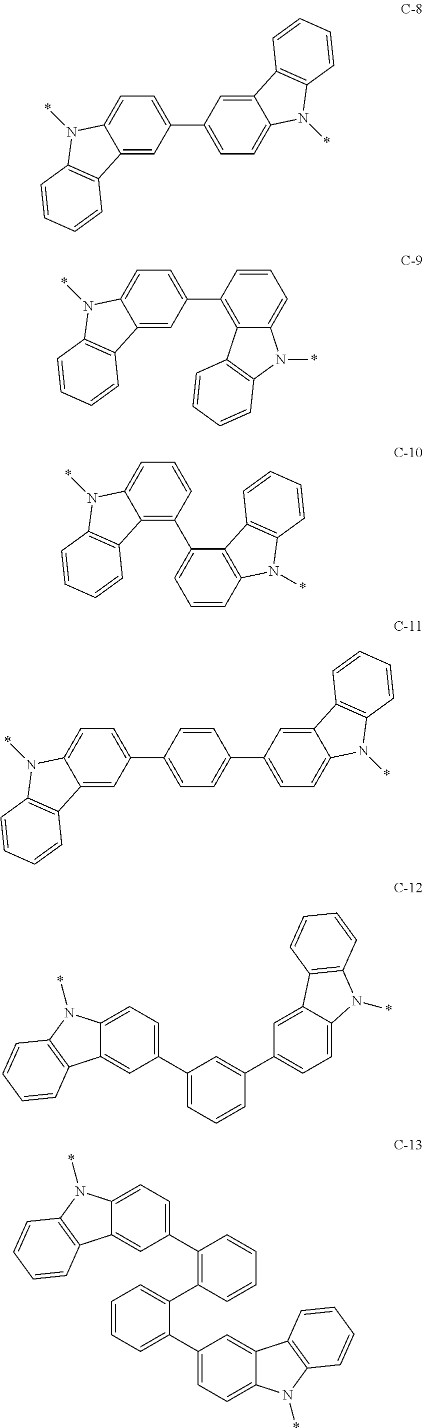 [SMILES: *n1c2ccccc2c2cc(-c3ccc4c(c3)c3ccccc3n4*)ccc21 ; *n1c2ccccc2c2cc(-c3cccc4c3c3ccccc3n4*)ccc21 ; *n1c2ccccc2c2c(-c3cccc4c3c3ccccc3n4*)cccc21 ; *n1c2ccccc2c2cc(-c3ccc(-c4ccc5c(c4)c4ccccc4n5*)cc3)ccc21 ; *n1c2ccccc2c2cc(-c3cccc(-c4ccc5c(c4)c4ccccc4n5*)c3)ccc21 ; *n1c2ccccc2c2cc(-c3ccccc3-c3ccccc3-c3ccc4c(c3)c3ccccc3n4*)ccc21]

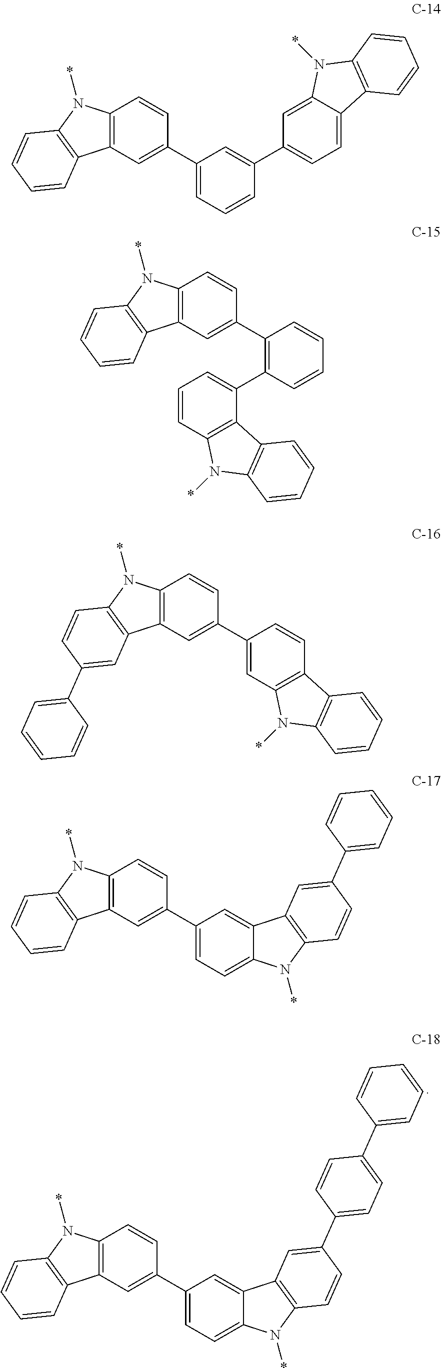

[Group 3]

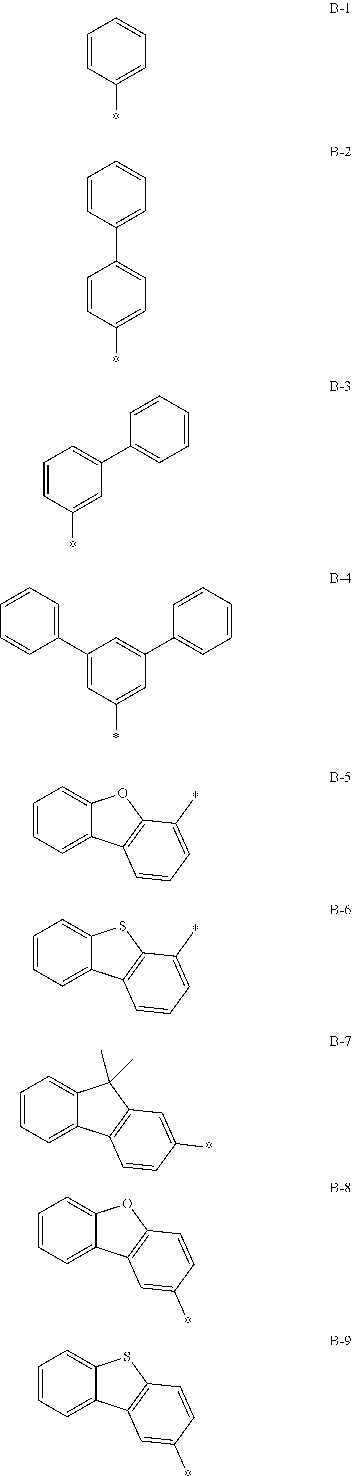

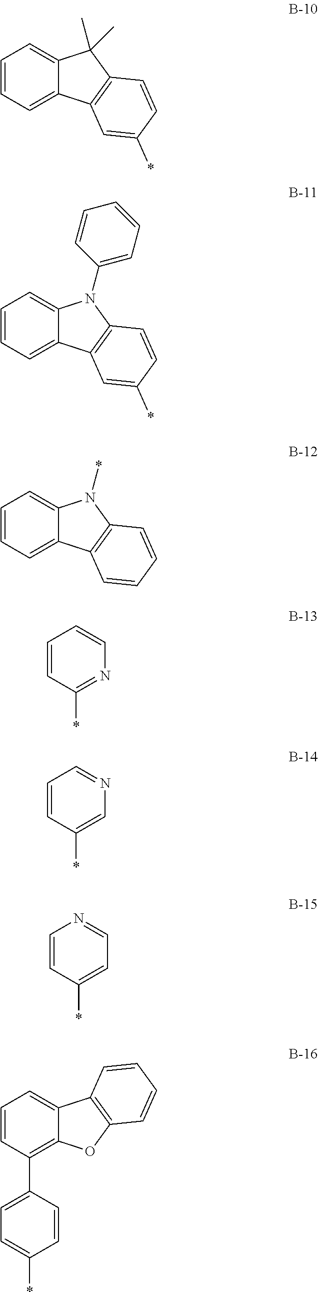

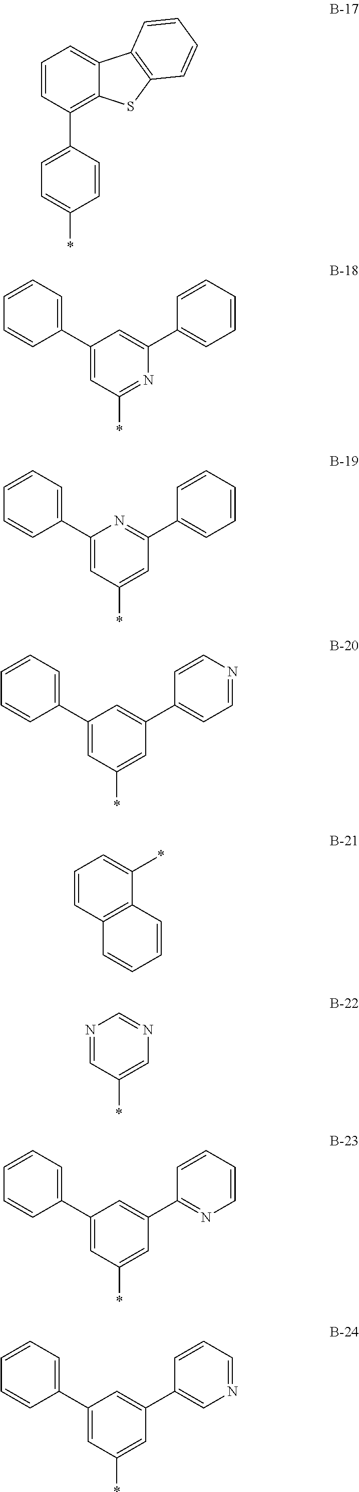

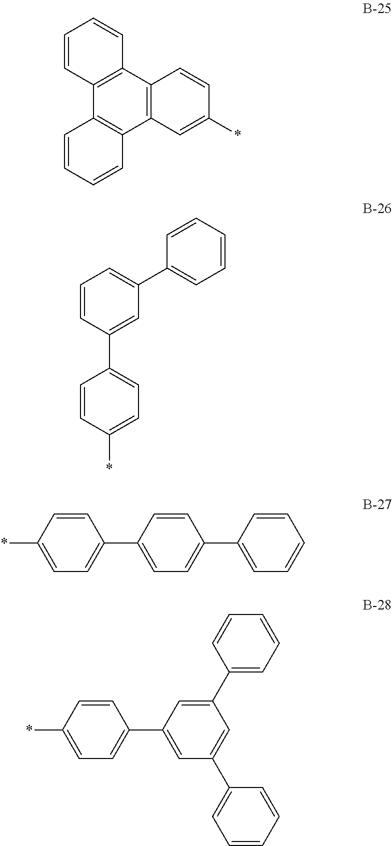

In Groups 2 and 3. * is a linking point.

For example, the compound represented by Chemical Formula 7A may be for example one of compounds listed in Group 4.

[Group 4]

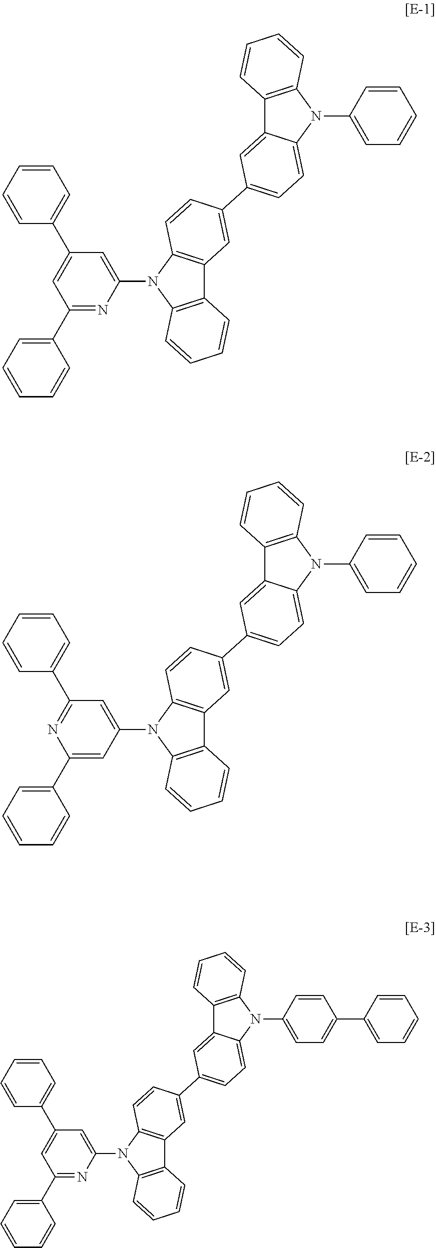 [SMILES: c1ccc(-c2cc(-c3ccccc3)nc(-n3c4ccccc4c4cc(-c5ccc6c(c5)c5ccccc5n6-c5ccccc5)ccc43)c2)cc1 ; c1ccc(-c2cc(-n3c4ccccc4c4cc(-c5ccc6c(c5)c5ccccc5n6-c5ccccc5)ccc43)cc(-c3ccccc3)n2)cc1 ; c1ccc(-c2ccc(-n3c4ccccc4c4cc(-c5ccc6c(c5)c5ccccc5n6-c5cc(-c6ccccc6)cc(-c6ccccc6)n5)ccc43)cc2)cc1]

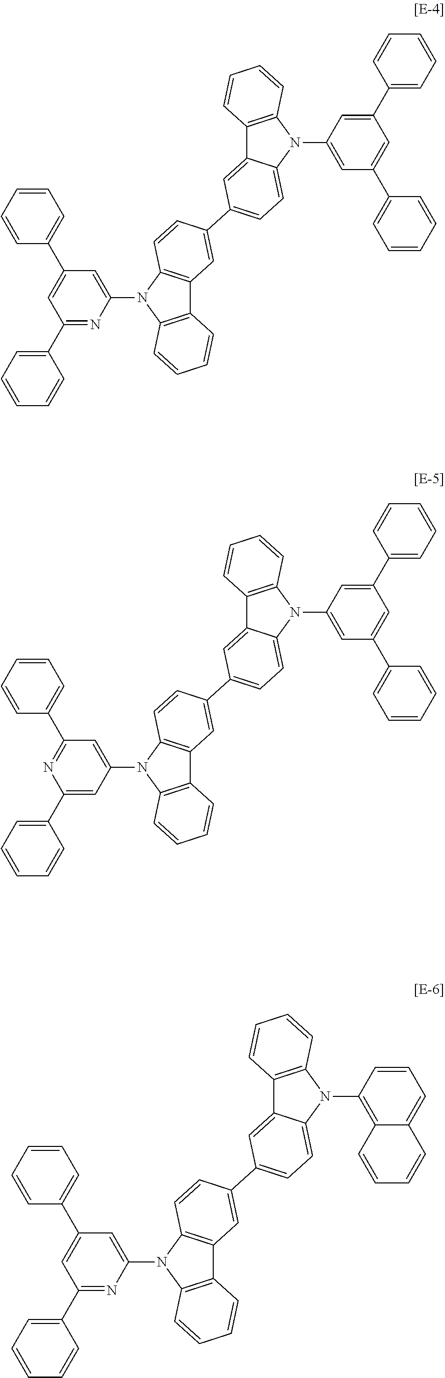 [SMILES: c1ccc(-c2cc(-c3ccccc3)cc(-n3c4ccccc4c4cc(-c5ccc6c(c5)c5ccccc5n6-c5cc(-c6ccccc6)cc(-c6ccccc6)n5)ccc43)c2)cc1 ; c1ccc(-c2cc(-c3ccccc3)cc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-c3ccccc3)nc(-n3c4ccccc4c4cc(-c5ccc6c(c5)c5ccccc5n6-c5cccc6ccccc56)ccc43)c2)cc1]

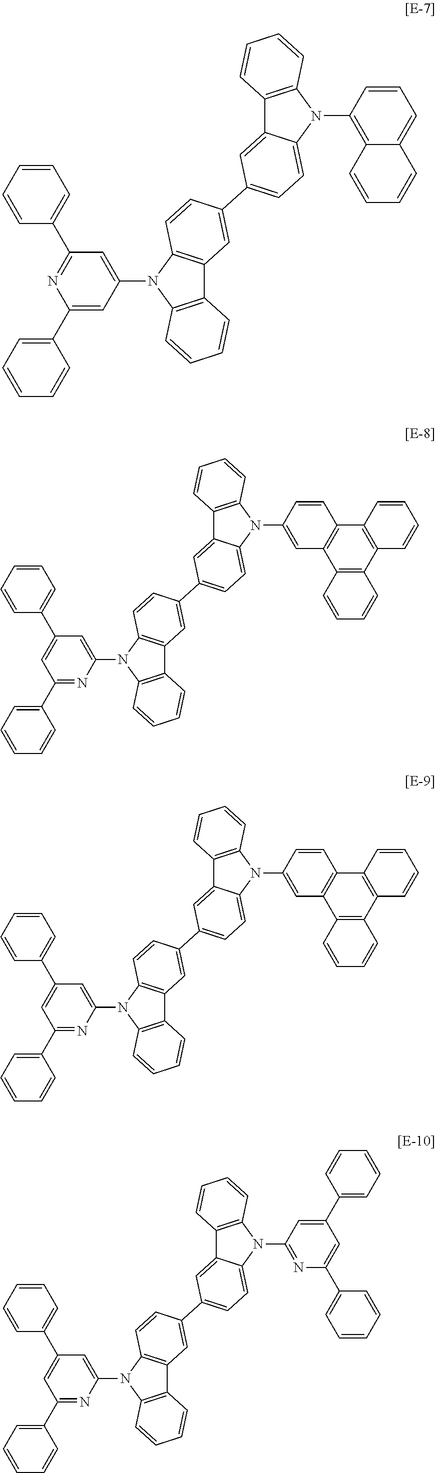 [SMILES: c1ccc(-c2cc(-n3c4ccccc4c4cc(-c5ccc6c(c5)c5ccccc5n6-c5cccc6ccccc56)ccc43)cc(-c3ccccc3)n2)cc1 ; c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1 ; c1ccc(-c2cc(-c3ccccc3)nc(-n3c4ccccc4c4cc(-c5ccc6c(c5)c5ccccc5n6-c5ccc6c7ccccc7c7ccccc7c6c5)ccc43)c2)cc1 ; c1ccc(-c2cc(-c3ccccc3)nc(-n3c4ccccc4c4cc(-c5ccc6c(c5)c5ccccc5n6-c5cc(-c6ccccc6)cc(-c6ccccc6)n5)ccc43)c2)cc1]

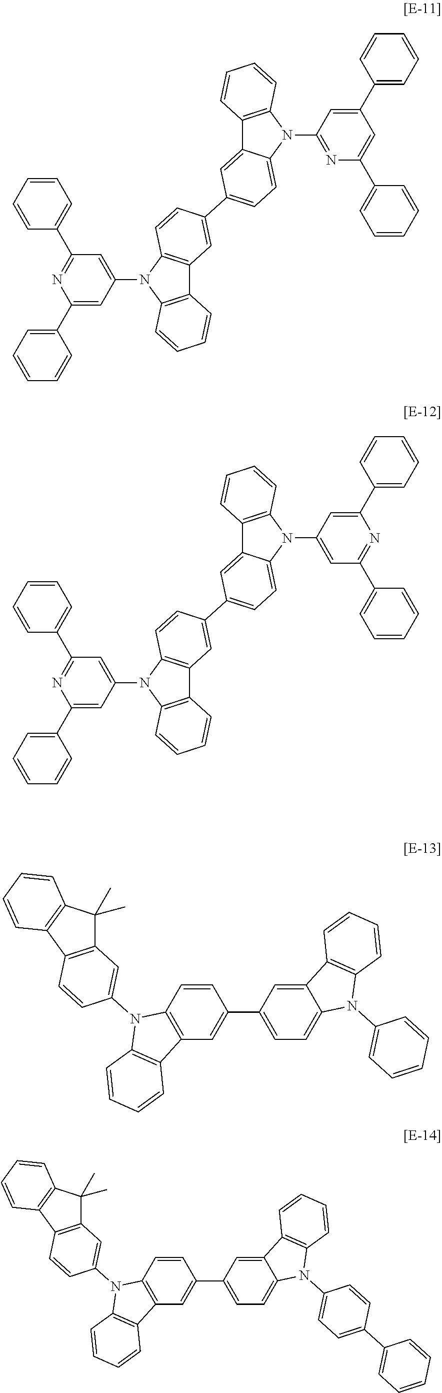 [SMILES: c1ccc(-c2cc(-c3ccccc3)nc(-n3c4ccccc4c4cc(-c5ccc6c(c5)c5ccccc5n6-c5cc(-c6ccccc6)nc(-c6ccccc6)c5)ccc43)c2)cc1 ; c1ccc(-c2cc(-n3c4ccccc4c4cc(-c5ccc6c(c5)c5ccccc5n6-c5cc(-c6ccccc6)nc(-c6ccccc6)c5)ccc43)cc(-c3ccccc3)n2)cc1 ; CC1(C)c2ccccc2-c2ccc(-n3c4ccccc4c4cc(-c5ccc6c(c5)c5ccccc5n6-c5ccccc5)ccc43)cc21 ; CC1(C)c2ccccc2-c2ccc(-n3c4ccccc4c4cc(-c5ccc6c(c5)c5ccccc5n6-c5ccc(-c6ccccc6)cc5)ccc43)cc21]

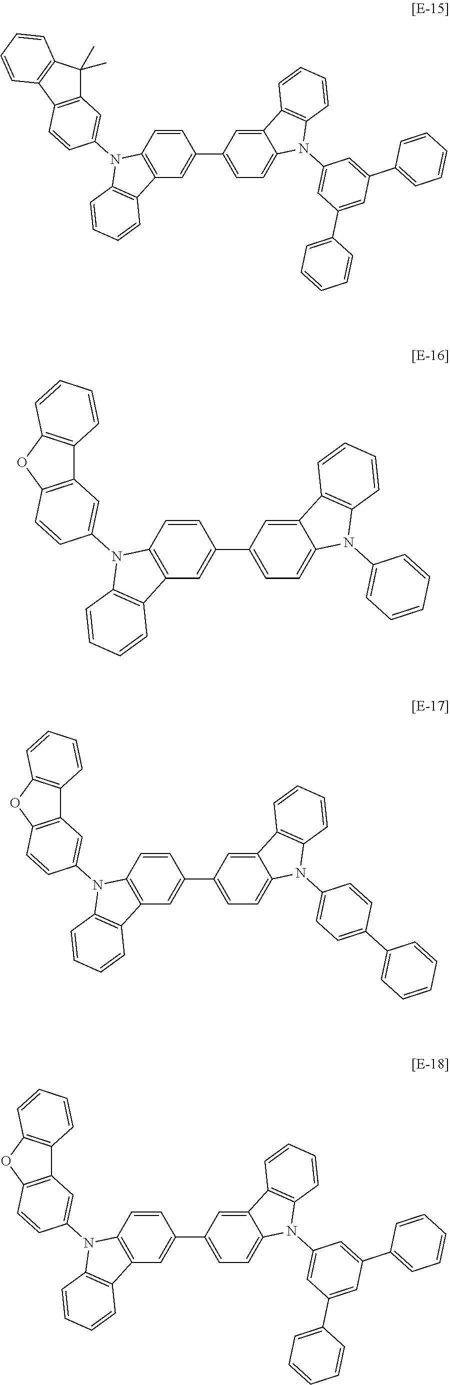 [SMILES: CC1(C)c2ccccc2-c2ccc(-n3c4ccccc4c4cc(-c5ccc6c(c5)c5ccccc5n6-c5cc(-c6ccccc6)cc(-c6ccccc6)c5)ccc43)cc21 ; c1ccc(-n2c3ccccc3c3cc(-c4ccc5c(c4)c4ccccc4n5-c4ccc5oc6ccccc6c5c4)ccc32)cc1 ; c1ccc(-c2ccc(-n3c4ccccc4c4cc(-c5ccc6c(c5)c5ccccc5n6-c5ccc6oc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6oc7ccccc7c6c5)ccc43)c2)cc1]

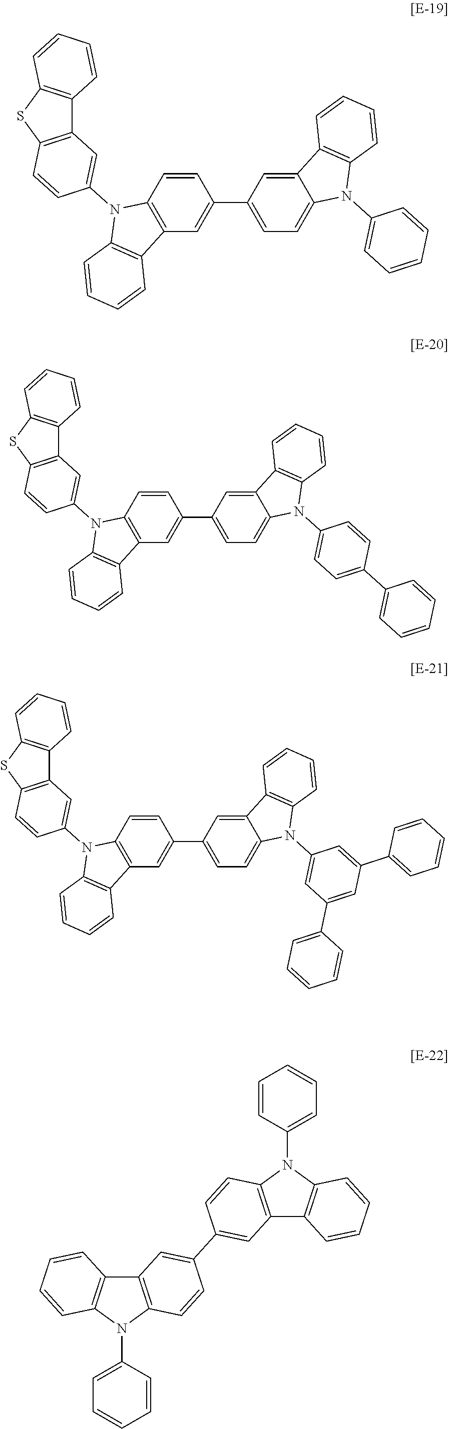 [SMILES: c1ccc(-n2c3ccccc3c3cc(-c4ccc5c(c4)c4ccccc4n5-c4ccc5sc6ccccc6c5c4)ccc32)cc1 ; c1ccc(-c2ccc(-n3c4ccccc4c4cc(-c5ccc6c(c5)c5ccccc5n6-c5ccc6sc7ccccc7c6c5)ccc43)cc2)cc1 ; c1ccc(-c2cc(-c3ccccc3)cc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccccc4)ccc32)cc1]

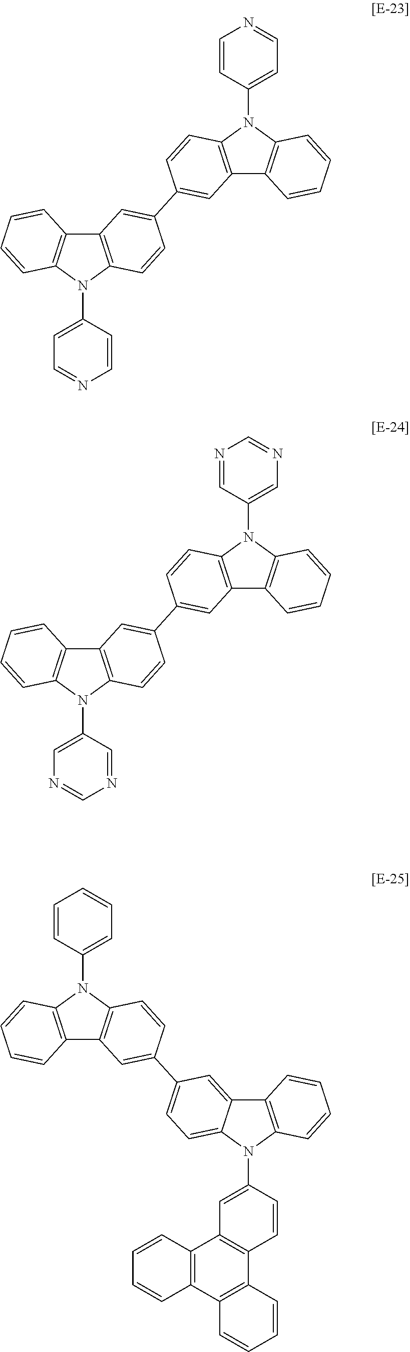 [SMILES: c1ccc2c(c1)c1cc(-c3ccc4c(c3)c3ccccc3n4-c3ccncc3)ccc1n2-c1ccncc1 ; c1ccc2c(c1)c1cc(-c3ccc4c(c3)c3ccccc3n4-c3cncnc3)ccc1n2-c1cncnc1 ; c1ccc(-n2c3ccccc3c3cc(-c4ccc5c(c4)c4ccccc4n5-c4ccc5c6ccccc6c6ccccc6c5c4)ccc32)cc1]

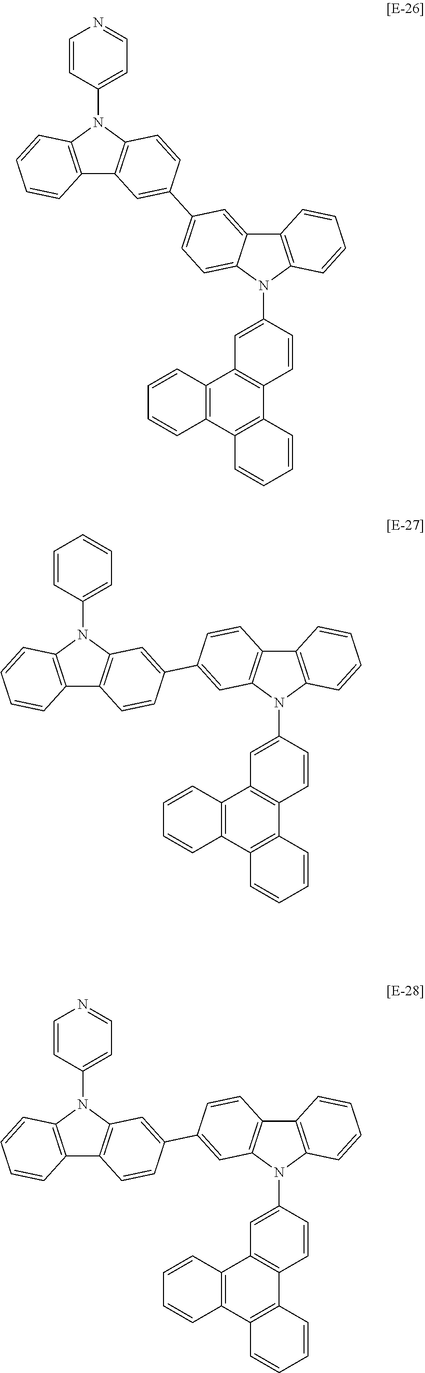 [SMILES: c1ccc2c(c1)c1ccccc1c1cc(-n3c4ccccc4c4cc(-c5ccc6c(c5)c5ccccc5n6-c5ccncc5)ccc43)ccc21 ; c1ccc(-n2c3ccccc3c3ccc(-c4ccc5c6ccccc6n(-c6ccc7c8ccccc8c8ccccc8c7c6)c5c4)cc32)cc1 ; c1ccc2c(c1)c1ccccc1c1cc(-n3c4ccccc4c4ccc(-c5ccc6c7ccccc7n(-c7ccncc7)c6c5)cc43)ccc21]

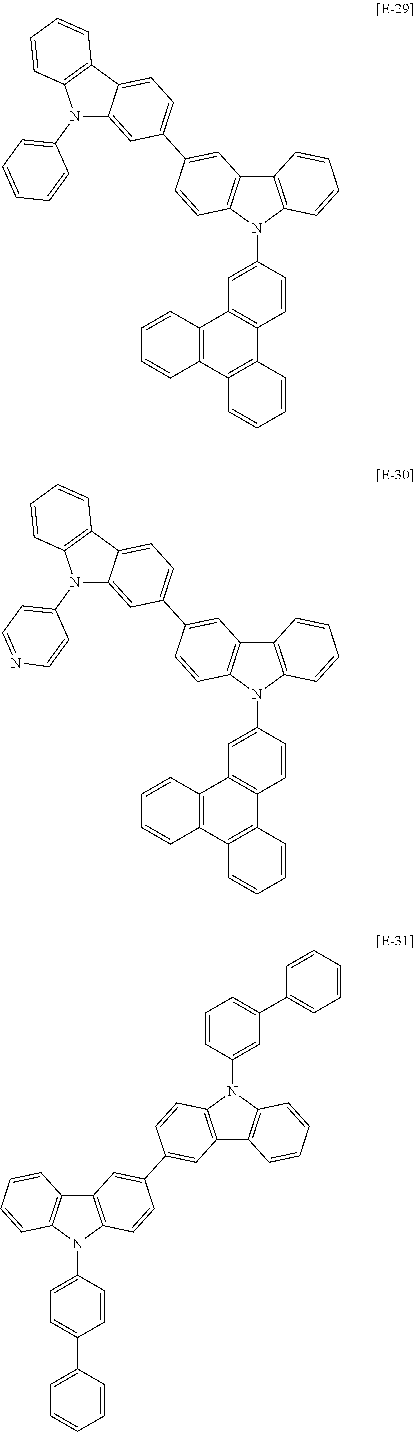 [SMILES: c1ccc(-n2c3ccccc3c3ccc(-c4ccc5c(c4)c4ccccc4n5-c4ccc5c6ccccc6c6ccccc6c5c4)cc32)cc1 ; c1ccc2c(c1)c1ccccc1c1cc(-n3c4ccccc4c4cc(-c5ccc6c7ccccc7n(-c7ccncc7)c6c5)ccc43)ccc21 ; c1ccc(-c2ccc(-n3c4ccccc4c4cc(-c5ccc6c(c5)c5ccccc5n6-c5cccc(-c6ccccc6)c5)ccc43)cc2)cc1]

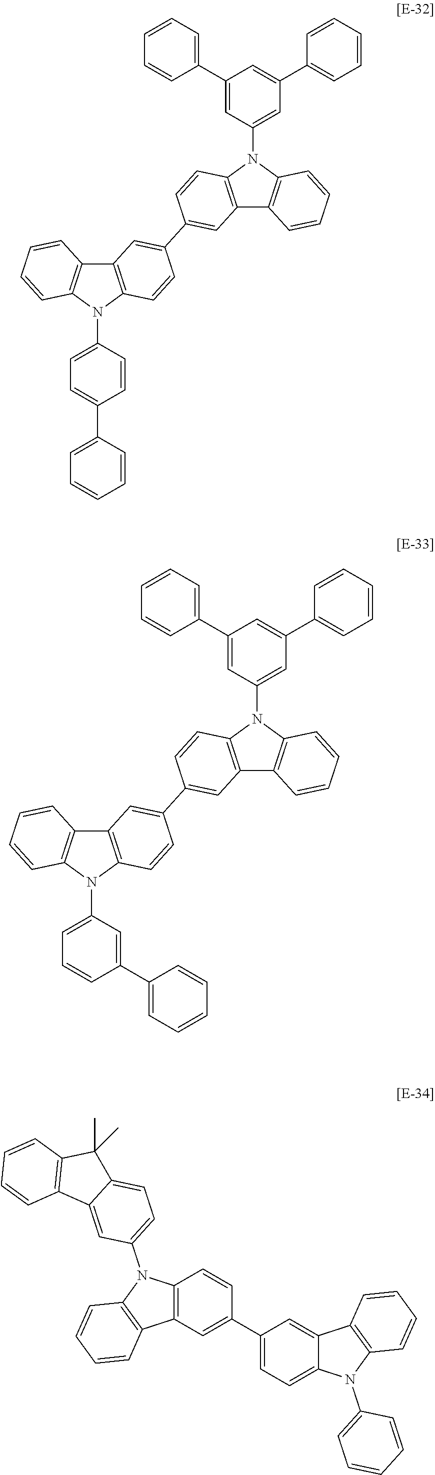 [SMILES: c1ccc(-c2ccc(-n3c4ccccc4c4cc(-c5ccc6c(c5)c5ccccc5n6-c5cc(-c6ccccc6)cc(-c6ccccc6)c5)ccc43)cc2)cc1 ; c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; CC1(C)c2ccccc2-c2cc(-n3c4ccccc4c4cc(-c5ccc6c(c5)c5ccccc5n6-c5ccccc5)ccc43)ccc21]

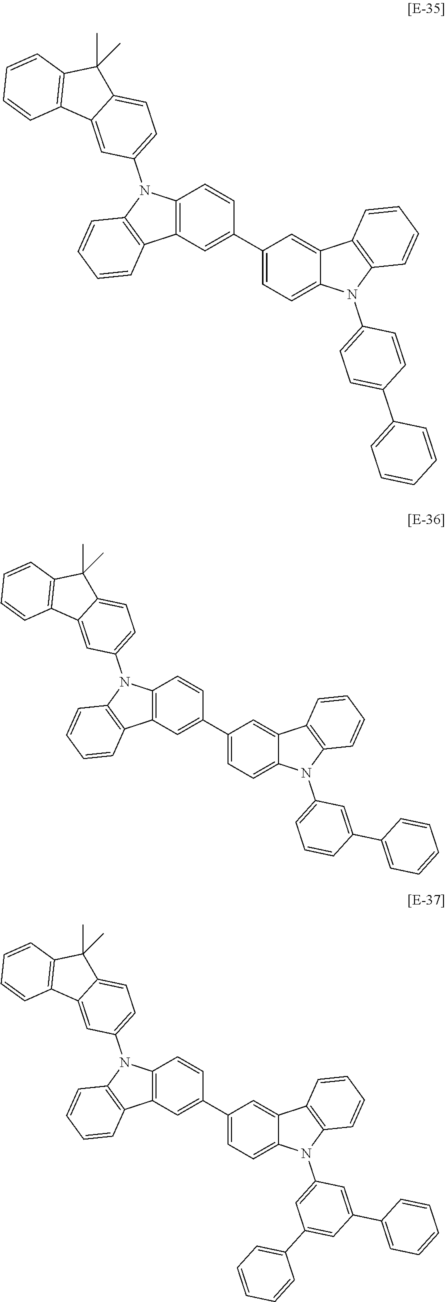 [SMILES: CC1(C)c2ccccc2-c2cc(-n3c4ccccc4c4cc(-c5ccc6c(c5)c5ccccc5n6-c5ccc(-c6ccccc6)cc5)ccc43)ccc21 ; CC1(C)c2ccccc2-c2cc(-n3c4ccccc4c4cc(-c5ccc6c(c5)c5ccccc5n6-c5cccc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccc6)c5)ccc43)ccc21]

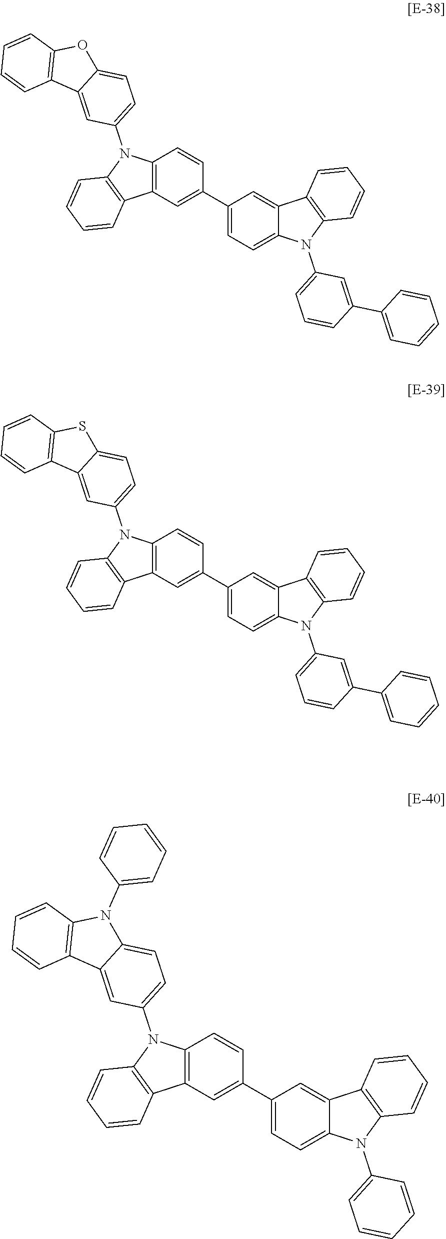 [SMILES: c1ccc(-c2cccc(-n3c4ccccc4c4cc(-c5ccc6c(c5)c5ccccc5n6-c5ccc6oc7ccccc7c6c5)ccc43)c2)cc1 ; c1ccc(-c2cccc(-n3c4ccccc4c4cc(-c5ccc6c(c5)c5ccccc5n6-c5ccc6sc7ccccc7c6c5)ccc43)c2)cc1 ; c1ccc(-n2c3ccccc3c3cc(-c4ccc5c(c4)c4ccccc4n5-c4ccc5c(c4)c4ccccc4n5-c4ccccc4)ccc32)cc1]

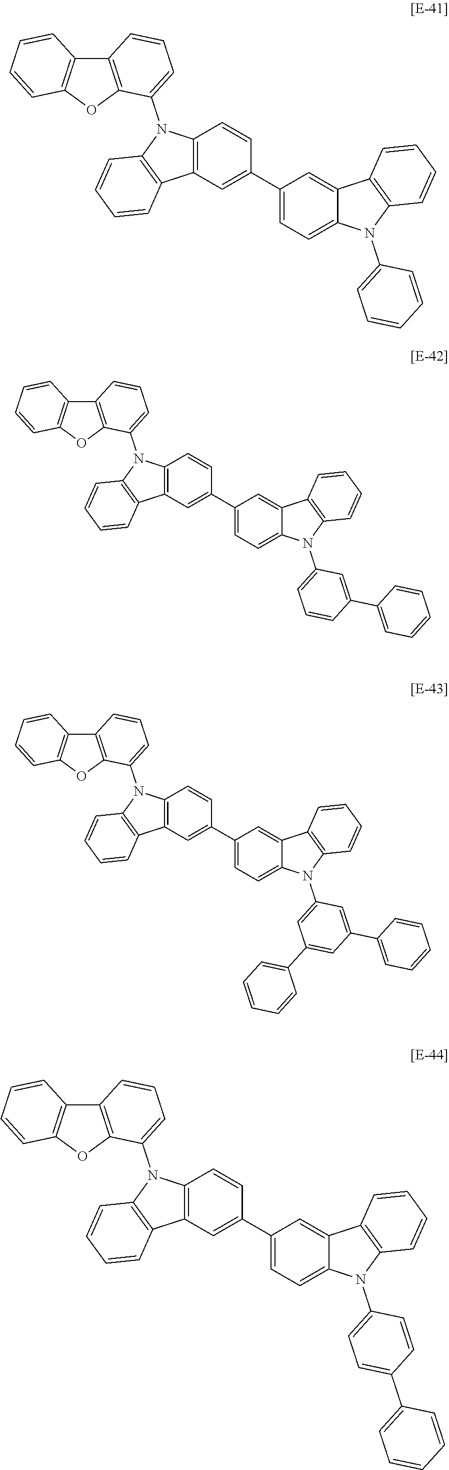 [SMILES: c1ccc(-n2c3ccccc3c3cc(-c4ccc5c(c4)c4ccccc4n5-c4cccc5c4oc4ccccc45)ccc32)cc1 ; c1ccc(-c2cccc(-n3c4ccccc4c4cc(-c5ccc6c(c5)c5ccccc5n6-c5cccc6c5oc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5oc5ccccc56)ccc43)c2)cc1 ; c1ccc(-c2ccc(-n3c4ccccc4c4cc(-c5ccc6c(c5)c5ccccc5n6-c5cccc6c5oc5ccccc56)ccc43)cc2)cc1]

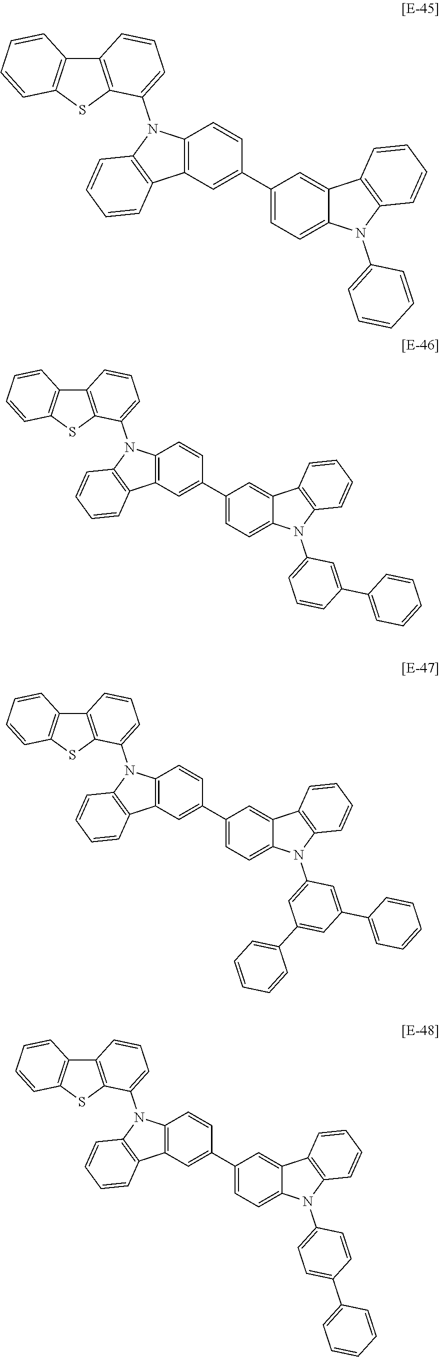 [SMILES: c1ccc(-n2c3ccccc3c3cc(-c4ccc5c(c4)c4ccccc4n5-c4cccc5c4sc4ccccc45)ccc32)cc1 ; c1ccc(-c2cccc(-n3c4ccccc4c4cc(-c5ccc6c(c5)c5ccccc5n6-c5cccc6c5sc5ccccc56)ccc43)c2)cc1 ; c1ccc(-c2cc(-c3ccccc3)cc(-n3c4ccccc4c4cc(-c5ccc6c(c5)c5ccccc5n6-c5cccc6c5sc5ccccc56)ccc43)c2)cc1 ; c1ccc(-c2ccc(-n3c4ccccc4c4cc(-c5ccc6c(c5)c5ccccc5n6-c5cccc6c5sc5ccccc56)ccc43)cc2)cc1]

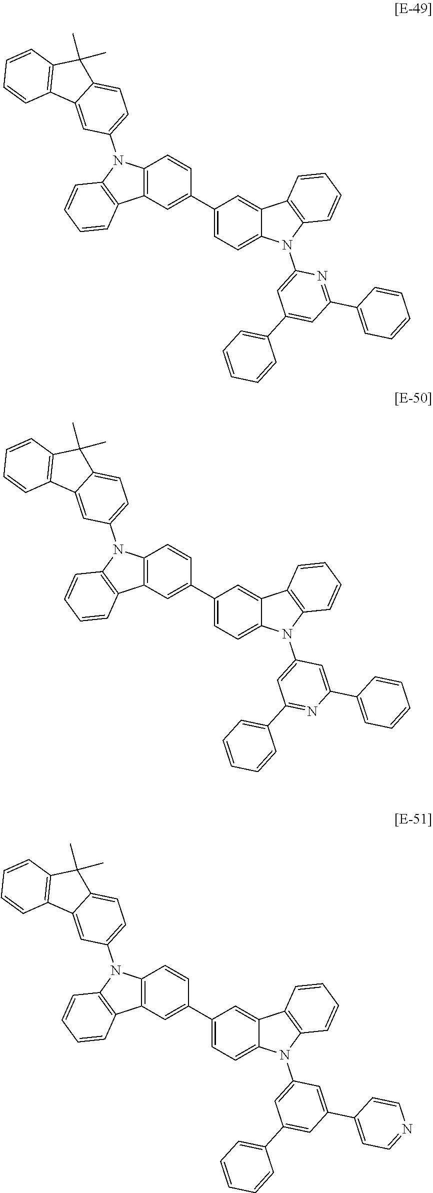 [SMILES: CC1(C)c2ccccc2-c2cc(-n3c4ccccc4c4cc(-c5ccc6c(c5)c5ccccc5n6-c5cc(-c6ccccc6)cc(-c6ccccc6)n5)ccc43)ccc21 ; CC1(C)c2ccccc2-c2cc(-n3c4ccccc4c4cc(-c5ccc6c(c5)c5ccccc5n6-c5cc(-c6ccccc6)nc(-c6ccccc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccncc6)c5)ccc43)ccc21]

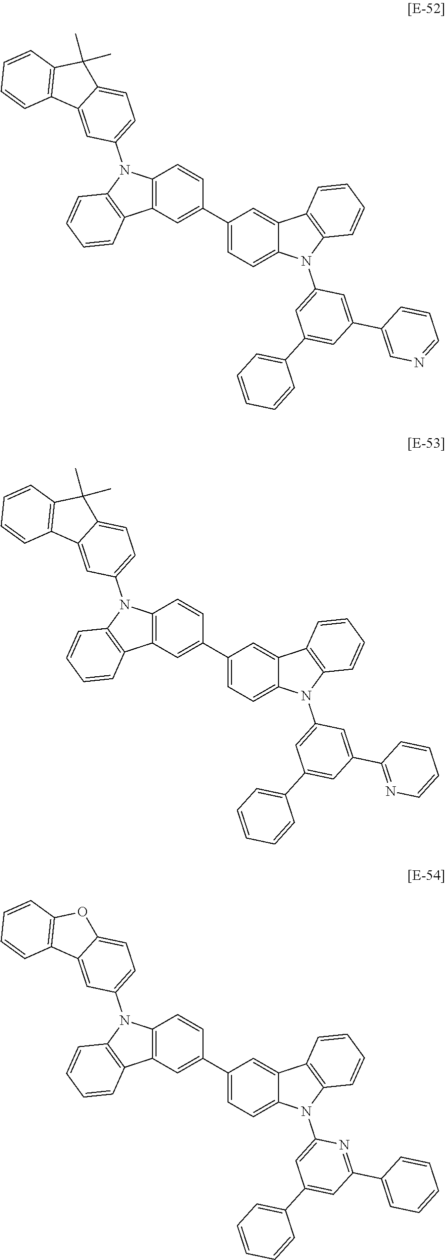 [SMILES: CC1(C)c2ccccc2-c2cc(-n3c4ccccc4c4cc(-c5ccc6c(c5)c5ccccc5n6-c5cc(-c6ccccc6)cc(-c6cccnc6)c5)ccc43)ccc21 ; CC1(C)c2ccccc2-c2cc(-n3c4ccccc4c4cc(-c5ccc6c(c5)c5ccccc5n6-c5cc(-c6ccccc6)cc(-c6ccccn6)c5)ccc43)ccc21 ; c1ccc(-c2cc(-c3ccccc3)nc(-n3c4ccccc4c4cc(-c5ccc6c(c5)c5ccccc5n6-c5ccc6oc7ccccc7c6c5)ccc43)c2)cc1]

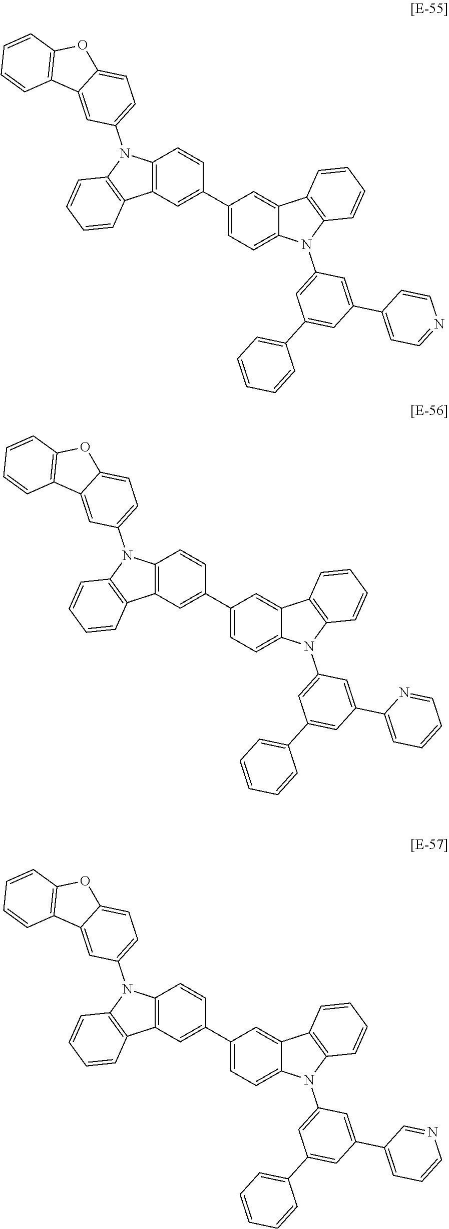 [SMILES: c1ccc(-c2cc(-c3ccncc3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6oc7ccccc7c6c5)ccc43)c2)cc1 ; c1ccc(-c2cc(-c3cccnc3)cc(-n3c4ccccc4c4cc(-c5ccc6c(c5)c5ccccc5n6-c5ccc6oc7ccccc7c6c5)ccc43)c2)cc1]

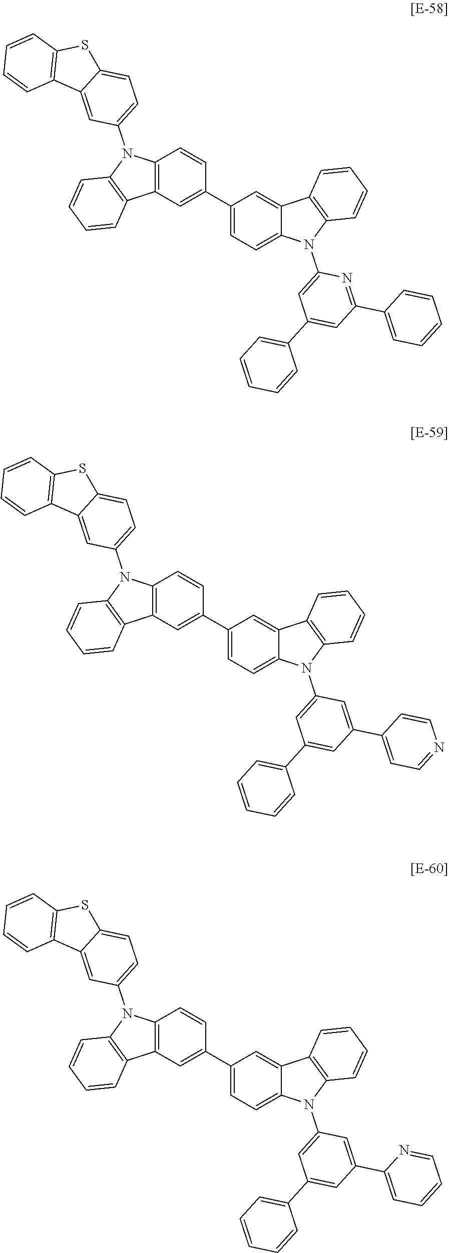 [SMILES: c1ccc(-c2cc(-c3ccccc3)nc(-n3c4ccccc4c4cc(-c5ccc6c(c5)c5ccccc5n6-c5ccc6sc7ccccc7c6c5)ccc43)c2)cc1 ; c1ccc(-c2cc(-c3ccncc3)cc(-n3c4ccccc4c4cc(-c5ccc6c(c5)c5ccccc5n6-c5ccc6sc7ccccc7c6c5)ccc43)c2)cc1 ; c1ccc(-c2cc(-c3ccccn3)cc(-n3c4ccccc4c4cc(-c5ccc6c(c5)c5ccccc5n6-c5ccc6sc7ccccc7c6c5)ccc43)c2)cc1]

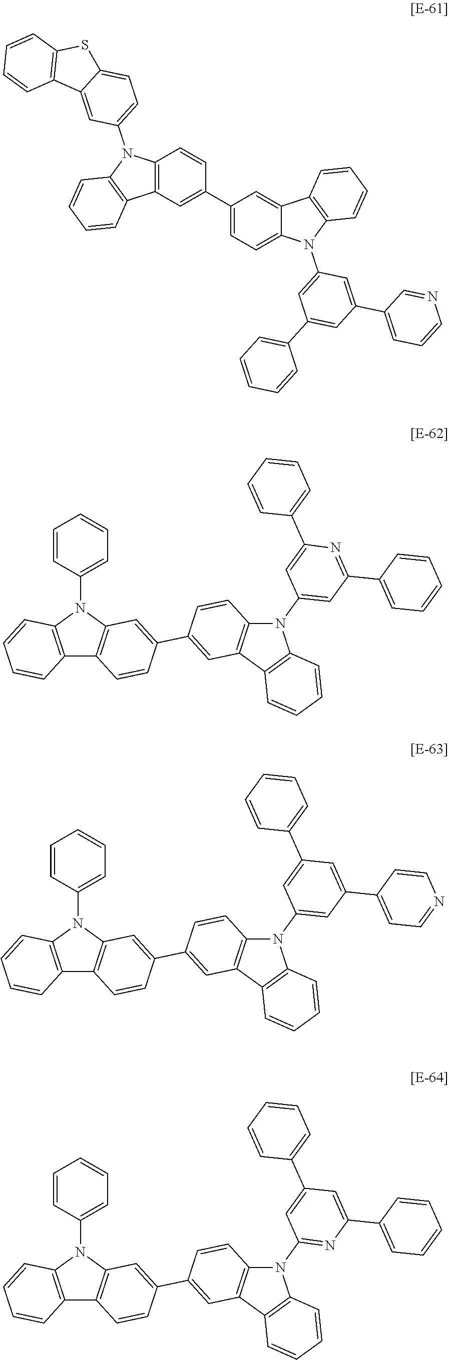 [SMILES: c1ccc(-c2cc(-c3cccnc3)cc(-n3c4ccccc4c4cc(-c5ccc6c(c5)c5ccccc5n6-c5ccc6sc7ccccc7c6c5)ccc43)c2)cc1 ; c1ccc(-c2cc(-n3c4ccccc4c4cc(-c5ccc6c7ccccc7n(-c7ccccc7)c6c5)ccc43)cc(-c3ccccc3)n2)cc1 ; c1ccc(-c2cc(-c3ccncc3)cc(-n3c4ccccc4c4cc(-c5ccc6c7ccccc7n(-c7ccccc7)c6c5)ccc43)c2)cc1 ; c1ccc(-c2cc(-c3ccccc3)nc(-n3c4ccccc4c4cc(-c5ccc6c7ccccc7n(-c7ccccc7)c6c5)ccc43)c2)cc1]

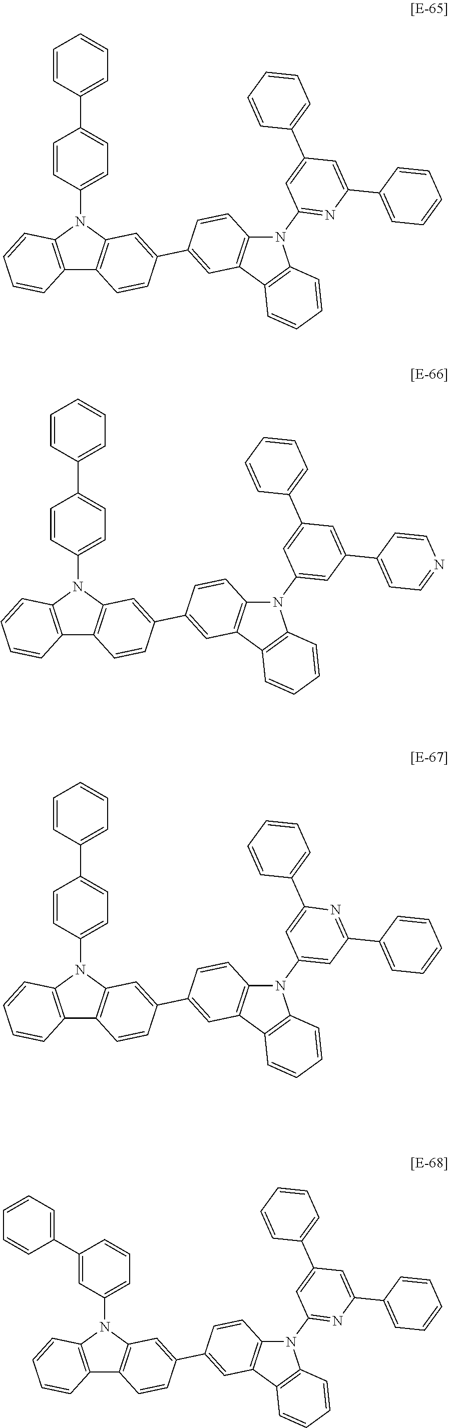 [SMILES: c1ccc(-c2ccc(-n3c4ccccc4c4ccc(-c5ccc6c(c5)c5ccccc5n6-c5cc(-c6ccccc6)cc(-c6ccccc6)n5)cc43)cc2)cc1 ; c1ccc(-c2ccc(-n3c4ccccc4c4ccc(-c5ccc6c(c5)c5ccccc5n6-c5cc(-c6ccccc6)cc(-c6ccncc6)c5)cc43)cc2)cc1 ; c1ccc(-c2ccc(-n3c4ccccc4c4ccc(-c5ccc6c(c5)c5ccccc5n6-c5cc(-c6ccccc6)nc(-c6ccccc6)c5)cc43)cc2)cc1 ; c1ccc(-c2cccc(-n3c4ccccc4c4ccc(-c5ccc6c(c5)c5ccccc5n6-c5cc(-c6ccccc6)cc(-c6ccccc6)n5)cc43)c2)cc1]

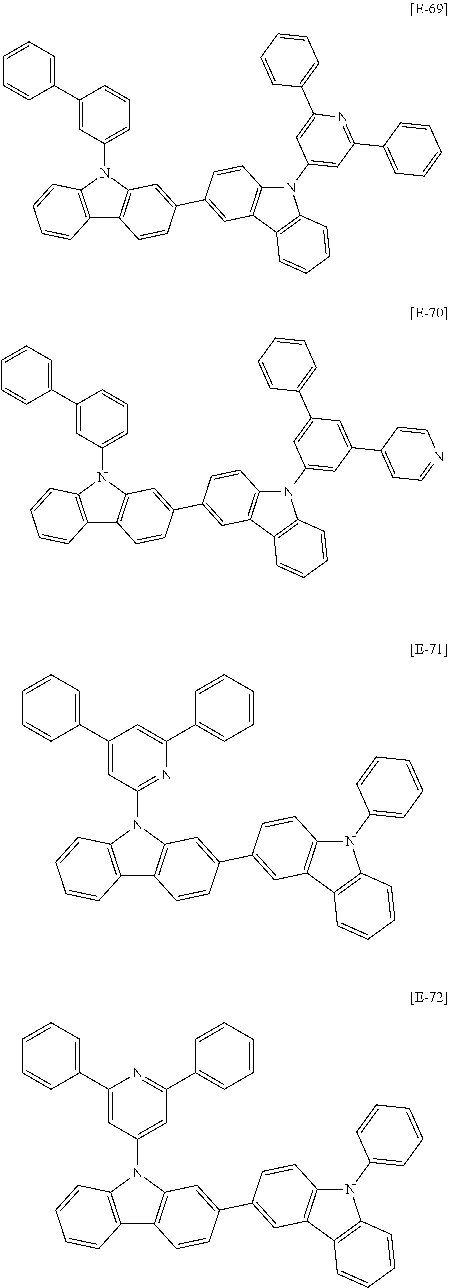 [SMILES: c1ccc(-c2cccc(-n3c4ccccc4c4ccc(-c5ccc6c(c5)c5ccccc5n6-c5cc(-c6ccccc6)nc(-c6ccccc6)c5)cc43)c2)cc1 ; c1ccc(-c2cccc(-n3c4ccccc4c4ccc(-c5ccc6c(c5)c5ccccc5n6-c5cc(-c6ccccc6)cc(-c6ccncc6)c5)cc43)c2)cc1 ; c1ccc(-c2cc(-c3ccccc3)nc(-n3c4ccccc4c4ccc(-c5ccc6c(c5)c5ccccc5n6-c5ccccc5)cc43)c2)cc1 ; c1ccc(-c2cc(-n3c4ccccc4c4ccc(-c5ccc6c(c5)c5ccccc5n6-c5ccccc5)cc43)cc(-c3ccccc3)n2)cc1]

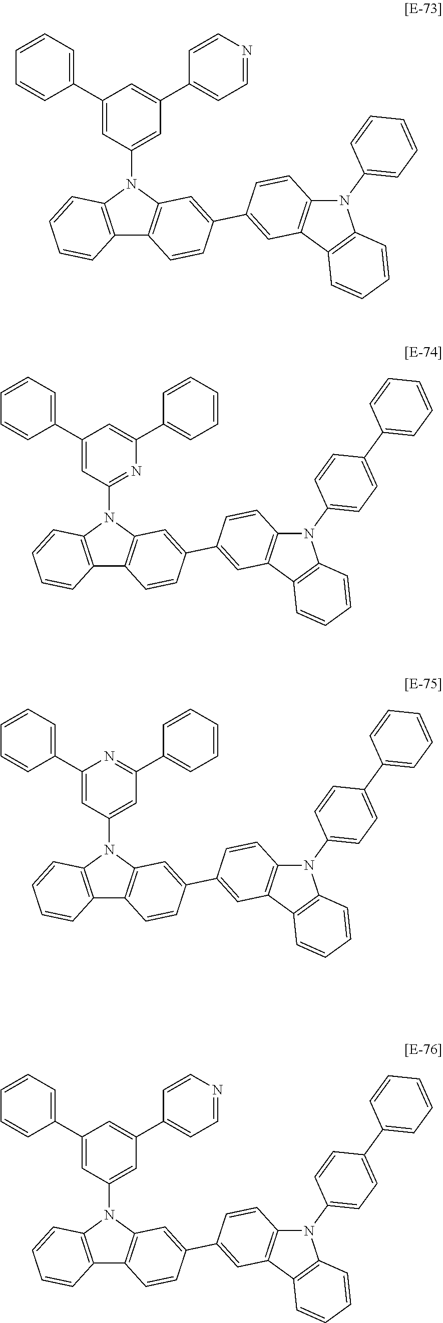 [SMILES: c1ccc(-c2cc(-c3ccncc3)cc(-n3c4ccccc4c4ccc(-c5ccc6c(c5)c5ccccc5n6-c5ccccc5)cc43)c2)cc1 ; c1ccc(-c2ccc(-n3c4ccccc4c4cc(-c5ccc6c7ccccc7n(-c7cc(-c8ccccc8)cc(-c8ccccc8)n7)c6c5)ccc43)cc2)cc1 ; c1ccc(-c2ccc(-n3c4ccccc4c4cc(-c5ccc6c7ccccc7n(-c7cc(-c8ccccc8)nc(-c8ccccc8)c7)c6c5)ccc43)cc2)cc1 ; c1ccc(-c2ccc(-n3c4ccccc4c4cc(-c5ccc6c7ccccc7n(-c7cc(-c8ccccc8)cc(-c8ccncc8)c7)c6c5)ccc43)cc2)cc1]

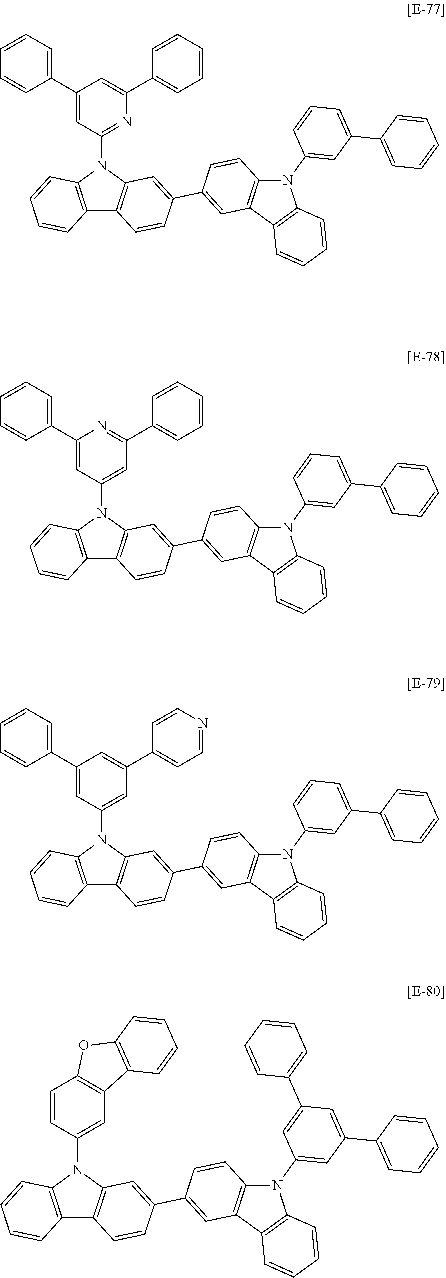 [SMILES: c1ccc(-c2cccc(-n3c4ccccc4c4cc(-c5ccc6c7ccccc7n(-c7cc(-c8ccccc8)cc(-c8ccccc8)n7)c6c5)ccc43)c2)cc1 ; c1ccc(-c2cccc(-n3c4ccccc4c4cc(-c5ccc6c7ccccc7n(-c7cc(-c8ccccc8)nc(-c8ccccc8)c7)c6c5)ccc43)c2)cc1 ; c1ccc(-c2cccc(-n3c4ccccc4c4cc(-c5ccc6c7ccccc7n(-c7cc(-c8ccccc8)cc(-c8ccncc8)c7)c6c5)ccc43)c2)cc1 ; c1ccc(-c2cc(-c3ccccc3)cc(-n3c4ccccc4c4cc(-c5ccc6c7ccccc7n(-c7ccc8oc9ccccc9c8c7)c6c5)ccc43)c2)cc1]

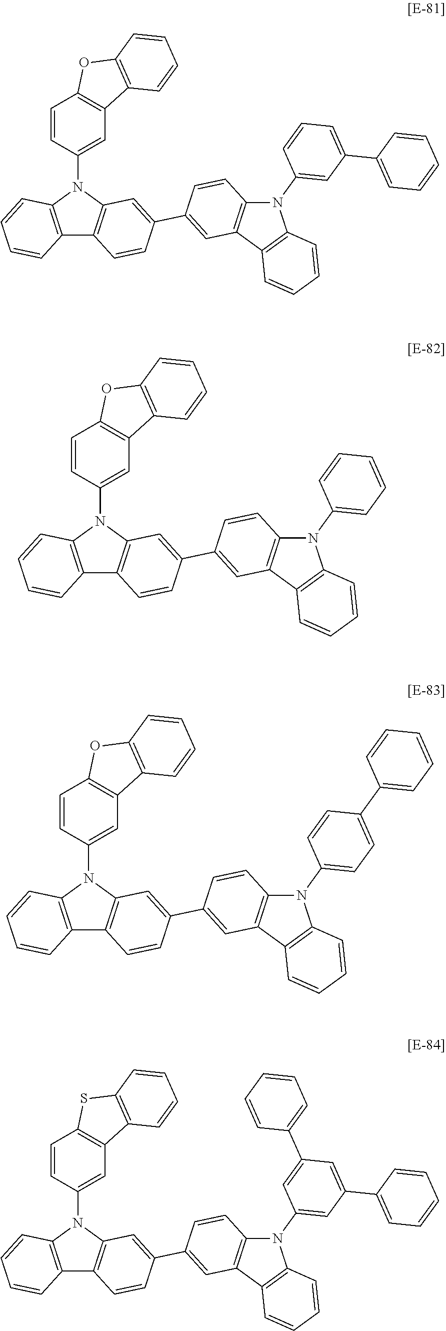 [SMILES: c1ccc(-c2cccc(-n3c4ccccc4c4cc(-c5ccc6c7ccccc7n(-c7ccc8oc9ccccc9c8c7)c6c5)ccc43)c2)cc1 ; c1ccc(-n2c3ccccc3c3cc(-c4ccc5c6ccccc6n(-c6ccc7oc8ccccc8c7c6)c5c4)ccc32)cc1 ; c1ccc(-c2ccc(-n3c4ccccc4c4cc(-c5ccc6c7ccccc7n(-c7ccc8sc9ccccc9c8c7)c6c5)ccc43)cc2)cc1 ; c1ccc(-c2cc(-c3ccccc3)cc(-n3c4ccccc4c4cc(-c5ccc6c7ccccc7n(-c7ccc8sc9ccccc9c8c7)c6c5)ccc43)c2)cc1]

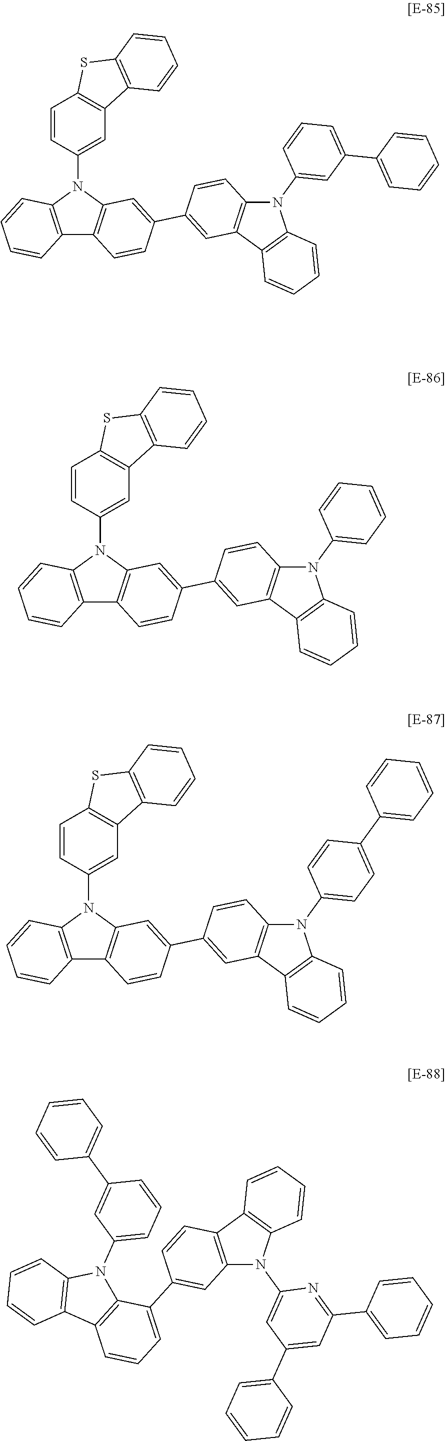 [SMILES: c1ccc(-c2cccc(-n3c4ccccc4c4cc(-c5ccc6c7ccccc7n(-c7ccc8sc9ccccc9c8c7)c6c5)ccc43)c2)cc1 ; c1ccc(-n2c3ccccc3c3cc(-c4ccc5c6ccccc6n(-c6ccc7sc8ccccc8c7c6)c5c4)ccc32)cc1 ; c1ccc(-c2ccc(-n3c4ccccc4c4cc(-c5ccc6c7ccccc7n(-c7ccc8sc9ccccc9c8c7)c6c5)ccc43)cc2)cc1 ; c1ccc(-c2cccc(-n3c4ccccc4c4cccc(-c5ccc6c7ccccc7n(-c7cc(-c8ccccc8)cc(-c8ccccc8)n7)c6c5)c43)c2)cc1]

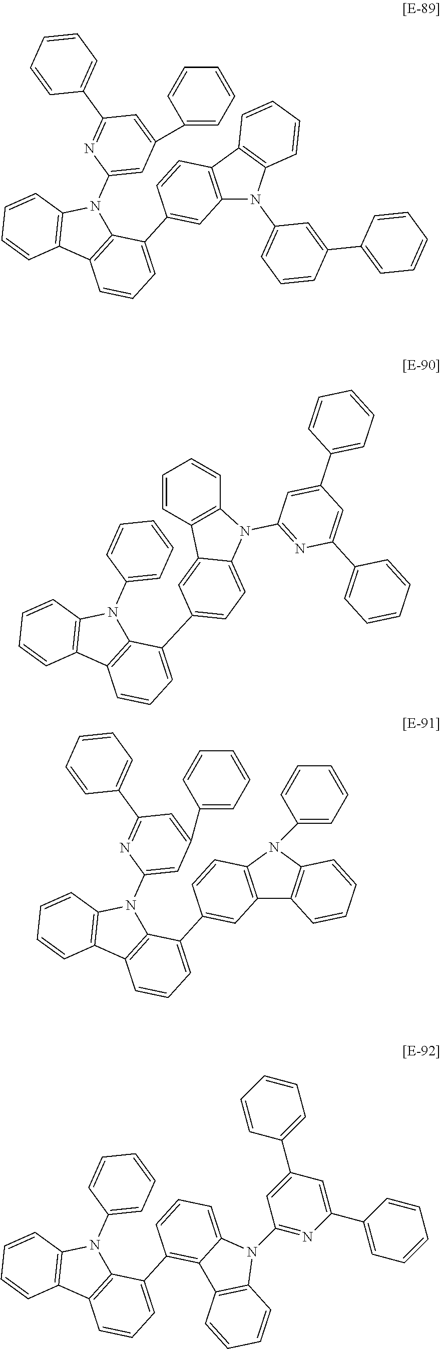

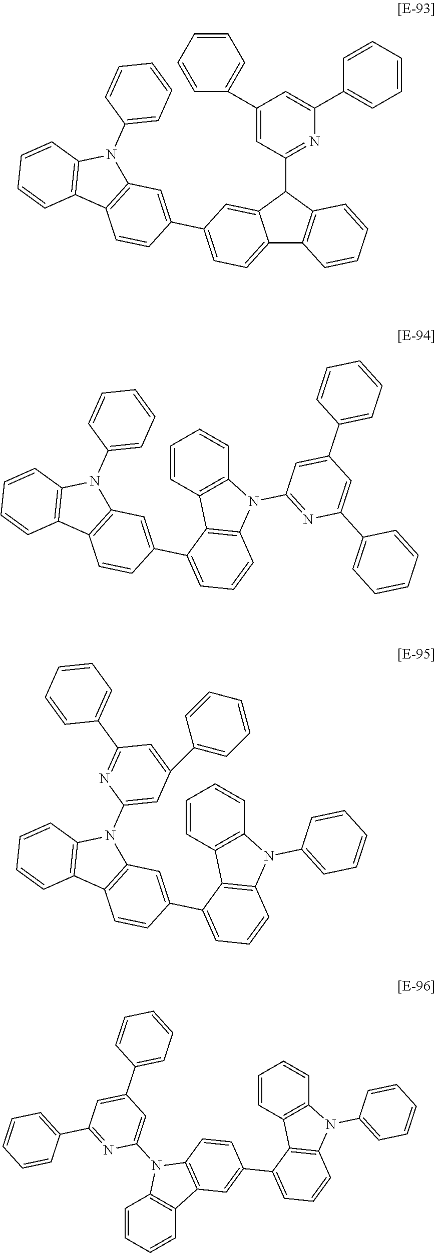 [SMILES: c1ccc(-c2cc(-c3ccccc3)nc(C3c4ccccc4-c4ccc(-c5ccc6c7ccccc7n(-c7ccccc7)c6c5)cc43)c2)cc1 ; c1ccc(-c2cc(-c3ccccc3)nc(-n3c4ccccc4c4c(-c5ccc6c7ccccc7n(-c7ccccc7)c6c5)cccc43)c2)cc1 ; c1ccc(-c2cc(-c3ccccc3)nc(-n3c4ccccc4c4ccc(-c5cccc6c5c5ccccc5n6-c5ccccc5)cc43)c2)cc1 ; c1ccc(-c2cc(-c3ccccc3)nc(-n3c4ccccc4c4cc(-c5cccc6c5c5ccccc5n6-c5ccccc5)ccc43)c2)cc1]

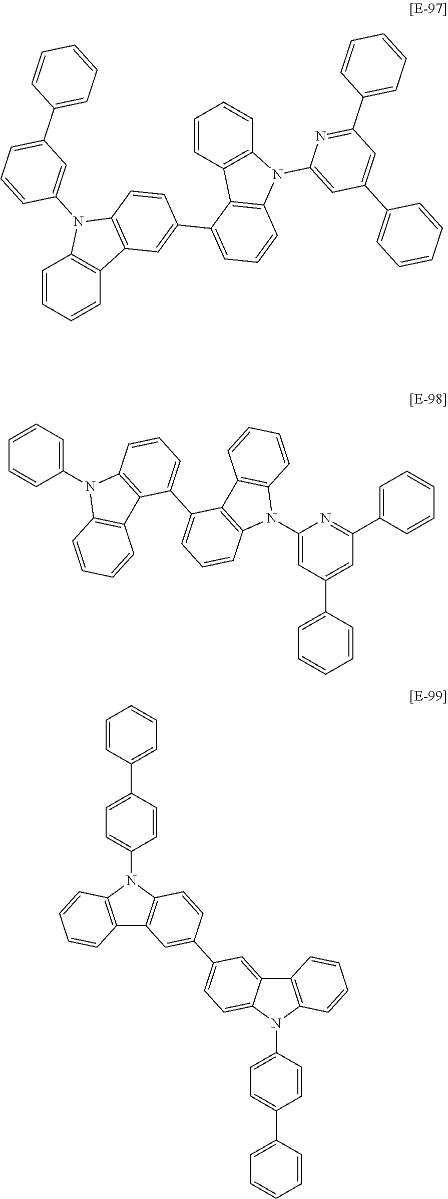 [SMILES: c1ccc(-c2cccc(-n3c4ccccc4c4cc(-c5cccc6c5c5ccccc5n6-c5cc(-c6ccccc6)cc(-c6ccccc6)n5)ccc43)c2)cc1 ; c1ccc(-c2cc(-c3ccccc3)nc(-n3c4ccccc4c4c(-c5cccc6c5c5ccccc5n6-c5ccccc5)cccc43)c2)cc1 ; c1ccc(-c2ccc(-n3c4ccccc4c4cc(-c5ccc6c(c5)c5ccccc5n6-c5ccc(-c6ccccc6)cc5)ccc43)cc2)cc1]

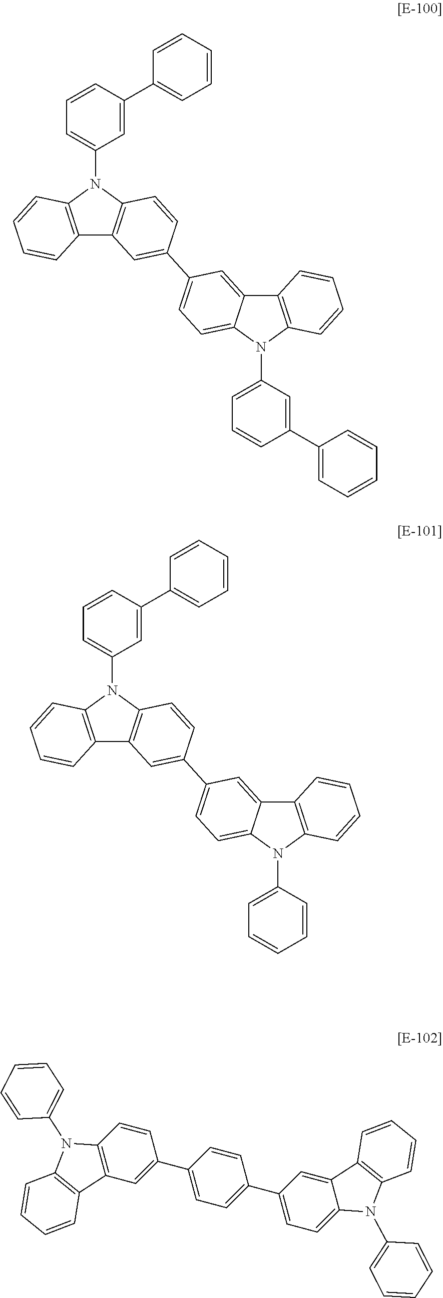 [SMILES: c1ccc(-c2cccc(-n3c4ccccc4c4cc(-c5ccc6c(c5)c5ccccc5n6-c5cccc(-c6ccccc6)c5)ccc43)c2)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc(-c5ccc6c(c5)c5ccccc5n6-c5ccccc5)cc4)ccc32)cc1]

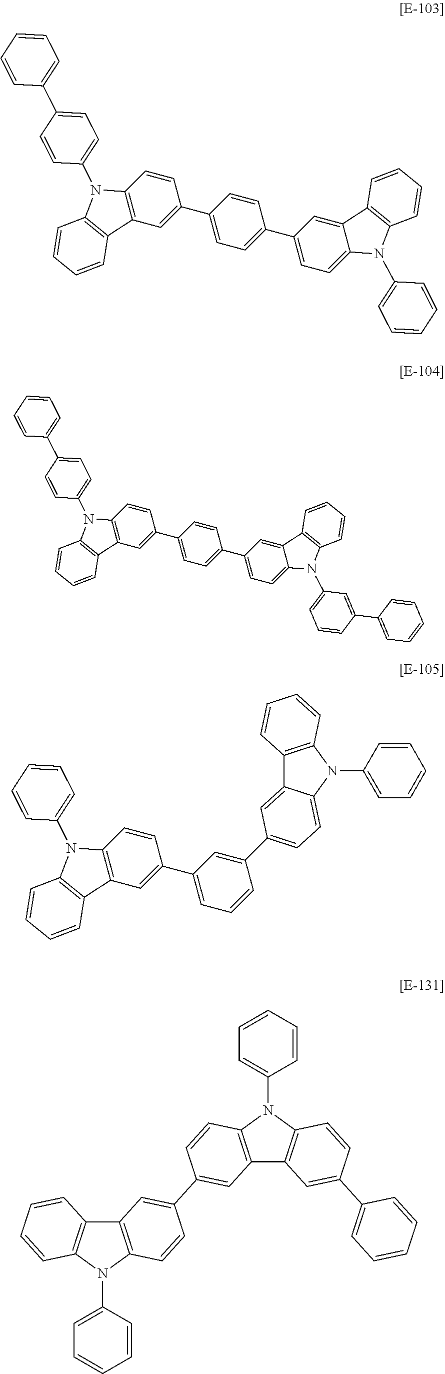 [SMILES: c1ccc(-c2ccc(-n3c4ccccc4c4cc(-c5ccc(-c6ccc7c(c6)c6ccccc6n7-c6ccccc6)cc5)ccc43)cc2)cc1 ; c1ccc(-c2ccc(-n3c4ccccc4c4cc(-c5ccc(-c6ccc7c(c6)c6ccccc6n7-c6cccc(-c7ccccc7)c6)cc5)ccc43)cc2)cc1 ; c1ccc(-n2c3ccccc3c3cc(-c4cccc(-c5ccc6c(c5)c5ccccc5n6-c5ccccc5)c4)ccc32)cc1 ; c1ccc(-c2ccc3c(c2)c2cc(-c4ccc5c(c4)c4ccccc4n5-c4ccccc4)ccc2n3-c2ccccc2)cc1]

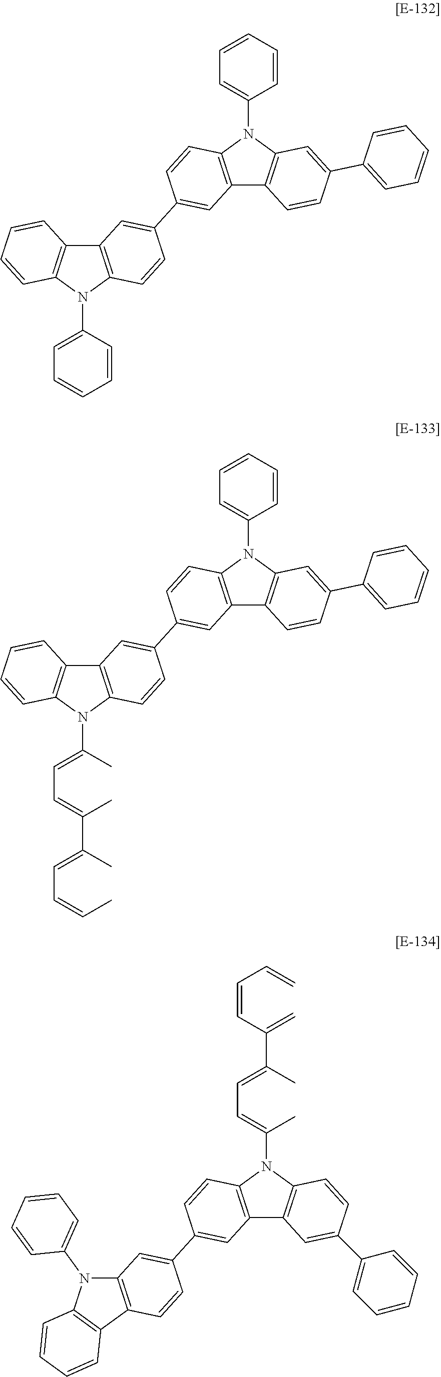 [SMILES: c1ccc(-c2ccc3c4cc(-c5ccc6c(c5)c5ccccc5n6-c5ccccc5)ccc4n(-c4ccccc4)c3c2)cc1 ; C\C=C/C=C(C)/C(C)=C/C=C(\C)n1c2ccccc2c2cc(-c3ccc4c(c3)c3ccc(-c5ccccc5)cc3n4-c3ccccc3)ccc21 ; C=C/C=C\C(=C)/C(C)=C/C=C(\C)n1c2ccc(-c3ccccc3)cc2c2cc(-c3ccc4c5ccccc5n(-c5ccccc5)c4c3)ccc21]

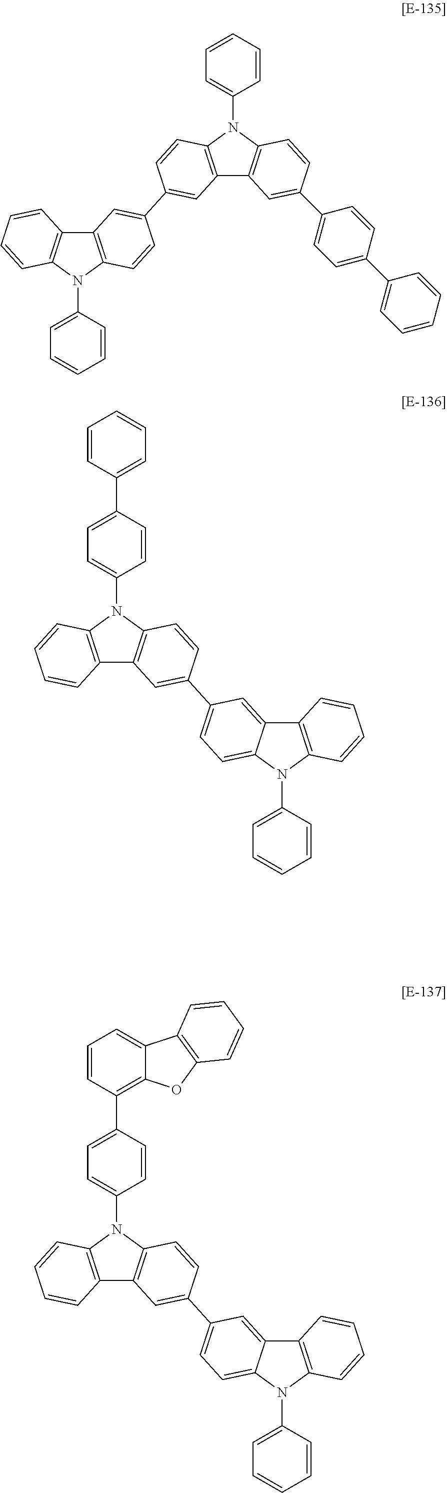 [SMILES: c1ccc(-c2ccc(-c3ccc4c(c3)c3cc(-c5ccc6c(c5)c5ccccc5n6-c5ccccc5)ccc3n4-c3ccccc3)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-n2c3ccccc3c3cc(-c4ccc5c(c4)c4ccccc4n5-c4ccc(-c5cccc6c5oc5ccccc56)cc4)ccc32)cc1]

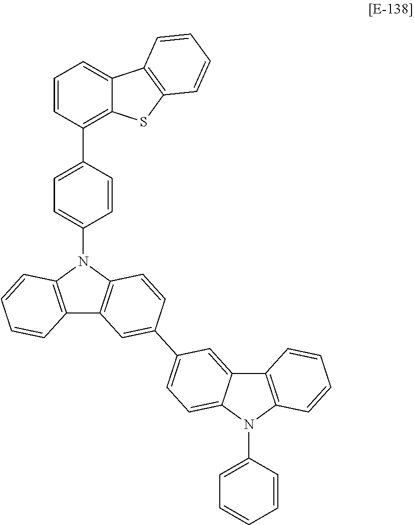 [SMILES: c1ccc(-n2c3ccccc3c3cc(-c4ccc5c(c4)c4ccccc4n5-c4ccc(-c5cccc6c5sc5ccccc56)cc4)ccc32)cc1]

For example, the second organic compound may be an indolocarbazole compound represented by a combination of Chemical Formulae 7B-1 and 7B-2:

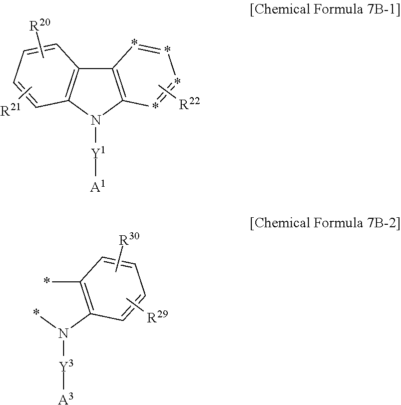

In Chemical Formulae 7B-1 and 7B-2, Y¹ and Y³ may independently be a single bond, a substituted or unsubstituted C6 to C30 arylene group, divalent substituted or unsubstituted C2 to C30 heterocyclic group, or a combination thereof,

A¹ and A³ may independently be a substituted or unsubstituted C6 to C30 aryl group, a substituted or unsubstituted C2 to C30 heterocyclic group, or a combination thereof,

R²⁰ to R²², R²⁹, and R³⁰ may independently be hydrogen, deuterium, a substituted or unsubstituted C1 to C20 alkyl group, a substituted or unsubstituted C6 to C30 aryl group, a substituted or unsubstituted C2 to C30 heterocyclic group, or a combination thereof, and

two adjacent *'s of Chemical Formula 7B-1 are bonded with two *'s of Chemical Formula 7B-2.

For example, Y¹ and Y³ of Chemical Formulae 7B-1 and 7B-2 may independently be a single bond, a substituted or unsubstituted phenylene group, or a substituted or unsubstituted biphenylene group.

For example, A¹ and A³ of Chemical Formulae 7B-1 and 7B-2 may independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, or a substituted or unsubstituted triphenylene group, a substituted or unsubstituted pyridinyl group, a substituted or unsubstituted dibenzothiophenyl group, a substituted or unsubstituted dibenzofuranyl group, a substituted or unsubstituted carbazolyl group, a substituted or unsubstituted fluorenyl group, or a combination thereof.

For example, the indolocarbazole compound represented by a combination of Chemical Formulae 7B-1 and 7B-2 may be represented by Chemical Formula 7B-c:

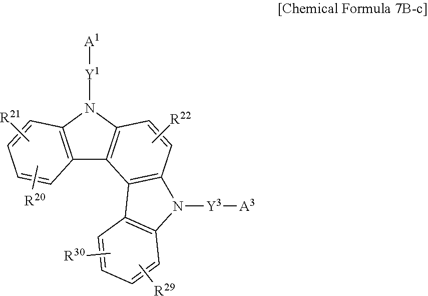

In Chemical Formula 7B-c, Y¹, Y³, A¹, A³, R²⁰ to R²², R²⁹, and R³⁰ are the same as described above.

For example, the compound represented by a combination of Chemical Formulae 7B-1 and 7B-2 may be one of compounds listed in Group 5:

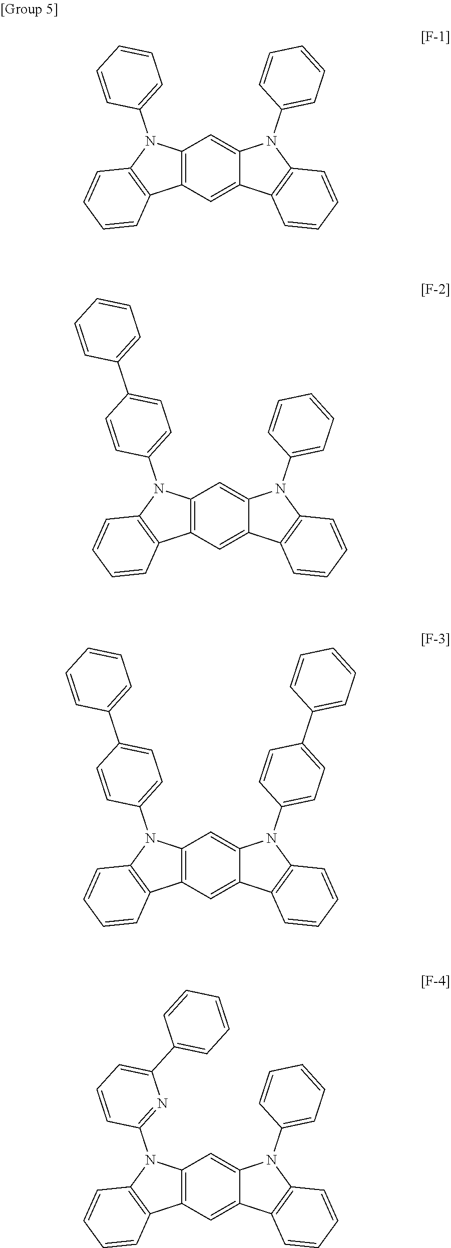 [SMILES: c1ccc(-n2c3ccccc3c3cc4c5ccccc5n(-c5ccccc5)c4cc32)cc1 ; c1ccc(-c2ccc(-n3c4ccccc4c4cc5c6ccccc6n(-c6ccccc6)c5cc43)cc2)cc1 ; c1ccc(-c2ccc(-n3c4ccccc4c4cc5c6ccccc6n(-c6ccc(-c7ccccc7)cc6)c5cc43)cc2)cc1 ; c1ccc(-c2cccc(-n3c4ccccc4c4cc5c6ccccc6n(-c6ccccc6)c5cc43)n2)cc1]

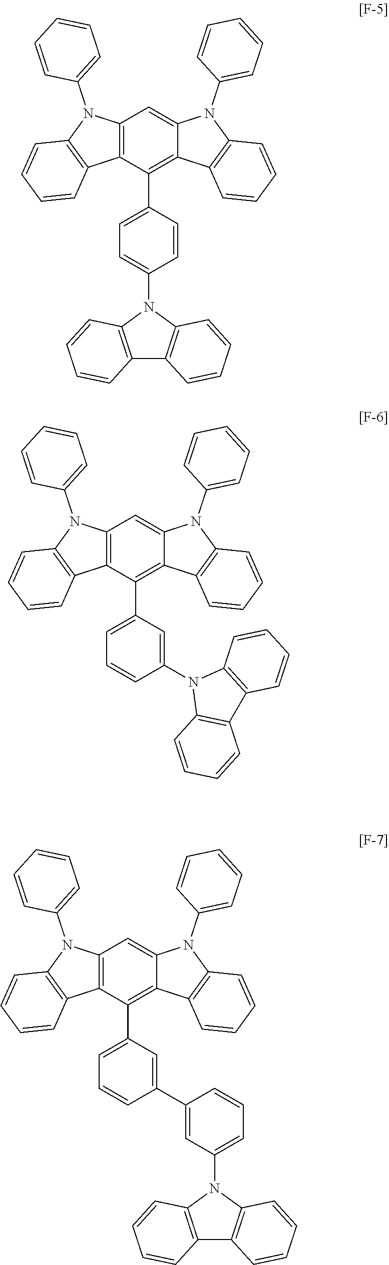 [SMILES: c1ccc(-n2c3ccccc3c3c(-c4ccc(-n5c6ccccc6c6ccccc65)cc4)c4c5ccccc5n(-c5ccccc5)c4cc32)cc1 ; c1ccc(-n2c3ccccc3c3c(-c4cccc(-n5c6ccccc6c6ccccc65)c4)c4c5ccccc5n(-c5ccccc5)c4cc32)cc1 ; c1ccc(-n2c3ccccc3c3c(-c4cccc(-c5cccc(-n6c7ccccc7c7ccccc76)c5)c4)c4c5ccccc5n(-c5ccccc5)c4cc32)cc1]

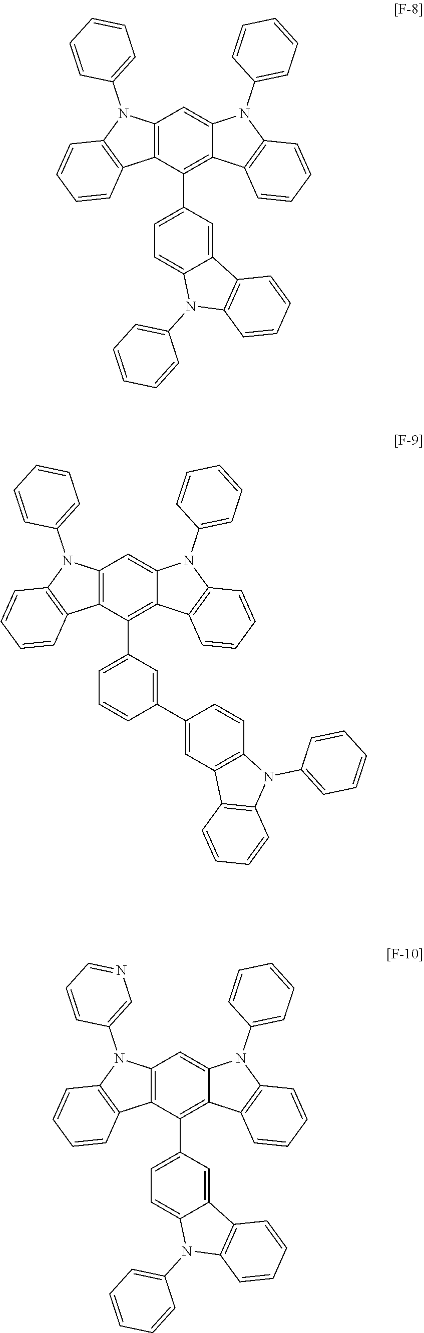 [SMILES: c1ccc(-n2c3ccccc3c3cc(-c4c5c6ccccc6n(-c6ccccc6)c5cc5c4c4ccccc4n5-c4ccccc4)ccc32)cc1 ; c1ccc(-n2c3ccccc3c3cc(-c4cccc(-c5c6c7ccccc7n(-c7ccccc7)c6cc6c5c5ccccc5n6-c5ccccc5)c4)ccc32)cc1 ; c1ccc(-n2c3ccccc3c3cc(-c4c5c6ccccc6n(-c6ccccc6)c5cc5c4c4ccccc4n5-c4cccnc4)ccc32)cc1]

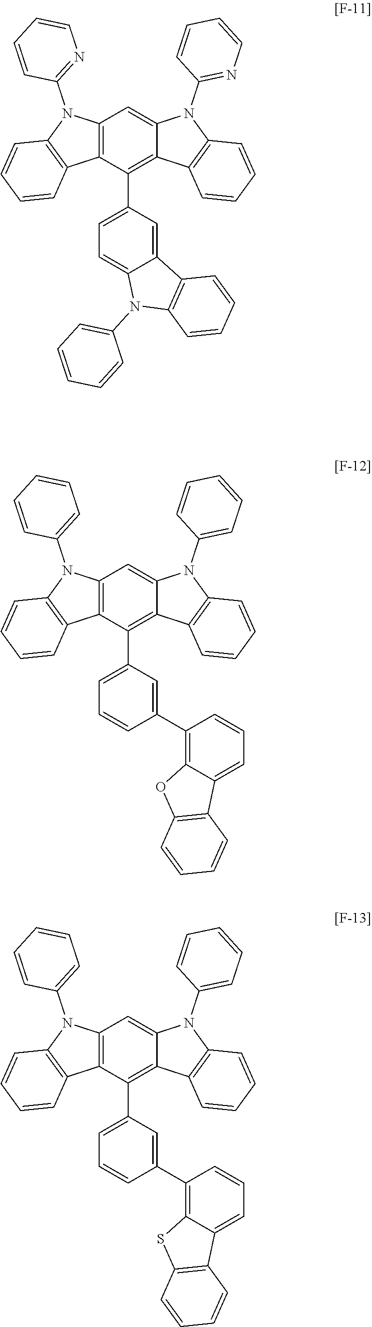 [SMILES: c1ccc(-n2c3ccccc3c3cc(-c4c5c6ccccc6n(-c6ccccn6)c5cc5c4c4ccccc4n5-c4ccccn4)ccc32)cc1 ; c1ccc(-n2c3ccccc3c3c(-c4cccc(-c5cccc6c5oc5ccccc56)c4)c4c5ccccc5n(-c5ccccc5)c4cc32)cc1 ; c1ccc(-n2c3ccccc3c3c(-c4cccc(-c5cccc6c5sc5ccccc56)c4)c4c5ccccc5n(-c5ccccc5)c4cc32)cc1]

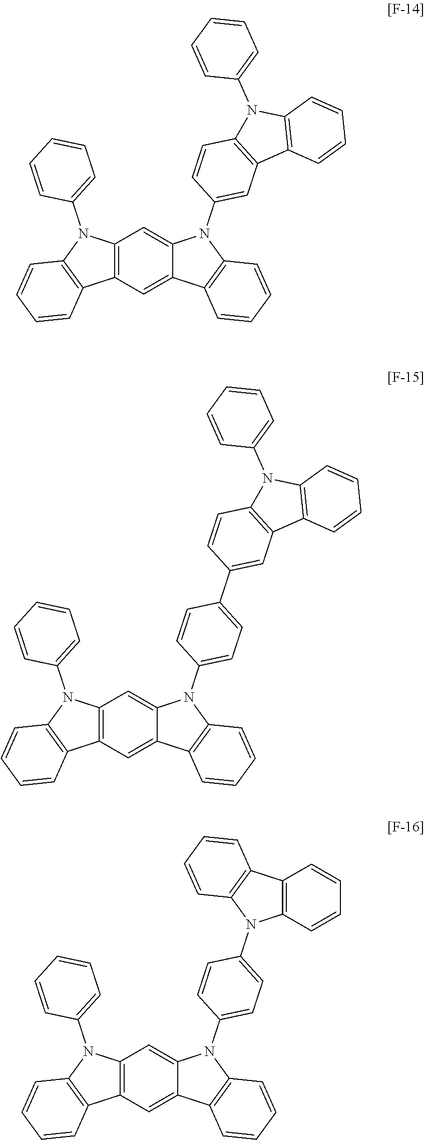 [SMILES: c1ccc(-n2c3ccccc3c3cc(-n4c5ccccc5c5cc6c7ccccc7n(-c7ccccc7)c6cc54)ccc32)cc1 ; c1ccc(-n2c3ccccc3c3cc(-c4ccc(-n5c6ccccc6c6cc7c8ccccc8n(-c8ccccc8)c7cc65)cc4)ccc32)cc1 ; c1ccc(-n2c3ccccc3c3cc4c5ccccc5n(-c5ccc(-n6c7ccccc7c7ccccc76)cc5)c4cc32)cc1]

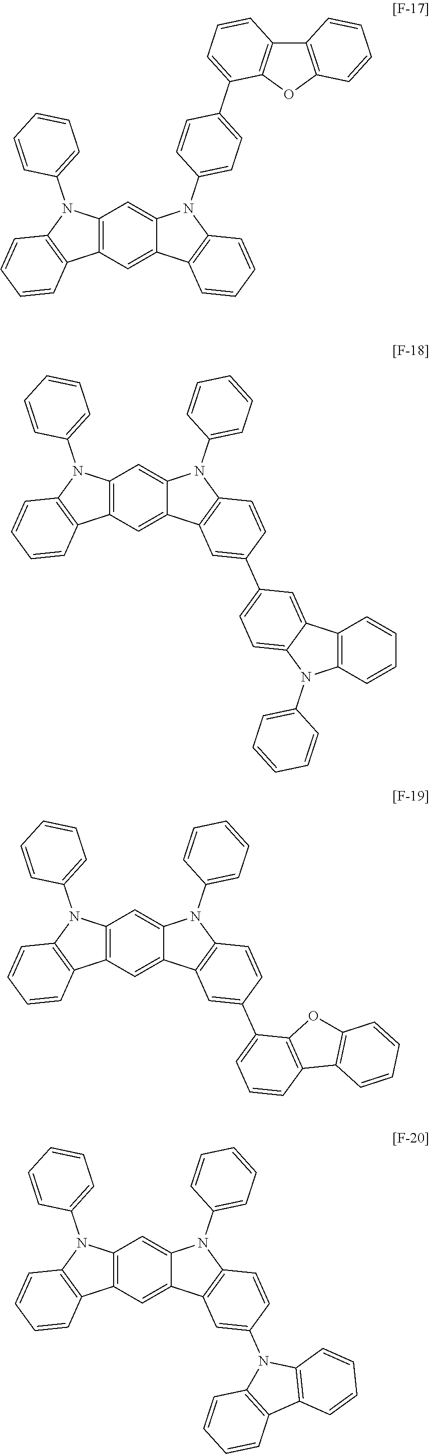 [SMILES: c1ccc(-n2c3ccccc3c3cc4c5ccccc5n(-c5ccc(-c6cccc7c6oc6ccccc67)cc5)c4cc32)cc1 ; c1ccc(-n2c3ccccc3c3cc(-c4ccc5c(c4)c4cc6c7ccccc7n(-c7ccccc7)c6cc4n5-c4ccccc4)ccc32)cc1 ; c1ccc(-n2c3ccccc3c3cc4c5cc(-c6cccc7c6oc6ccccc67)ccc5n(-c5ccccc5)c4cc32)cc1 ; c1ccc(-n2c3ccccc3c3cc4c5cc(-n6c7ccccc7c7ccccc76)ccc5n(-c5ccccc5)c4cc32)cc1]

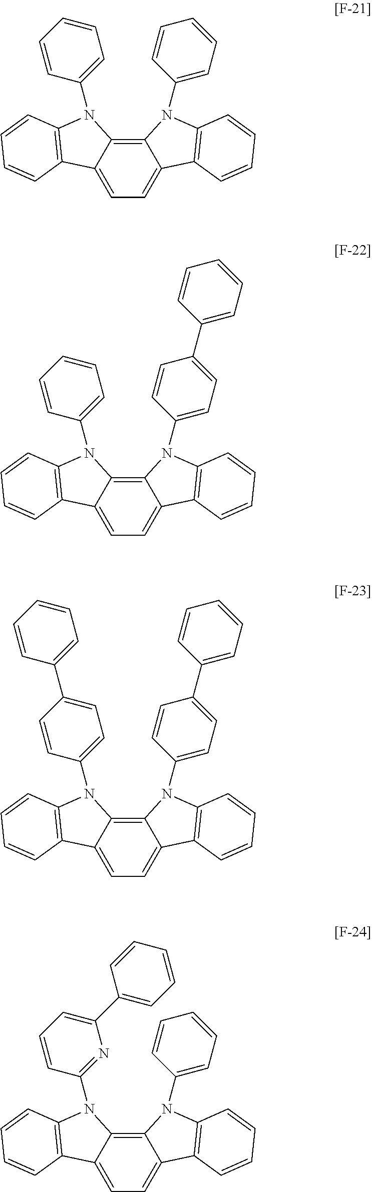 [SMILES: c1ccc(-n2c3ccccc3c3ccc4c5ccccc5n(-c5ccccc5)c4c32)cc1 ; c1ccc(-c2ccc(-n3c4ccccc4c4ccc5c6ccccc6n(-c6ccccc6)c5c43)cc2)cc1 ; c1ccc(-c2ccc(-n3c4ccccc4c4ccc5c6ccccc6n(-c6ccc(-c7ccccc7)cc6)c5c43)cc2)cc1 ; c1ccc(-c2cccc(-n3c4ccccc4c4ccc5c6ccccc6n(-c6ccccc6)c5c43)n2)cc1]

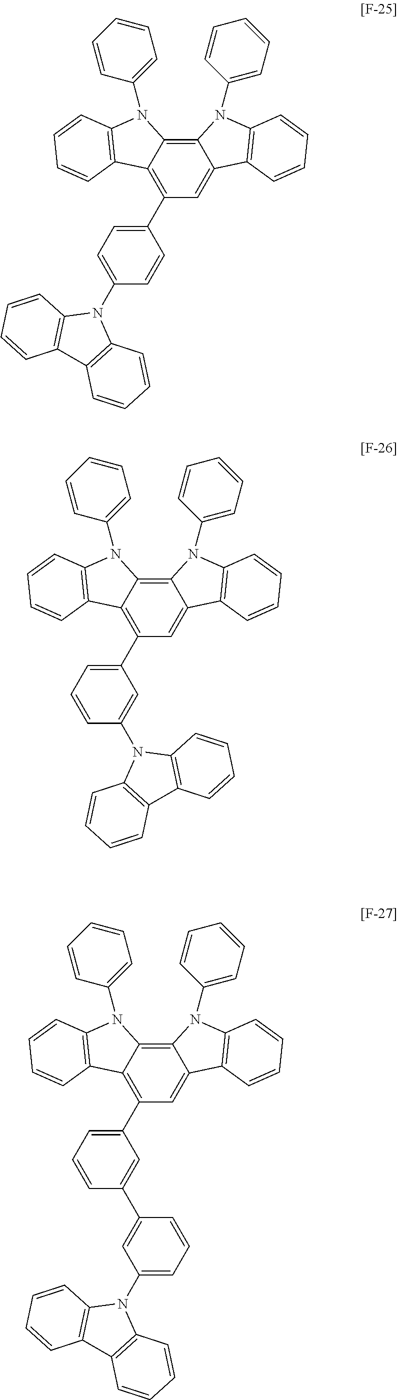 [SMILES: c1ccc(-n2c3ccccc3c3cc(-c4ccc(-n5c6ccccc6c6ccccc65)cc4)c4c5ccccc5n(-c5ccccc5)c4c32)cc1 ; c1ccc(-n2c3ccccc3c3cc(-c4cccc(-n5c6ccccc6c6ccccc65)c4)c4c5ccccc5n(-c5ccccc5)c4c32)cc1 ; c1ccc(-n2c3ccccc3c3cc(-c4cccc(-c5cccc(-n6c7ccccc7c7ccccc76)c5)c4)c4c5ccccc5n(-c5ccccc5)c4c32)cc1]

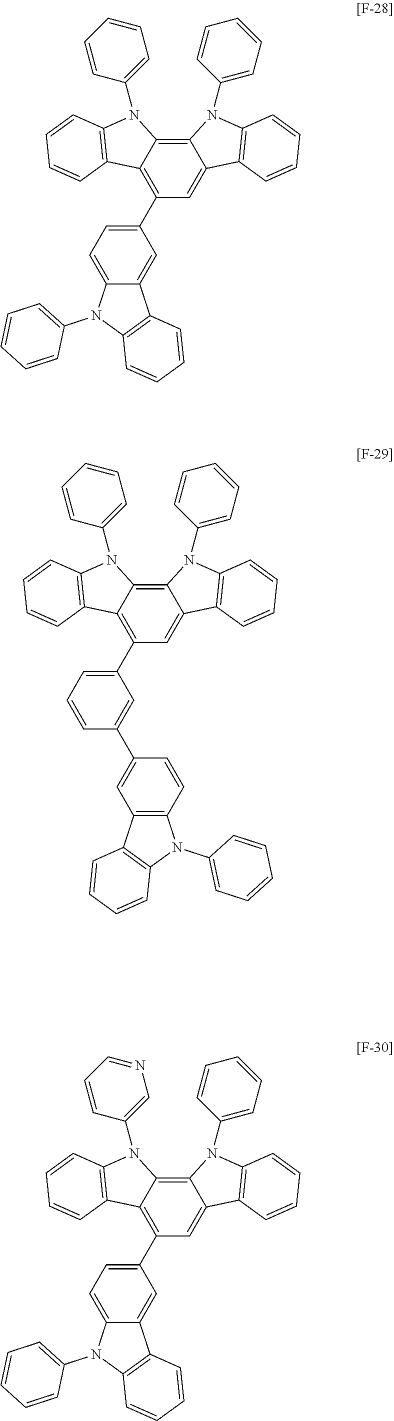 [SMILES: c1ccc(-n2c3ccccc3c3cc(-c4cc5c6ccccc6n(-c6ccccc6)c5c5c4c4ccccc4n5-c4ccccc4)ccc32)cc1 ; c1ccc(-n2c3ccccc3c3cc(-c4cccc(-c5cc6c7ccccc7n(-c7ccccc7)c6c6c5c5ccccc5n6-c5ccccc5)c4)ccc32)cc1 ; c1ccc(-n2c3ccccc3c3cc(-c4cc5c6ccccc6n(-c6ccccc6)c5c5c4c4ccccc4n5-c4cccnc4)ccc32)cc1]

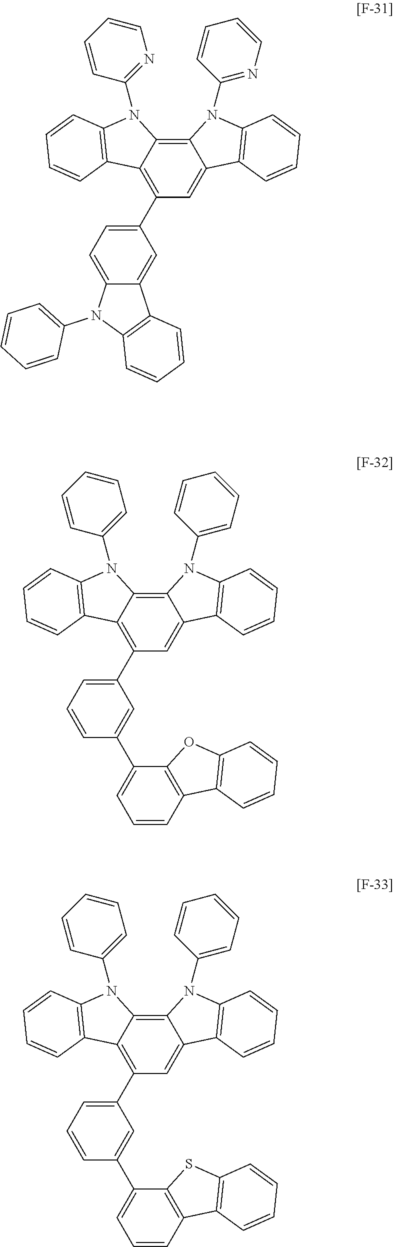 [SMILES: c1ccc(-n2c3ccccc3c3cc(-c4cc5c6ccccc6n(-c6ccccn6)c5c5c4c4ccccc4n5-c4ccccn4)ccc32)cc1 ; c1ccc(-n2c3ccccc3c3cc(-c4cccc(-c5cccc6c5oc5ccccc56)c4)c4c5ccccc5n(-c5ccccc5)c4c32)cc1 ; c1ccc(-n2c3ccccc3c3cc(-c4cccc(-c5cccc6c5sc5ccccc56)c4)c4c5ccccc5n(-c5ccccc5)c4c32)cc1]

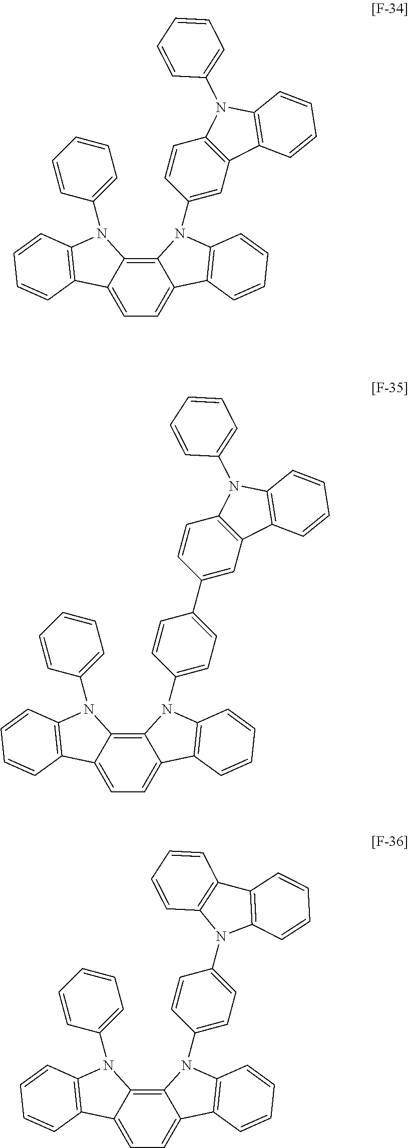 [SMILES: c1ccc(-n2c3ccccc3c3cc(-n4c5ccccc5c5ccc6c7ccccc7n(-c7ccccc7)c6c54)ccc32)cc1 ; c1ccc(-n2c3ccccc3c3cc(-c4ccc(-n5c6ccccc6c6ccc7c8ccccc8n(-c8ccccc8)c7c65)cc4)ccc32)cc1 ; c1ccc(-n2c3ccccc3c3ccc4c5ccccc5n(-c5ccc(-n6c7ccccc7c7ccccc76)cc5)c4c32)cc1]

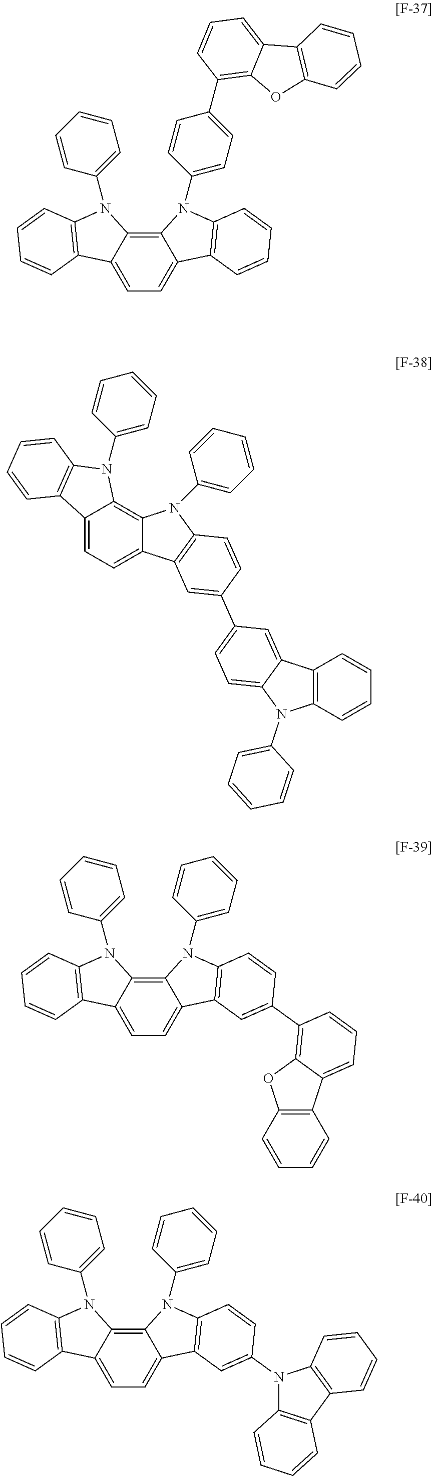 [SMILES: c1ccc(-n2c3ccccc3c3ccc4c5ccccc5n(-c5ccc(-c6cccc7c6oc6ccccc67)cc5)c4c32)cc1 ; c1ccc(-n2c3ccccc3c3cc(-c4ccc5c(c4)c4ccc6c7ccccc7n(-c7ccccc7)c6c4n5-c4ccccc4)ccc32)cc1 ; c1ccc(-n2c3ccccc3c3ccc4c5cc(-c6cccc7c6oc6ccccc67)ccc5n(-c5ccccc5)c4c32)cc1 ; c1ccc(-n2c3ccccc3c3ccc4c5cc(-n6c7ccccc7c7ccccc76)ccc5n(-c5ccccc5)c4c32)cc1]

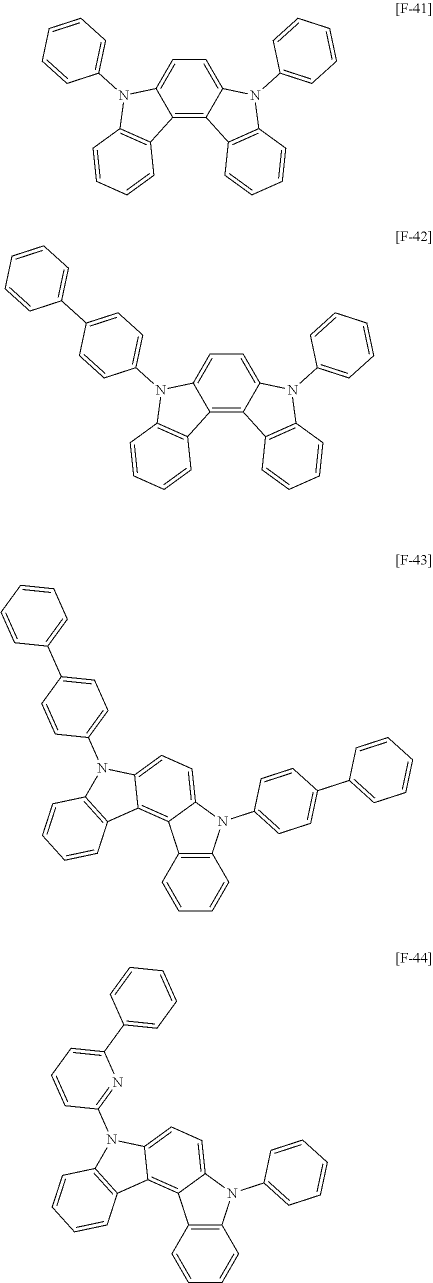 [SMILES: c1ccc(-n2c3ccccc3c3c4c5ccccc5n(-c5ccccc5)c4ccc32)cc1 ; c1ccc(-c2ccc(-n3c4ccccc4c4c5c6ccccc6n(-c6ccccc6)c5ccc43)cc2)cc1 ; c1ccc(-c2ccc(-n3c4ccccc4c4c5c6ccccc6n(-c6ccc(-c7ccccc7)cc6)c5ccc43)cc2)cc1 ; c1ccc(-c2cccc(-n3c4ccccc4c4c5c6ccccc6n(-c6ccccc6)c5ccc43)n2)cc1]

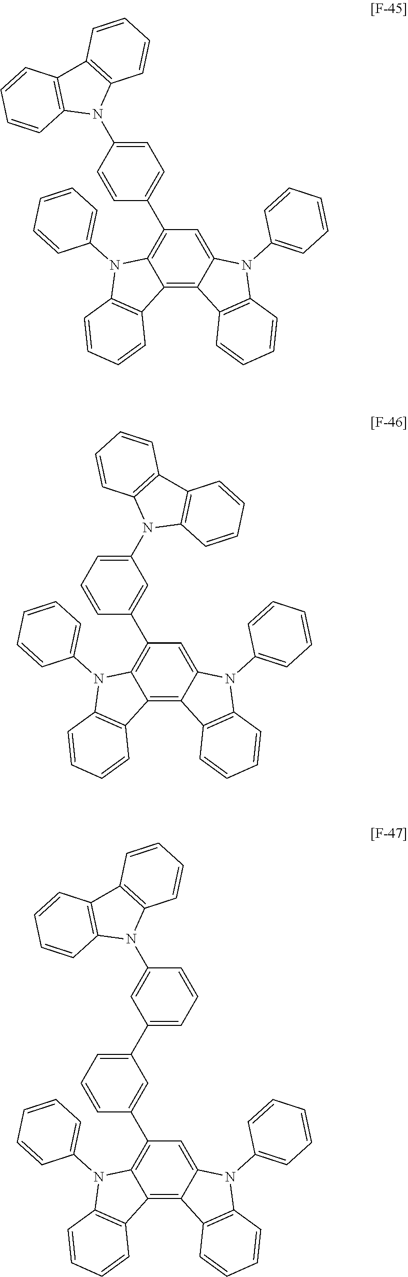 [SMILES: c1ccc(-n2c3ccccc3c3c4c5ccccc5n(-c5ccccc5)c4c(-c4ccc(-n5c6ccccc6c6ccccc65)cc4)cc32)cc1 ; c1ccc(-n2c3ccccc3c3c4c5ccccc5n(-c5ccccc5)c4c(-c4cccc(-n5c6ccccc6c6ccccc65)c4)cc32)cc1 ; c1ccc(-n2c3ccccc3c3c4c5ccccc5n(-c5ccccc5)c4c(-c4cccc(-c5cccc(-n6c7ccccc7c7ccccc76)c5)c4)cc32)cc1]

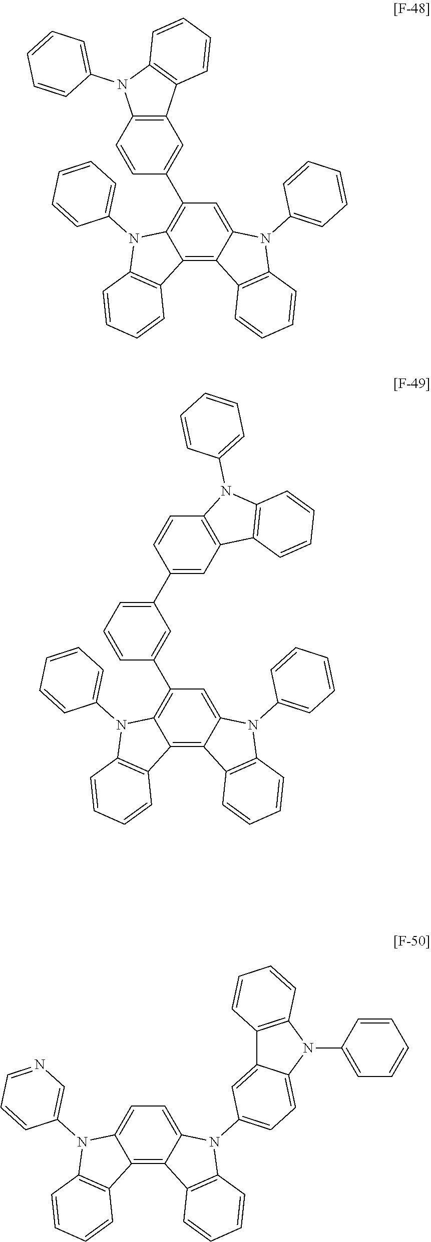 [SMILES: c1ccc(-n2c3ccccc3c3cc(-c4cc5c(c6ccccc6n5-c5ccccc5)c5c6ccccc6n(-c6ccccc6)c45)ccc32)cc1 ; c1ccc(-n2c3ccccc3c3cc(-c4cccc(-c5cc6c(c7ccccc7n6-c6ccccc6)c6c7ccccc7n(-c7ccccc7)c56)c4)ccc32)cc1 ; c1ccc(-n2c3ccccc3c3cc(-n4c5ccccc5c5c6c7ccccc7n(-c7cccnc7)c6ccc54)ccc32)cc1]

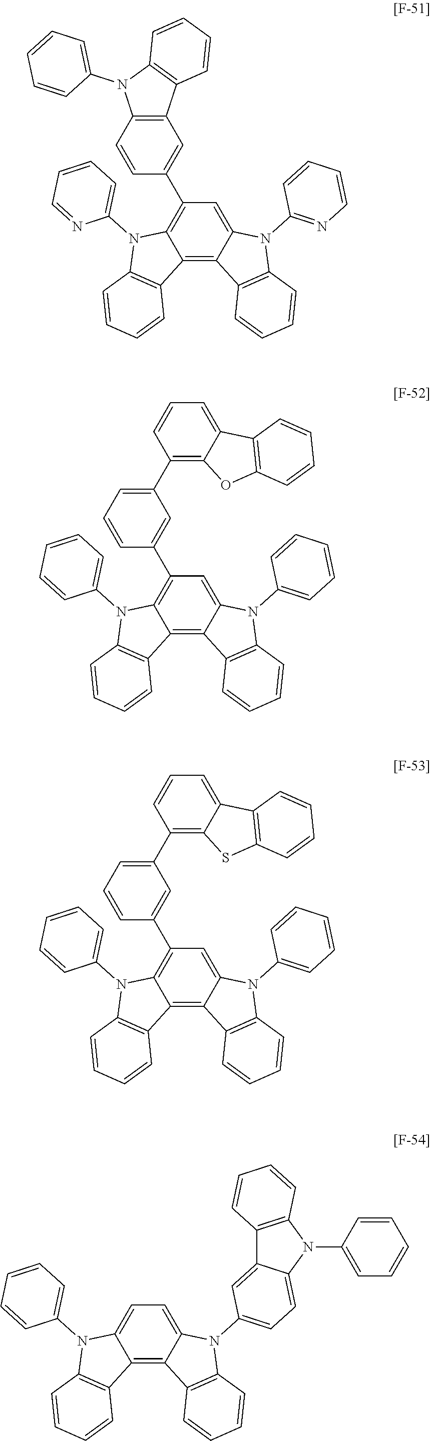 [SMILES: c1ccc(-n2c3ccccc3c3cc(-c4cc5c(c6ccccc6n5-c5ccccn5)c5c6ccccc6n(-c6ccccn6)c45)ccc32)cc1 ; c1ccc(-n2c3ccccc3c3c4c5ccccc5n(-c5ccccc5)c4c(-c4cccc(-c5cccc6c5oc5ccccc56)c4)cc32)cc1 ; c1ccc(-n2c3ccccc3c3c4c5ccccc5n(-c5ccccc5)c4c(-c4cccc(-c5cccc6c5sc5ccccc56)c4)cc32)cc1 ; c1ccc(-n2c3ccccc3c3cc(-n4c5ccccc5c5c6c7ccccc7n(-c7ccccc7)c6ccc54)ccc32)cc1]

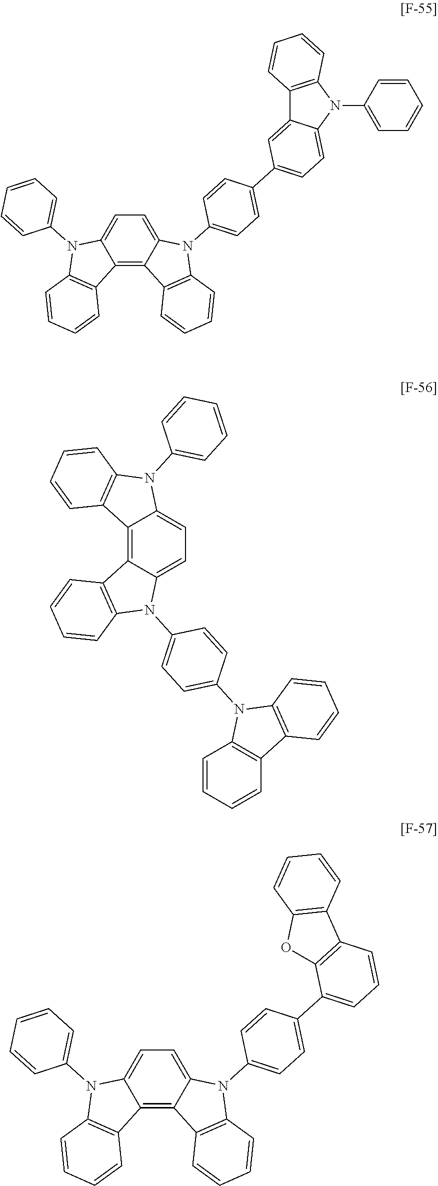 [SMILES: c1ccc(-n2c3ccccc3c3cc(-c4ccc(-n5c6ccccc6c6c7c8ccccc8n(-c8ccccc8)c7ccc65)cc4)ccc32)cc1 ; c1ccc(-n2c3ccccc3c3c4c5ccccc5n(-c5ccc(-n6c7ccccc7c7ccccc76)cc5)c4ccc32)cc1 ; c1ccc(-n2c3ccccc3c3c4c5ccccc5n(-c5ccc(-c6cccc7c6oc6ccccc67)cc5)c4ccc32)cc1]

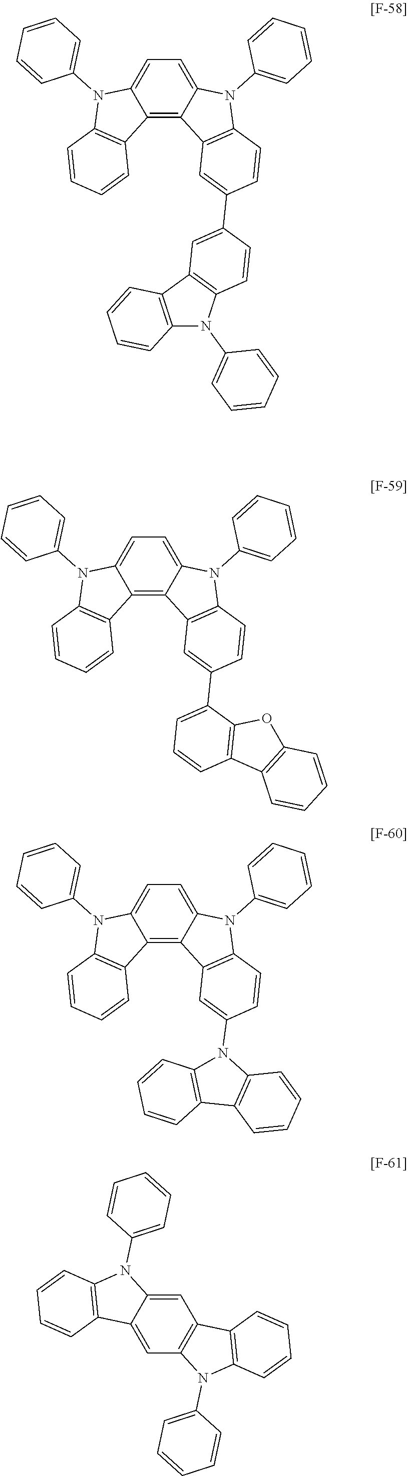

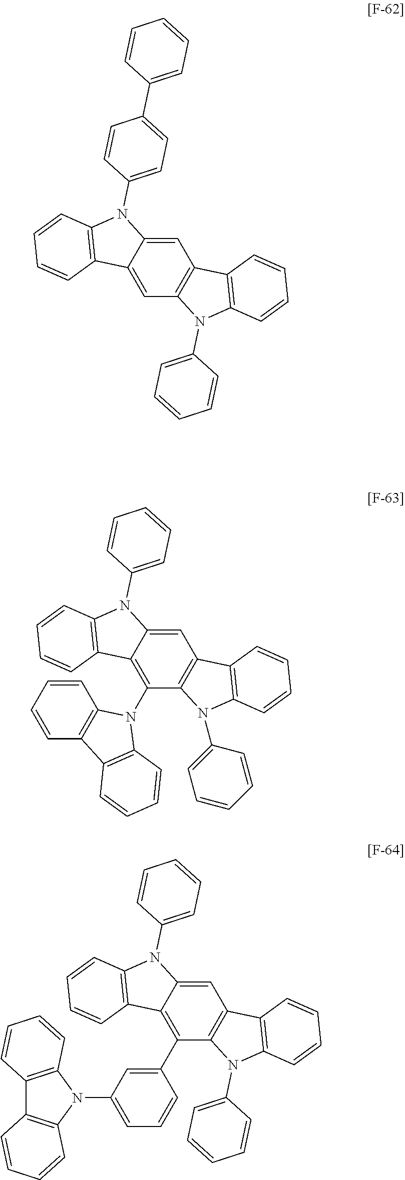 [SMILES: c1ccc(-c2ccc(-n3c4ccccc4c4cc5c(cc43)c3ccccc3n5-c3ccccc3)cc2)cc1 ; c1ccc(-n2c3ccccc3c3c(-n4c5ccccc5c5ccccc54)c4c(cc32)c2ccccc2n4-c2ccccc2)cc1 ; c1ccc(-n2c3ccccc3c3c(-c4cccc(-n5c6ccccc6c6ccccc65)c4)c4c(cc32)c2ccccc2n4-c2ccccc2)cc1]

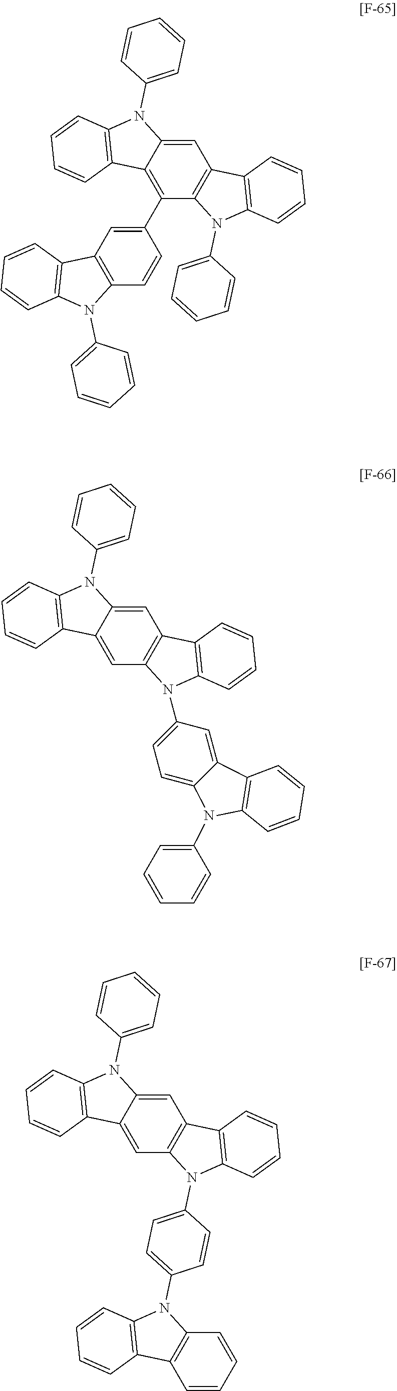 [SMILES: c1ccc(-n2c3ccccc3c3cc(-c4c5c6ccccc6n(-c6ccccc6)c5cc5c6ccccc6n(-c6ccccc6)c45)ccc32)cc1 ; c1ccc(-n2c3ccccc3c3cc(-n4c5ccccc5c5cc6c(cc54)c4ccccc4n6-c4ccccc4)ccc32)cc1 ; c1ccc(-n2c3ccccc3c3cc4c(cc32)c2ccccc2n4-c2ccc(-n3c4ccccc4c4ccccc43)cc2)cc1]

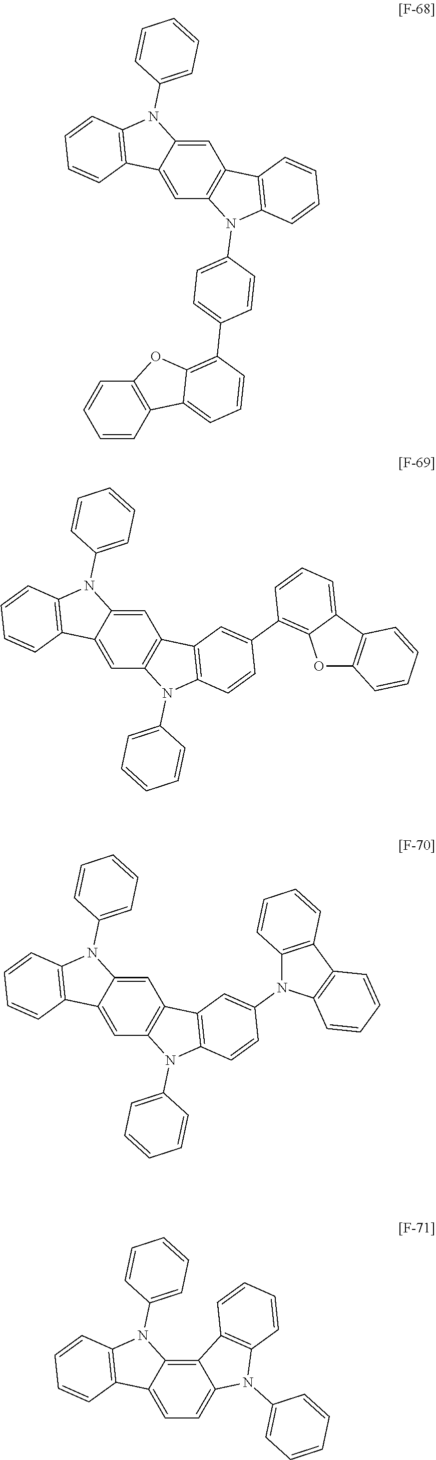 [SMILES: c1ccc(-n2c3ccccc3c3cc4c(cc32)c2ccccc2n4-c2ccc(-c3cccc4c3oc3ccccc34)cc2)cc1 ; c1ccc(-n2c3ccccc3c3cc4c(cc32)c2cc(-c3cccc5c3oc3ccccc35)ccc2n4-c2ccccc2)cc1 ; c1ccc(-n2c3ccccc3c3cc4c(cc32)c2cc(-n3c5ccccc5c5ccccc53)ccc2n4-c2ccccc2)cc1 ; c1ccc(-n2c3ccccc3c3c2ccc2c4ccccc4n(-c4ccccc4)c23)cc1]

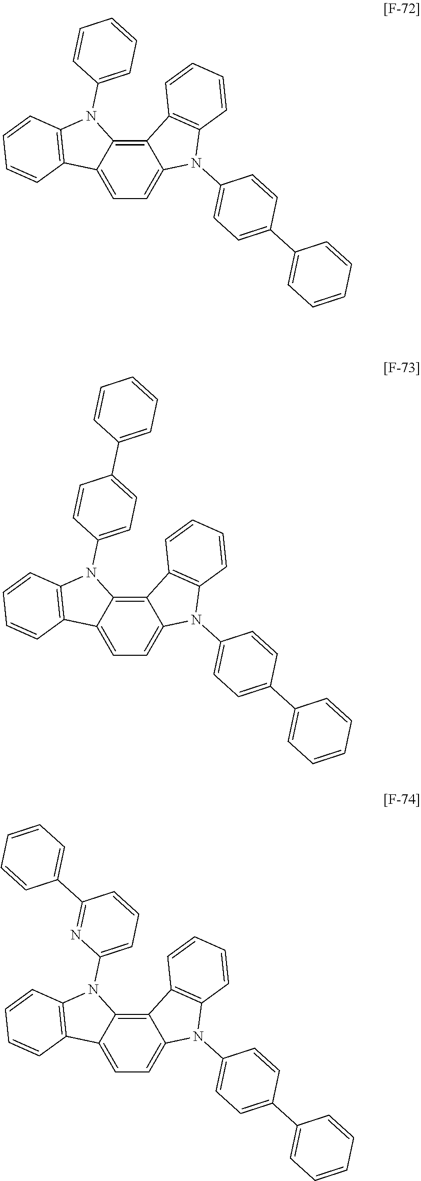 [SMILES: c1ccc(-c2ccc(-n3c4ccccc4c4c3ccc3c5ccccc5n(-c5ccccc5)c34)cc2)cc1 ; c1ccc(-c2ccc(-n3c4ccccc4c4c3ccc3c5ccccc5n(-c5ccc(-c6ccccc6)cc5)c34)cc2)cc1 ; c1ccc(-c2ccc(-n3c4ccccc4c4c3ccc3c5ccccc5n(-c5cccc(-c6ccccc6)n5)c34)cc2)cc1]

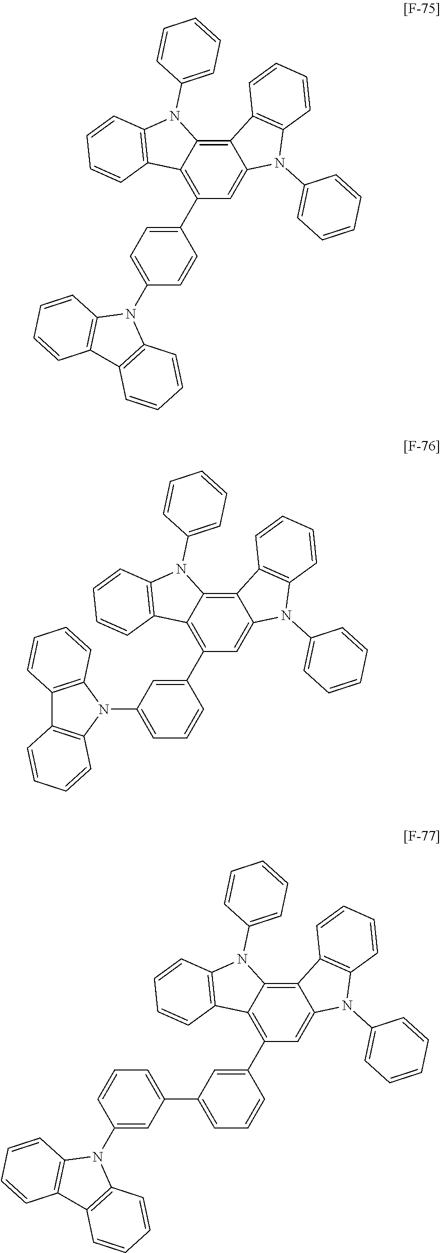 [SMILES: c1ccc(-n2c3ccccc3c3c2cc(-c2ccc(-n4c5ccccc5c5ccccc54)cc2)c2c4ccccc4n(-c4ccccc4)c23)cc1 ; c1ccc(-n2c3ccccc3c3c2cc(-c2cccc(-n4c5ccccc5c5ccccc54)c2)c2c4ccccc4n(-c4ccccc4)c23)cc1 ; c1ccc(-n2c3ccccc3c3c2cc(-c2cccc(-c4cccc(-n5c6ccccc6c6ccccc65)c4)c2)c2c4ccccc4n(-c4ccccc4)c23)cc1]

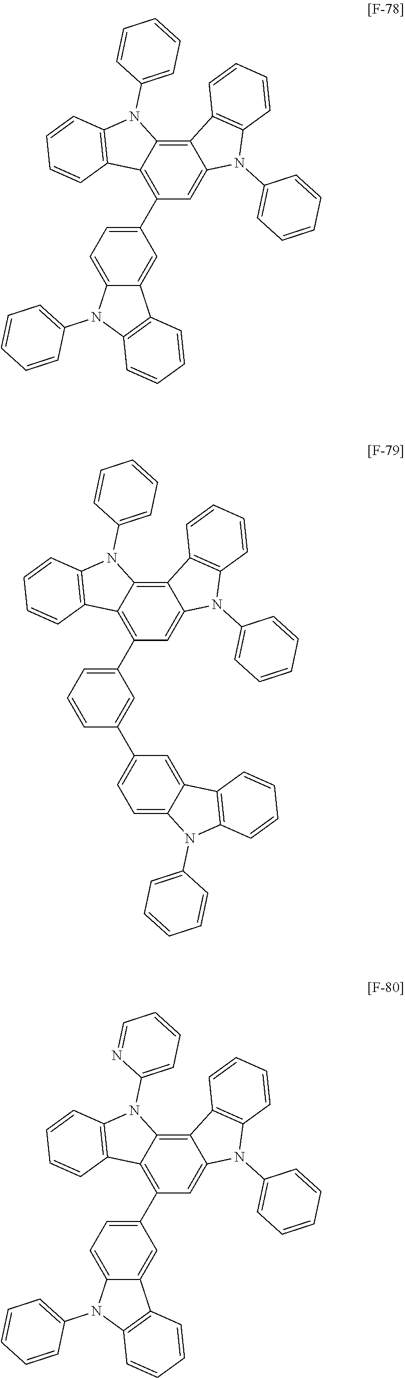 [SMILES: c1ccc(-n2c3ccccc3c3cc(-c4cc5c(c6ccccc6n5-c5ccccc5)c5c4c4ccccc4n5-c4ccccc4)ccc32)cc1 ; c1ccc(-n2c3ccccc3c3cc(-c4cccc(-c5cc6c(c7ccccc7n6-c6ccccc6)c6c5c5ccccc5n6-c5ccccc5)c4)ccc32)cc1 ; c1ccc(-n2c3ccccc3c3cc(-c4cc5c(c6ccccc6n5-c5ccccc5)c5c4c4ccccc4n5-c4ccccn4)ccc32)cc1]

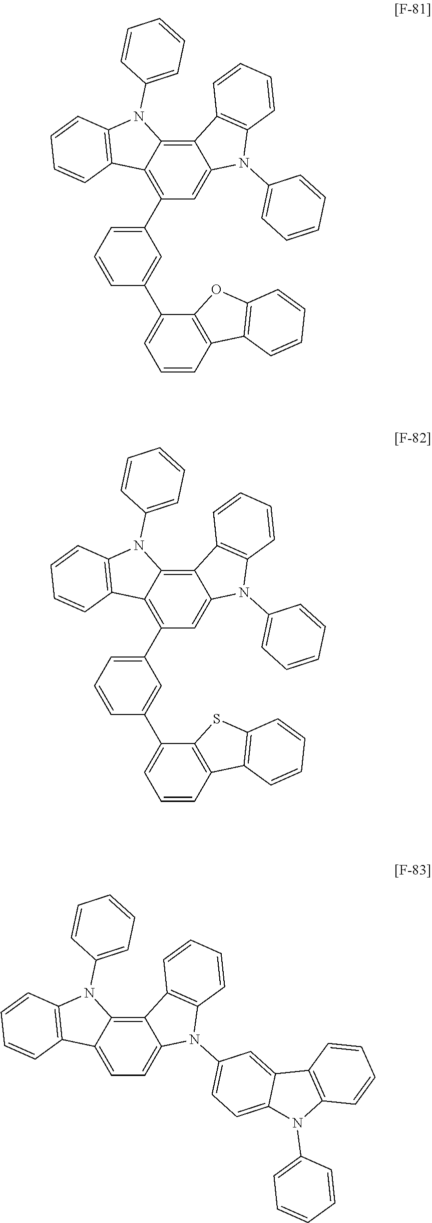 [SMILES: c1ccc(-n2c3ccccc3c3c2cc(-c2cccc(-c4cccc5c4oc4ccccc45)c2)c2c4ccccc4n(-c4ccccc4)c23)cc1 ; c1ccc(-n2c3ccccc3c3c2cc(-c2cccc(-c4cccc5c4sc4ccccc45)c2)c2c4ccccc4n(-c4ccccc4)c23)cc1 ; c1ccc(-n2c3ccccc3c3cc(-n4c5ccccc5c5c4ccc4c6ccccc6n(-c6ccccc6)c45)ccc32)cc1]

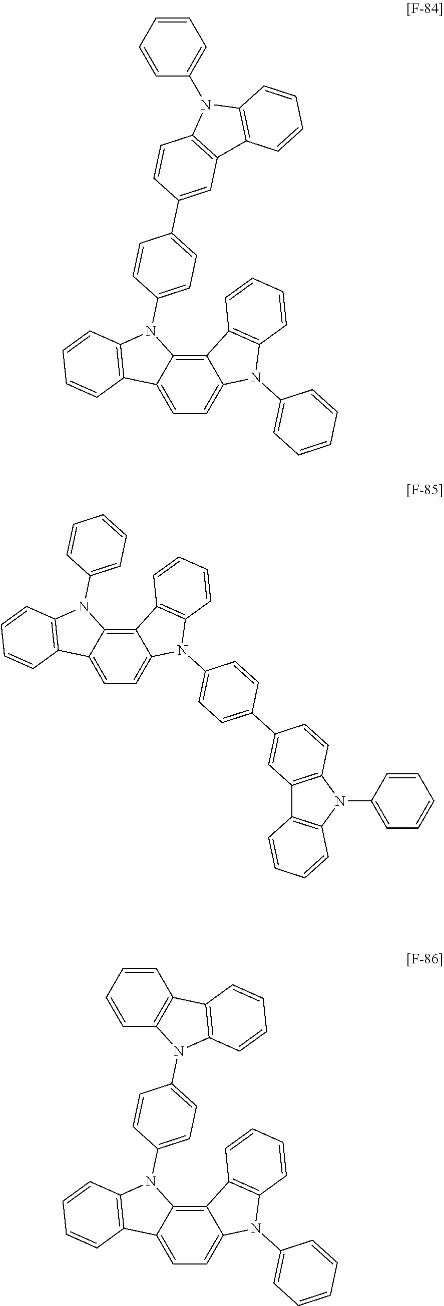 [SMILES: c1ccc(-n2c3ccccc3c3cc(-c4ccc(-n5c6ccccc6c6ccc7c(c8ccccc8n7-c7ccccc7)c65)cc4)ccc32)cc1 ; c1ccc(-n2c3ccccc3c3cc(-c4ccc(-n5c6ccccc6c6c5ccc5c7ccccc7n(-c7ccccc7)c56)cc4)ccc32)cc1 ; c1ccc(-n2c3ccccc3c3c2ccc2c4ccccc4n(-c4ccc(-n5c6ccccc6c6ccccc65)cc4)c23)cc1]

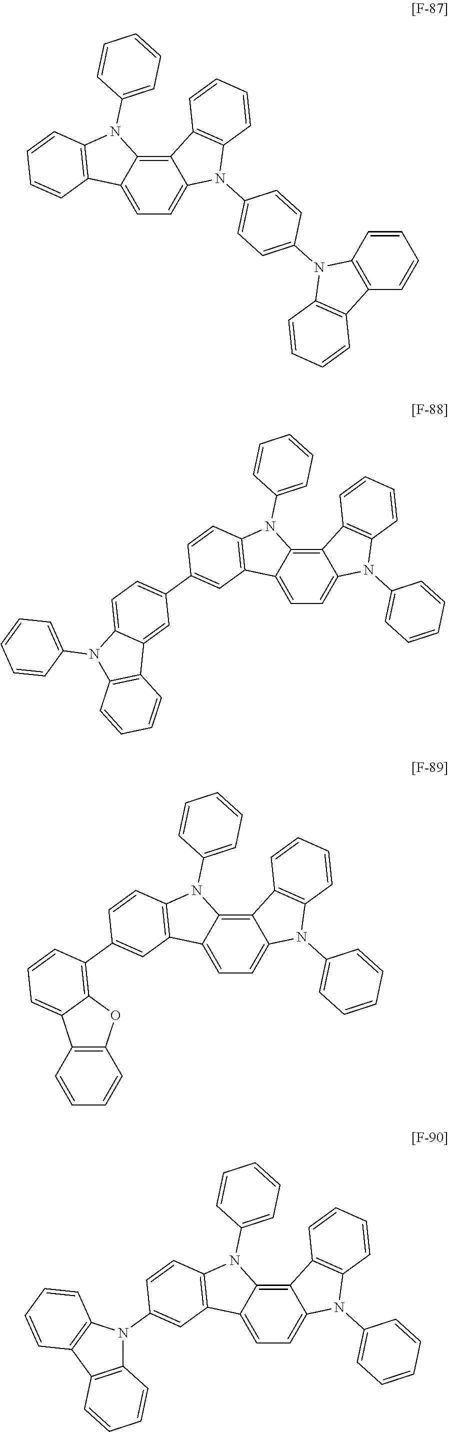 [SMILES: c1ccc(-n2c3ccccc3c3ccc4c(c5ccccc5n4-c4ccc(-n5c6ccccc6c6ccccc65)cc4)c32)cc1 ; c1ccc(-n2c3ccccc3c3cc(-c4ccc5c(c4)c4ccc6c(c7ccccc7n6-c6ccccc6)c4n5-c4ccccc4)ccc32)cc1 ; c1ccc(-n2c3ccccc3c3c2ccc2c4cc(-c5cccc6c5oc5ccccc56)ccc4n(-c4ccccc4)c23)cc1 ; c1ccc(-n2c3ccccc3c3c2ccc2c4cc(-n5c6ccccc6c6ccccc65)ccc4n(-c4ccccc4)c23)cc1]

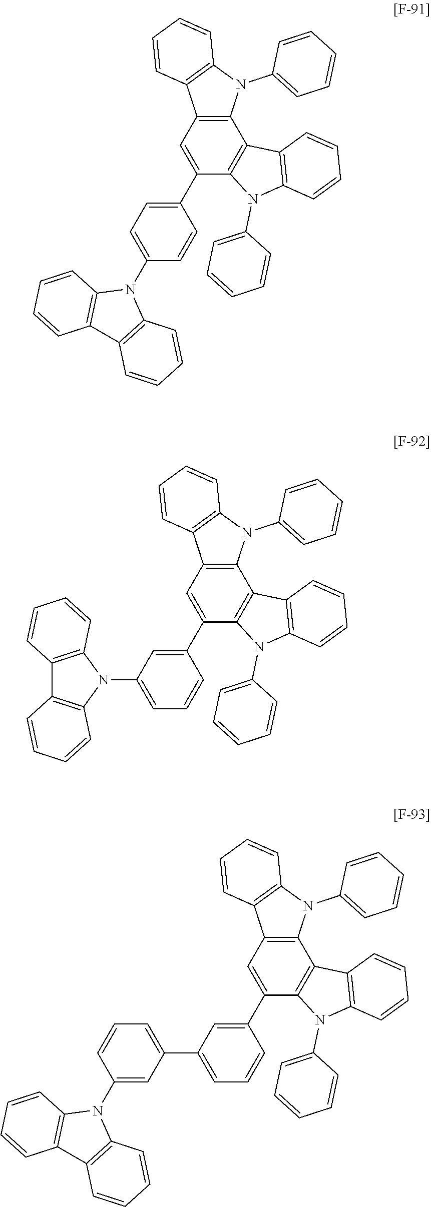 [SMILES: c1ccc(-n2c3ccccc3c3c2c(-c2ccc(-n4c5ccccc5c5ccccc54)cc2)cc2c4ccccc4n(-c4ccccc4)c23)cc1 ; c1ccc(-n2c3ccccc3c3c2c(-c2cccc(-n4c5ccccc5c5ccccc54)c2)cc2c4ccccc4n(-c4ccccc4)c23)cc1 ; c1ccc(-n2c3ccccc3c3c2c(-c2cccc(-c4cccc(-n5c6ccccc6c6ccccc65)c4)c2)cc2c4ccccc4n(-c4ccccc4)c23)cc1]

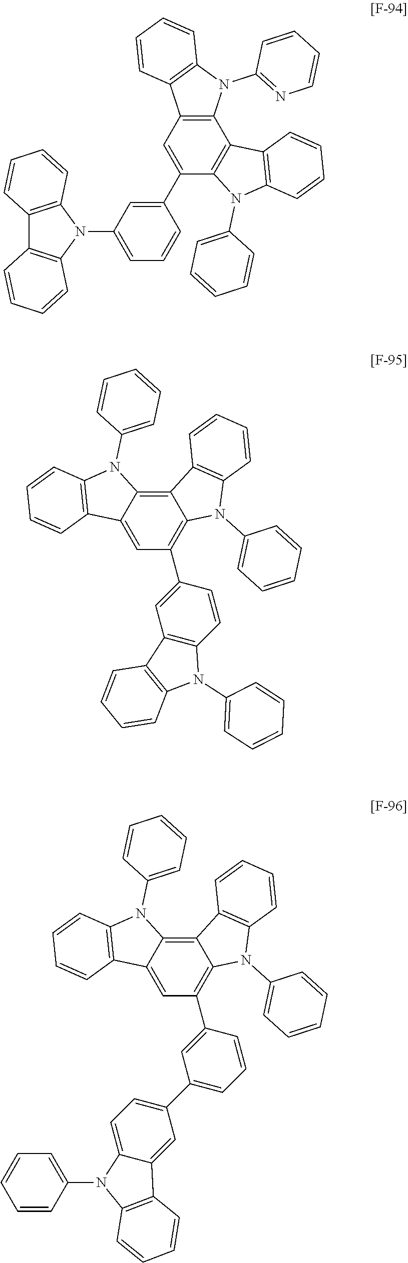 [SMILES: c1ccc(-n2c3ccccc3c3c2c(-c2cccc(-n4c5ccccc5c5ccccc54)c2)cc2c4ccccc4n(-c4ccccn4)c23)cc1 ; c1ccc(-n2c3ccccc3c3cc(-c4cc5c6ccccc6n(-c6ccccc6)c5c5c6ccccc6n(-c6ccccc6)c45)ccc32)cc1 ; c1ccc(-n2c3ccccc3c3cc(-c4cccc(-c5cc6c7ccccc7n(-c7ccccc7)c6c6c7ccccc7n(-c7ccccc7)c56)c4)ccc32)cc1]

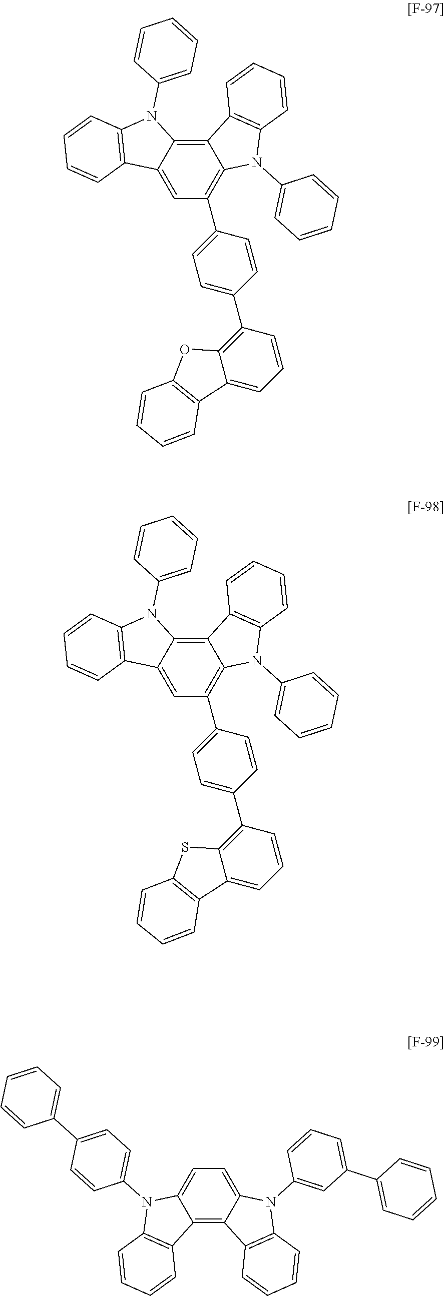 [SMILES: c1ccc(-n2c3ccccc3c3c2c(-c2ccc(-c4cccc5c4oc4ccccc45)cc2)cc2c4ccccc4n(-c4ccccc4)c23)cc1 ; c1ccc(-n2c3ccccc3c3c2c(-c2ccc(-c4cccc5c4sc4ccccc45)cc2)cc2c4ccccc4n(-c4ccccc4)c23)cc1 ; c1ccc(-c2ccc(-n3c4ccccc4c4c5c6ccccc6n(-c6cccc(-c7ccccc7)c6)c5ccc43)cc2)cc1]

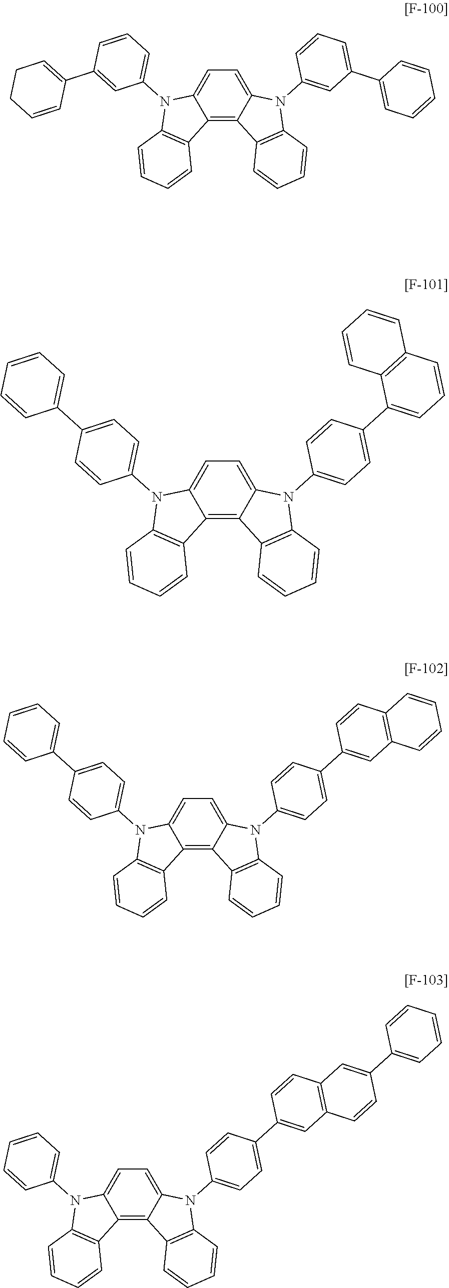 [SMILES: C1=CC(c2cccc(-n3c4ccccc4c4c5c6ccccc6n(-c6cccc(-c7ccccc7)c6)c5ccc43)c2)=CCC1 ; c1ccc(-c2ccc(-n3c4ccccc4c4c5c6ccccc6n(-c6ccc(-c7cccc8ccccc78)cc6)c5ccc43)cc2)cc1 ; c1ccc(-c2ccc(-n3c4ccccc4c4c5c6ccccc6n(-c6ccc(-c7ccc8ccccc8c7)cc6)c5ccc43)cc2)cc1 ; c1ccc(-c2ccc3cc(-c4ccc(-n5c6ccccc6c6c7c8ccccc8n(-c8ccccc8)c7ccc65)cc4)ccc3c2)cc1]

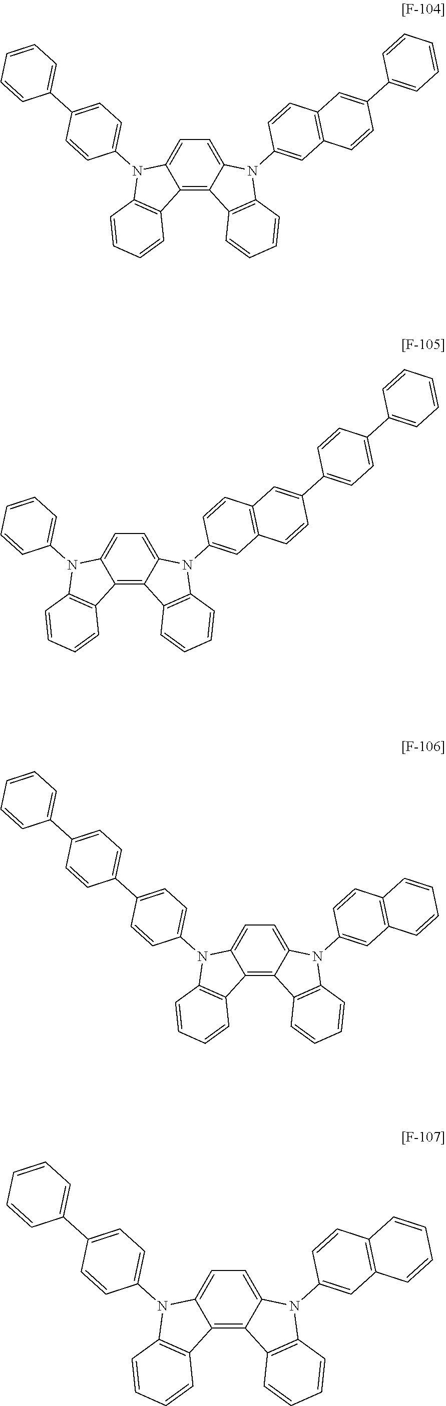 [SMILES: c1ccc(-c2ccc(-n3c4ccccc4c4c5c6ccccc6n(-c6ccc7cc(-c8ccccc8)ccc7c6)c5ccc43)cc2)cc1 ; c1ccc(-c2ccc(-c3ccc4cc(-n5c6ccccc6c6c7c8ccccc8n(-c8ccccc8)c7ccc65)ccc4c3)cc2)cc1 ; c1ccc(-c2ccc(-c3ccc(-n4c5ccccc5c5c6c7ccccc7n(-c7ccc8ccccc8c7)c6ccc54)cc3)cc2)cc1 ; c1ccc(-c2ccc(-n3c4ccccc4c4c5c6ccccc6n(-c6ccc7ccccc7c6)c5ccc43)cc2)cc1]

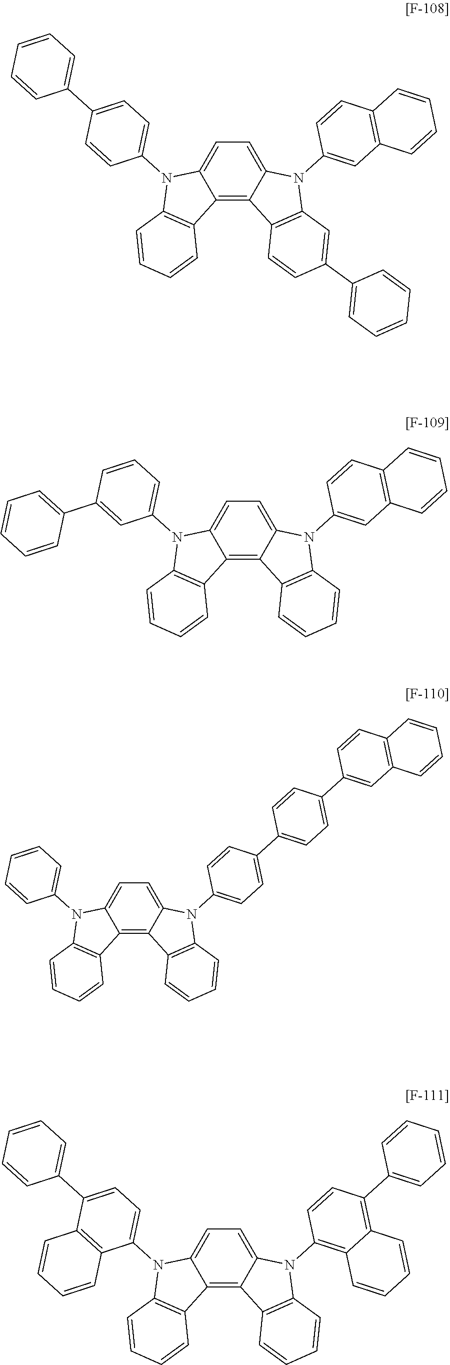 [SMILES: c1ccc(-c2ccc(-n3c4ccccc4c4c5c6ccc(-c7ccccc7)cc6n(-c6ccc7ccccc7c6)c5ccc43)cc2)cc1 ; c1ccc(-c2cccc(-n3c4ccccc4c4c5c6ccccc6n(-c6ccc7ccccc7c6)c5ccc43)c2)cc1 ; c1ccc(-n2c3ccccc3c3c4c5ccccc5n(-c5ccc(-c6ccc(-c7ccc8ccccc8c7)cc6)cc5)c4ccc32)cc1 ; c1ccc(-c2ccc(-n3c4ccccc4c4c5c6ccccc6n(-c6ccc(-c7ccccc7)c7ccccc67)c5ccc43)c3ccccc23)cc1]

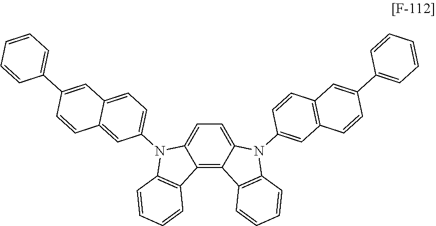 [SMILES: c1ccc(-c2ccc3cc(-n4c5ccccc5c5c6c7ccccc7n(-c7ccc8cc(-c9ccccc9)ccc8c7)c6ccc54)ccc3c2)cc1]

The first organic compound and the second organic compound may be combined to provide various compositions. The composition may include the first organic compound and the second compound in a weight ratio of about 1:99 to about 99:1, for example about 10:90 to about 90:10, about 20:80 to about 80:20, about 30:70 to about 70:30, about 40:60 to about 60:40, or about 50:50.

The composition may further include at least one organic compound in addition to the first organic compound and the second organic compound.

The composition may further include a dopant. The dopant may be, for example, a red, green, or blue dopant. The dopant may be used to cause light emission, and may be, for example, a material such as a metal complex that emits light by multiple excitation into a triplet state. The dopant may be, for example an inorganic, organic, or organic/inorganic compound, and one or more kinds thereof may be used. The dopant may be included in an amount of about 0.1 wt % to about 20 wt % based on a total amount of the composition.

The dopant may be, for example, a phosphorescent dopant and examples of the phosphorescent dopant may be an organometal compound including Ir, Pt, Os, Ti, Zr, Hf, Eu, Tb, Tm, Fe, Co, Ni, Ru, Rh, Pd, or a combination thereof. The phosphorescent dopant may be, for example a compound represented by Chemical Formula Z:

L₂MX [Chemical Formula Z]

In Chemical Formula Z, M is a metal, and L and X are the same or different, and are a ligand to form a complex compound with M.

The M may be for example Ir, Pt, Os, Ti, Zr, Hf, Eu, Tb, Tm, Fe, Co, Ni, Ru, Rh, Pd, or a combination thereof. L and X may be, for example, a bidendate ligand.

Hereinafter, an organic optoelectronic device including the organic compound or the composition is described.

The organic optoelectronic device may be, for example, an organic light emitting diode, an organic photoelectric device, or an organic solar cell. Example of an organic optoelectronic device may be an organic light emitting diode.

The organic optoelectronic device includes an anode and a cathode facing each other, and an organic layer disposed between the anode and the cathode, wherein the organic layer includes the organic compound or the composition.

The organic layer may include an active layer such as a light emitting layer or a light absorbing layer, and the organic compound or the composition may be included in the active layer.

The organic layer may include an auxiliary layer between the anode and the active layer, and/or between the cathode and the active layer, and the organic compound or the composition may be included in the auxiliary layer.

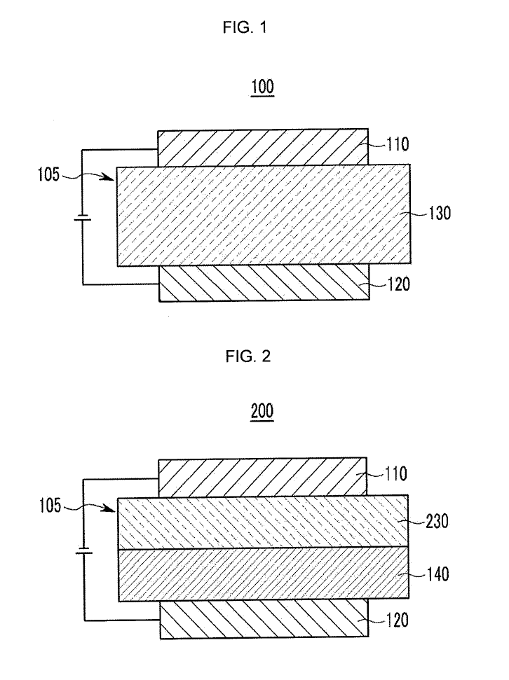

FIG. 1 is a cross-sectional view showing an organic light emitting diode as an example of the organic optoelectronic device according to an example embodiment.

Referring to FIG. 1, an organic light emitting diode 100 according to an example embodiment includes an anode 110 and a cathode 120 facing each other and an organic layer 105 disposed between the anode 110 and cathode 120.

The anode 110 may be made of a conductor having a high work function to help hole injection, and may be, for example, a metal, a metal oxide and/or a conductive polymer. The anode 110 may be, for example a metal such as nickel, platinum, vanadium, chromium, copper, zinc, gold, and the like or an alloy thereof; metal oxide such as zinc oxide, indium oxide, indium tin oxide (ITO), indium zinc oxide (IZO), and the like; a combination of metal and oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline.

The cathode 120 may be made of a conductor having a low work function to help electron injection, and may be, for example, a metal, a metal oxide and/or a conductive polymer. The cathode 120 may be, for example, a metal such as magnesium, calcium, sodium, potassium, titanium, indium, yttrium, lithium, gadolinium, aluminum silver, tin, lead, cesium, barium, and the like or an alloy thereof; a multi-layer structure material such as LiF/Al, LiO₂/Al, LiF/Ca, LiF/Al and BaF₂/Ca.

The organic layer 105 may include the organic compound or the composition.

The organic layer 105 may include a light emitting layer 130. The light emitting layer 130 may include the organic compound or the composition as a host. The light emitting layer 130 may further include other organic compounds as a host. The light emitting layer 130 may further include a dopant and the dopant may be, for example, a phosphorescent dopant.

The organic layer 105 may include an auxiliary layer between the anode 110 and the light emitting layer 130 and/or the cathode 120 and the light emitting layer 130. The auxiliary layer may be, for example, a hole injection layer, a hole transport layer, an electron blocking layer, an electron injection layer, an electron transport layer, a hole blocking layer, or a combination thereof. In an example embodiment, the auxiliary layer may include the organic compound or the composition.

FIG. 2 is a cross-sectional view of an organic light emitting diode according to another example embodiment.

Referring to FIG. 2, an organic light emitting diode 200 according to an example embodiment includes an anode 110 and a cathode 120 facing each other, and an organic layer 105 disposed between the anode 110 and the cathode 120.

The organic layer 105 includes an electron auxiliary layer 140 disposed between the light emitting layer 230 and the cathode 120. The electron auxiliary layer 140 may be, for example, an electron injection layer, an electron transport layer, and/or a hole blocking layer and may help injection and transport of electrons between the cathode 120 and the light emitting layer 230.

For example, the organic compound or the composition may be included in the light emitting layer 230. The light emitting layer 230 may further include other organic compounds as a host. The light emitting layer 230 may further include a dopant and the dopant may be, for example, a phosphorescent dopant.

For example, the organic compound may be included in the electron auxiliary layer 140. The electron auxiliary layer 140 may include the organic compound alone, at least two kinds of the organic compounds, or a mixture of the organic compound and other organic compound.

In FIG. 2, at least one layer of a hole auxiliary layer may be further included in an organic layer 105 between the anode 110 and the light emitting layer 230.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

First Compound for Organic Optoelectronic Device

### Synthesis Example 1: Synthesis of Compound 1

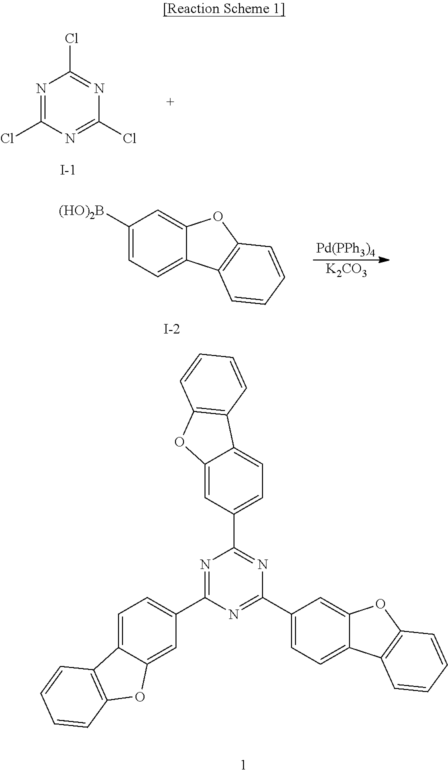

Intermediate I-1 (5.0 g, 27.11 mmol), Intermediate I-2 (18.39 g, 86.76 mmol), potassium carbonate (9.37 g, 67.78 mmol), and tetrakis(triphenylphosphine)palladium(0) (0.94 g, 0.81 mmol) were added to 180 mL of 1,4-dioxane and 90 mL of water in a 500 mL flask, and the mixture was heated at 100° C. under a nitrogen flow for 12 hours. Subsequently, an organic layer was separated therefrom and added to 500 mL of methanol, and a solid crystallized therein was filtered, dissolved in monochlorobenzene, filtered with silica gel/Celite, and then, after removing an appropriate amount of an organic solvent, recrystallized with monochlorobenzene to obtain Compound 1 (10.06 g, a yield of 64%).

calcd. C39H21N3O3: C, 80.82; H, 3.65; N, 7.25; 0, 8.28; found: C, 80.82; H, 3.64; N, 7.25; 0, 8.28.

### Synthesis Example 2: Synthesis of Compound 2

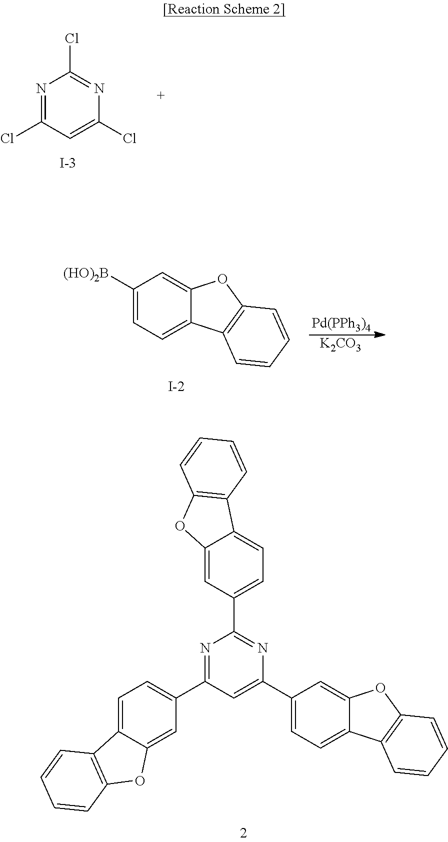

Compound 2 (8.30 g, a yield of 69%) was obtained according to the same method as Synthesis Example 1.

calcd. C40H22N2O3: C, 83.03; H, 3.83; N, 4.84; O, 8.30; found: C, 83.03; H, 3.83; N, 4.84; O, 8.30.

### Synthesis Example 3: Synthesis of Compound 3

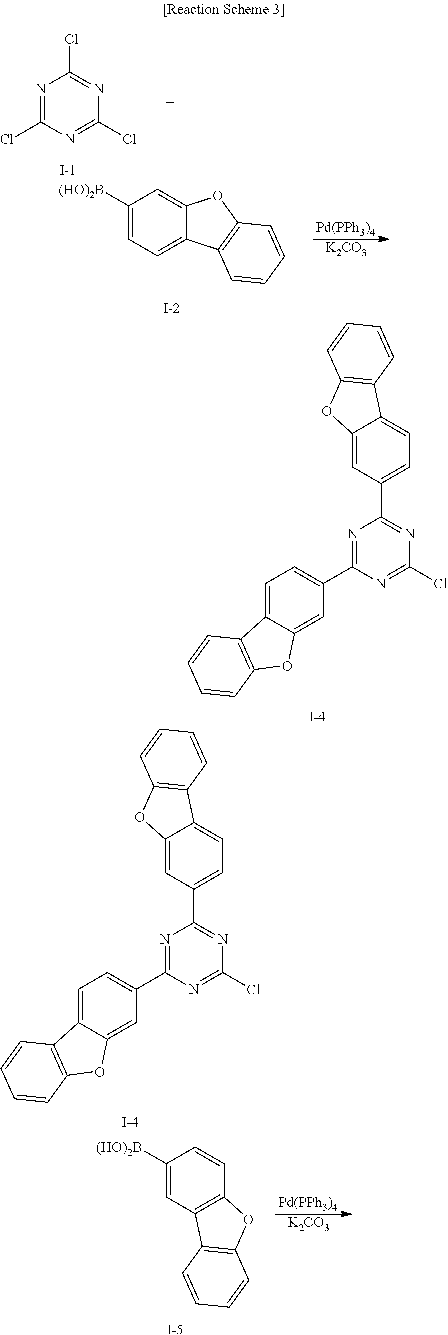

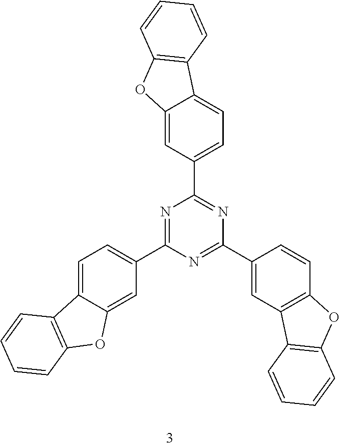 [SMILES: c1ccc2c(c1)oc1cc(-c3nc(-c4ccc5c(c4)oc4ccccc45)nc(-c4ccc5oc6ccccc6c5c4)n3)ccc12 ; "3"]

### Synthesis of Intermediate I-4

Intermediate I-1 (50.0 g, 271.1 mmol), Intermediate I-2 (120.7 g, 549.4 mmol), potassium carbonate (93.7 g, 677.8 mmol), and tetrakis(triphenylphosphine) palladium(0) (9.4 g, 8.4 mmol) were added to 1800 mL of 1,4-dioxane and 900 mL of water in a 5000 mL flask, and the mixture was heated at 100° C. under a nitrogen flow for 12 hours. Subsequently, an organic layer was separated therefrom and appropriately volatilized, and a solid crystallized by adding 2000 mL of methanol is filtered, dissolved in monochlorobenzene, filtered with silica gel/Celite, and then, after removing an appropriate amount of an organic solvent therefrom, recrystallized with monochlorobenzene to obtain Intermediate I-4 (45 g, a yield of 54.6%).

### Synthesis of Compound 3

Compound 3 (5.68 g, a yield of 63%) is obtained according to the same method as Synthesis Example 1.

calcd. C39H21N3O3: C, 80.82; H, 3.65; N, 7.25; O, 8.28; found: C, 80.82; H, 3.65; N, 7.25; O, 8.28.

### Synthesis Example 4: Synthesis of Compound 9

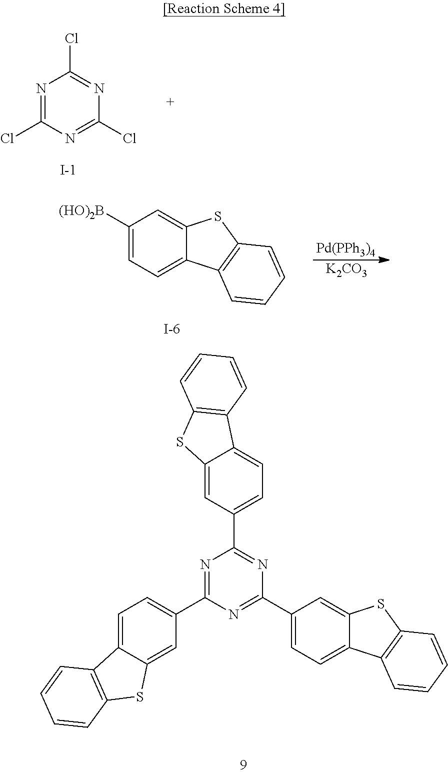

Compound 9 (12.5 g, a yield of 53%) is obtained according to the same method as Synthesis Example 1.

calcd. C39H21N3S3: C, 74.61; H, 3.37; N, 6.69; S, 15.32; found: C, 74.61; H, 3.37; N, 6.69; S, 15.32.

### Synthesis Example 5: Synthesis of Compound 10

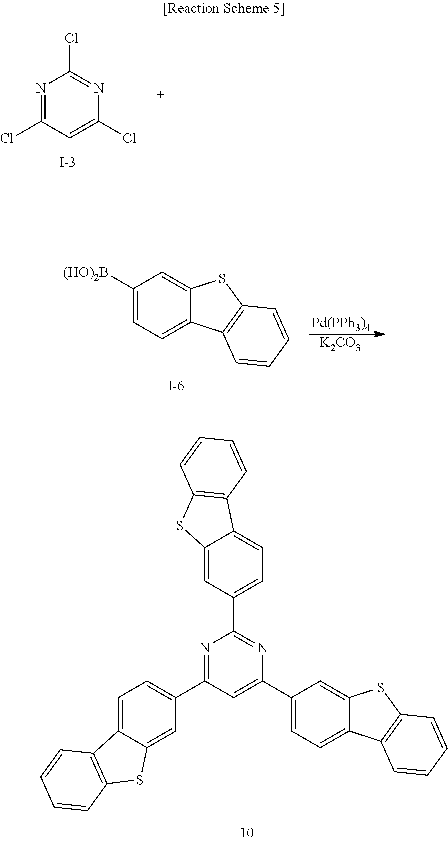

Compound 10 (7.7 g, a yield of 63%) is obtained according to the same method as Synthesis Example 2.

calcd. C40H22N2S3: C, 76.65; H, 3.54; N, 4.47; S, 15.35; found: C, 76.65; H, 3.54; N, 4.47; S, 15.35.

### Synthesis Example 6: Synthesis of Compound 11

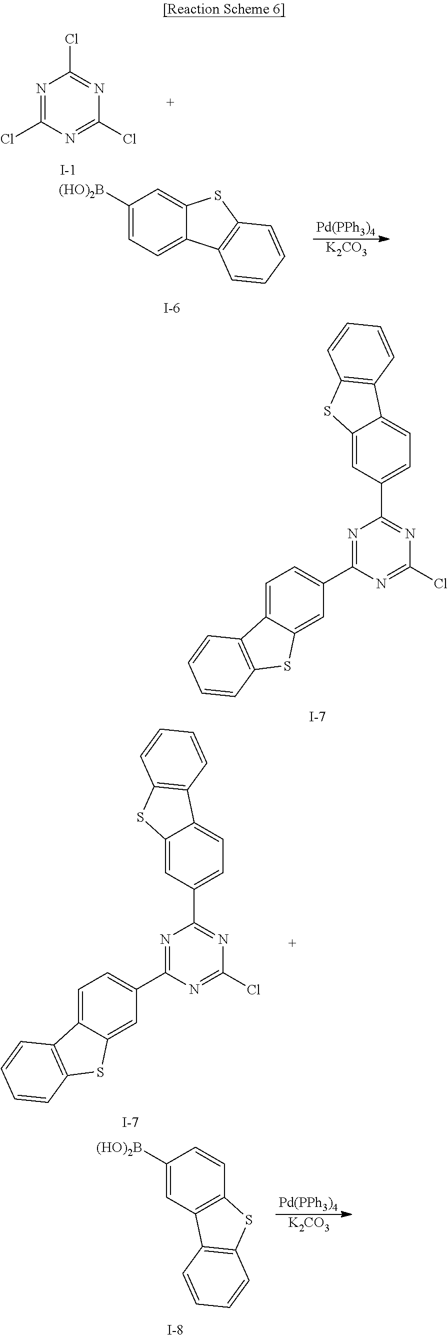

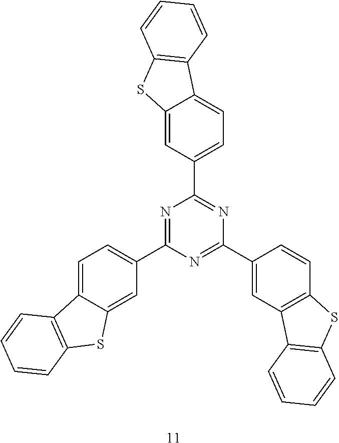 [SMILES: c1ccc2c(c1)sc1cc(-c3nc(-c4ccc5c(c4)sc4ccccc45)nc(-c4ccc5sc6ccccc6c5c4)n3)ccc12 ; "11"]

Compound 11 (11.0 g, a yield of 73%) is obtained according to the same method as Synthesis Example 3.

calcd. C39H21N3S3: C, 74.61; H, 3.37; N, 6.69; S, 15.32; found: C, 74.61; 1-1, 3.37; N, 6.69; S, 15.31.

### Synthesis Example 7: Synthesis of Compound 17

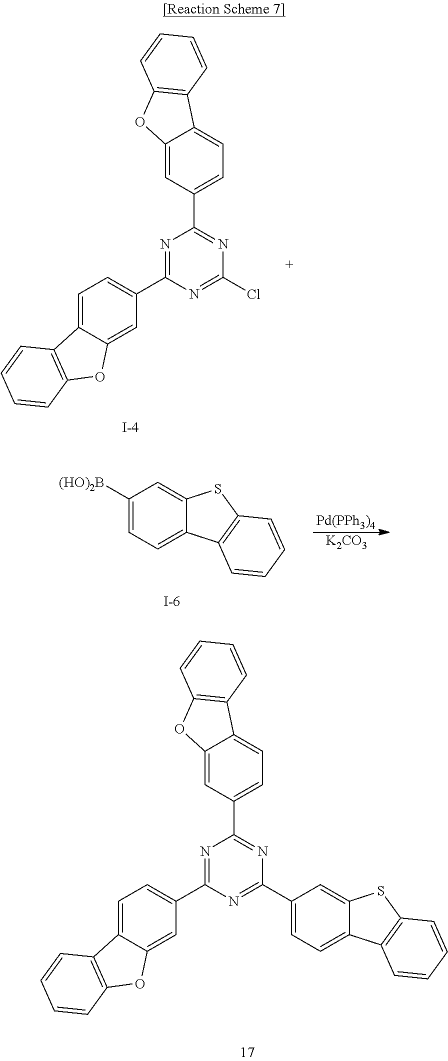

Compound 17 (8.9 g, a yield of 70%) was obtained according to the same method as Synthesis Example 3.

calcd. C39H21N3O2S: C, 78.64; H, 3.55; N, 7.05; O, 5.37; S, 5.38; found: C, 78.64; H, 3.55; N, 7.05; O, 5.37; S, 5.38.

### Synthesis Example 8: Synthesis of Compound 51

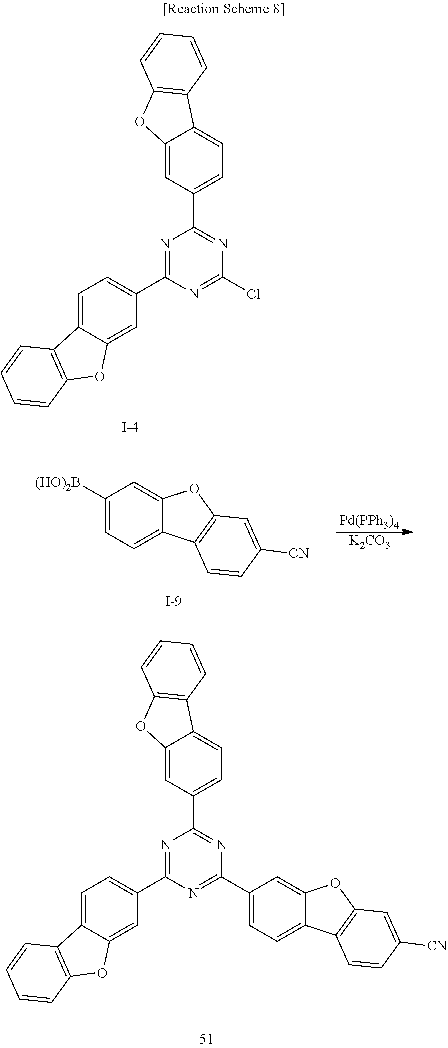

Compound 51 (4.7 g. a yield of 66%) was obtained according to the same method as Synthesis Example 3.

calcd. C40H20N4O3: C, 79.46; H, 3.33; N, 9.27; O, 7.94; found: C, 79.46; H, 3.33; N, 9.27; O, 7.94.

### Synthesis Example 9: Synthesis of Compound 52

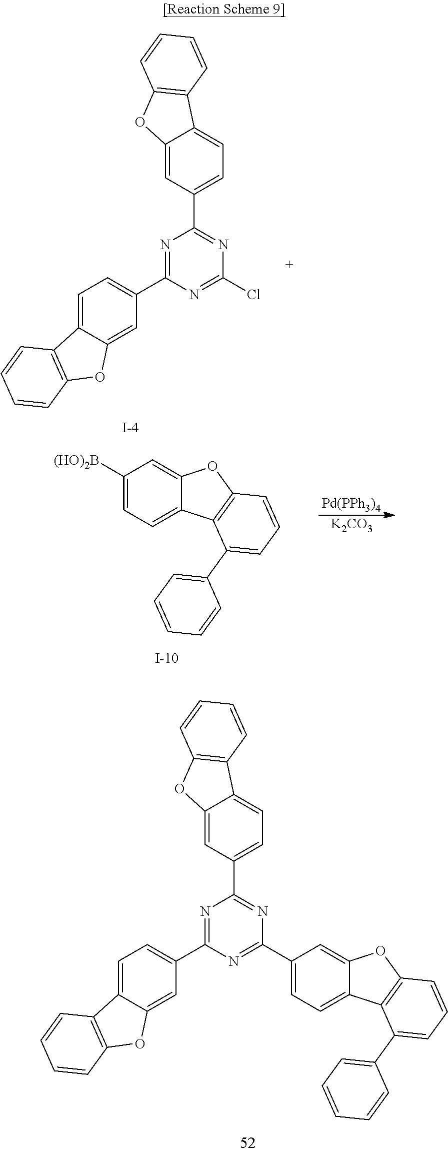

Compound 52 (3.9 g, a yield of 63%) was obtained according to the same method as Synthesis Example 3.

calcd. C45H25N3O3: C, 82.43; H, 3.84; N, 6.41; O, 7.32; found: C, 82.43; H, 3.84; N, 6.41; O, 7.32.

### Synthesis Example 10: Synthesis of Compound 78

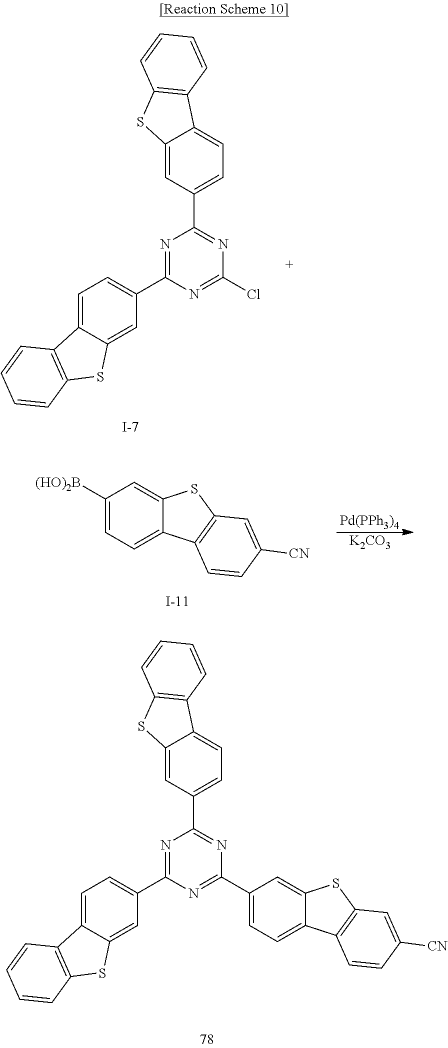

Compound 78 (5.0 g, a yield of 68%) was obtained according to the same method as Synthesis Example 3.

calcd. C40H20N4S3: C, 73.59; 14, 3.09; N, 8.58; S, 14.74; found: C, 73.59; H, 3.09; N, 8.58; S, 14.74.

### Synthesis Example 11: Synthesis of Compound 80

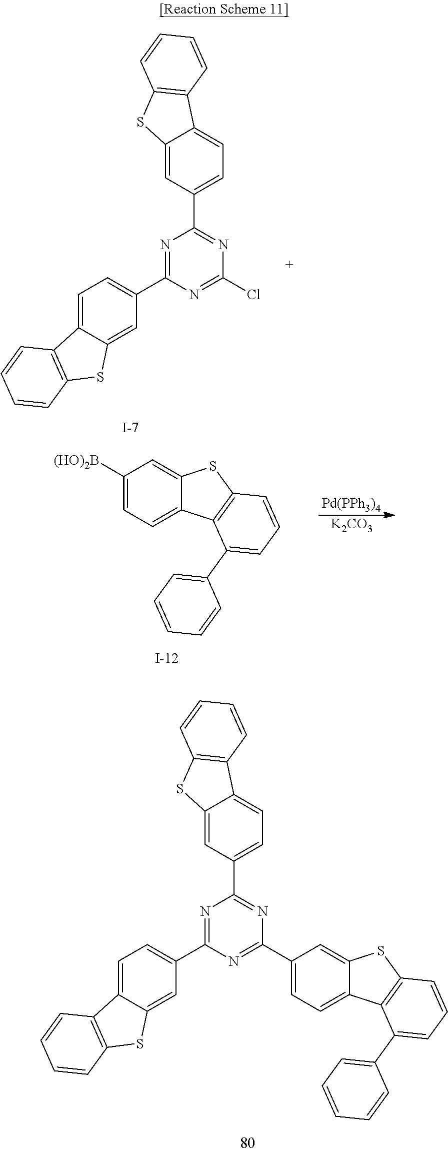

Compound 80 (8.5 g, a yield of 65%) was obtained according to the same method as Synthesis Example 6.

calcd. C45H25N3S3: C, 76.78; H, 3.58; N, 5.97; S, 13.67; found: C, 76.78; H, 3.58; N, 5.97; S, 13.67.

### Comparative Synthesis Examples 1 to 6

Comparative compounds 1 to 6 were synthesized according to the same method as Synthesis Examples 1 to 11.

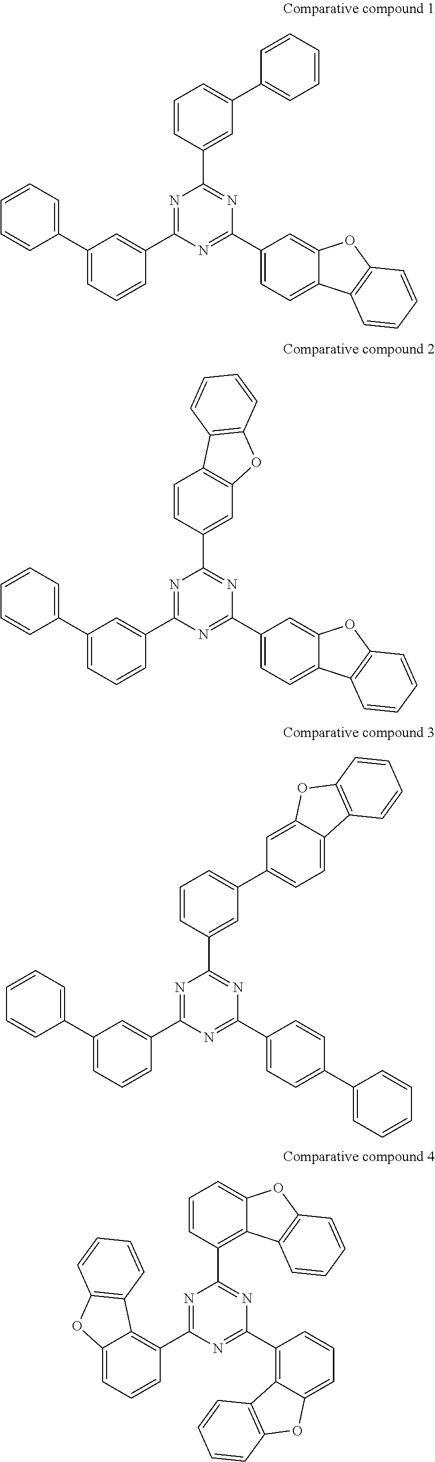 [SMILES: c1ccc(-c2cccc(-c3nc(-c4cccc(-c5ccccc5)c4)nc(-c4ccc5c(c4)oc4ccccc45)n3)c2)cc1 ; c1ccc(-c2cccc(-c3nc(-c4ccc5c(c4)oc4ccccc45)nc(-c4ccc5c(c4)oc4ccccc45)n3)c2)cc1 ; c1ccc(-c2ccc(-c3nc(-c4cccc(-c5ccccc5)c4)nc(-c4cccc(-c5ccc6c(c5)oc5ccccc56)c4)n3)cc2)cc1 ; c1ccc2c(c1)oc1cccc(-c3nc(-c4cccc5oc6ccccc6c45)nc(-c4cccc5oc6ccccc6c45)n3)c12]

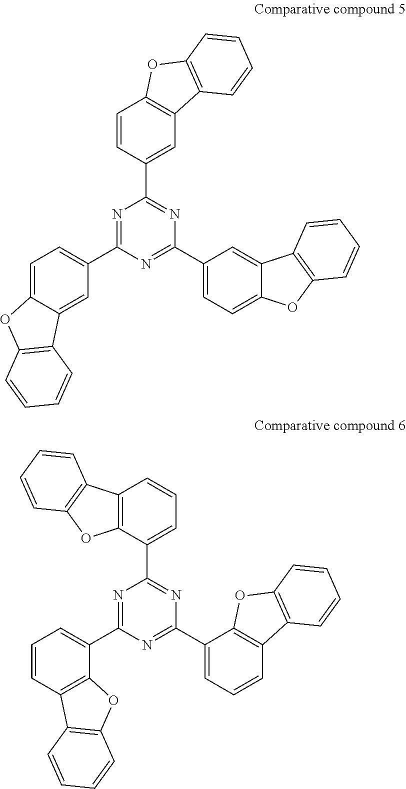

### Second Compound for Organic Optoelectronic Device

### Synthesis Example 12: Synthesis of Compound E-22

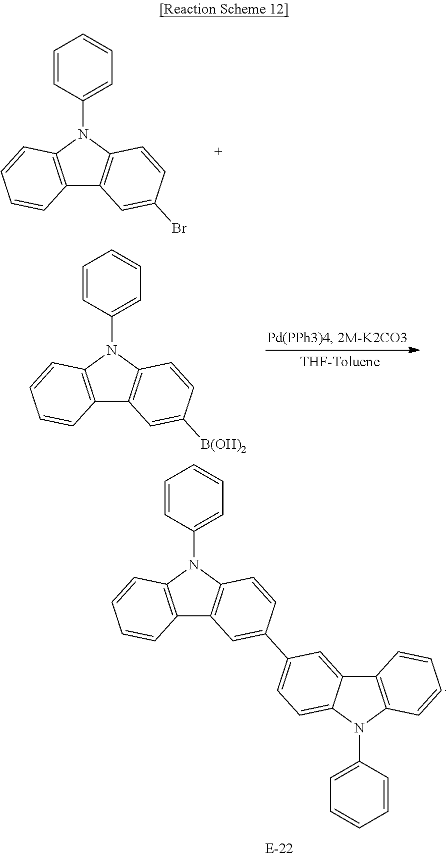

16.62 g (51.59 mmol) of 3-bromo-N-phenylcarbazole, 17.77 g (61.91 mmol) of N-phenylcarbazole-3-yl boronic acid, 200 mL of tetrahydrofuran:toluene (1:1), and 100 mL of a 2 M potassium carbonate aqueous solution were mixed in a 500 mL round-bottomed flask equipped with an agitator under a nitrogen atmosphere, 2.98 g (2.58 mmol) of tetrakistriphenyl phosphine palladium(0) was added thereto, and the mixture was heated and refluxed under a nitrogen flow for 12 hours. When a reaction was complete, the reactants were poured into methanol, and a solid produced therein was filtered, washed with water and methanol, and dried. Subsequently, a resulting material obtained therefrom was heated and dissolved in 1 L of chlorobenzene, the solution was silica gel-filtered, and then, after completely removing a solvent therefrom, a product therefrom was heated and dissolved in 500 mL of toluene and then, recrystallized to obtain 16.05 g of Compound E-22 (a yield of 64%).

calcd. C₃₆H₂₄N₂: C, 89.23; H, 4.99; N, 5.78; found: C, 89.45; H, 4.89; N, 5.65.

### Synthesis of Synthesis Examples 13 to 18

Each compound according to Synthesis Examples 13 to 18 was synthesized according to the same method as Synthesis Example 12 by using a starting material and a reactant as shown in

TABLE 1

| Synthesis | Starting | | Amount | Properties data of |
|---|---|---|---|---|
| Examples | materials | Final products | (yield) | final product |
| Synthesis Example 13 | 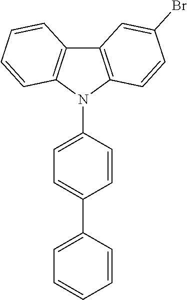 | 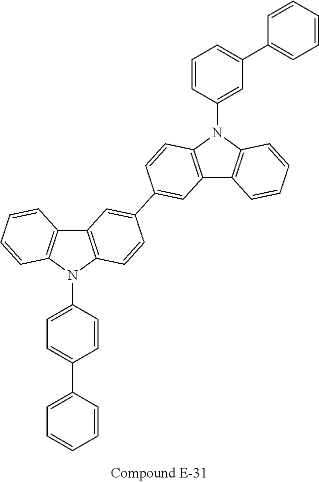 [SMILES: c1ccc(-c2ccc(-n3c4ccccc4c4cc(-c5ccc6c(c5)c5ccccc5n6-c5cccc(-c6ccccc6)c5)ccc43)cc2)cc1] | 6.57 g, 83% | calcd. C48H32N2: C, 90.54; H, 5.07; N, 4.40; found: C, 90.54; H, 5.07; N, 4.40 |
| Synthesis Example 14 | 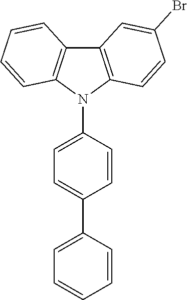 | 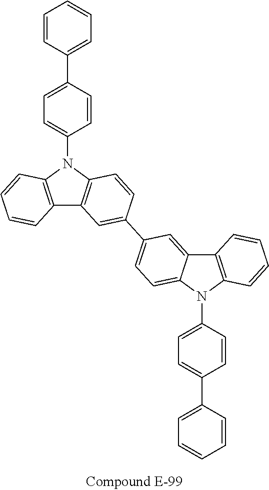 [SMILES: c1ccc(-c2ccc(-n3c4ccccc4c4cc(-c5ccc6c(c5)c5ccccc5n6-c5ccc(-c6ccccc6)cc5)ccc43)cc2)cc1] | 5.68 g, 75% | calcd. C48H32N2: C, 90.54; H, 5.07; N, 4.40; found: C, 90.54; H, 5.06; N, 4.40 |
| Synthesis Example 15 | 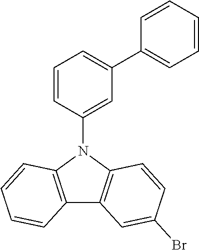 | 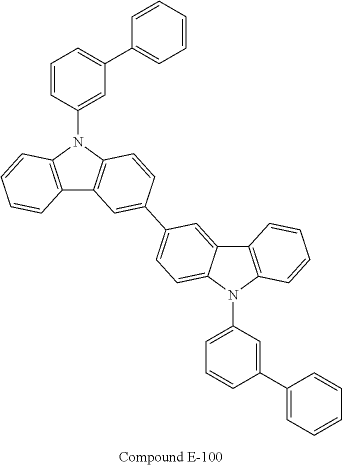 [SMILES: c1ccc(-c2cccc(-n3c4ccccc4c4cc(-c5ccc6c(c5)c5ccccc5n6-c5cccc(-c6ccccc6)c5)ccc43)c2)cc1] | 6.79 g, 79% | calcd. C48H32N2: C, 90.54; H, 5.07; N, 4.40; found: C, 90.54; H, 5.07; N, 4.40 |
| Synthesis Example 16 | 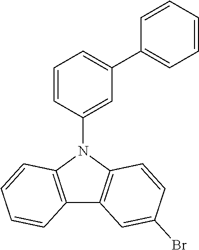 | 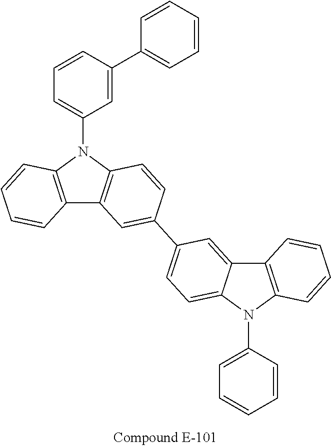 [SMILES: c1ccc(-c2cccc(-n3c4ccccc4c4cc(-c5ccc6c(c5)c5ccccc5n6-c5ccccc5)ccc43)c2)cc1] | 5.06 g, 75% | calcd. C42H28N2: C, 89.97; H, 5.03; N, 5.00; found: C, 89.97; H, 5.03; N, 5.00 |
| Synthesis Example 17 | 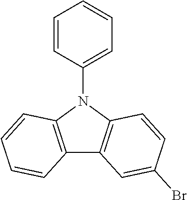 | 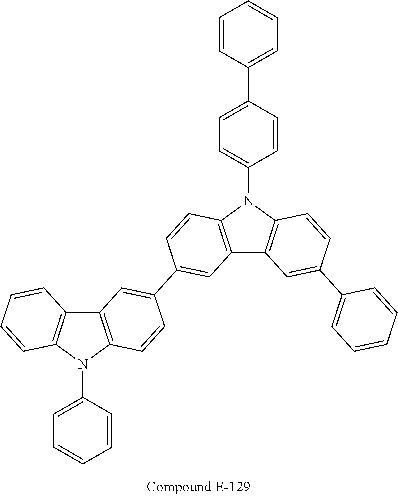 [SMILES: c1ccc(-c2ccc(-n3c4ccc(-c5ccccc5)cc4c4cc(-c5ccc6c(c5)c5ccccc5n6-c5ccccc5)ccc43)cc2)cc1] | 4.81 g, 69% | calcd. C48H32N2: C. 90.54; H, 5.07; N, 4.40; found: C, 90.54; H, 5.07; N, 4.39 |
| Synthesis Example 18 | 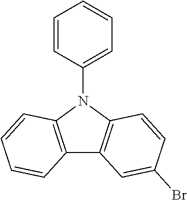 | 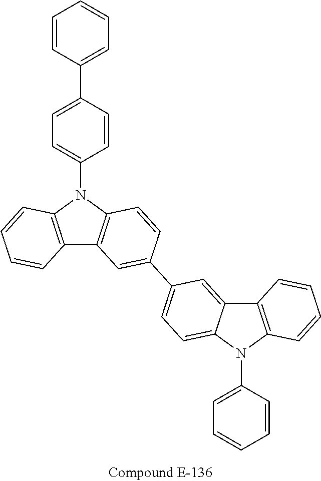 [SMILES: c1ccc(-c2ccc(-n3c4ccccc4c4cc(-c5ccc6c(c5)c5ccccc5n6-c5ccccc5)ccc43)cc2)cc1] | 5.84 g, 70% | calcd. C42H28N2: C, 89.97; H, 5.03; N, 5.00; found: C, 89.97; H, 5.03; N, 5.00 |

### Synthesis Example 19: Synthesis of Compound F-21

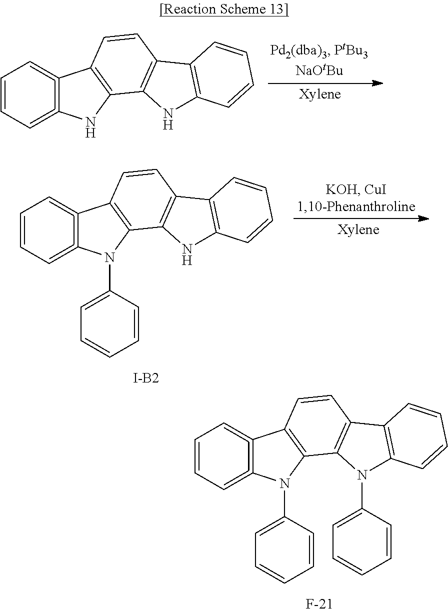

### Synthesis of Intermediate I-B2

39.99 g (156.01 mmol) of indolocarbazole, 26.94 g (171.61 mmol) of bromobenzene, 22.49 g (234.01 mmol) of sodium t-butoxide, 4.28 g (4.68 mmol) of tris(dibenzylideneacetone)dipalladium, and 2.9 mL of tri t-butylphosphine (50% in toluene) were mixed with 500 mL of xylene in a 1000 mL round flask, and the mixture was heated and refluxed under a nitrogen flow for 15 hours. The obtained mixture was added to 1000 mL of methanol, and a solid crystallized therein was filtered, dissolved in dichlorobenzene, filtered with silica gel/Celite, and then, after removing an appropriate amount of an organic solvent, recrystallized with methanol to obtain Intermediate I-B2 (23.01 g, a yield of 44%).

calcd. C₂₄H₁₆N₂: C, 86.72; H, 4.85; N, 8.43; found: C, 86.72; H, 4.85; N, 8.43.

### Synthesis of Compound F-21

22.93 g (69.03 mmol) of Intermediate B2, 11.38 g (72.49 mmol) of bromobenzene, 4.26 g (75.94 mmol) of potassium hydroxide, 13.14 g (69.03 mmol) of copper iodide, and 6.22 g (34.52 mmol) of 1,10-phenanthroline were mixed with 230 mL of DMF in a 500 mL round flask, and the mixture was heated and refluxed under a nitrogen flow for 15 hours. The obtained mixture was added to 1000 mL of methanol, and a solid crystallized therein was filtered, dissolved in dichlorobenzene, filtered with silica gel/Celite, and then, after removing appropriate amount of an organic solvent therefrom was recrystallized with methanol to obtain Compound F-21 (12.04 g, a yield of 43%).

calcd. C₃₀H₂₀N₂: C, 88.21; H, 4.93; N, 6.86; found: C, 88.21; H, 4.93; N, 6.86.

### Synthesis Example 20: Synthesis of Intermediate I

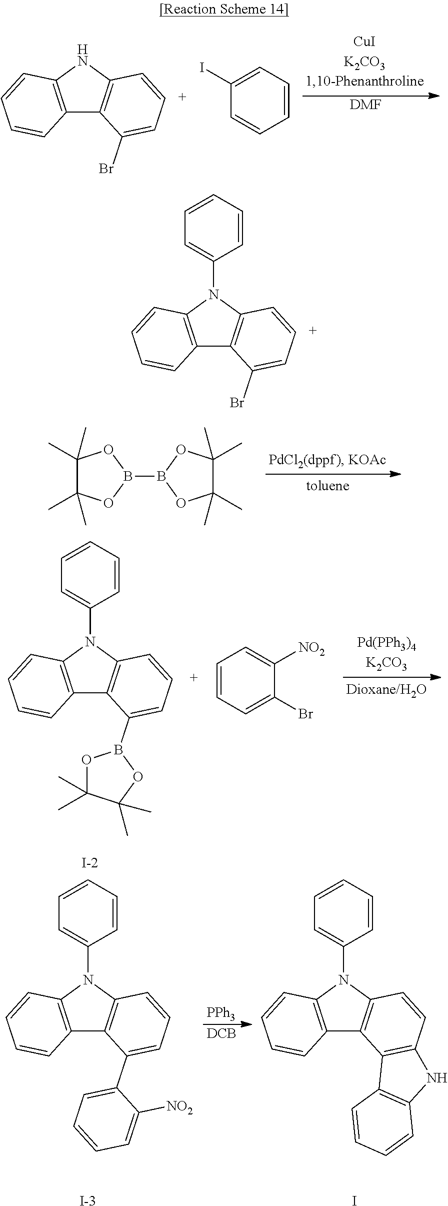

### Synthesis of Intermediate I-1

200.0 g (0.8 mol) of an intermediate of 4-bromo-9H-carbazole, 248.7 g (1.2 mol) of iodo benzene, 168.5 g (1.2 mol) of potassium carbonate, 31.0 g (0.2 mol) of copper(I) iodide, and 29.3 g (0.2 mol) of 1,10-phenanthroline were mixed with 2.5 L of N,N-dimethylformamide in a 5 L flask, and the mixture was refluxed under a nitrogen flow for 24 hours. The obtained mixture was added to 4 L of distilled water, and a solid crystallized therein was filtered and washed with water, methanol, and hexane. Subsequently, the solid was extracted with water and dichloromethane, and an organic layer obtained therefrom was treated by using magnesium sulfate to remove moisture and then, concentrated and purified through column chromatography to obtain Intermediate I-1 as a white solid (216.2 g, a yield of 83%).

calcd. C27H18ClN3: C, 67.10; H, 3.75; Br, 24.80; N, 4.35; found: C C, 67.12; H, 3.77; Br, 24.78; N, 4.33.

### Synthesis of Intermediate I-2

Intermediate I-1 (216.0 g, 0.7 mol), 4,4,4′,4′,5,5,5′,5′-octamethyl-2,2′-bi(1,3,2-dioxaborolane (212.8 g, 0.8 mol), potassium acetate (KOAc, 197.4 g, 2.0 mol), 1,1′-bis(diphenylphosphino) ferrocene-palladium(II)dichloride (21.9 g, 0.03 mol), and tricyclohexylphosphine (45.1 g, 0.2 mol) were added to 3 L of N,N-dimethylformamide in a 5 L flask, and the mixture was stirred at 130° C. for 12 hours. When a reaction was complete, an organic layer obtained by extracting the reaction solution with water and ethyl acetate (EA) was treated with magnesium sulfate to remove moisture therefrom, concentrated, and purified through column chromatography to obtain Intermediate I-2 as a white solid (205.5 g, a yield of 83%).

calcd. C26H25BN2O2: C, 78.06; H, 6.55; B, 2.93; N, 3.79; O, 8.67; found: C, 78.08; H, 6.57; B, 2.91; N, 3.77; O, 8.67.

### Synthesis of Intermediate I-3

150.0 g (0.4 mol) of Intermediate I-2, 164.1 g (0.8 mol) of an intermediate of 1-bromo-2-nitrobenzene, 278.1 g (2.01 mol) of potassium carbonate, and 23.5 g (0.02 mol) of tetrakis(triphenylphosphine)palladium(0) were added to 2 L of 1,4-dioxane and 1 L of water in a 5 L flask, and the mixture was heated at 90° C. under a nitrogen flow for 16 hours. After removing a reaction solvent therefrom, a resultant therefrom was dissolved in dichloromethane, filtered with silica gel/Celite, and then, after removing an appropriate amount of an organic solvent, recrystallized with methanol to obtain Intermediate I-3 as a yellow solid (86.3 g, a yield of 58%).

calcd. C18H12N2O2: C, 79.11; H, 4.43; N, 7.69; O, 8.78; found: C, 79.13; H, 4.45; N, 7.67; O, 8.76.

### Synthesis of Intermediate I

Intermediate I-3 (86.0 g, 0.23 mol) and triphenylphosphine (309.5 g, 1.18 mol) were added to 600 mL of dichlorobenzene in a 1000 ml flask, nitrogen was substituted for inside of the flask, and the mixture was stirred at 160° C. for 12 hours. When a reaction was complete, a solvent was removed therefrom, and a resultant therefrom was purified with hexane through column chromatography to obtain Intermediate I as a yellow solid (57.3 g, a yield of 73%).

Calcd. C18H12N2: C, 86.72; H, 4.85; N, 8.43; found: C, 86.70; H, 4.83; N, 8.47.

### Synthesis of Synthesis Examples 21 to 33

Each compound was synthesized according to the same method as that of preparing Compound F-21 and Intermediate I according to Synthesis Examples 19 and 20 by using a starting material and a reactant as shown in Table 2.

TABLE 2

| Synthesis | Starting | | Amount | Properties data of |
|---|---|---|---|---|
| Examples | materials | Final products | (yield) | final product |
| Synthesis Example 21 | 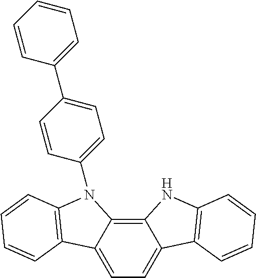 | 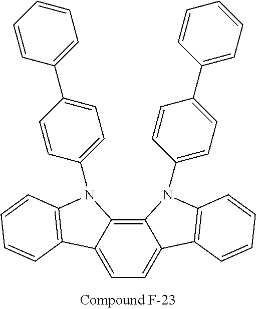 [SMILES: c1ccc(-c2ccc(-n3c4ccccc4c4ccc5c6ccccc6n(-c6ccc(-c7ccccc7)cc6)c5c43)cc2)cc1] | 10.23 g, 45% | calcd. C42H28N2: C, 89.97; H, 5.03; N, 5.00; found: C, 89.97; H, 5.03; N, 5.00 |
| Synthesis Example 22 | 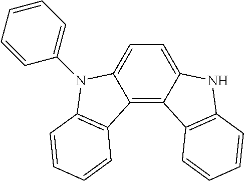 | 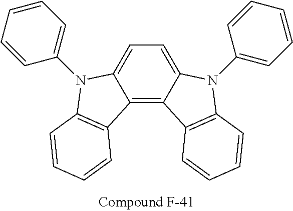 [SMILES: c1ccc(-n2c3ccccc3c3c4c5ccccc5n(-c5ccccc5)c4ccc32)cc1] | 7.31 g, 77% | calcd. C30H20N2: C, 88.21; H, 4.93; N, 6.86; found: C, 88.21; H, 4.93; N, 6.86 |
| Synthesis Example 23 | 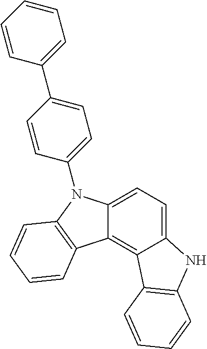 | 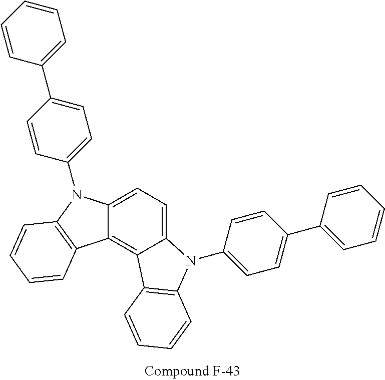 [SMILES: c1ccc(-c2ccc(-n3c4ccccc4c4c5c6ccccc6n(-c6ccc(-c7ccccc7)cc6)c5ccc43)cc2)cc1] | 6.33 g, 76% | calcd. C42H28N2: C, 89.97; H, 5.03; N, 5.00; found: C, 89.97; H, 5.03; N, 5.00 |
| Synthesis Example 24 | 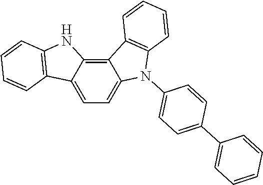 | 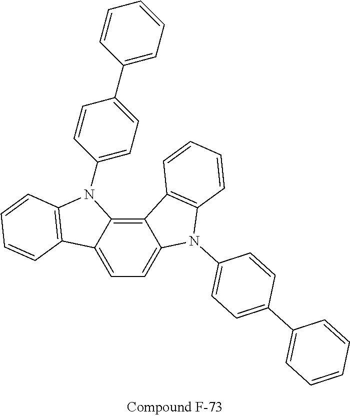 [SMILES: c1ccc(-c2ccc(-n3c4ccccc4c4c3ccc3c5ccccc5n(-c5ccc(-c6ccccc6)cc5)c34)cc2)cc1] | 8.33 g, 74% | calcd. C42H28N2: C, 89.97; H, 5.03; N, 5.00; found: C, 89.97; H. 5.03; N, 5.00 |
| Synthesis Example 25 | 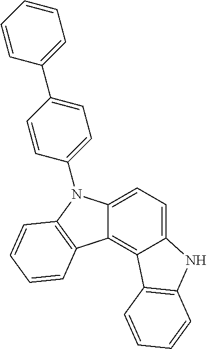 | 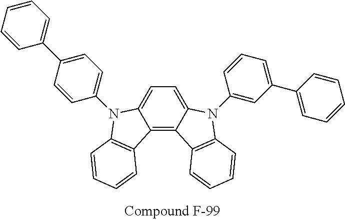 [SMILES: c1ccc(-c2ccc(-n3c4ccccc4c4c5c6ccccc6n(-c6cccc(-c7ccccc7)c6)c5ccc43)cc2)cc1] | 5.53 g, 79% | calcd. C42H28N2: C, 89.97; H, 5.03; N, 5.00; found: C, 89.97; H, 5.03; N, 5.00 |
| Synthesis Example 26 | 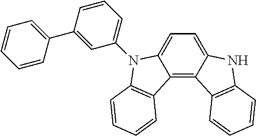 | 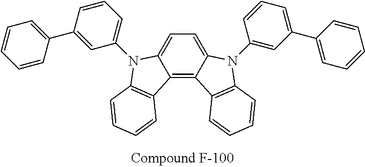 [SMILES: c1ccc(-c2cccc(-n3c4ccccc4c4c5c6ccccc6n(-c6cccc(-c7ccccc7)c6)c5ccc43)c2)cc1] | 7.41 g, 73% | calcd. C42H28N2: C, 89.97; H, 5.03; N, 5.00; found: C, 89.97; H, 5.03; N, 5.00 |
| Synthesis Example 27 | 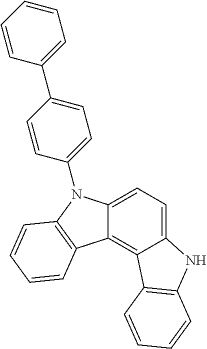 | 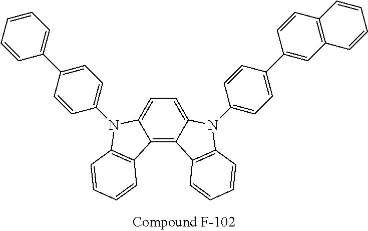 [SMILES: c1ccc(-c2ccc(-n3c4ccccc4c4c5c6ccccc6n(-c6ccc(-c7ccc8ccccc8c7)cc6)c5ccc43)cc2)cc1] | 5.94 g, 76% | calcd. C46H30N2: C, 90.46; H, 4.95; N, 4.59; found: C, 90.46; H, 4.95; N, 4.59 |
| Synthesis Example 28 | 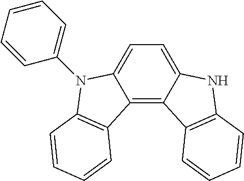 | 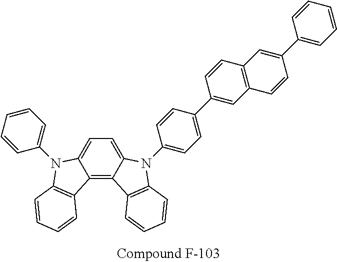 [SMILES: c1ccc(-c2ccc3cc(-c4ccc(-n5c6ccccc6c6c7c8ccccc8n(-c8ccccc8)c7ccc65)cc4)ccc3c2)cc1] | 6.37 g, 76% | calcd. C46H30N2: C, 90.46; H, 4.95; N, 4.59; found: C, 90.46; H, 4.95; N, 4.59 |
| Synthesis Example 29 | 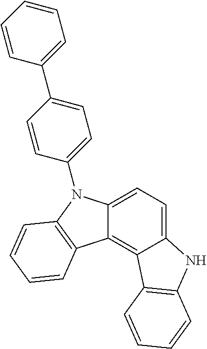 | 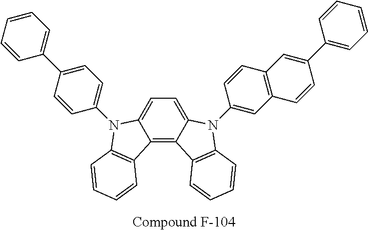 [SMILES: c1ccc(-c2ccc(-n3c4ccccc4c4c5c6ccccc6n(-c6ccc7cc(-c8ccccc8)ccc7c6)c5ccc43)cc2)cc1] | 10.39 g, 79% | calcd. C46H30N2: C, 90.46; H, 4.95; N, 4.59; found: C, 90.46; H, 4.95; N, 4.59 |
| Synthesis Example 30 | 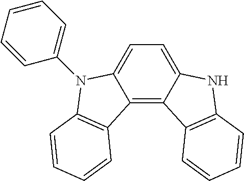 | 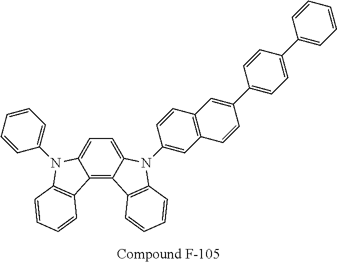 [SMILES: c1ccc(-c2ccc(-c3ccc4cc(-n5c6ccccc6c6c7c8ccccc8n(-c8ccccc8)c7ccc65)ccc4c3)cc2)cc1] | 5.33 g, 69% | calcd. C46H30N2: C, 90.46; H, 4.95; N, 4.59; found: C, 90.46; H, 4.95; N, 4.59 |
| Synthesis Example 31 | 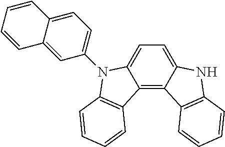 | 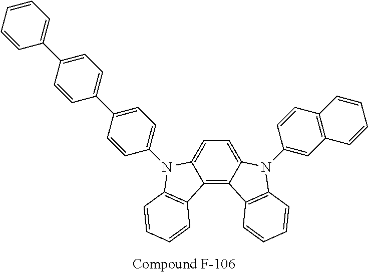 [SMILES: c1ccc(-c2ccc(-c3ccc(-n4c5ccccc5c5c6c7ccccc7n(-c7ccc8ccccc8c7)c6ccc54)cc3)cc2)cc1] | 6.96 g, 77% | calcd. C46H30N2: C, 90.46; H, 4.95; N, 4.59; found: C, 90.46; H, 4.95; N, 4.59 |
| Synthesis Example 32 | 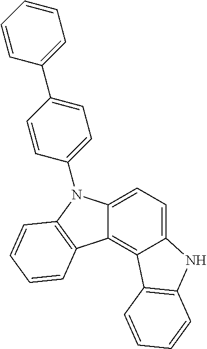 | 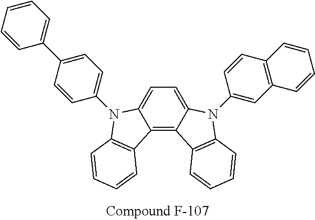 [SMILES: c1ccc(-c2ccc(-n3c4ccccc4c4c5c6ccccc6n(-c6ccc7ccccc7c6)c5ccc43)cc2)cc1] | 6.11 g, 77% | calcd. C40H26N2: C, 89.86; H, 4.90; N, 5.24; found: C, 89.86; H, 4.90; N, 5.24 |
| Synthesis Example 33 | 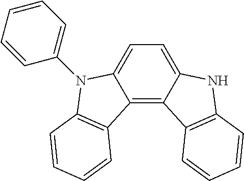 | 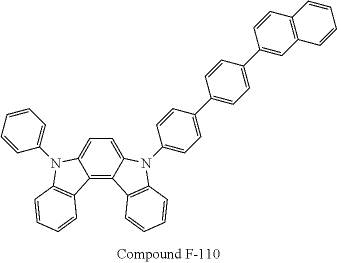 [SMILES: c1ccc(-n2c3ccccc3c3c4c5ccccc5n(-c5ccc(-c6ccc(-c7ccc8ccccc8c7)cc6)cc5)c4ccc32)cc1] | 9.44 g, 75% | calcd. C46H30N2: C, 90.46; H, 4.95; N, 4.59; found: C, 90.46; H, 4.95; N, 4.59 |

Manufacture of Organic Light Emitting Diode I

### Example 1

A glass substrate disposed with an ITO electrode was cut into a size of 50 mm×50 mm×0.5 mm and then, ultrasonic wave cleaned with acetone isopropyl alcohol and pure water respectively for 15 minutes and UV ozone cleaned for 30 minutes. On the ITO electrode, m-MTDATA was vacuum-deposited at 1 Å/sec to form a 600 Å-thick hole injection layer, and on the hole injection layer, α-NPB was vacuum-deposited at 1 Å/sec to form a 300 Å thick hole transport layer. Subsequently, on the hole transport layer, Ir(ppy)₃ (a dopant), Compound 1 according to Synthesis Example 1, and Compound ETH-1 were codeposited at each deposition rate of 0.1 Å/sec and 1 Å/sec to form a 400 Å-thick light emitting layer. On the light emitting layer, bis(2-methyl-8-quinolinolate)-4-(phenylphenolato)aluminum [BAlq] was vacuum-deposited at 1 Å/sec to form a 50 Å-thick hole-blocking layer, and on the hole blocking layer, Alq₃ was vacuum-deposited to form a 300 Å-thick electron transport layer. On the electron transport layer, LiF 10 Å (an electron injection layer) and Al 2000 Å (a cathode) were sequentially vacuum-deposited to manufacture an organic light emitting diode.

### Examples 2 to 11

Each organic light emitting diode according to Examples 2 to II was manufactured according to the same method as Example 1 by respectively using a compound shown in Table 3 instead of Compound 1 as a host for forming a light emitting layer.

### Comparative Example 1 to 6

Each organic light emitting diode according to Comparative Synthesis Examples 1 to 6 was manufactured according to the same method as Example 1 by using each compound according to Comparative Synthesis Examples 1 to 6 instead of Compound 1 as a host for forming a light emitting layer.

Evaluation 1

A driving voltage, efficiency, luminance, and a life-span of each organic light emitting diode according to Examples 1 to 11 and Comparative Examples 1 to 6 were measured by supplying power from a current voltage meter (Keithley SMU 236) and using a luminance meter, PR650 Spectroscan Source Measurement Unit (Photo Research Inc.). The results are shown in Table 3.

Specific measurement methods are as follows.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000A), while the voltages of the organic light emitting diodes were increased from 0 V to 10 V.

(3) Measurement of Luminous Efficiency

Current efficiency (cd/A) at the same current density (10 mA/cm²) were calculated by using the luminance, current density, and voltages (V) from the items (1) and (2).

TABLE 3

| | | | Driv- | | | |
|---|---|---|---|---|---|---|
| | | | ing | Current | | T₉₅ |
| | | | volt- | effi- | Lumi- | life- |
| | | Dop- | age | ciency | nance | span |
| | Host | ant | (V) | (cd/A) | (cd/m²) | (hr) |
| Example 1 | Compound 1 | Ir(ppy)₃ | 4.2 | 47 | 6000 | 77 |
| Example 2 | Compound 2 | Ir(ppy)₃ | 4.5 | 48 | 6000 | 72 |
| Example 3 | Compound 3 | Ir(ppy)₃ | 4.3 | 47 | 6000 | 76 |
| Example 4 | Compound 9 | Ir(ppy)₃ | 4.2 | 48 | 6000 | 76 |
| Example 5 | Compound 10 | Ir(ppy)₃ | 4.6 | 49 | 6000 | 73 |
| Example 6 | Compound 11 | Ir(ppy)₃ | 4.4 | 48 | 6000 | 76 |
| Example 7 | Compound 17 | Ir(ppy)₃ | 4.3 | 48 | 6000 | 76 |
| Example 8 | Compound 51 | Ir(ppy)₃ | 4.4 | 46 | 6000 | 81 |
| Example 9 | Compound 52 | Ir(ppy)₃ | 4.1 | 49 | 6000 | 80 |
| Example 10 | Compound 78 | Ir(ppy)₃ | 4.5 | 47 | 6000 | 77 |
| Example 11 | Compound 80 | Ir(ppy)₃ | 4.3 | 49 | 6000 | 78 |
| Comparative | Comparative | Ir(ppy)₃ | 4.9 | 40 | 6000 | 65 |
| Example 1 | Compound 1 | | | | | |
| Comparative | Comparative | Ir(ppy)₃ | 4.7 | 44 | 6000 | 68 |
| Example 2 | Compound 2 | | | | | |
| Comparative | Comparative | Ir(ppy)₃ | 5.0 | 42 | 6000 | 65 |
| Example 3 | Compound 3 | | | | | |
| Comparative | Comparative | Ir(ppy)₃ | 4.8 | 44 | 6000 | 68 |
| Example 4 | Compound 4 | | | | | |
| Comparative | Comparative | Ir(ppy)₃ | 4.7 | 43 | 6000 | 68 |
| Example 5 | Compound 5 | | | | | |
| Comparative | Comparative | Ir(ppy)₃ | 4.7 | 43 | 6000 | 69 |
| Example 6 | Compound 6 | | | | | |

Referring to Table 3, the organic light emitting diode according to Examples 1 to 11 showed a low driving voltage, high efficiency, and a long life-span compared with the organic light emitting diodes according to Comparative Examples 1 to 6.

Accordingly, a host material used in the organic light emitting diodes according to Examples 1 to 11 had excellent charge transport characteristics, was well overlapped with an absorption spectrum of a dopant, and turned out to improve performance such as an efficiency increase and a driving voltage decrease and show maximized capability as an OLED material.

Manufacture of Organic Light Emitting Diode II

### Example 12

An organic light emitting diode was manufactured according to the same method as Example 1 by codepositing Ir(ppy)₃ (a dopant), Compound 1 (a first host), and Compound E-31 (a second host) in a weight ratio of 10:45:45 on a hole transport layer (HTL) to form a 400 Å-thick light emitting layer.

### Examples 13 to 35

An organic light emitting diode was manufactured according to the same method as Example 12 except for using each first and second host shown in Table 4.

### Comparative Examples 7 to 12

An organic light emitting diode was manufactured according to the same method as Example 12 except for using each first and second host shown in Table 4.

Evaluation 2

A driving voltage, efficiency, luminance, and a life-span of each organic light emitting diode according to Examples 12 to 35 and Comparative Examples 7 to 12 were measured by supplying power from a current voltage meter (Keithley SMU 236) and using a luminance meter, PR650 Spectroscan Source Measurement Unit (Photo Research Inc.). The results are shown in Table 4.

A T₉₅ life-span is to evaluate how long (hr) it takes for an organic light emitting diode to reach 95% of luminance relative to initial luminance of 100%.

TABLE 4

| | | | | Driving | Current | | T₉₅ |
|---|---|---|---|---|---|---|---|
| | | Second | | voltage | efficiency | Luminance | life-span |
| | First host | host | Dopant | (V) | (cd/A) | (cd/m²) | (hr) |
| Example 12 | Compound 1 | E-31 | Ir(ppy)3 | 4.0 | 51 | 6000 | 80 |
| Example 13 | Compound 2 | E-31 | Ir(ppy)3 | 4.4 | 49 | 6000 | 74 |
| Example 14 | Compound 3 | E-31 | Ir(ppy)3 | 4.1 | 49 | 6000 | 77 |
| Example 15 | Compound 9 | E-31 | Ir(ppy)3 | 4.2 | 51 | 6000 | 81 |
| Example 16 | Compound 10 | E-31 | Ir(ppy)3 | 4.4 | 50 | 6000 | 75 |
| Example 17 | Compound 11 | E-31 | Ir(ppy)3 | 4.3 | 48 | 6000 | 77 |
| Example 18 | Compound 17 | E-31 | Ir(ppy)3 | 4.1 | 50 | 6000 | 79 |
| Example 19 | Compound 51 | E-31 | Ir(ppy)3 | 4.3 | 46 | 6000 | 83 |
| Example 20 | Compound 52 | E-31 | Ir(ppy)3 | 3.9 | 51 | 6000 | 82 |
| Example 21 | Compound 78 | E-31 | Ir(ppy)3 | 4.3 | 48 | 6000 | 80 |
| Example 22 | Compound 80 | E-31 | Ir(ppy)3 | 4.2 | 49 | 6000 | 80 |
| Example 23 | Compound 1 | E-99 | Ir(ppy)3 | 3.9 | 50 | 6000 | 79 |
| Example 24 | Compound 9 | E-99 | Ir(ppy)3 | 4.1 | 50 | 6000 | 79 |
| Example 25 | Compound 51 | E-99 | Ir(ppy)3 | 4.1 | 48 | 6000 | 83 |
| Example 26 | Compound 52 | E-99 | Ir(ppy)3 | 3.8 | 52 | 6000 | 83 |
| Example 27 | Compound 1 | F-43 | Ir(ppy)3 | 3.7 | 49 | 6000 | 78 |
| Example 28 | Compound 51 | F-43 | Ir(ppy)3 | 4.0 | 47 | 6000 | 82 |
| Example 29 | Compound 52 | F-43 | Ir(ppy)3 | 3.7 | 51 | 6000 | 82 |
| Example 30 | Compound 1 | F-99 | Ir(ppy)3 | 3.6 | 48 | 6000 | 78 |
| Example 31 | Compound 51 | F-99 | Ir(ppy)3 | 3.9 | 47 | 6000 | 80 |
| Example 32 | Compound 52 | F-99 | Ir(ppy)3 | 3.7 | 50 | 6000 | 81 |
| Example 33 | Compound 1 | F-73 | Ir(ppy)3 | 3.9 | 50 | 6000 | 77 |
| Example 34 | Compound 51 | F-73 | Ir(ppy)3 | 4.1 | 49 | 6000 | 81 |
| Example 35 | Compound 52 | F-73 | Ir(ppy)3 | 3.8 | 51 | 6000 | 81 |
| Comparative | Comparative | E-31 | Ir(ppy)3 | 4.8 | 42 | 6000 | 68 |
| Example 7 | Compound 1 | | | | | | |
| Comparative | Comparative | E-31 | Ir(ppy)3 | 4.5 | 46 | 6000 | 70 |
| Example 8 | Compound 2 | | | | | | |
| Comparative | Comparative | E-31 | Ir(ppy)3 | 4.8 | 43 | 6000 | 67 |
| Example 9 | Compound 3 | | | | | | |
| Comparative | Comparative | E-31 | Ir(ppy)3 | 4.6 | 44 | 6000 | 69 |
| Example 10 | Compound 4 | | | | | | |
| Comparative | Comparative | E-43 | Ir(ppy)3 | 4.6 | 45 | 6000 | 70 |
| Example 11 | Compound 5 | | | | | | |
| Comparative | Comparative | E-43 | Ir(ppy)3 | 4.6 | 44 | 6000 | 70 |
| Example 12 | Compound 6 | | | | | | |

Referring to Table 4, each organic light emitting diode according to Examples 12 to 35 showed a low driving voltage, high efficiency, and a long life-span compared with the organic light emitting diodes according to Comparative Examples 7 to 12.

Manufacture of Organic Light Emitting Diode III

### Example 36

An organic light emitting diode was manufactured by using Compound 1 of Synthesis Example 1 as a host and (piq)₂Ir(acac) as a dopant.

As for an anode, 1000 Å-thick ITO was used, and as for a cathode, 1000 Å-thick aluminum was used. Specifically, illustrating a method of manufacturing the organic light emitting diode, the anode was manufactured by cutting an ITO glass substrate having 15 Ω/cm² of a sheet resistance into a size of 50 mm×50 mm×0.7 mm, ultrasonic wave-cleaning them in each acetone, isopropyl alcohol, and pure water for 15 minutes respectively, and UV ozone cleaning them for 30 minutes.

On the substrate, an 800 Å-thick hole transport layer was formed by depositing N4,N4′-di(naphthalene-1-yl)-N4,N4′-diphenylbiphenyl-4,4′-diamine (NPB) (80 nm) under a vacuum degree of 650×10⁻⁷ Pa at a deposition rate of 0.1 to 0.3 nm/s. Subsequently, a 300 Å-thick light emitting layer was formed by using Compound 1 of Synthesis Example 1 under the same vacuum deposition condition, and a phosphorescent dopant of (piq)₂Ir(acac) was simultaneously deposited. Herein, the phosphorescent dopant was deposited to be 3 wt % based on 100 wt % of a total weight of the light emitting layer by adjusting the deposition rate.

On the light emitting layer, a 50 Å-thick hole blocking layer was formed by depositing bis(2-methyl-8-quinolinolate)-4-(phenylphenolato)aluminum (BAlq) under the same vacuum deposition condition. Subsequently, a 200 Å-thick electron transport layer was formed by depositing Alq3 under the same vacuum deposition condition. On the electron transport layer, a cathode was formed by sequentially depositing LiF and Al to manufacture an organic organic light emitting diode.

A structure of the organic light emitting diode was ITO/NPB (80 nm)/EML (Compound 1 (97 wt %)+(piq)₂Ir(acac) (3 wt %), 30 nm)/Balq (5 nm)/Alq3 (20 nm)/LiF (1 nm)/Al (100 nm).

### Examples 37 to 42

Organic light emitting diodes were respectively manufactured according to the same method as Example 36 except for using each of Compound 3, Compound 9, Compound 11, Compound 17, Compound 52, or Compound 80 instead of Compound 1 as a host for forming a light emitting layer.

### Comparative Examples 13 to 18

Organic light emitting diodes were respectively manufactured according to the same method as Example 36 except for using each of Comparative Compounds 1, 2, 3, 4, 5, or 6 instead of Compound 1 as a host for forming a light emitting layer.

Evaluation 3

Luminous efficiency and life-span characteristics of each organic light emitting diode according to Examples 36 to 42 and Comparative Examples 13 to 18 were evaluated.

Specific measurement methods are as follows, and the results are shown in Table 5.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value was divided by area to provide the results.

(2) Measurement of Luminance Change Depending on Voltage Change

Luminance was measured by using a luminance meter (Minolta Cs-1000A), while the voltages of the organic light emitting diodes were increased from 0 V to 10 V.

(3) Measurement of Luminous Efficiency

Current efficiency (cd/A) at the same current density (10 mA/cm²) were calculated by using the luminance, current density, and voltages (V) from the items (1) and (2).

(4) Roll-Off Measurement

Roll-off was measured by calculating the falling amount of efficiency as % according to (Max measurement−Measurement at 6000 cd/m²/Max measurement) from the characteristic measurements of the (3).

(5) Measurement of Life-Span

Life-span was obtained by measuring time taken until current efficiency (cd/A) decreased down to 90%, while luminance (cd/m²) was maintained at 5000 cd/m².

TABLE 5

| | | Driving | Luminous | | |
|---|---|---|---|---|---|
| | | voltage | efficiency | Roll-off | Life-span |
| | First host | (V) | (cd/A) | (%) | T90 (h) |
| Example 36 | Compound 1 | 4.7 | 14.8 | 11.1 | 135 |
| Example 37 | Compound 3 | 4.7 | 14.5 | 11.3 | 131 |
| Example 38 | Compound 9 | 4.7 | 14.6 | 11.0 | 133 |
| Example 39 | Compound 11 | 4.8 | 14.5 | 10.8 | 130 |
| Example 40 | Compound 17 | 4.8 | 14.6 | 10.7 | 134 |
| Example 41 | Compound 52 | 4.5 | 14.8 | 10.5 | 139 |
| Example 42 | Compound 80 | 4.6 | 14.7 | 10.4 | 136 |
| Comparative | Comparative | 5.5 | 12.4 | 10.4 | 105 |
| Example 13 | Compound 1 | | | | |
| Comparative | Comparative | 5.3 | 12.8 | 10.5 | 115 |
| Example 14 | Compound 2 | | | | |
| Comparative | Comparative | 5.7 | 12.3 | 10.3 | 110 |
| Example 15 | Compound 3 | | | | |
| Comparative | Comparative | 5.4 | 12.7 | 10.3 | 119 |
| Example 16 | Compound 4 | | | | |
| Comparative | Comparative | 5.3 | 12.6 | 10.5 | 119 |
| Example 17 | Compound 5 | | | | |
| Comparative | Comparative | 5.3 | 12.8 | 10.7 | 120 |
| Example 18 | Compound 6 | | | | |

Referring to Table 5, the organic light emitting diodes according to Examples 36 to 42 showed a driving voltage, high efficiency, and a long life-span compared with the organic light emitting diodes according to Comparative Examples 13 to 18.

Accordingly, the first hosts had excellent charge transport characteristics as a phosphorescent host material, was well overlapped with an absorption spectrum of a dopant, and turned out to improve performance such as an efficiency increase, a driving voltage decrease, and a long life-span and show maximized capability as an OLED material.

Manufacture of Organic Light Emitting Diode IV

### Example 43

An organic light emitting diode was manufactured according to the same method as Example 36 except for codepositing (piq)₂Ir(acac) (a dopant), Compound 1 (a first host), and Compound E-31 (a second host) in a weight ratio of 3:48.5:48.5 on a hole transport layer (HTL) to form a 400 Å-thick light emitting layer.

### Examples 44 to 52 and Comparative Examples 19 to 24

An organic light emitting diode was manufactured according to the same method as Example 43 except for using each first and second host shown in Table 6 to form a light emitting layer.

### Evaluation Example 4

A driving voltage, efficiency, luminance, and a life-span of each organic light emitting diode according to Examples 43 to 52 and Comparative Examples 19 to 24 were measured by supplying power from a current voltage meter (Keithley SMU 236) and using a luminance meter, PR650 Spectroscan Source Measurement Unit (Photo Research Inc.). The results are shown in Table 6.

Roll-off is measured in the above Roll-off measurement method.

TABLE 6

| | | | Driving | Light emitting | | |
|---|---|---|---|---|---|---|
| | | | voltage | efficiency | Roll-off | Life-span |
| | First host | Second host | (V) | (cd/A) | (%) | T90 (h) |
| Example 43 | Compound 1 | E-31 | 4.4 | 16.2 | 11.0 | 155 |
| Example 44 | Compound 1 | E-99 | 4.3 | 16.5 | 10.3 | 152 |
| Example 45 | Compound 1 | F-104 | 4.0 | 18.2 | 10.2 | 151 |
| Example 46 | Compound 1 | F-106 | 3.9 | 18.5 | 10.0 | 157 |
| Example 47 | Compound 1 | F-107 | 4.0 | 18.0 | 10.0 | 157 |
| Example 48 | Compound 1 | F-110 | 4.0 | 18.3 | 9.8 | 160 |
| Example 49 | Compound 9 | F-104 | 3.9 | 19.3 | 10.1 | 164 |
| Example 50 | Compound 51 | F-104 | 3.9 | 19.2 | 10.0 | 160 |
| Example 51 | Compound 9 | F-106 | 3.9 | 19.4 | 10.2 | 170 |
| Example 52 | Compound 51 | F-106 | 4.0 | 19.2 | 10.1 | 167 |
| Comparative | Comparative | F-106 | 5.2 | 14.3 | 10.4 | 131 |
| Example 19 | Compound 1 | | | | | |
| Comparative | Comparative | F-106 | 4.8 | 14.6 | 10.5 | 140 |
| Example 20 | Compound 2 | | | | | |
| Comparative | Comparative | F-106 | 5.4 | 14.4 | 10.3 | 132 |
| Example 21 | Compound 3 | | | | | |
| Comparative | Comparative | F-106 | 5.0 | 14.8 | 10.3 | 135 |
| Example 22 | Compound 4 | | | | | |
| Comparative | Comparative | F-106 | 5.1 | 14.7 | 10.5 | 138 |
| Example 23 | Compound 5 | | | | | |
| Comparative | Comparative | F-106 | 5.1 | 14.8 | 10.7 | 139 |
| Example 24 | Compound 6 | | | | | |

Referring to Table 6, the organic light emitting diodes according to Examples 43 to 52 showed a low driving voltage or high efficiency and a long life-span compared with the organic light emitting diodes according to Comparative Examples 19 to 24.

As described above, embodiments may provide an organic compound capable of realizing an organic optoelectronic device having high efficiency and a long life-span. Embodiments may also provide a composition capable of realizing an organic optoelectronic device having high efficiency and a long life-span.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. An organic compound represented by Chemical Formula 1: 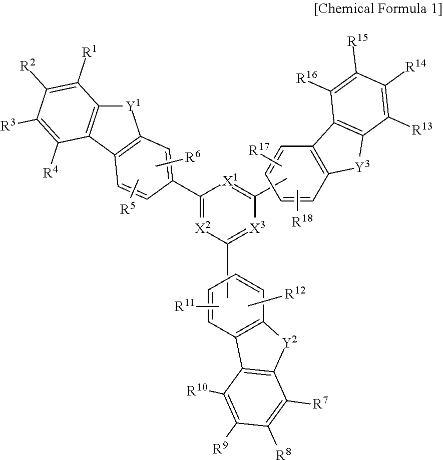
wherein, in Chemical Formula 1,
X¹ to X³ are independently N or CR^{a}, provided that at least two of X¹ to X³ are N,
Y¹ to Y³ are independently O or S,
R¹ to R¹⁸ and R^{a} are independently hydrogen, deuterium, a substituted or unsubstituted C1 to C20 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof, and
R¹ to R¹⁸ are separate or adjacent groups of R¹ to R¹⁸ are linked with each other to form a ring.

2. The organic compound as claimed in claim 1, wherein the organic compound is represented by Chemical Formula 2 or 3: 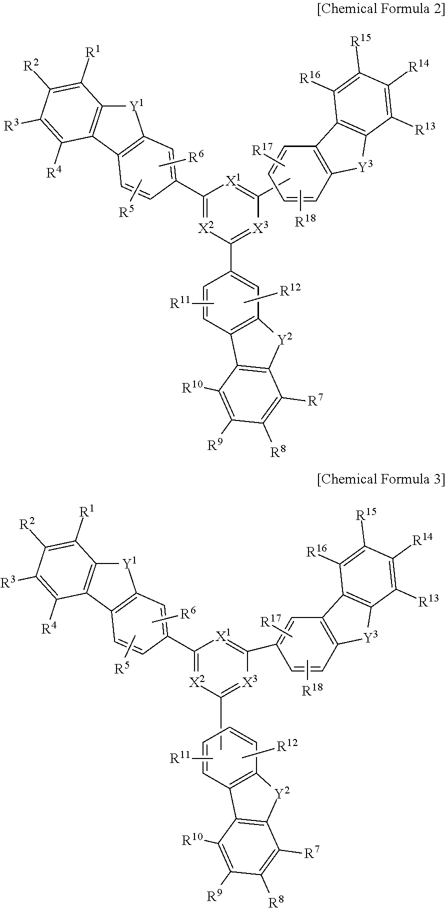
wherein, in Chemical Formula 2 or 3,
X¹ to X³ are independently N or CR^{a}, provided that at least two of X¹ to X³ are N,
Y¹ to Y³ are independently O or S,
R¹ to R¹⁸ and R^{a} are independently hydrogen, deuterium, a substituted or unsubstituted C1 to C20 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof, and
R¹ to R¹⁸ are separate or adjacent groups of R¹ to R¹⁸ are linked with each other to form a ring.

3. The organic compound as claimed in claim 2, wherein the organic compound is represented by one of Chemical Formulae 4 to 6: 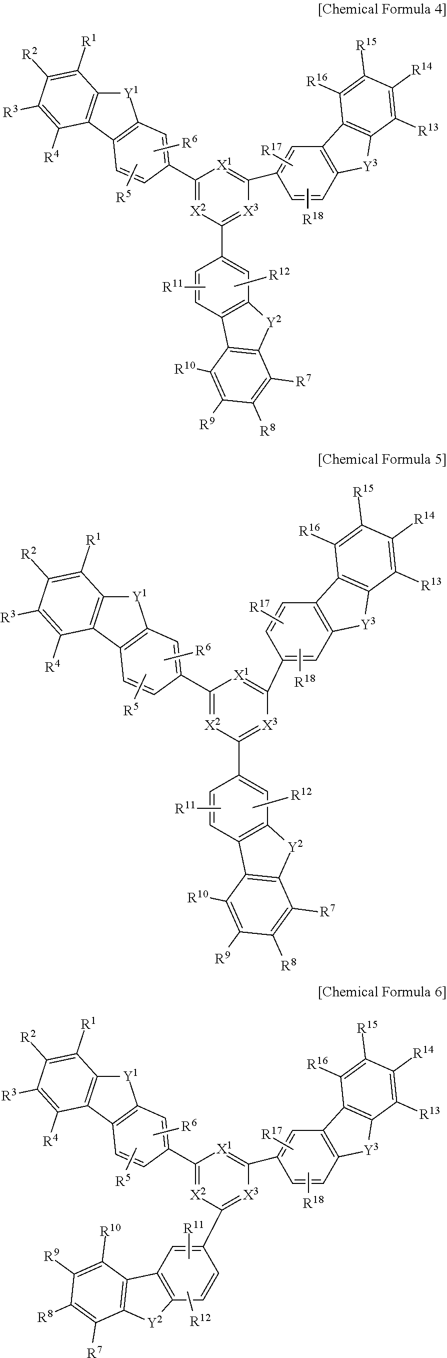
wherein, in Chemical Formulae 4 to 6,
X¹ to X³ are independently N or CR^{a}, provided that at least two of X¹ to X³ are N,
Y¹ to Y³ are independently O or S,
R¹ to R¹⁸ and R^{a} are independently hydrogen, deuterium, a substituted or unsubstituted C1 to C20 alkyl group, a substituted or unsubstituted C6 to C30 aryl group, a substituted or unsubstituted C3 to C30 heterocyclic group, a substituted or unsubstituted silyl group, a substituted or unsubstituted amine group, a halogen, a cyano group, or a combination thereof, and
R¹ to R¹⁸ are separate or adjacent groups of R¹ to R¹⁸ are linked with each other to form a ring.

4. The organic compound as claimed in claim 1, wherein X¹ to X³ are each N.

5. The organic compound as claimed in claim 1, wherein the organic compound is one of compounds listed in Group 1,
[Group 1]

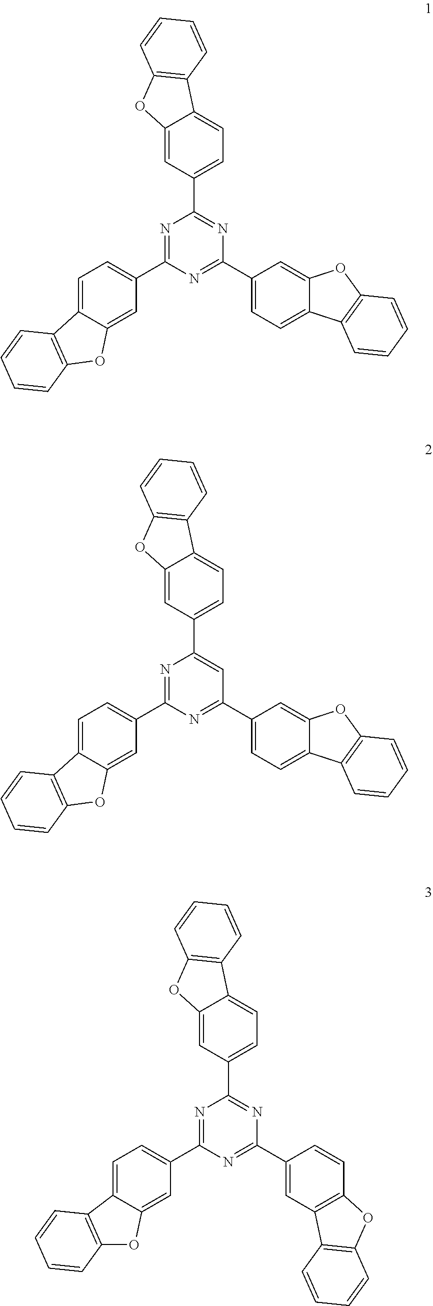 [SMILES: c1ccc2c(c1)oc1cc(-c3nc(-c4ccc5c(c4)oc4ccccc45)nc(-c4ccc5c(c4)oc4ccccc45)n3)ccc12 ; c1ccc2c(c1)oc1cc(-c3cc(-c4ccc5c(c4)oc4ccccc45)nc(-c4ccc5c(c4)oc4ccccc45)n3)ccc12 ; c1ccc2c(c1)oc1cc(-c3nc(-c4ccc5c(c4)oc4ccccc45)nc(-c4ccc5oc6ccccc6c5c4)n3)ccc12]

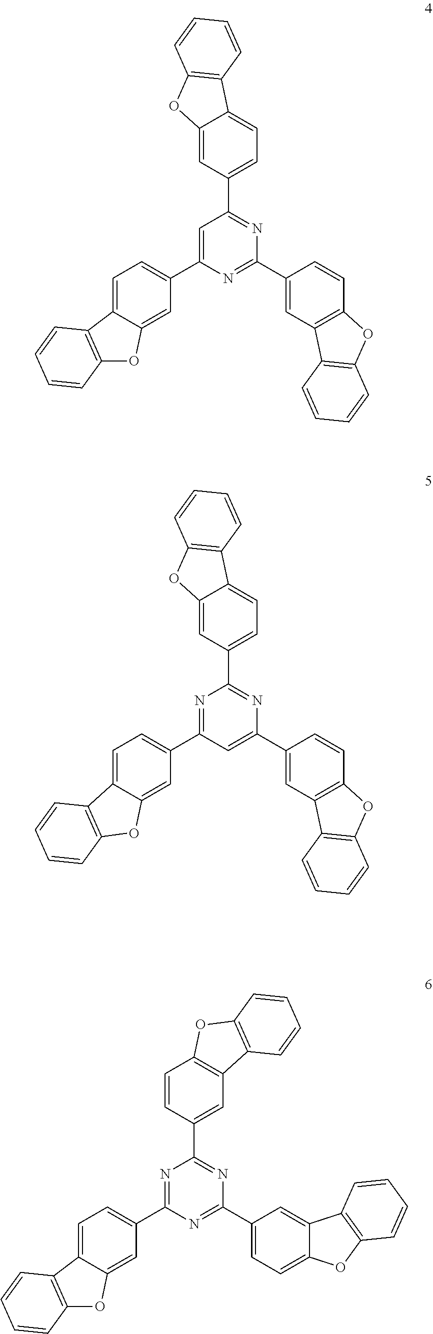 [SMILES: c1ccc2c(c1)oc1cc(-c3cc(-c4ccc5c(c4)oc4ccccc45)nc(-c4ccc5oc6ccccc6c5c4)n3)ccc12 ; c1ccc2c(c1)oc1cc(-c3cc(-c4ccc5oc6ccccc6c5c4)nc(-c4ccc5c(c4)oc4ccccc45)n3)ccc12 ; c1ccc2c(c1)oc1cc(-c3nc(-c4ccc5oc6ccccc6c5c4)nc(-c4ccc5oc6ccccc6c5c4)n3)ccc12]

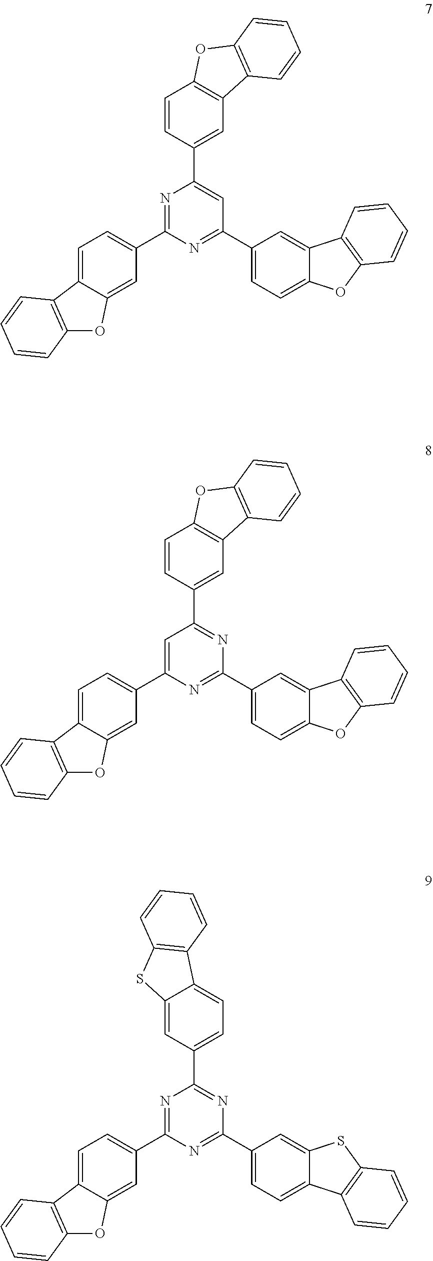 [SMILES: c1ccc2c(c1)oc1cc(-c3nc(-c4ccc5oc6ccccc6c5c4)cc(-c4ccc5oc6ccccc6c5c4)n3)ccc12 ; c1ccc2c(c1)oc1cc(-c3cc(-c4ccc5oc6ccccc6c5c4)nc(-c4ccc5oc6ccccc6c5c4)n3)ccc12 ; c1ccc2c(c1)oc1cc(-c3nc(-c4ccc5c(c4)sc4ccccc45)nc(-c4ccc5c(c4)sc4ccccc45)n3)ccc12]

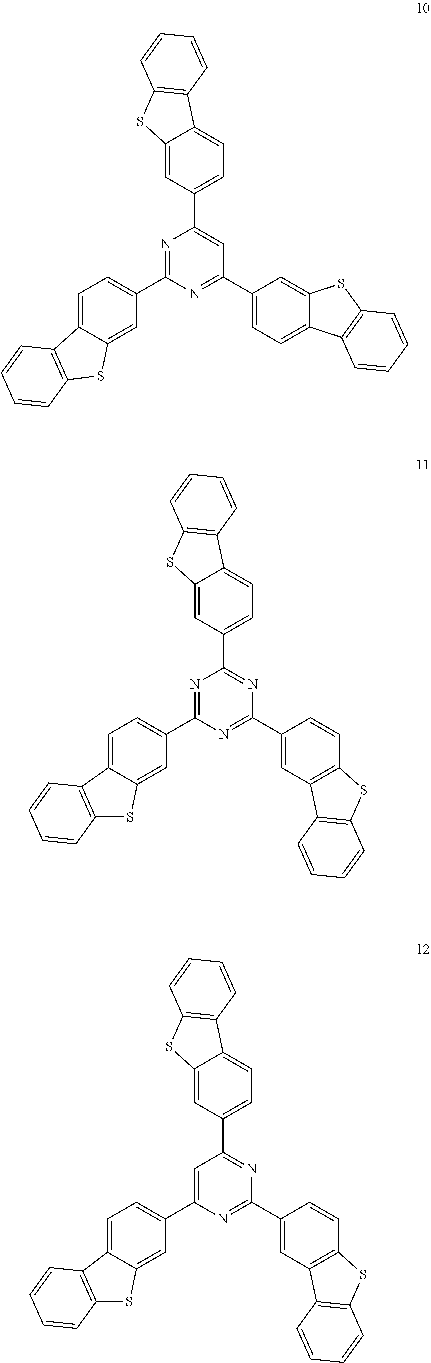 [SMILES: c1ccc2c(c1)sc1cc(-c3cc(-c4ccc5c(c4)sc4ccccc45)nc(-c4ccc5c(c4)sc4ccccc45)n3)ccc12 ; c1ccc2c(c1)sc1cc(-c3nc(-c4ccc5c(c4)sc4ccccc45)nc(-c4ccc5sc6ccccc6c5c4)n3)ccc12 ; c1ccc2c(c1)sc1cc(-c3cc(-c4ccc5c(c4)sc4ccccc45)nc(-c4ccc5sc6ccccc6c5c4)n3)ccc12]

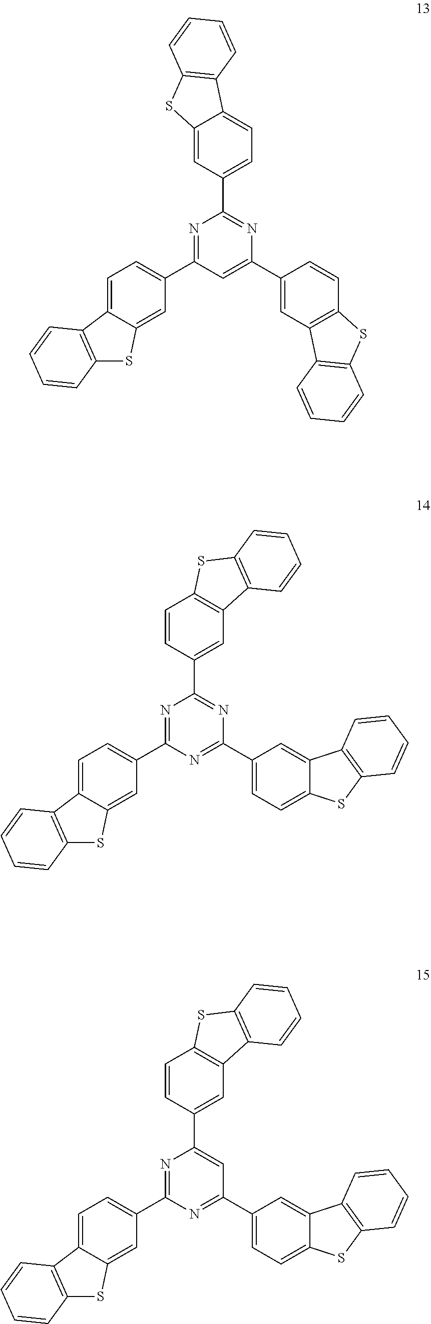 [SMILES: c1ccc2c(c1)sc1cc(-c3cc(-c4ccc5sc6ccccc6c5c4)nc(-c4ccc5c(c4)sc4ccccc45)n3)ccc12 ; c1ccc2c(c1)sc1cc(-c3nc(-c4ccc5sc6ccccc6c5c4)nc(-c4ccc5sc6ccccc6c5c4)n3)ccc12 ; c1ccc2c(c1)sc1cc(-c3nc(-c4ccc5sc6ccccc6c5c4)cc(-c4ccc5sc6ccccc6c5c4)n3)ccc12]

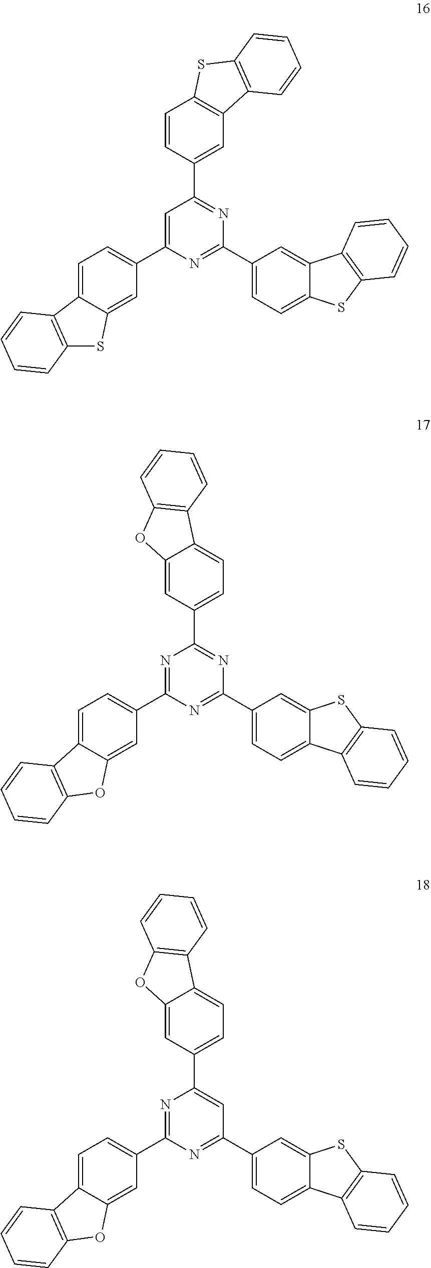 [SMILES: c1ccc2c(c1)sc1cc(-c3cc(-c4ccc5sc6ccccc6c5c4)nc(-c4ccc5sc6ccccc6c5c4)n3)ccc12 ; c1ccc2c(c1)oc1cc(-c3nc(-c4ccc5c(c4)oc4ccccc45)nc(-c4ccc5c(c4)sc4ccccc45)n3)ccc12 ; c1ccc2c(c1)oc1cc(-c3cc(-c4ccc5c(c4)sc4ccccc45)nc(-c4ccc5c(c4)oc4ccccc45)n3)ccc12]

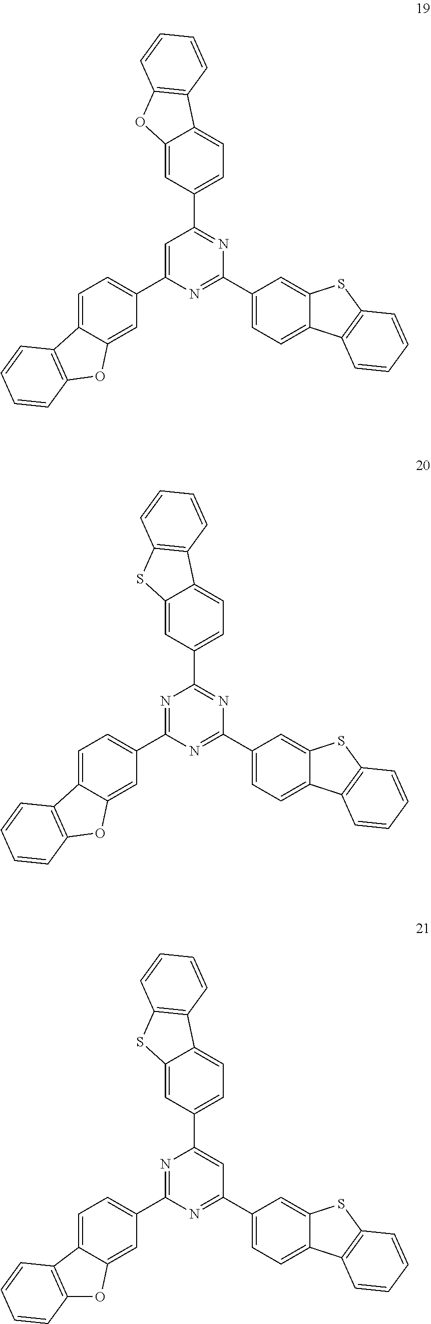 [SMILES: c1ccc2c(c1)oc1cc(-c3cc(-c4ccc5c(c4)oc4ccccc45)nc(-c4ccc5c(c4)sc4ccccc45)n3)ccc12 ; c1ccc2c(c1)oc1cc(-c3nc(-c4ccc5c(c4)sc4ccccc45)nc(-c4ccc5c(c4)sc4ccccc45)n3)ccc12 ; c1ccc2c(c1)oc1cc(-c3nc(-c4ccc5c(c4)sc4ccccc45)cc(-c4ccc5c(c4)sc4ccccc45)n3)ccc12]

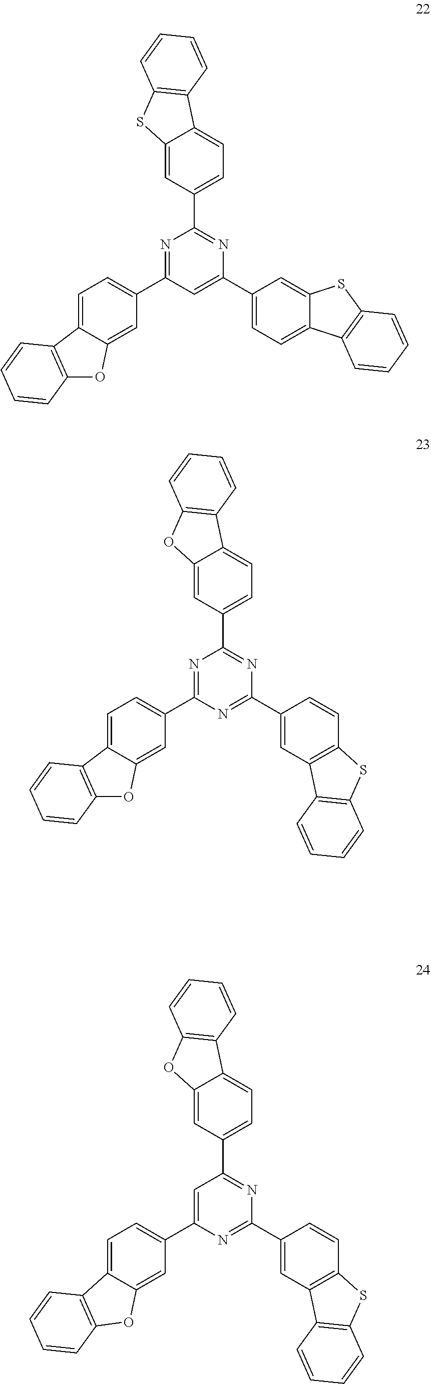 [SMILES: c1ccc2c(c1)oc1cc(-c3cc(-c4ccc5c(c4)sc4ccccc45)nc(-c4ccc5c(c4)sc4ccccc45)n3)ccc12 ; c1ccc2c(c1)oc1cc(-c3nc(-c4ccc5c(c4)oc4ccccc45)nc(-c4ccc5sc6ccccc6c5c4)n3)ccc12 ; c1ccc2c(c1)oc1cc(-c3cc(-c4ccc5c(c4)oc4ccccc45)nc(-c4ccc5sc6ccccc6c5c4)n3)ccc12]

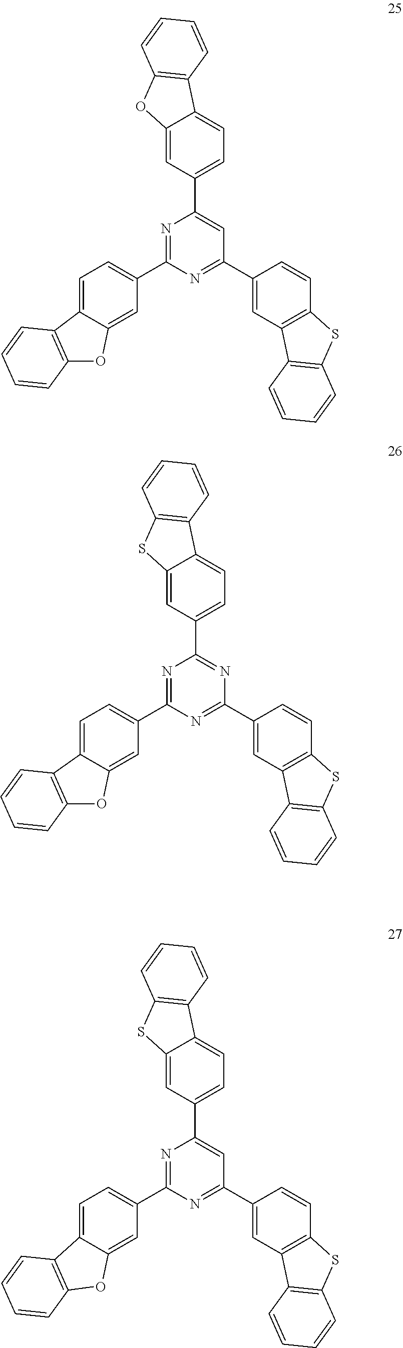 [SMILES: c1ccc2c(c1)oc1cc(-c3cc(-c4ccc5sc6ccccc6c5c4)nc(-c4ccc5c(c4)oc4ccccc45)n3)ccc12 ; c1ccc2c(c1)oc1cc(-c3nc(-c4ccc5c(c4)sc4ccccc45)nc(-c4ccc5sc6ccccc6c5c4)n3)ccc12 ; c1ccc2c(c1)oc1cc(-c3nc(-c4ccc5c(c4)sc4ccccc45)cc(-c4ccc5sc6ccccc6c5c4)n3)ccc12]

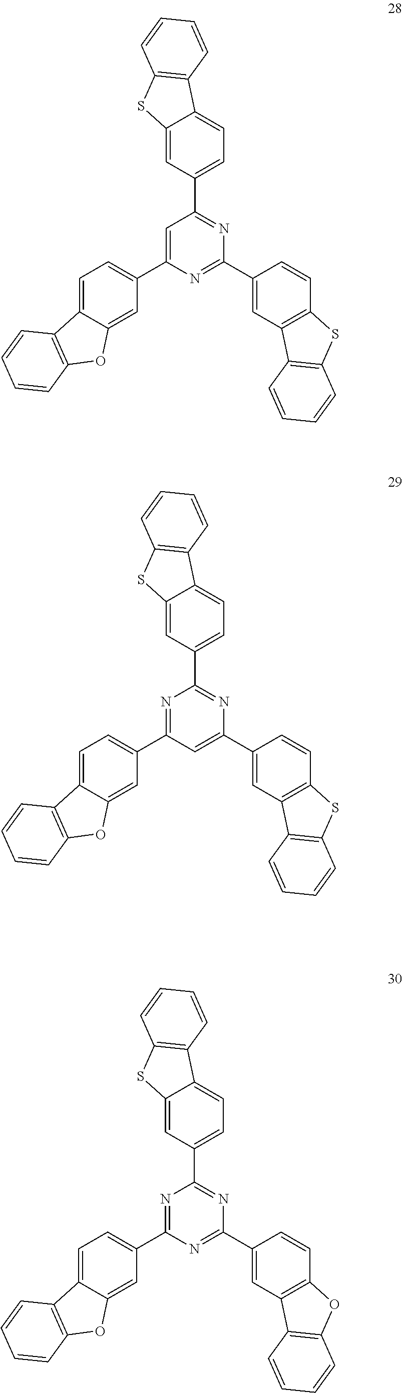 [SMILES: c1ccc2c(c1)oc1cc(-c3cc(-c4ccc5c(c4)sc4ccccc45)nc(-c4ccc5sc6ccccc6c5c4)n3)ccc12 ; c1ccc2c(c1)oc1cc(-c3cc(-c4ccc5sc6ccccc6c5c4)nc(-c4ccc5c(c4)sc4ccccc45)n3)ccc12 ; c1ccc2c(c1)oc1cc(-c3nc(-c4ccc5c(c4)sc4ccccc45)nc(-c4ccc5oc6ccccc6c5c4)n3)ccc12]

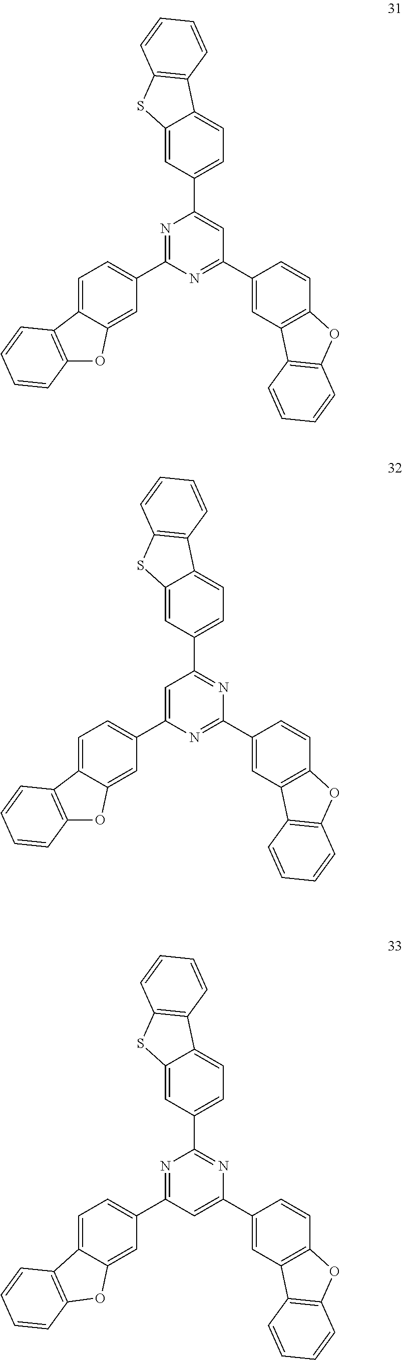 [SMILES: c1ccc2c(c1)oc1cc(-c3nc(-c4ccc5c(c4)sc4ccccc45)cc(-c4ccc5oc6ccccc6c5c4)n3)ccc12 ; c1ccc2c(c1)oc1cc(-c3cc(-c4ccc5c(c4)sc4ccccc45)nc(-c4ccc5oc6ccccc6c5c4)n3)ccc12 ; c1ccc2c(c1)oc1cc(-c3cc(-c4ccc5oc6ccccc6c5c4)nc(-c4ccc5c(c4)sc4ccccc45)n3)ccc12]

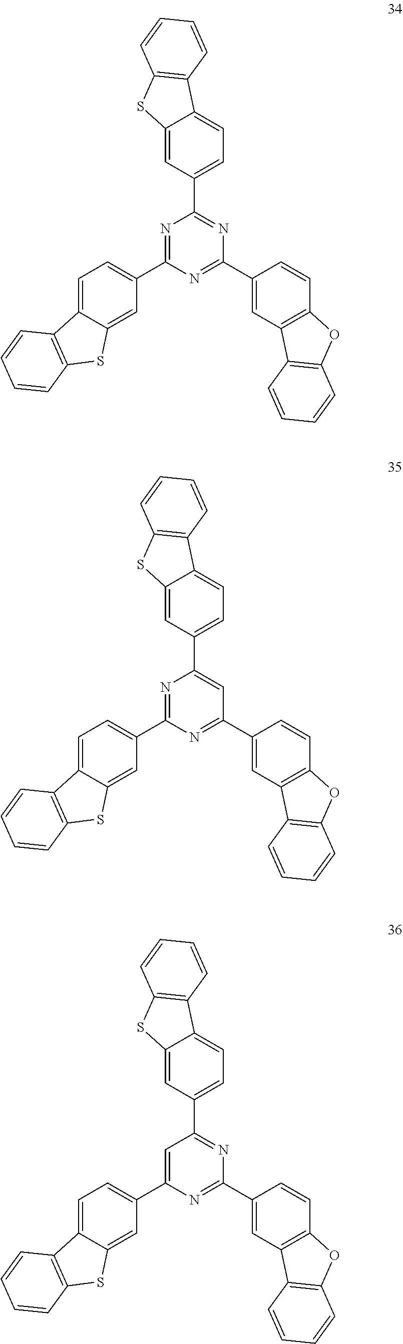 [SMILES: c1ccc2c(c1)oc1ccc(-c3nc(-c4ccc5c(c4)sc4ccccc45)nc(-c4ccc5c(c4)sc4ccccc45)n3)cc12 ; c1ccc2c(c1)oc1ccc(-c3cc(-c4ccc5c(c4)sc4ccccc45)nc(-c4ccc5c(c4)sc4ccccc45)n3)cc12 ; c1ccc2c(c1)oc1ccc(-c3nc(-c4ccc5c(c4)sc4ccccc45)cc(-c4ccc5c(c4)sc4ccccc45)n3)cc12]

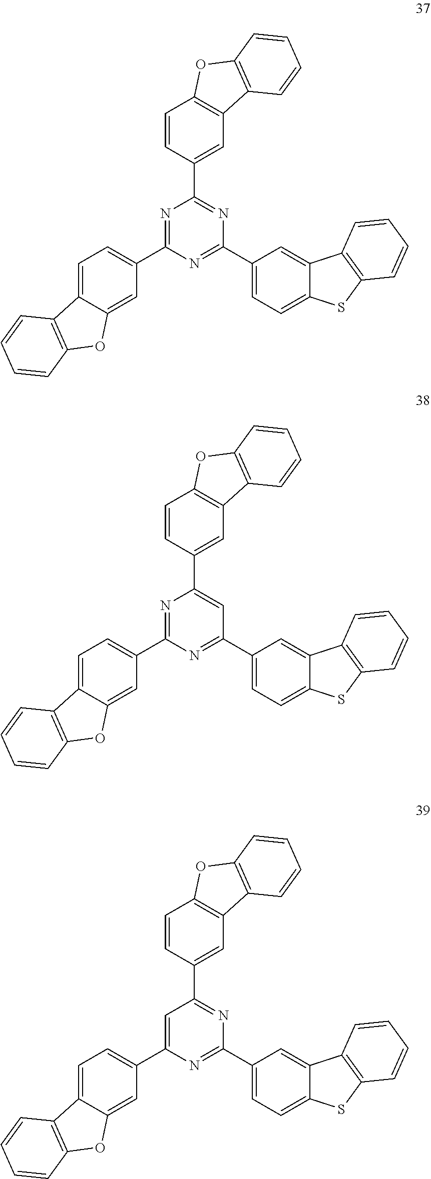 [SMILES: c1ccc2c(c1)oc1cc(-c3nc(-c4ccc5oc6ccccc6c5c4)nc(-c4ccc5sc6ccccc6c5c4)n3)ccc12 ; c1ccc2c(c1)oc1cc(-c3nc(-c4ccc5oc6ccccc6c5c4)cc(-c4ccc5sc6ccccc6c5c4)n3)ccc12 ; c1ccc2c(c1)oc1cc(-c3cc(-c4ccc5oc6ccccc6c5c4)nc(-c4ccc5sc6ccccc6c5c4)n3)ccc12]

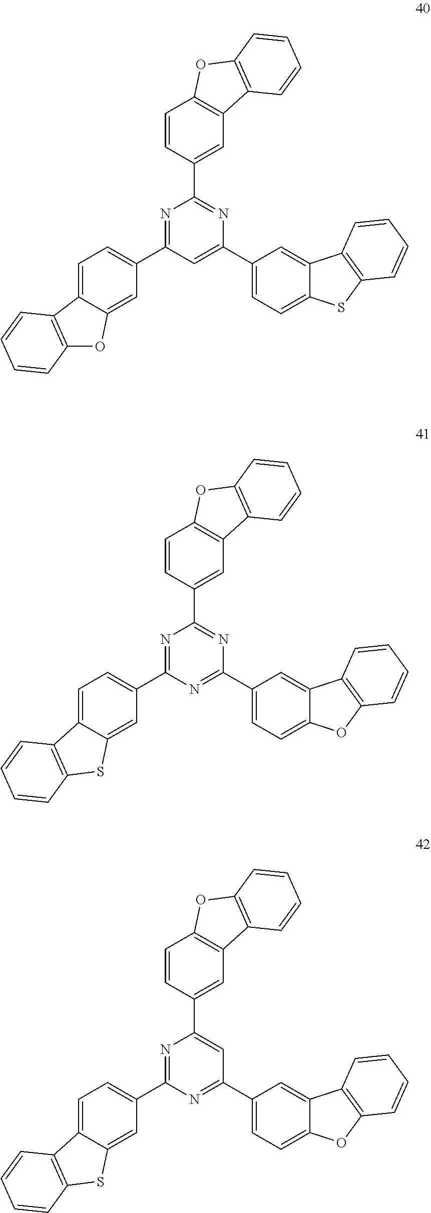 [SMILES: c1ccc2c(c1)oc1cc(-c3cc(-c4ccc5sc6ccccc6c5c4)nc(-c4ccc5oc6ccccc6c5c4)n3)ccc12 ; c1ccc2c(c1)oc1ccc(-c3nc(-c4ccc5c(c4)sc4ccccc45)nc(-c4ccc5oc6ccccc6c5c4)n3)cc12 ; c1ccc2c(c1)oc1ccc(-c3cc(-c4ccc5oc6ccccc6c5c4)nc(-c4ccc5c(c4)sc4ccccc45)n3)cc12]

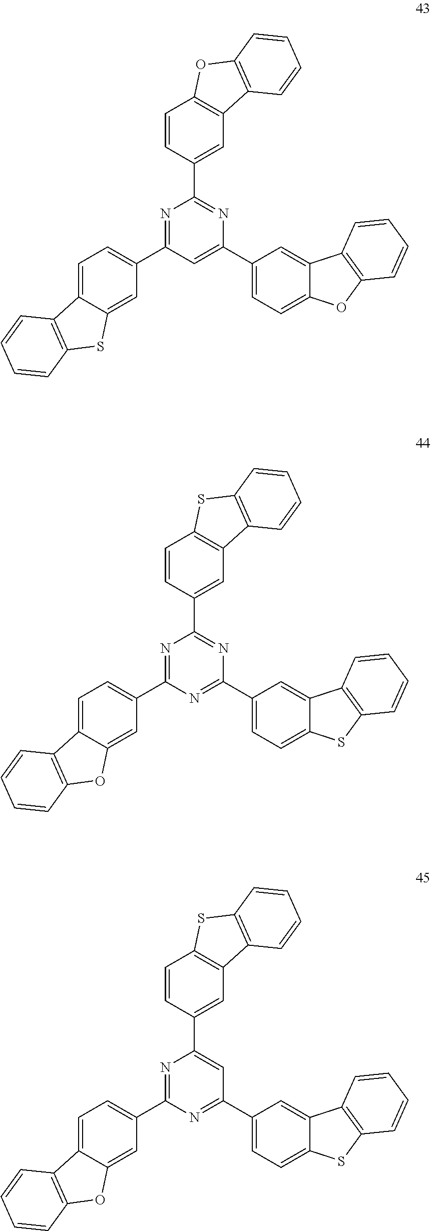 [SMILES: c1ccc2c(c1)oc1ccc(-c3cc(-c4ccc5c(c4)sc4ccccc45)nc(-c4ccc5oc6ccccc6c5c4)n3)cc12 ; c1ccc2c(c1)oc1cc(-c3nc(-c4ccc5sc6ccccc6c5c4)nc(-c4ccc5sc6ccccc6c5c4)n3)ccc12 ; c1ccc2c(c1)oc1cc(-c3nc(-c4ccc5sc6ccccc6c5c4)cc(-c4ccc5sc6ccccc6c5c4)n3)ccc12]

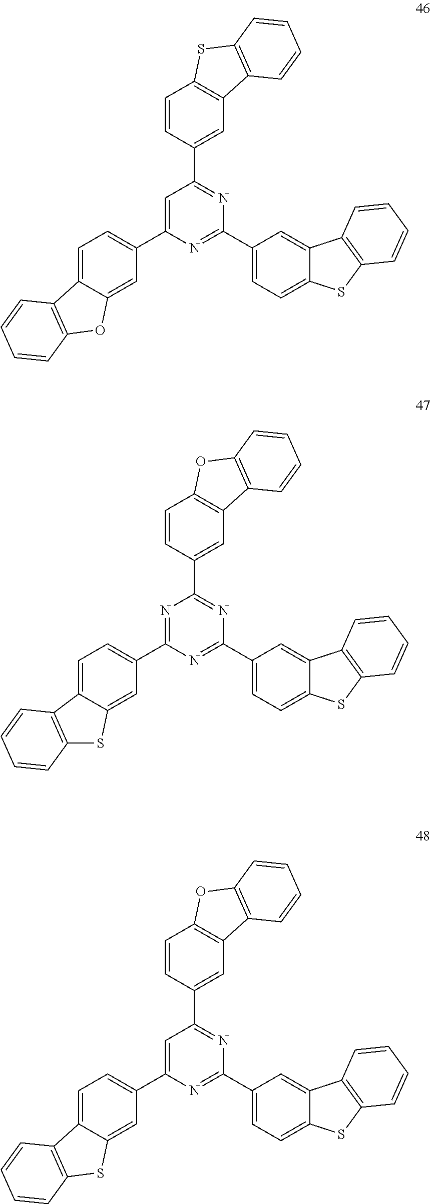 [SMILES: c1ccc2c(c1)oc1cc(-c3cc(-c4ccc5sc6ccccc6c5c4)nc(-c4ccc5sc6ccccc6c5c4)n3)ccc12 ; c1ccc2c(c1)oc1ccc(-c3nc(-c4ccc5c(c4)sc4ccccc45)nc(-c4ccc5sc6ccccc6c5c4)n3)cc12 ; c1ccc2c(c1)oc1ccc(-c3cc(-c4ccc5c(c4)sc4ccccc45)nc(-c4ccc5sc6ccccc6c5c4)n3)cc12]

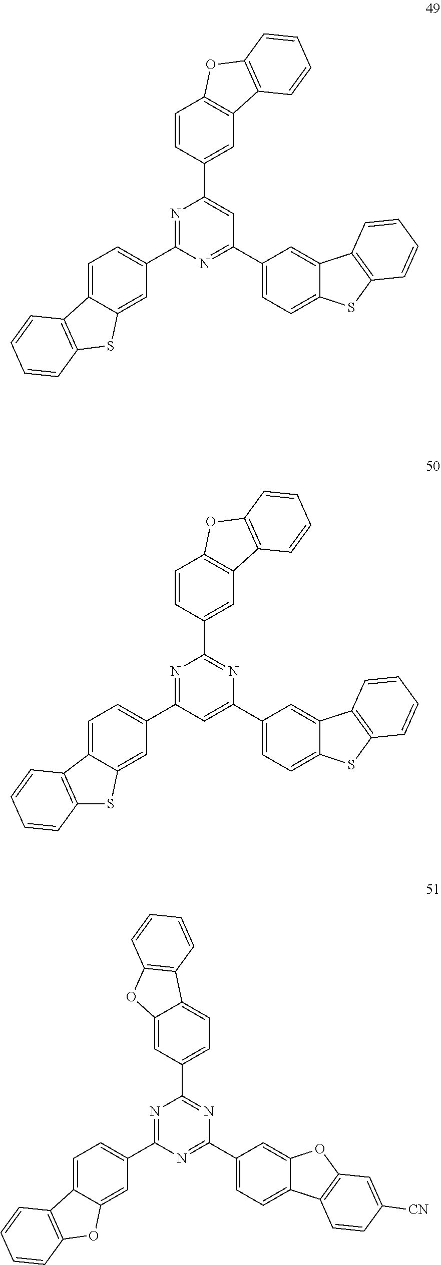 [SMILES: c1ccc2c(c1)oc1ccc(-c3cc(-c4ccc5sc6ccccc6c5c4)nc(-c4ccc5c(c4)sc4ccccc45)n3)cc12 ; c1ccc2c(c1)oc1ccc(-c3nc(-c4ccc5c(c4)sc4ccccc45)cc(-c4ccc5sc6ccccc6c5c4)n3)cc12 ; N#Cc1ccc2c(c1)oc1cc(-c3nc(-c4ccc5c(c4)oc4ccccc45)nc(-c4ccc5c(c4)oc4ccccc45)n3)ccc12]

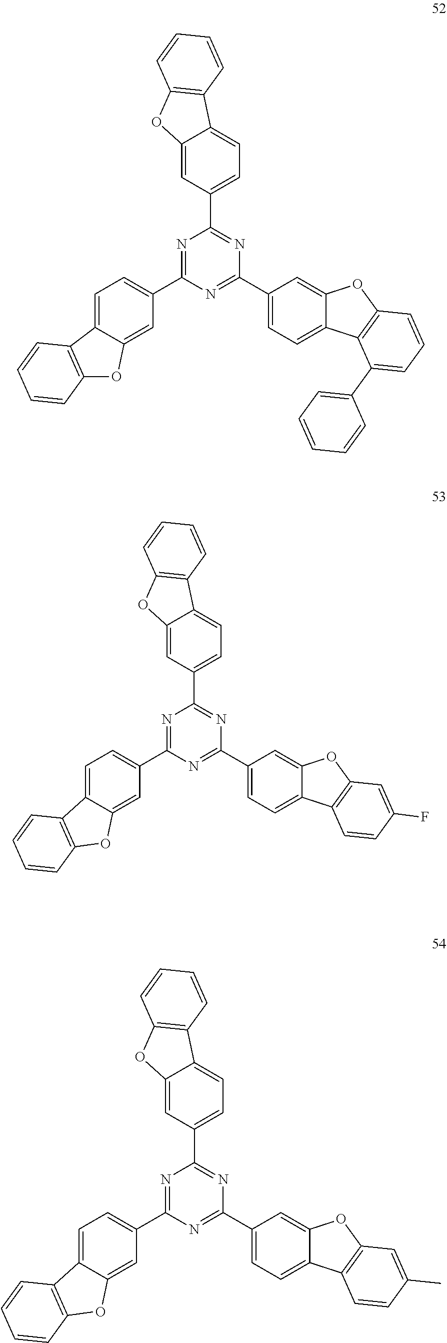 [SMILES: c1ccc(-c2cccc3oc4cc(-c5nc(-c6ccc7c(c6)oc6ccccc67)nc(-c6ccc7c(c6)oc6ccccc67)n5)ccc4c23)cc1 ; Fc1ccc2c(c1)oc1cc(-c3nc(-c4ccc5c(c4)oc4ccccc45)nc(-c4ccc5c(c4)oc4ccccc45)n3)ccc12 ; Cc1ccc2c(c1)oc1cc(-c3nc(-c4ccc5c(c4)oc4ccccc45)nc(-c4ccc5c(c4)oc4ccccc45)n3)ccc12]

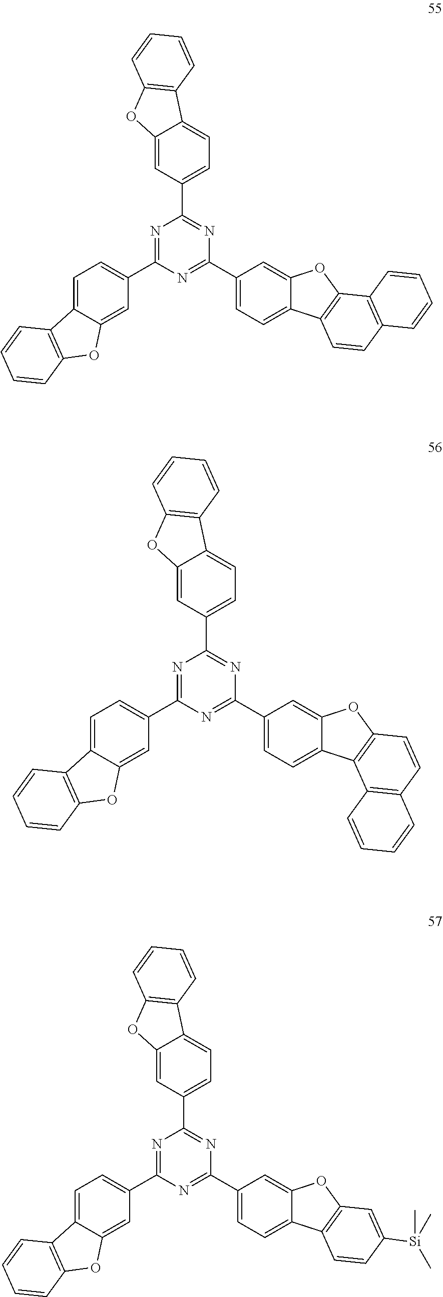 [SMILES: c1ccc2c(c1)ccc1c3ccc(-c4nc(-c5ccc6c(c5)oc5ccccc56)nc(-c5ccc6c(c5)oc5ccccc56)n4)cc3oc21 ; c1ccc2c(c1)ccc1oc3cc(-c4nc(-c5ccc6c(c5)oc5ccccc56)nc(-c5ccc6c(c5)oc5ccccc56)n4)ccc3c12 ; C[Si](C)(C)c1ccc2c(c1)oc1cc(-c3nc(-c4ccc5c(c4)oc4ccccc45)nc(-c4ccc5c(c4)oc4ccccc45)n3)ccc12]

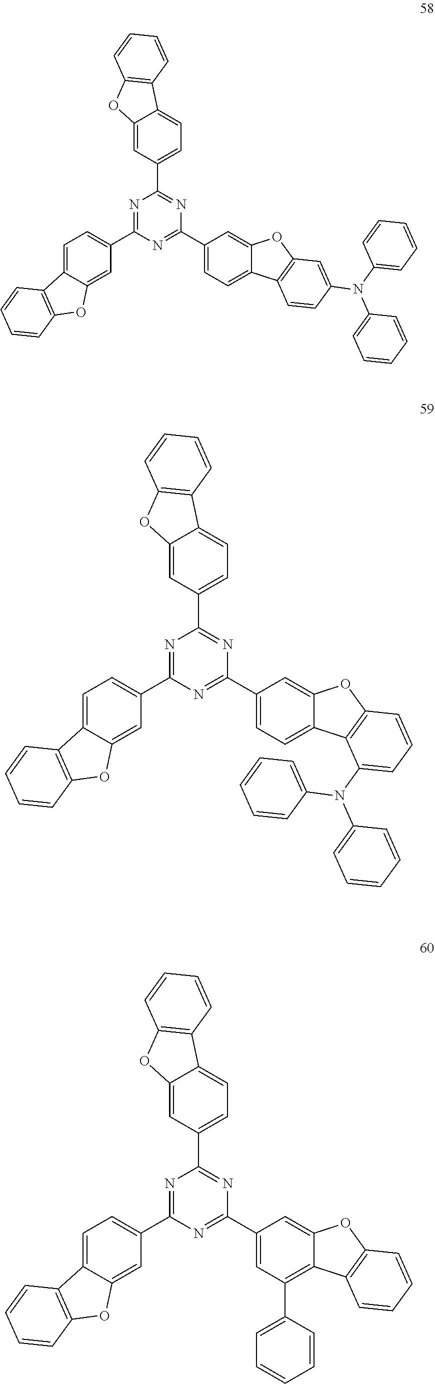 [SMILES: c1ccc(N(c2ccccc2)c2ccc3c(c2)oc2cc(-c4nc(-c5ccc6c(c5)oc5ccccc56)nc(-c5ccc6c(c5)oc5ccccc56)n4)ccc23)cc1 ; c1ccc(N(c2ccccc2)c2cccc3oc4cc(-c5nc(-c6ccc7c(c6)oc6ccccc67)nc(-c6ccc7c(c6)oc6ccccc67)n5)ccc4c23)cc1 ; c1ccc(-c2cc(-c3nc(-c4ccc5c(c4)oc4ccccc45)nc(-c4ccc5c(c4)oc4ccccc45)n3)cc3oc4ccccc4c23)cc1]

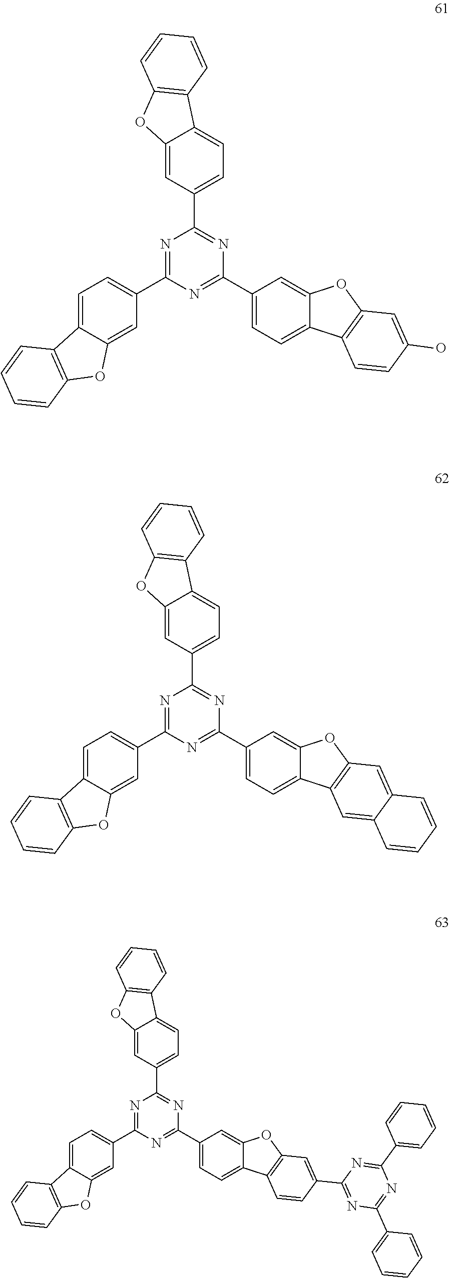 [SMILES: Oc1ccc2c(c1)oc1cc(-c3nc(-c4ccc5c(c4)oc4ccccc45)nc(-c4ccc5c(c4)oc4ccccc45)n3)ccc12 ; c1ccc2cc3c(cc2c1)oc1cc(-c2nc(-c4ccc5c(c4)oc4ccccc45)nc(-c4ccc5c(c4)oc4ccccc45)n2)ccc13 ; c1ccc(-c2nc(-c3ccccc3)nc(-c3ccc4c(c3)oc3cc(-c5nc(-c6ccc7c(c6)oc6ccccc67)nc(-c6ccc7c(c6)oc6ccccc67)n5)ccc34)n2)cc1]

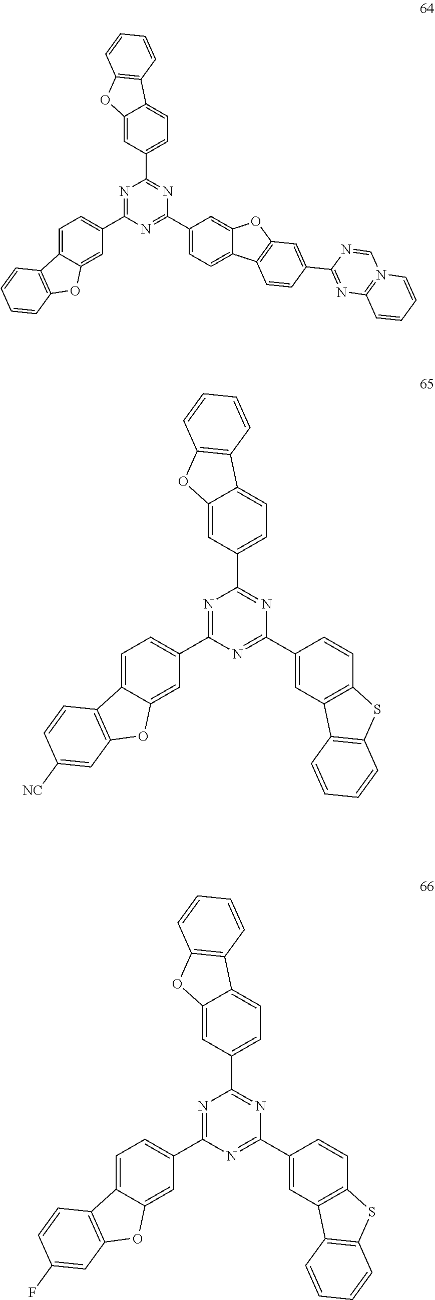

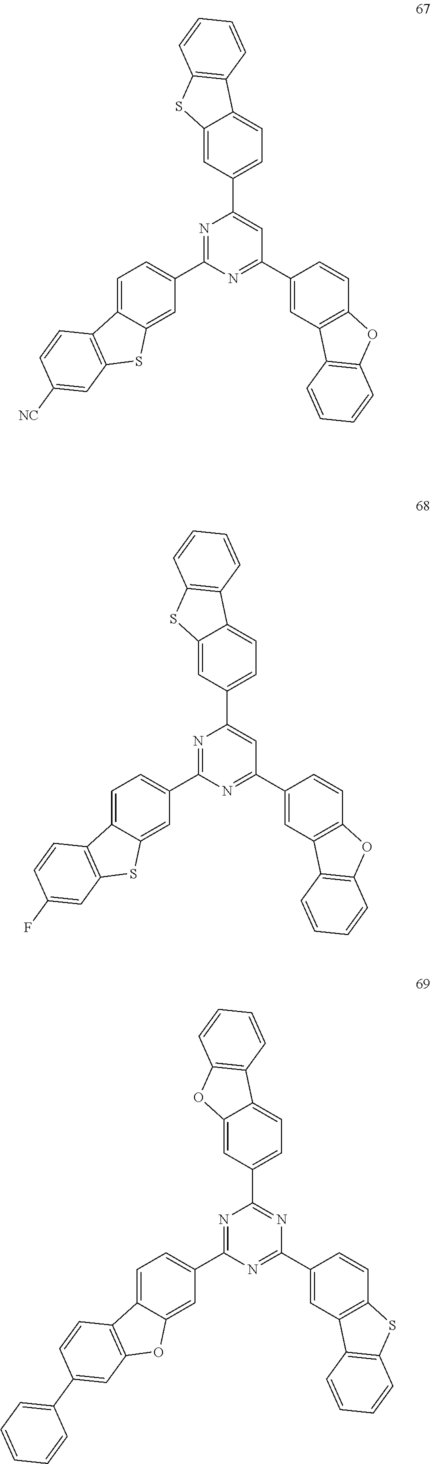 [SMILES: N#Cc1ccc2c(c1)sc1cc(-c3nc(-c4ccc5c(c4)sc4ccccc45)cc(-c4ccc5oc6ccccc6c5c4)n3)ccc12 ; Fc1ccc2c(c1)sc1cc(-c3nc(-c4ccc5c(c4)sc4ccccc45)cc(-c4ccc5oc6ccccc6c5c4)n3)ccc12 ; c1ccc(-c2ccc3c(c2)oc2cc(-c4nc(-c5ccc6c(c5)oc5ccccc56)nc(-c5ccc6sc7ccccc7c6c5)n4)ccc23)cc1]

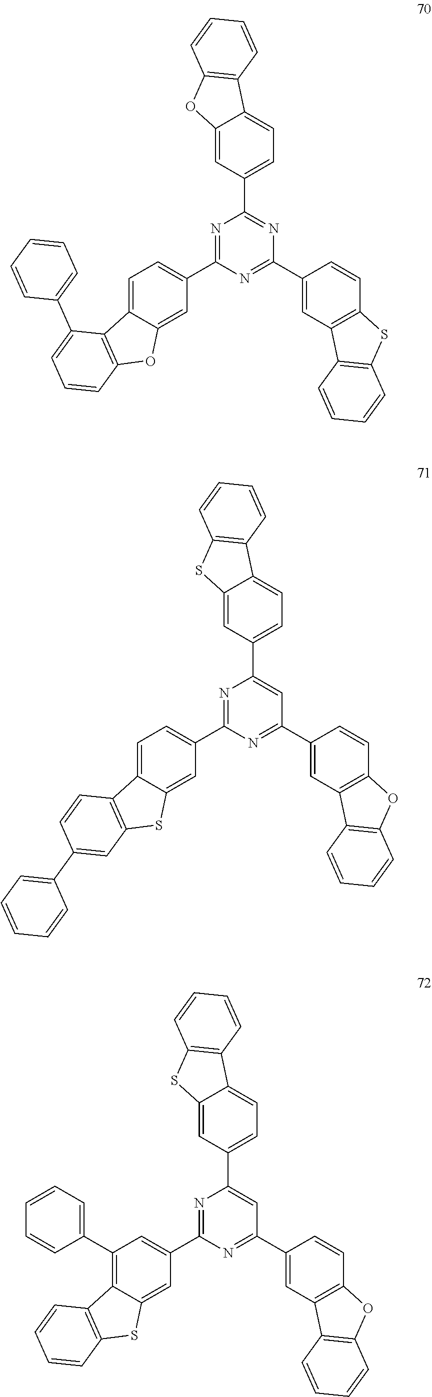 [SMILES: c1ccc(-c2cccc3oc4cc(-c5nc(-c6ccc7c(c6)oc6ccccc67)nc(-c6ccc7sc8ccccc8c7c6)n5)ccc4c23)cc1 ; c1ccc(-c2ccc3c(c2)sc2cc(-c4nc(-c5ccc6c(c5)sc5ccccc56)cc(-c5ccc6oc7ccccc7c6c5)n4)ccc23)cc1 ; c1ccc(-c2cc(-c3nc(-c4ccc5c(c4)sc4ccccc45)cc(-c4ccc5oc6ccccc6c5c4)n3)cc3sc4ccccc4c23)cc1]

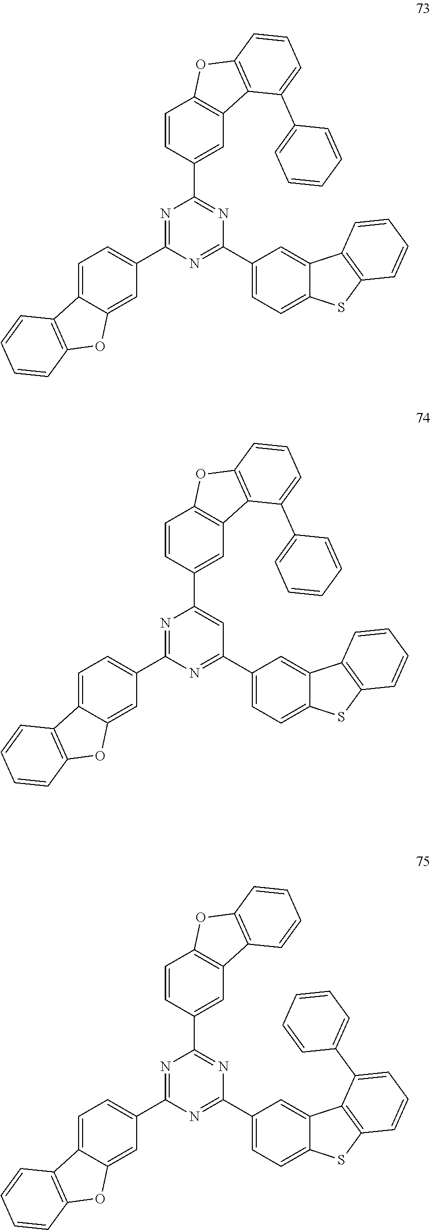 [SMILES: c1ccc(-c2cccc3oc4ccc(-c5nc(-c6ccc7c(c6)oc6ccccc67)nc(-c6ccc7sc8ccccc8c7c6)n5)cc4c23)cc1 ; c1ccc(-c2cccc3oc4ccc(-c5cc(-c6ccc7sc8ccccc8c7c6)nc(-c6ccc7c(c6)oc6ccccc67)n5)cc4c23)cc1 ; c1ccc(-c2cccc3sc4ccc(-c5nc(-c6ccc7c(c6)oc6ccccc67)nc(-c6ccc7oc8ccccc8c7c6)n5)cc4c23)cc1]

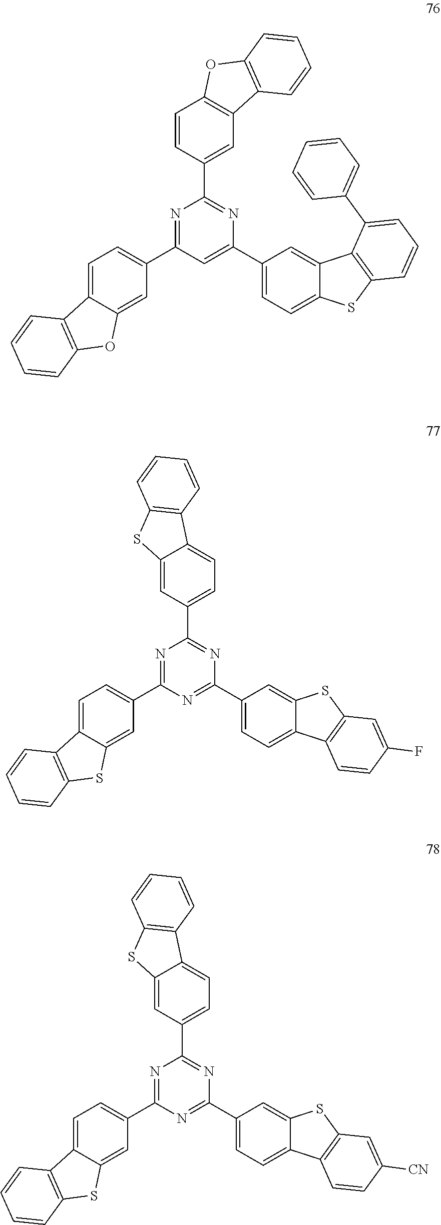 [SMILES: c1ccc(-c2cccc3sc4ccc(-c5cc(-c6ccc7c(c6)oc6ccccc67)nc(-c6ccc7oc8ccccc8c7c6)n5)cc4c23)cc1 ; Fc1ccc2c(c1)sc1cc(-c3nc(-c4ccc5c(c4)sc4ccccc45)nc(-c4ccc5c(c4)sc4ccccc45)n3)ccc12 ; N#Cc1ccc2c(c1)sc1cc(-c3nc(-c4ccc5c(c4)sc4ccccc45)nc(-c4ccc5c(c4)sc4ccccc45)n3)ccc12]

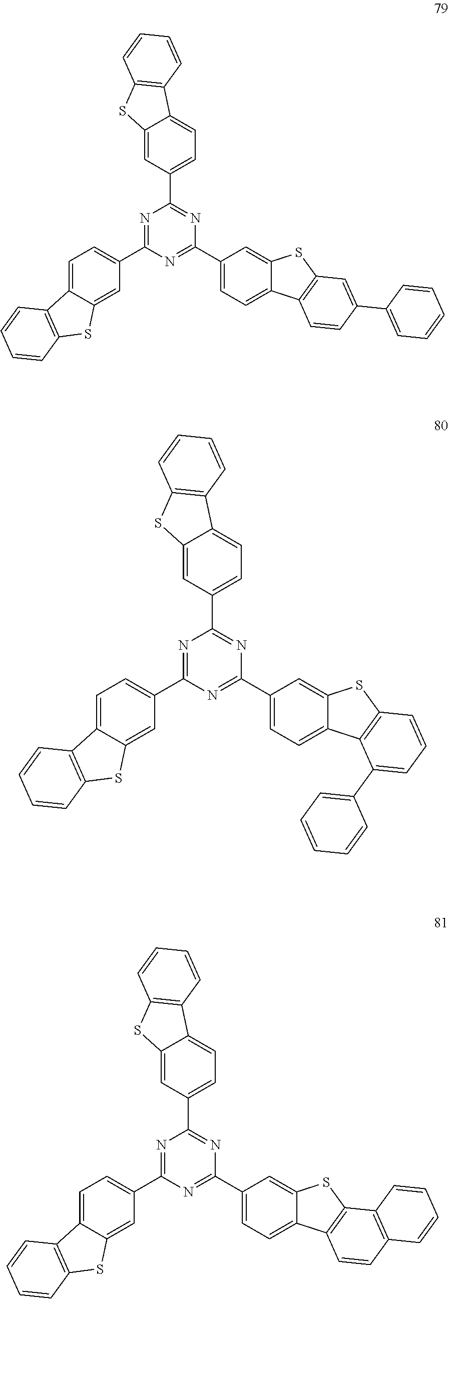

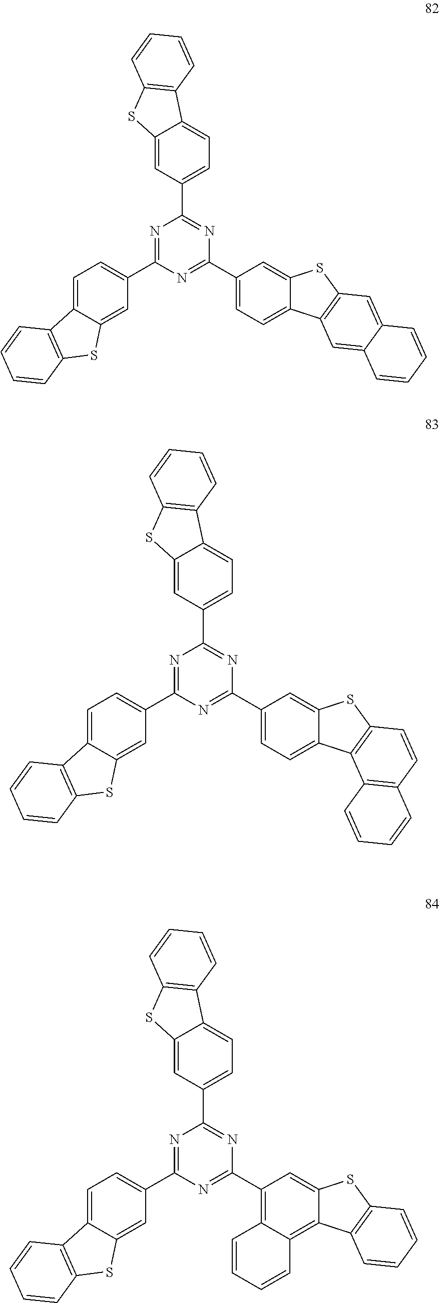 [SMILES: c1ccc2cc3c(cc2c1)sc1cc(-c2nc(-c4ccc5c(c4)sc4ccccc45)nc(-c4ccc5c(c4)sc4ccccc45)n2)ccc13 ; c1ccc2c(c1)ccc1sc3cc(-c4nc(-c5ccc6c(c5)sc5ccccc56)nc(-c5ccc6c(c5)sc5ccccc56)n4)ccc3c12 ; c1ccc2c(c1)sc1cc(-c3nc(-c4ccc5c(c4)sc4ccccc45)nc(-c4cc5sc6ccccc6c5c5ccccc45)n3)ccc12]

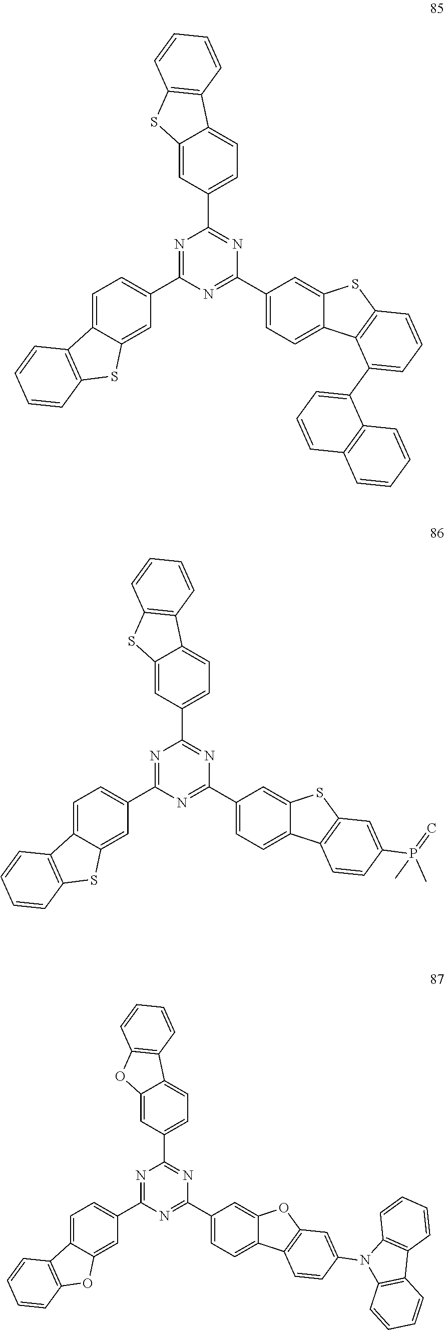 [SMILES: c1ccc2c(-c3cccc4sc5cc(-c6nc(-c7ccc8c(c7)sc7ccccc78)nc(-c7ccc8c(c7)sc7ccccc78)n6)ccc5c34)cccc2c1 ; C=P(C)(C)c1ccc2c(c1)sc1cc(-c3nc(-c4ccc5c(c4)sc4ccccc45)nc(-c4ccc5c(c4)sc4ccccc45)n3)ccc12 ; c1ccc2c(c1)oc1cc(-c3nc(-c4ccc5c(c4)oc4ccccc45)nc(-c4ccc5c(c4)oc4cc(-n6c7ccccc7c7ccccc76)ccc45)n3)ccc12]

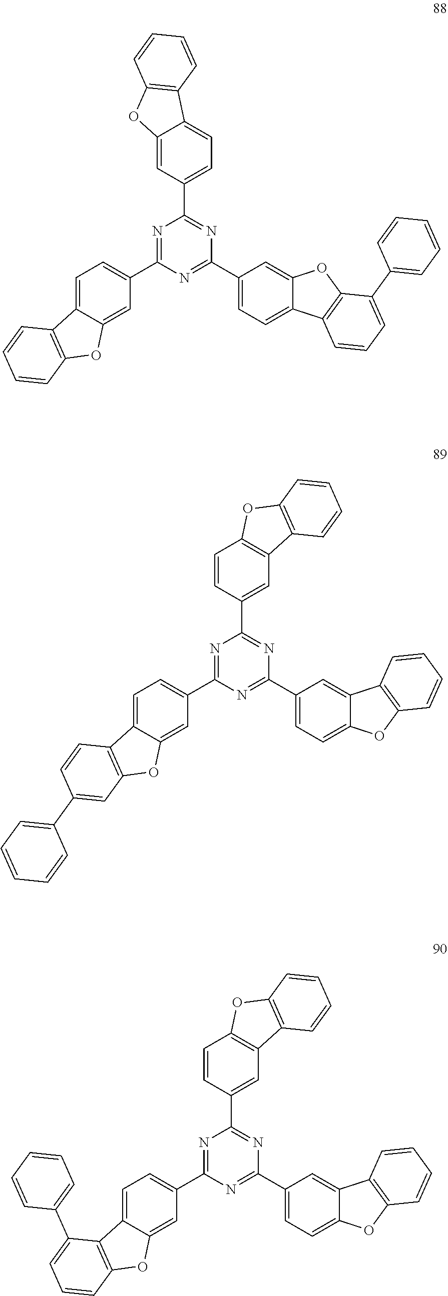 [SMILES: c1ccc(-c2cccc3c2oc2cc(-c4nc(-c5ccc6c(c5)oc5ccccc56)nc(-c5ccc6c(c5)oc5ccccc56)n4)ccc23)cc1 ; c1ccc(-c2ccc3c(c2)oc2cc(-c4nc(-c5ccc6oc7ccccc7c6c5)nc(-c5ccc6oc7ccccc7c6c5)n4)ccc23)cc1 ; c1ccc(-c2cccc3oc4cc(-c5nc(-c6ccc7oc8ccccc8c7c6)nc(-c6ccc7oc8ccccc8c7c6)n5)ccc4c23)cc1]

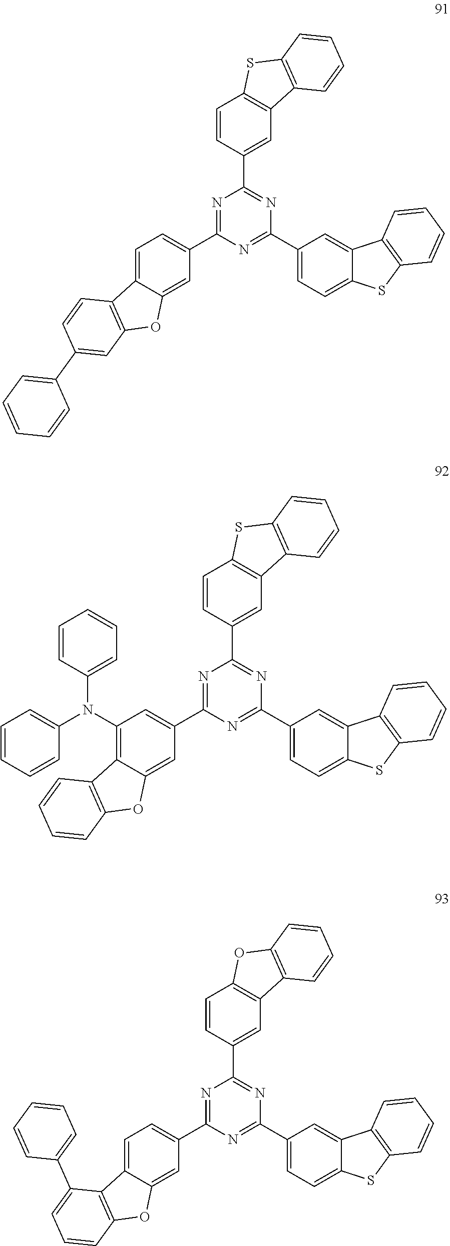 [SMILES: c1ccc(-c2ccc3c(c2)oc2cc(-c4nc(-c5ccc6sc7ccccc7c6c5)nc(-c5ccc6sc7ccccc7c6c5)n4)ccc23)cc1 ; c1ccc(N(c2ccccc2)c2cc(-c3nc(-c4ccc5sc6ccccc6c5c4)nc(-c4ccc5sc6ccccc6c5c4)n3)cc3oc4ccccc4c23)cc1 ; c1ccc(-c2cccc3oc4cc(-c5nc(-c6ccc7oc8ccccc8c7c6)nc(-c6ccc7sc8ccccc8c7c6)n5)ccc4c23)cc1]

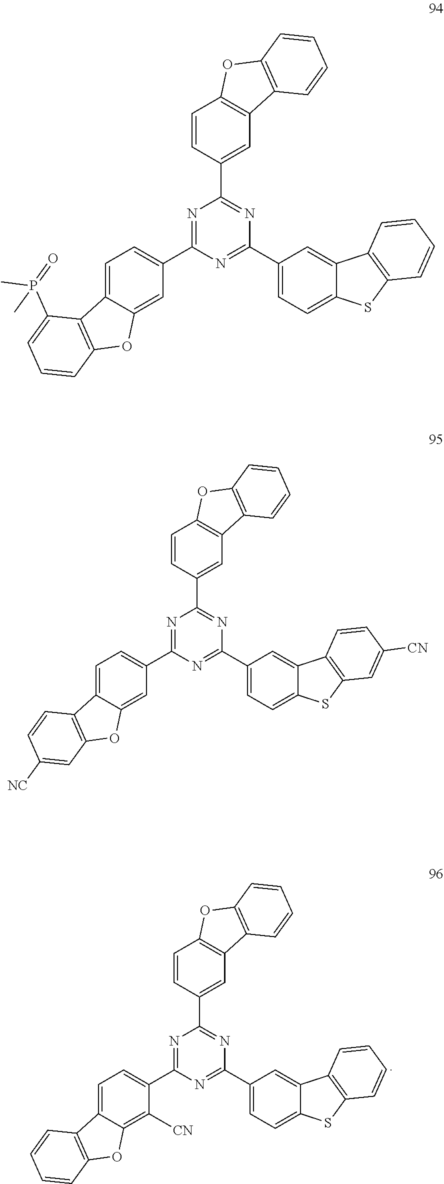 [SMILES: CP(C)(=O)c1cccc2oc3cc(-c4nc(-c5ccc6oc7ccccc7c6c5)nc(-c5ccc6sc7ccccc7c6c5)n4)ccc3c12 ; N#Cc1ccc2c(c1)oc1cc(-c3nc(-c4ccc5oc6ccccc6c5c4)nc(-c4ccc5sc6cc(C#N)ccc6c5c4)n3)ccc12 ; N#Cc1c(-c2nc(-c3ccc4oc5ccccc5c4c3)nc(-c3ccc4sc5ccccc5c4c3)n2)ccc2c1oc1ccccc12]

6. A composition, comprising:
the organic compound as claimed in claim 1 as a first organic compound, and
a second organic compound represented by Chemical Formula 7: 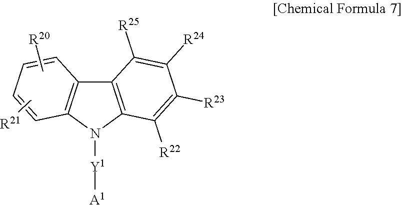
wherein, in Chemical Formula 7,
Y¹ is a single bond, a substituted or unsubstituted C6 to C30 arylene group, or a divalent substituted or unsubstituted C2 to C30 heterocyclic group,
A¹ is a substituted or unsubstituted C6 to C30 aryl group, or a substituted or unsubstituted C2 to C30 heterocyclic group,
R²⁰ to R²⁵ are independently hydrogen, deuterium, a substituted or unsubstituted C1 to C20 alkyl group, a substituted or unsubstituted C6 to C30 aryl group, or a substituted or unsubstituted C2 to C30 heterocyclic group, and
R²² to R²⁵ are separate or adjacent groups of R²² to R²⁵ are linked with each other to form a ring.

7. The composition as claimed in claim 6, wherein the second organic compound is represented by Chemical Formula 7A or a combination of Chemical Formulae 7B-1 and 7B-2: 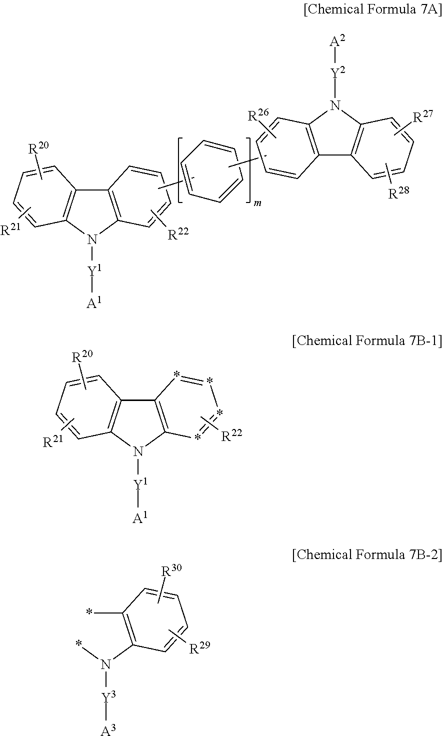
wherein, in Chemical Formula 7A, Chemical Formula 7B-1, and Chemical Formula 7B-2,
Y¹ to Y³ are independently a single bond, a substituted or unsubstituted C6 to C30 arylene group, divalent substituted or unsubstituted C2 to C30 heterocyclic group, or a combination thereof,
A¹ to A³ are independently substituted or unsubstituted C6 to C30 aryl group, a substituted or unsubstituted C2 to C30 heterocyclic group, or a combination thereof,
R²⁰ to R²² and R²⁶ to R³⁰ are independently hydrogen, deuterium, a substituted or unsubstituted C1 to C20 alkyl group, a substituted or unsubstituted C6 to C30 aryl group, a substituted or unsubstituted C2 to C30 heterocyclic group, or a combination thereof,
m is an integer of 0 to 2, and
two adjacent *'s of Chemical Formula 7B-1 are bonded with two *'s of Chemical Formula 7B-2.

8. The composition as claimed in claim 7, wherein A¹ to A³ of Chemical Formula 7A, Chemical Formula 7B-1, and Chemical Formula 7B-2 are independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, or a substituted or unsubstituted triphenylene group, a substituted or unsubstituted pyridinyl group, a substituted or unsubstituted dibenzothiophenyl group, a substituted or unsubstituted dibenzofuranyl group, a substituted or unsubstituted carbazolyl group, a substituted or unsubstituted fluorenyl group, or a combination thereof.

9. The composition as claimed in claim 7, wherein the second organic compound is represented by Chemical Formula 7A-1 or 7B-c: 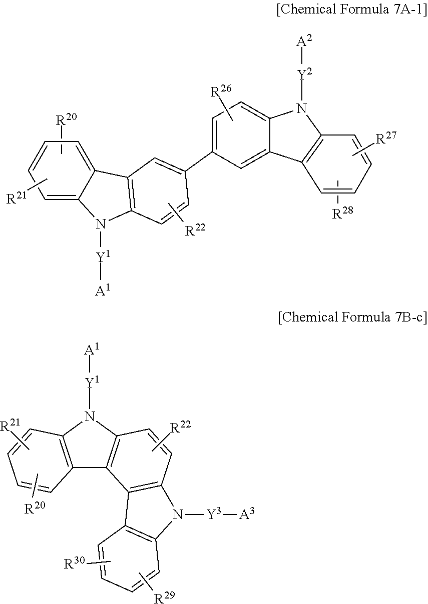
wherein, in Chemical Formula 7A-1 and Chemical Formula 7B-c,
Y¹ to Y³ are independently a single bond, a substituted or unsubstituted C6 to C30 arylene group, divalent substituted or unsubstituted C2 to C30 heterocyclic group, or a combination thereof,
A¹ to A³ are independently substituted or unsubstituted phenyl group, a substituted or unsubstituted biphenyl group, a substituted or unsubstituted terphenyl group, a substituted or unsubstituted naphthyl group, a substituted or unsubstituted anthracenyl group, or a substituted or unsubstituted triphenylene group, a substituted or unsubstituted pyridinyl group, a substituted or unsubstituted dibenzothiophenyl group, a substituted or unsubstituted dibenzofuranyl group, a substituted or unsubstituted carbazolyl group, a substituted or unsubstituted fluorenyl group, or a combination thereof, and
R²⁰ to R²² and R²⁶ to R³⁰ are independently hydrogen, deuterium, a substituted or unsubstituted C1 to C20 alkyl group, a substituted or unsubstituted C6 to C30 aryl group, a substituted or unsubstituted C2 to C30 heterocyclic group or a combination thereof.

10. An organic optoelectronic device, comprising:
an anode and a cathode facing each other, and
an organic layer between the anode and the cathode,
wherein the organic layer includes the organic compound as claimed in claim 1.

11. The organic optoelectronic device as claimed in claim 10, wherein the organic layer includes a light emitting layer, and the organic compound is a host of the light emitting layer.

12. The organic optoelectronic device as claimed in claim 10, wherein:
the organic layer includes a light emitting layer, and an electron auxiliary layer between the cathode and the light emitting layer, and
the electron auxiliary layer includes the organic compound.

13. An organic optoelectronic device, comprising:
an anode and a cathode facing each other, and
an organic layer between the anode and the cathode,
wherein the organic layer includes the composition as claimed in claim 6.

14. The organic optoelectronic device as claimed in claim 13, wherein the organic layer includes a light emitting layer, and the composition is a host of the light emitting layer.

15. The organic optoelectronic device as claimed in claim 13, wherein:
the organic layer includes a light emitting layer, and an electron auxiliary layer between the cathode and the light emitting layer, and
the electron auxiliary layer includes the composition.

16. A display device comprising the organic optoelectronic device as claimed in claim 10.

17. A display device comprising the organic optoelectronic device as claimed in claim 13.
